SEQUENCE LISTING (651 sequences)

SEQ ID NO: 1 - protein, 1070 aa, Bacillus sp.
  REGION 1..1070: note = source = /note="APG05083.1"
MRELDYRIGLDIGTNSIGWGIIELSWNKDREQYEKARIVDKGVRMFDKAEIPKTGASLAEPRRIARSSRRRLNRKSQRKKDIRNLLVQHEIISQKELASLYPLTKSSMDIWDIRLDGLDRLLDRFEWTRLLIHLAQRRGFKSNRKSELKDVETGKVLSSIQANEKRLSLYRTVGEMWMKNEDFSKYDKRRNSSNEYVFSVSRADLEKEIVTLFEAQRKFQSSYASADLQKTYLQIWAHQLPFASGNAIVNKVGYCSLLKGKEKRVPKATYTFQYFSTLDQINRTRLGPNFQPFTKEQRDVILDEMFNRTDYYKKKTIPEVTYYDIRKWLALDETIQFKGLTYDPNEELKKIELKSFINLKPFYEIKKVVTNYAKKTNEAFSTLDYDTFAYALTVYKTDKDIRSYLKKSNNLSKCCYDDQLIEELLTLSYTKFGHLSFKAINHVLPIMQEGRTYQEAIHQLGYDATNLKKENRSMFLPLFPDEITNPIVKRALTQARKVVNAIIRRYGSPNSVHIELARELSKSHDERTKIMKAHDENYKKNKGAISILIENGILNPTGYDIVRYKLWKEQGERCAYSLKQIPANTFFNEMKKERSGSPVLEIDHILPYSQSFIDSYHNKVLVYGDENQKKGNRIPYTYFLEGNKDWESFESYVRLNSFFSKKKRGYLLKKAYLPRESNMIKERHLNDTRYASSYLKNFIEKNLKFKEVEGSTRKKHVQTVNGIITAHLRKRWGLEKDRQETYLHHAMDAIIVACTDHHMVTKVTEYYQIKESNKSIRKPYFPLPWVGFREEILSHLARQPIARKISEELKIGYQSFDYILVSRMPKRSVTGAAHEQTIMKKGGIDKKGKTIIIKRVYLKDIKFDENGDFKMVGKEQDLATYEAIKQRYIEYGKESKKAFETPLYKPSKKGKGNLIKKIKVEVQTKSFVREVNGGVAQNGDLVRVDLFEKDNRYYMIPIYVMDTVHSELPNKAVTSSKGYEQWLTIDNSFTFKFSLYPYDLVRLVKGNEDRFLYFSTLDINSDRLNFKDVNKPSKQAENRYSLKTIENLEKYEVGVLGDLRFVRQEIRKNF

SEQ ID NO: 2 - RNA, 19 nt, Bacillus sp.
  misc_feature 1..19: note = source = /note="crRNA"
  source 1..19: mol_type = other RNA
gtcatagttccattattgc

SEQ ID NO: 3 - RNA, 84 nt, Bacillus sp.
  misc_feature 1..84: note = source = /note="tracrRNA"
  source 1..84: mol_type = other RNA
gctttgatgtttctatgataagggcttaggcccgtggcgttggggatcgcctgcccattttaatgggcttctccccatctattt

SEQ ID NO: 4 - RNA, 137 nt, synthetic construct
  misc_feature 1..137: note = source = /note="Description of Artificial Sequence: Syntheticpolynucleotide"
  misc_feature 1..137: note = source = /note="sgRNA L1"
  source 1..137: mol_type = other RNA
gagcggacagcagcttcctatatctcgtacgtcatagttccattattgcaaaggctttgatgtttctatgataagggcttaggcccgtggcgttggggatcgcctgcccattttaatgggcttctccccatctattt

SEQ ID NO: 5 - RNA, 137 nt, synthetic construct
  misc_feature 1..137: note = source = /note="Description of Artificial Sequence: Syntheticpolynucleotide"
  misc_feature 1..137: note = source = /note="sgRNA L2"
  source 1..137: mol_type = other RNA
ccatgatatagacgttgtggctgttgtagtgtcatagttccattattgcaaaggctttgatgtttctatgataagggcttaggcccgtggcgttggggatcgcctgcccattttaatgggcttctccccatctattt

SEQ ID NO: 6-7 - skipped (2 entries)

SEQ ID NO: 8 - DNA, 38 bp, synthetic construct
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 1"
  source 1..38: mol_type = other DNA
gagcggacagcagcttcctatatctcgtacnnnnnnnn

SEQ ID NO: 9 - DNA, 38 bp, synthetic construct
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 2"
  source 1..38: mol_type = other DNA
ccatgatatagacgttgtggctgttgtagtnnnnnnnn

SEQ ID NO: 10 - RNA, 132 nt, synthetic construct
  misc_difference 1..25: note = a,c,t,g, unknown, or other
  misc_feature 1..132: note = source = /note="Description of Artificial Sequence: Syntheticpolynucleotide"
  misc_feature 1..132: note = source = /note="sgRNA with N's"
  source 1..132: mol_type = other RNA
nnnnnnnnnnnnnnnnnnnnnnnnngtcatagttccattattgcaaaggctttgatgtttctatgataagggcttaggcccgtggcgttggggatcgcctgcccattttaatgggcttctccccatctattt

SEQ ID NO: 11 - protein, 1071 aa, Bacillus sp.
  REGION 1..1071: note = source = /note="APG07433.1"
MRELDYRIGLDIGTNSIGWGVIELSWNKDRERYEKVRIVDQGVRMFDRAEMPKTGASLAEPRRIARSSRRRLNRKSQRKKNIRNLLVQHGVITQEELDSLYPLSKKSMDIWGIRLDGLDRLLNHFEWARLLIHLAQRRGFKSNRKSELKDTETGKVLSSIQLNEKRLSLYRTVGEMWMKDPDFSKYDRKRNSPNEYVFSVSRAELEKEIVTLFAAQRRFQSPYASKDLQETYLQIWTHQLPFASGNAILNKVGYCSLLKGKERRIPKATYTFQYFSALDQVNRTRLGPDFQPFTKEQREIILNNMFQRTDYYKKKTIPEVTYYDIRKWLELDETIQFKGLNYDPNEELKKIEKKPFINLKAFYEINKVVANYSERTNETFSTLDYDGIGYALTVYKTDKDIRSYLKSSHNLPKRCYDDQLIEELLSLSYTKFGHLSLKAINHVLSIMQKGNTYKEAVDQLGYDTSGLKKEKRSKFLPPISDEITNPIVKRALTQARKVVNAIIRRHGSPHSVHIELARELSKNHDERTKIVSAQDENYKKNKGAISILSEHGILNPTGYDIVRYKLWKEQGERCAYSLKEIPADTFFNELKKERNGAPILEVDHILPYSQSFIDSYHNKVLVYSDENRKKGNRIPYTYFLETNKDWEAFERYVRSNKFFSKKKREYLLKRAYLPRESELIKERHLNDTRYASTFLKNFIEQNLQFKEAEDNPRKRRVQTVNGVITAHFRKRWGLEKDRQETYLHHAMDAIIVACTDHHMVTRVTEYYQIKESNKSVKKPYFPMPWEGFRDELLSHLASQPIAKKISEELKAGYQSLDYIFVSRMPKRSITGAAHKQTIMRKGGIDKKGKTIIIERLHLKDIKFDENGDFKMVGKEQDMATYEAIKQRYLEHGKNSKKAFETPLYKPSKKGTGNLIKRVKVEGQAKSFVREVNGGVAQNGDLVRVDLFEKDDKYYMVPIYVPDTVCSELPKKVVASSKGYEQWLTLDNSFTFKFSLYPYDLVRLVKGDEDRFLYFGTLDIDSDRLNFKDVNKPSKKNEYRYSLKTIEDLEKYEVGVLGDLRLVRKETRRNFH

SEQ ID NO: 12 - RNA, 21 nt, Bacillus sp.
  misc_feature 1..21: note = source = /note="crRNA"
  source 1..21: mol_type = other RNA
gtcatagttccattaaagcca

SEQ ID NO: 13 - RNA, 85 nt, Bacillus sp.
  misc_feature 1..85: note = source = /note="tracrRNA"
  source 1..85: mol_type = other RNA
tggctttgatgtttctatgataagggtttcgacccgtggcgtcggggatcgcctgcccattgaaatgggcttctccccatttatt

SEQ ID NO: 14 - RNA, 140 nt, synthetic construct
  misc_feature 1..140: note = source = /note="Description of Artificial Sequence: Syntheticpolynucleotide"
  misc_feature 1..140: note = source = /note="sgRNA L1"
  source 1..140: mol_type = other RNA
gagcggacagcagcttcctatatctcgtacgtcatagttccattaaagccagaaatggctttgatgtttctatgataagggtttcgacccgtggcgtcggggatcgcctgcccattgaaatgggcttctccccatttatt

SEQ ID NO: 15 - RNA, 140 nt, synthetic construct
  misc_feature 1..140: note = source = /note="Description of Artificial Sequence: Syntheticpolynucleotide"
  misc_feature 1..140: note = source = /note="sgRNA L2"
  source 1..140: mol_type = other RNA
ccatgatatagacgttgtggctgttgtagtgtcatagttccattaaagccagaaatggctttgatgtttctatgataagggtttcgacccgtggcgtcggggatcgcctgcccattgaaatgggcttctccccatttatt

SEQ ID NO: 16 - DNA, 38 bp, synthetic construct; identical to SEQ ID NO: 8
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 1"
  source 1..38: mol_type = other DNA

SEQ ID NO: 17 - DNA, 38 bp, synthetic construct; identical to SEQ ID NO: 9
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 2"
  source 1..38: mol_type = other DNA

SEQ ID NO: 18 - RNA, 135 nt, synthetic construct
  misc_difference 1..25: note = a,c,t,g, unknown, or other
  misc_feature 1..135: note = source = /note="Description of Artificial Sequence: Syntheticpolynucleotide"
  misc_feature 1..135: note = source = /note="sgRNA with N's"
  source 1..135: mol_type = other RNA
nnnnnnnnnnnnnnnnnnnnnnnnngtcatagttccattaaagccaaaagtggctttgatgtttctatgataagggtttcgacccgtggcgtcggggatcgcctgcccattgaaatgggcttctccccatttatt

SEQ ID NO: 19 - protein, 1071 aa, Bacillus sp.
  REGION 1..1071: note = source = /note="APG07513.1"
MRELDYRIGLDIGTNSIGWGVIELSWNKDREQYEKTRIVDKGVRMFDKAEIPKTGASLAEPRRIARSSRRRLNRKSQRKKDIRNLLVQHEIISQKELTSLYPLSKSSMDIWDIRLDGLDRLLDRFEWARLLIHLAQRRGFKSNRKSELKDVETGKVLSSIQVNEKRLSLYRTVGEMWMKNADCSKYGKRRNSPNEYVFSVSRADLEKEIVTLFEAQRKFHSSYASVDLQKTYIQIWAHQLPFASGNAIVNKVGYCSLLKGKEKRVPKATYTFQYFNTLDQINRTRLGPNFQPFTKEQRDIILDKMFQRTDYYKKKTIPEVTYYDIRKWLALDETIQFKGLTYDPNEELKKIEMKPFINLKPFYEIKKVVTNYAKKTNEVFSALDYDTVAYALTVYKTDKDIRSYLKRSNNLSKRCYDDQLIEELLTLSYTKFGHLSFKAINHVLPIMQEGRTYQEAIHQLGYDTTNLKKENRSMFLPIIPDEITNPIVKRALTQARKVVNAIIRRYGSPNSVHIELARELSKSHDERKKIMTAHDENYKKNKGAVSILIDNGILNPTGYDIVRYKLWKEQGERCAYSLKKIPANTFFNELKKERSGPPVLEVDHILPYSQSFIDSYHNKVLVYGDENQKKGNRIPYTFFSEEDKEWESFESYVRSNSFFSKKKRGYLLKKAYLPRESNLIKERHLNDTRYASSYLKNFIEKNLKFKEAVGITRKKYVQTVNGVITAHLRKRWGLEKDRQETYLHHAMDAIIVACTDHHMVTKVTEYYQIKEGNKSIKKPYFPLPWMGFREEILSHLESQPIARKISEELKIGYQSPDYILVSRMPKRSVTGSAHDQTVMKKGDIDKKGKTIIIKRVHLKDIKFDENGDFKMVGKEQDLATYEAIKQRYLEYRKESKKAFETPLYKPSKKGKGNLIKKIKVEVQTKSFVREINGGVAQNGDLVRVDLFEKDNRYYMVPIYVVDTVRSELPNKAVTSSKGYEQWLSIDNSFTFKFSLYPYDLVRLVKGDEDRFLYFSTLDINSDRLNFKDVNKPSKQAEYRYSLKTIENLEKYEIGVLGDLRLVRQETRKIFK

SEQ ID NO: 20 - RNA, 27 nt, Bacillus sp.
  misc_feature 1..27: note = source = /note="crRNA"
  source 1..27: mol_type = other RNA
gtcatagttccattaaagccattgctg

SEQ ID NO: 21 - RNA, 93 nt, Bacillus sp.
  misc_feature 1..93: note = source = /note="tracrRNA"
  source 1..93: mol_type = other RNA
acagcaatggctttgatgtttctatgataagggcttcggcccgtggcgttggggatcgcctgcccattttaatgggcttctccccatctattt

SEQ ID NO: 22 - RNA, 154 nt, synthetic construct
  misc_feature 1..154: note = source = /note="Description of Artificial Sequence: Syntheticpolynucleotide"
  misc_feature 1..154: note = source = /note="sgRNA L1"
  source 1..154: mol_type = other RNA
gagcggacagcagcttcctatatctcgtacgtcatagttccattaaagccattgctgaaagacagcaatggctttgatgtttctatgataagggcttcggcccgtggcgttggggatcgcctgcccattttaatgggcttctccccatctattt

SEQ ID NO: 23 - RNA, 154 nt, synthetic construct
  misc_feature 1..154: note = source = /note="Description of Artificial Sequence: Syntheticpolynucleotide"
  misc_feature 1..154: note = source = /note="sgRNA L2"
  source 1..154: mol_type = other RNA
ccatgatatagacgttgtggctgttgtagtgtcatagttccattaaagccattgctgaaagacagcaatggctttgatgtttctatgataagggcttcggcccgtggcgttggggatcgcctgcccattttaatgggcttctccccatctattt

SEQ ID NO: 24 - DNA, 38 bp, synthetic construct; identical to SEQ ID NO: 8
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 1"
  source 1..38: mol_type = other DNA

SEQ ID NO: 25 - DNA, 38 bp, synthetic construct; identical to SEQ ID NO: 9
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 2"
  source 1..38: mol_type = other DNA

SEQ ID NO: 26 - RNA, 149 nt, synthetic construct
  misc_difference 1..25: note = a,c,t,g, unknown, or other
  misc_feature 1..149: note = source = /note="Description of Artificial Sequence: Syntheticpolynucleotide"
  misc_feature 1..149: note = source = /note="sgRNA with N's"
  source 1..149: mol_type = other RNA
nnnnnnnnnnnnnnnnnnnnnnnnngtcatagttccattaaagccattgctgaaagacagcaatggctttgatgtttctatgataagggcttcggcccgtggcgttggggatcgcctgcccattttaatgggcttctccccatctattt

SEQ ID NO: 27 - protein, 1072 aa, Bacillus sp.
  REGION 1..1072: note = source = /note="APG08290.1"
MSELDYRIGLDIGTNSIGWGVIELFWNKDRERYEKVRIVDKGVRMFDKAEIPNKGASLAEPRRIARSSRRRLNRKSQRKKEIRNLLVQHGMITQEELDLLYPLSKKSIDIWDIRLDGLDRLLNHLEWARLLIHLAQRRGFKSNRKSELKDAETGKVLSSIQVNEKRLFLYRTVGEMWIKDAEFSKYDRRRNSPNEYVFSVSRADLEKEIVTLFEAQRKFQSSYASKNLQETYLQIWAHQLPFASGNAILNKVGYCSLLKGKERRIPKATYTFQYFSALDQVNRTRLGPDFQPFTQEQKEIILDKMFQRTDYYKKKTIPEVSYYDIRKWLELDETIQFKGLNYDPNEELKKIEKKPFINLKAFYEIKKVVANYAERTNEAFSTLDYDAIAYALTVYKTDKDIRSYLKKSNNLSKRCYDDQLIEELFTLSYTKFGHLSFKAINHVLPIMQEGRTYQEAIHQLGYDTTNLKKENRSMFLPLIPDEITNPIVKRAITQARKVVNAIIRRYGSPNSVHIELARELSKSHDERKKIMTAHDENYKKNKGAISILIENGILNPTGYDIVRYKLWKEQGERCAYSLKEIPPDTFFNELKKERNGSPILEVDHILPYSQSFIDSYHNKVLVYSDENRNKGNRIPYTYFLETNKDWEAFERYVRSNKLFSKKKREYLLKKTYLPRESELIKERHLNDTRYASTFLKNFIEQNLQFKEVEVNLRKKRVQTVNGVITAHLRKRWGLEKNRQETYLHHAMDAIIVACTDHHMVTRITEYYQIKESNKSVKKPYFPMPWEGFRDELLSHLASQPIAKKISEELKAGYQSSDYIFVSRMPKRSVTGAAHDQTIRRKGGIDKKGKTIIIKRVRLKDIKFDENGDFKMVGKEQDLATYEAIKQRYLEHRKNSKKAFETPLYKPSKKGTGNLIKRVKIEGQTKAFVREVNGGVAQNSDLVRVDLFEKDDKYYMVPIYVPDTVCSELPKKVVKSGKGYEQWLTLDNSFTFKSSLYPYDLVRLVKGNEDRFLYFGTLDIDSDRLNFKDVNKPSKQNEYRYSLKTIENLEKYEVGVLGDLRLVKQETRRIFNR

SEQ ID NO: 28 - RNA, 21 nt, Bacillus sp.
  misc_feature 1..21: note = source = /note="crRNA"
  source 1..21: mol_type = other RNA
gtcatagttccatgaaagcca

SEQ ID NO: 29 - RNA, 85 nt, Bacillus sp.
  misc_feature 1..85: note = source = /note="tracrRNA"
  source 1..85: mol_type = other RNA
tggctttgatgtttctatgataagggtttcggcccgtggcgtcggggatcgcctgcccattccgatgggcttctccccatttatt

SEQ ID NO: 30 - RNA, 140 nt, synthetic construct
  misc_feature 1..140: note = source = /note="Description of Artificial Sequence: Syntheticpolynucleotide"
  misc_feature 1..140: note = source = /note="sgRNA L1"
  source 1..140: mol_type = other RNA
gagcggacagcagcttcctatatctcgtacgtcatagttccatgaaagccaaaagtggctttgatgtttctatgataagggtttcggcccgtggcgtcggggatcgcctgcccattccgatgggcttctccccatttatt

SEQ ID NO: 31 - RNA, 140 nt, synthetic construct
  misc_feature 1..140: note = source = /note="Description of Artificial Sequence: Syntheticpolynucleotide"
  misc_feature 1..140: note = source = /note="sgRNA L2"
  source 1..140: mol_type = other RNA
ccatgatatagacgttgtggctgttgtagtgtcatagttccatgaaagccaaaagtggctttgatgtttctatgataagggtttcggcccgtggcgtcggggatcgcctgcccattccgatgggcttctccccatttatt

SEQ ID NO: 32 - skipped

SEQ ID NO: 33 - DNA, 38 bp, synthetic construct; identical to SEQ ID NO: 8
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 1"
  source 1..38: mol_type = other DNA

SEQ ID NO: 34 - DNA, 38 bp, synthetic construct; identical to SEQ ID NO: 9
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 2"
  source 1..38: mol_type = other DNA

SEQ ID NO: 35 - RNA, 135 nt, synthetic construct
  misc_difference 1..25: note = a,c,t,g, unknown, or other
  misc_feature 1..135: note = source = /note="Description of Artificial Sequence: Syntheticpolynucleotide"
  misc_feature 1..135: note = source = /note="sgRNA with N's"
  source 1..135: mol_type = other RNA
nnnnnnnnnnnnnnnnnnnnnnnnngtcatagttccatgaaagccaaaagtggctttgatgtttctatgataagggtttcggcccgtggcgtcggggatcgcctgcccattccgatgggcttctccccatttatt

SEQ ID NO: 36 - protein, 1337 aa, Enterococcus sp.
  REGION 1..1337: note = source = /note="APG05459.1"
MKKDYVIGLDIGTNSVGWAVMTEDYQLVKKKMPIYGNTEKKKIKKNFWGVRLFEEGHTAEDRRLKRTARRRISRRRNRLRYLQAFFEEAMTALDENFFARLQESFLVPEDKKWHRHPIFAKLEDEVAYHETYPTIYHLRKKLADSSEQADLRLIYLALAHIVKYRGHFLIEGKLSTENISVKEQFQQFMIIYNQTFVNGESRLVSAPLPESVLIEEELTEKASRTKKSEKVLQQFPQEKANGLFGQFLKLMVGNKADFKKVFGLEEEAKITYASESYEEDLEGILAKVGDEYSDVFLAAKNVYDAVELSTILADSDKKSHAKLSSSMIVRFTEHQEDLKKFKRFIRENCPDEYDNLFKNEQKDGYAGYIAHAGKVSQLKFYQYVKKIIQDIAGAEYFLEKIAQENFLRKQRTFDNGVIPHQIHLAELQAIIHRQAAYYPFLKENQEKIEQLVTFRIPYYVGPLSKGDASTFAWLKRQSEEPIRPWNLQETVDLDQSATAFIERMTNFDTYLPSEKVLPKHSLLYEKFMVFNELTKISYTDDRGIKANFSGKEKEKIFDYLFKTRRKVKKKDIIQFYRNEYNTEIVTLSGLEEDQFNASFSTYQDLLKCGLTRAELDHPDNAEKLEDIIKILTIFEDRQRIRTQLSTFKGQFSAEVLKKLERKHYTGWGRLSKKLINGIYDKESGKTILDYLIKDDGVSKHYNRNFMQLINDSQLSFKNAIQKAQSSEHEETLSETVNELAGSPAIKKGIYQSLKIVDELVAIMGYAPKRIVVEMARENQTTSTGKRRSIQRLKIVEKAMAEIGSNLLKEQPTTNEQLRDTRLFLYYMQNGKDMYTGDELSLHRLSHYDIDHIIPQSFMKDDSLDNLVLVGSTENRGKSDDVPSKEVVKDMKAYWEKLYAAGLISQRKFQRLTKGEQGGLTLEDKAHFIQRQLVETRQITKNVAGILDQRYNANSKEKKVQIITLKASLTSQFRSIFGLYKVREVNDYHHGQDAYLNCVVATTLLKVYPNLAPEFVYGEYPKFQAFKENKATAKTIIYTNLMRFFTEDEPRFMKDGEILWSNSYLKNIKKELNYHQMNIVKKVEVQKGGFSKESIKPKGPSNKLIPVKNGLDPQKYGGFDSPVVAYTVLFTHEKGKKPLIKQEILGITIMEKTRFEQNPILFLEEKGFLRPRVLMKLPKYTLYEFPEGRRRLLASAKEAQKGNQMVLPEHLLTLLYHAKQCLLPNQSESLAYVEQHQPEFQEILERVVDFAEVHTLAKSKVQQIVKLFEANQTADVKEIAASFIQLMQFNAMGAPSTFKFFQKDIERARYTSIKEIFDATIIYQSTTGLYETRRKVVD

SEQ ID NO: 37 - RNA, 16 nt, Enterococcus sp.
  misc_feature 1..16: note = source = /note="crRNA"
  source 1..16: mol_type = other RNA
gttttagagtcatgtt

SEQ ID NO: 38 - RNA, 74 nt, Enterococcus sp.
  misc_feature 1..74: note = source = /note="tracrRNA"
  source 1..74: mol_type = other RNA
aacatagcaagttaaaataaggttttaaccgtaatcaactgtaaagtggcgctgtttcggcgctttttttgttt

SEQ ID NO: 39 - RNA, 124 nt, synthetic construct
  misc_feature 1..124: note = source = /note="Description of Artificial Sequence: Syntheticpolynucleotide"
  misc_feature 1..124: note = source = /note="sgRNA L1"
  source 1..124: mol_type = other RNA
gagcggacagcagcttcctatatctcgtacgttttagagtcatgttaaagaacatagcaagttaaaataaggttttaaccgtaatcaactgtaaagtggcgctgtttcggcgctttttttgttt

SEQ ID NO: 40 - RNA, 124 nt, synthetic construct
  misc_feature 1..124: note = source = /note="Description of Artificial Sequence: Syntheticpolynucleotide"
  misc_feature 1..124: note = source = /note="sgRNA L2"
  source 1..124: mol_type = other RNA
ccatgatatagacgttgtggctgttgtagtgttttagagtcatgttaaagaacatagcaagttaaaataaggttttaaccgtaatcaactgtaaagtggcgctgtttcggcgctttttttgttt

SEQ ID NO: 41 - skipped

SEQ ID NO: 42 - DNA, 38 bp, synthetic construct; identical to SEQ ID NO: 8
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 1"
  source 1..38: mol_type = other DNA

SEQ ID NO: 43 - DNA, 38 bp, synthetic construct; identical to SEQ ID NO: 9
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 2"
  source 1..38: mol_type = other DNA

SEQ ID NO: 44 - RNA, 119 nt, synthetic construct
  misc_difference 1..25: note = a,c,t,g, unknown, or other
  misc_feature 1..119: note = source = /note="Description of Artificial Sequence: Syntheticpolynucleotide"
  misc_feature 1..119: note = source = /note="sgRNA with N's"
  source 1..119: mol_type = other RNA
nnnnnnnnnnnnnnnnnnnnnnnnngttttagagtcatgttaaagaacatagcaagttaaaataaggttttaaccgtaatcaactgtaaagtggcgctgtttcggcgctttttttgttt

SEQ ID NO: 45 - protein, 1436 aa, Enterococcus sp.
  REGION 1..1436: note = source = /note="APG04583.1"
MAKNILGLDLGTNSIGWALVQQDFENKEGNILGMGSRIIPMSQDILGEFGKGNSISQTAERTGYRGVRRLRERHLLRRERLHRVLHLLGFLPKHYDEKIDFTQRFGKFINQAEPKLAFDSEFLFKDSFHEMLADFKQNQPEFLKDKNGEDCLVPYDWTIYYLRKKALTQKIEKYELAWLILNFNQKRGYYQLRGEEEKENPNKLVGFHSLKIVDVIPDAETNKKGETWYSLHLENGWVYRRSSKISLADWKDKVRDFIVTTDLNDDGSEKLDKDGIVKRSFRAPSADDWTLLKKKTEKDIDNSNKTVGTYIYDNLLLNPKQKIKGKMVRTIERKFYKQELEQILKTQKEFHSELQSENLLQDCVRELYRNNEQHQQMLEAKDFVHLFLNDIIFYQRPLRSQKSSISNCTLEFRKSKNENGEEVIHRLKVIAKSNPYYQEFRLLQWVQNLAIYTKDDDKNVTNEFLKSTQDWEDLLRWLHSKKEIKQDALIKFLIEKKGLKGKALTIEVAKYRWNYVQDKDYPGNETRYLIQSRLDKVEYAPKDFLTYENEMALWHIIYSINDKIEYEKALKSFANKKGLDEVTFVEAFKKFPPFKSDYGSFSEKAIKKLLPLMRFGTQWNWDNIDQNSKERIGKILTGEYDENIKGRVREKARHLNSETDFQALPLWLAQYVVYGRHSEADIAGKWNSVDDLKQFLDDFKQHSLRNPIVEQVITETLRAVKDIWNFYGKGAKDFFSEIHIELGREMKNTADERKRITTMVTDNENTNLRIKALLAEMALDQNVDNVRPYSPMQQEILKIYEEGVLNAEENIDDDILKISKTAQPSATDLKRYKLWLEQKYRSPYTGQMIPLNKLFTPEYEIEHIIPQSRYFDDSMSNKVICEAAVNKLKDNQIGLVFIKNHHGEVVDFGMGKQVKILEVSDYEDFVKQNYNKNRGKRNKLLLEDIPEKMIERQLNDTRYISKYITQVLSNIVRDDKEGSKDDGVNSKNIVPGNGKITTRLKQDWGLNDVWNDLVLPRFERMNTLTNSNDFTSKNTHGKTIPTVPIELSKGFSKKRIDHRHHAMDALVIACATRDHVNLLNNESSKSDTKRYDLNRKLRKYEKVAYNDPKTGERIEKEVPKDFIKPWETFTEDTRTLLENIVISFKQNLRVINKATNYYEKIENGKKVKVEQKGINWAVRKALHKETVSGQVHLDRIKVAKGKILTATRKTLDASFNEKTIESITDTGIQKILLNYLKSKDNNPEVAFSPEGIEELNKNIRLYNDGKAHQPILKVRVFEQGSKFTLGETGNKTTKFVEAAKGTNLFFGIYEDKSGKRSYETIPLNIVIERQKQGLQAVPETNEKGKQLLFTLSPNDLVYVPEEGVFDENNIKVDRIYKVVSFSTYQCFFVRNDVSTSVVNKVEYSALNKMEKSIDNIMIKENCVKLNVDRLGKISKA

SEQ ID NO: 46 - RNA, 16 nt, Enterococcus sp.
  misc_feature 1..16: note = source = /note="crRNA"
  source 1..16: mol_type = other RNA
gttgtgagttcctttc

SEQ ID NO: 47 - RNA, 78 nt, Enterococcus sp.
  misc_feature 1..78: note = source = /note="tracrRNA"
  source 1..78: mol_type = other RNA
gaaaggaactcacaataaggattattccgttgtgaaaacatttagcgcctcgactatcttcggggcttttttattttt

SEQ ID NO: 48 - RNA, 128 nt, synthetic construct
  misc_feature 1..128: note = source = /note="Description of Artificial Sequence: Syntheticpolynucleotide"
  misc_feature 1..128: note = source = /note="sgRNA L1"
  source 1..128: mol_type = other RNA
gagcggacagcagcttcctatatctcgtacgttgtgagttcctttcacttgaaaggaactcacaataaggattattccgttgtgaaaacatttagcgcctcgactatcttcggggcttttttattttt

SEQ ID NO: 49 - RNA, 128 nt, synthetic construct
  misc_feature 1..128: note = source = /note="Description of Artificial Sequence: Syntheticpolynucleotide"
  misc_feature 1..128: note = source = /note="sgRNA L2"
  source 1..128: mol_type = other RNA
ccatgatatagacgttgtggctgttgtagtgttgtgagttcctttcacttgaaaggaactcacaataaggattattccgttgtgaaaacatttagcgcctcgactatcttcggggcttttttattttt

SEQ ID NO: 50 - skipped

SEQ ID NO: 51 - DNA, 38 bp, synthetic construct; identical to SEQ ID NO: 8
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 1"
  source 1..38: mol_type = other DNA

SEQ ID NO: 52 - DNA, 38 bp, synthetic construct; identical to SEQ ID NO: 9
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 2"
  source 1..38: mol_type = other DNA

SEQ ID NO: 53 - RNA, 123 nt, synthetic construct
  misc_difference 1..25: note = a,c,t,g, unknown, or other
  misc_feature 1..123: note = source = /note="Description of Artificial Sequence: Syntheticpolynucleotide"
  misc_feature 1..123: note = source = /note="sgRNA with N's"
  source 1..123: mol_type = other RNA
nnnnnnnnnnnnnnnnnnnnnnnnngttgtgagttcctttcaaaggaaaggaactcacaataaggattattccgttgtgaaaacatttagcgcctcgactatcttcggggcttttttattttt

SEQ ID NO: 54 - protein, 1456 aa, Empedobacter sp.
  REGION 1..1456: note = source = /note="APG01688.1"
MMIKNILGLDLGTNSIGWALIKQDFENKHGEILGMGSRIIPMSQDILGDFGKGNSISQTADRTKYRSVRRLRERFLLRRERLHRVLHLLNFLPQHYASQIDFEKKFGKFKSETEPKLAWENWGGKFSFLFQNSFNEMLEDFKAAGQGLKIPYDWTIYYLRKKALSQKIEKEELAWILLNFNQKRGYYQLRGEEEEENPNKLVEFYSLKIVDVVADEPQKGKSDIWYSLILENGWVYRRASKIPLFDWKDKTRDFIVTTDLNDDRSVKTDKEGNEKRSFRAPSENDWTLVKKKTEQEIDQSHKTVGTYIYETLLLNPKQKIKGKLVRTIERKFYKDELKQILEKQKEFHQELKNDDLYNDCIRELYRNNEAHQLTLSKKDFVHLLMDDLIFYQRPLRSQKSSISNCTLEFRKYKDENGIEHTQYLKAIPKSNPYYQEFRLWQWMYNLNIYRKDDEANVTKEFLNTNKDFESLFEFLNNRKEIEQKPLIKFLLEQKDINKKLLNAEAEKYRWNYVEDKKYPCNETKTMISSRLDKVENISDDFLTRDIEQKIWHIIYSVNDKIEYEKALKSFATRNDLDENSFIEAFKKFSPFKSEYGSFSEKAIKKLLPLMRLGKYWYEDEIVKHSDIYFKNIENLLGDFSNRDKKISEEDKEKWNKSINLKLQEELKDFQTAEIDLFQGLRLHIAQYLVYGRHSEASMIGKWNSAEDLEEFLKDFKQHSLRNPIVEQVITETLRVVKDIWLKYGNGAKDFFNEIHIELGREMKLPADDRKKLTNQISENENTNFRIKALLAEMMNDSSVENVRPFSPMQQEILKIYEDDVLKSDIEIEDDILKISKTAQPSPSDLKRYKLWLEQKYKSPYTGQIIPLNKLFTPEYEIEHIIPQSRYFDDSFSNKVICESAVNKLKDNYIGLEFIKQFGGTIIELGFGKSIKVFETKEYEDFVKKHYANNQGKRNKLLMEDIPEKMIERQMNDTRYISKYISGVLSNIVRVEDGSDEGVNSKNIVPGNGKITTQLKQDWGLNDVWNDLILPRFERMNQLTNSKVFTAWNENYQKFLPTVPIEYSKGFSKKRIDHRHHALDALVIACATKDHVNLLNNQSAKSDTKRYDLKKKSMKFEKVVYNDAKTGEKIEREVPKQFLKPWENFTLDVKHNLETIIVSFKQNLRVINKATNYYEKYVEKDGTKNKERVEQTGTNWAIRKPMHKDTVSGKVDLPWVKVPKGKILTATRKSLDSSFDLKSIGSITDTGIQKILKNYLAFKDGNPELAFSPEGIDDLNKNIEKYNDGKPHQPINKVRVFELGSKFQVGQSGNKKDKYVEAAKGTNLFFAVYEDEKGKRNYETIPLNEVIERQKQGLSVVDLKGTNDFYLCPNDFVYIPSGDELENINNVDFKDIKKEINERIYKVVSFTGNRLSCIPYMVATTIVNKLEFTQLNKIEFTKEKEICIKLNVDRLGNISKA

SEQ ID NO: 55 - RNA, 18 nt, Empedobacter sp.
  misc_feature 1..18: note = source = /note="crRNA"
  source 1..18: mol_type = other RNA
gttgtgaattgctttcaa

SEQ ID NO: 56 - RNA, 75 nt, Empedobacter sp.
  misc_feature 1..75: note = source = /note="tracrRNA"
  source 1..75: mol_type = other RNA
ttgaaaagcaattcacaataaggattattccgttgtgaaaacattcaaggcggggcaactcgtcttttttctttt

SEQ ID NO: 57 - RNA, 127 nt, synthetic construct
  misc_feature 1..127: note = source = /note="Description of Artificial Sequence: Syntheticpolynucleotide"
  misc_feature 1..127: note = source = /note="sgRNA L1"
  source 1..127: mol_type = other RNA
gagcggacagcagcttcctatatctcgtacgttgtgaattgctttcaaaaagttgaaaagcaattcacaataaggattattccgttgtgaaaacattcaaggcggggcaactcgtcttttttctttt

SEQ ID NO: 58 - RNA, 127 nt, synthetic construct
  misc_feature 1..127: note = source = /note="Description of Artificial Sequence: Syntheticpolynucleotide"
  misc_feature 1..127: note = source = /note="sgRNA L2"
  source 1..127: mol_type = other RNA
ccatgatatagacgttgtggctgttgtagtgttgtgaattgctttcaaaaagttgaaaagcaattcacaataaggattattccgttgtgaaaacattcaaggcggggcaactcgtcttttttctttt

SEQ ID NO: 59 - skipped

SEQ ID NO: 60 - DNA, 38 bp, synthetic construct; identical to SEQ ID NO: 8
  misc_difference 31..38: note = a,c,t,g, unknown, or other
  misc_feature 1..38: note = source = /note="Description of Artificial Sequence: Syntheticoligonucleotide"
  misc_feature 1..38: note = source = /note="Target seq and PAM region of plasmid library 1"
  source 1..38: mol_type = other DNA

SEQ ID NO: 61 - DNA, 38 bp, synthetic construct; identical to SEQ ID NO: 9
  misc_difference 31..38: note = a,c,t,g, unknown,or other
  misc_feature 1..38: note = source = /note="Description of Artificial Sequence: Syntheticoligonucleotide"
  misc_feature 1..38: note = source = /note="Target seq and PAM region of plasmid library 2"
  source 1..38: mol_type = other DNA

SEQ ID NO: 62 - RNA, 122 nt, synthetic construct
  misc_difference 1..25: note = a,c,t,g, unknown, or other
  misc_feature 1..122: note = source = /note="Description of Artificial Sequence: Syntheticpolynucleotide"
  misc_feature 1..122: note = source = /note="sgRNA with N's"
  source 1..122: mol_type = other RNA
nnnnnnnnnnnnnnnnnnnnnnnnngttgtgaattgctttcaaaaagttgaaaagcaattcacaataaggattattccgttgtgaaaacattcaaggcggggcaactcgtcttttttctttt

SEQ ID NO: 63-66 - skipped (4 entries)

SEQ ID NO: 67 - protein, 7 aa, synthetic construct
  REGION 1..7: note = source = /note="Description of Artificial Sequence: Syntheticpeptide"
  REGION 1..7: note = source = /note="NLS"
PKKKRKV

SEQ ID NO: 68-72 - skipped (5 entries)

SEQ ID NO: 73 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatatctcgtac

SEQ ID NO: 74 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatatctcgtag

SEQ ID NO: 75 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatatctcgttc

SEQ ID NO: 76 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatatctcgaac

SEQ ID NO: 77 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatatctcctac

SEQ ID NO: 78 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatatctggtac

SEQ ID NO: 79 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatatcacgtac

SEQ ID NO: 80 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatatgtcgtac

SEQ ID NO: 81 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctataactcgtac

SEQ ID NO: 82 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctatttctcgtac

SEQ ID NO: 83 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctaaatctcgtac

SEQ ID NO: 84 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcctttatctcgtac

SEQ ID NO: 85 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttccaatatctcgtac

SEQ ID NO: 86 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttcgtatatctcgtac

SEQ ID NO: 87 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcttgctatatctcgtac

SEQ ID NO: 88 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagctacctatatctcgtac

SEQ ID NO: 89 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcagcatcctatatctcgtac

SEQ ID NO: 90 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcaggttcctatatctcgtac

SEQ ID NO: 91 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagcaccttcctatatctcgtac

SEQ ID NO: 92 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacagctgcttcctatatctcgtac

SEQ ID NO: 93 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacaggagcttcctatatctcgtac

SEQ ID NO: 94 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggacaccagcttcctatatctcgtac

SEQ ID NO: 95 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggactgcagcttcctatatctcgtac

SEQ ID NO: 96 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggagagcagcttcctatatctcgtac

SEQ ID NO: 97 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcggtcagcagcttcctatatctcgtac

SEQ ID NO: 98 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagcgcacagcagcttcctatatctcgtac

SEQ ID NO: 99 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gagccgacagcagcttcctatatctcgtac

SEQ ID NO: 100 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gaggggacagcagcttcctatatctcgtac

SEQ ID NO: 101 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gaccggacagcagcttcctatatctcgtac

SEQ ID NO: 102 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
gtgcggacagcagcttcctatatctcgtac

SEQ ID NO: 103 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 4"
  source 1..30: mol_type = other DNA
cagcggacagcagcttcctatatctcgtac

SEQ ID NO: 104 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatatctcgtat

SEQ ID NO: 105 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatatctcgtgc

SEQ ID NO: 106 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatatctcgcac

SEQ ID NO: 107 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatatctcatac

SEQ ID NO: 108 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatatcttgtac

SEQ ID NO: 109 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatatcccgtac

SEQ ID NO: 110 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatatttcgtac

SEQ ID NO: 111 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatacctcgtac

SEQ ID NO: 112 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctatgtctcgtac

SEQ ID NO: 113 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctacatctcgtac

SEQ ID NO: 114 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcctgtatctcgtac

SEQ ID NO: 115 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcccatatctcgtac

SEQ ID NO: 116 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcttcttatatctcgtac

SEQ ID NO: 117 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagctttctatatctcgtac

SEQ ID NO: 118 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagctccctatatctcgtac

SEQ ID NO: 119 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagcctcctatatctcgtac

SEQ ID NO: 120 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcagtttcctatatctcgtac

SEQ ID NO: 121 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcaacttcctatatctcgtac

SEQ ID NO: 122 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagcggcttcctatatctcgtac

SEQ ID NO: 123 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacagtagcttcctatatctcgtac

SEQ ID NO: 124 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacaacagcttcctatatctcgtac

SEQ ID NO: 125 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggacggcagcttcctatatctcgtac

SEQ ID NO: 126 - DNA, 30 bp, synthetic construct
  misc_feature 1..30: note = source = /note="Description of Artificial Sequence: Syntheticoligonucleotide"
  misc_feature 1..30: note = source = /note="mismatch repair Table 6"
  source 1..30: mol_type = other DNA
gagcggatagcagcttcctatatctcgtac

SEQ ID NO: 127 - DNA, 3210 bp, synthetic construct
  misc_feature 1..3210: note = source = /note="Description of Artificial Sequence: Syntheticpolynucleotide"
  misc_feature 1..3210: note = source = /note="APG05083.1 mammalian codon optimized sequence"
  source 1..3210: mol_type = other DNA
atgagagagctggattaccggatcggcctggatatcgggaccaactccattggatggggcatcatcgagctgtcttggaacaaagacagagaacagtacgagaaggcaagaatcgtggacaagggcgtgagaatgttcgacaaggccgaaatacccaagaccggagccagcctggccgagcctagaagaattgccagatcaagcagaagacggctcaacagaaagtctcagagaaaaaaagatattcggaacctgctggtccagcacgagatcatcagccaaaaggaactcgctagcctgtatcctctgaccaagagcagcatggacatttgggacatcagactggacggcctggacagactgctggatagattcgagtggaccagattgctgatccacctggctcagcggagaggcttcaaaagcaaccggaagagcgagctgaaggacgtggaaaccggcaaggtgctctccagcatccaggctaatgagaagcggctgtccctgtacagaactgtgggcgagatgtggatgaagaatgaagattttagcaagtacgacaaaagaagaaatagtagcaacgaatacgtgttctctgtgtcccgggccgacctggaaaaggaaatcgtgacactgtttgaagctcagcggaagttccagagcagctatgccagcgccgatcttcaaaaaacctacctccagatctgggcccatcaactgcctttcgcctctggcaacgccatcgttaacaaggtgggctactgcagcctactgaaaggcaaggagaagagagttcctaaggccacctacaccttccaatacttcagcaccctggatcagatcaacagaaccagactgggcccaaacttccagcccttcaccaaggaacagagagatgtgatcctggacgagatgtttaaccggaccgattattacaagaagaagaccatccctgaggtgacgtactacgatatcagaaaatggctggccctggacgagacaatccagttcaagggcctgacctacgaccctaatgaagaactgaagaaaattgagctaaagtcttttatcaatctgaagcctttctacgagataaagaaggtggtgacaaactacgccaagaagacaaatgaggcctttagcacactggactatgacacctttgcctacgccctgacagtgtacaagaccgacaaggacatccgctcctacctgaaaaagagtaacaacctgtccaaatgctgctacgacgaccaactgatcgaggaattgctgacactgagctacaccaaattcggccacctgagcttcaaggctatcaaccacgttctgcctatcatgcaggagggcagaacctaccaggaggccattcaccagctcggctacgatgccacaaacctcaaaaaagagaaccggtctatgttcctgcctctgttccctgacgagatcaccaaccccatcgtgaagagggccctgacccaggccaggaaggtggtcaacgccatcatcagacgatacgggtctccaaacagcgtgcacatcgagctggccagagagctgagcaagagccacgacgagagaacaaagatcatgaaagctcatgatgaaaactacaaaaagaacaagggcgctatcagcatcctgatcgagaacggtattctgaatcctacaggttatgacatcgtccggtacaagctgtggaaggaacagggcgagagatgcgcctattctctgaaacagatccccgccaacaccttcttcaacgaaatgaagaaggagcggtccggcagccctgtgctggaaatcgatcacatcctgccctacagccagagcttcatcgacagctaccacaacaaagtgctggtgtacggggatgagaaccagaaaaagggcaatagaatcccgtacacctacttcctggaaggcaacaaggactgggagtctttcgagagctatgtgcgcctgaactcctttttcagcaagaaaaagcgaggatatctgctgaagaaggcttacctgccaagagagagtaacatgatcaaggaacggcacctcaacgacacccggtacgccagctcctacctgaagaacttcatcgagaagaatctgaagttcaaggaggtggaaggctctacccggaagaagcacgtgcaaaccgtgaacggcataatcacagcccacctgagaaagagatggggcctggaaaaggaccgccaggagacatatcttcatcacgctatggacgccatcatcgtggcatgcaccgaccaccacatggtgacaaaggtgaccgagtactaccagatcaaagaaagcaataaatctattagaaagccttacttccccctgccttgggtgggctttagagaggaaattctgtcccacctggctcggcagcctatcgccagaaagatctctgaagagctgaagatcggataccagagcttcgattacatcctcgtgtctagaatgcctaaaagatcagtgaccggcgccgcccacgagcagaccattatgaaaaagggaggcatcgacaaaaaaggaaaaaccatcatcattaagcgggtctacctgaaggatatcaagttcgacgagaatggcgatttcaagatggttggaaaggaacaggacctggctacctacgaggccatcaagcagagatacatcgagtacggcaaggaatccaagaaggccttcgagacccctctgtataagcccagcaagaaaggcaaaggcaacctgatcaagaagatcaaagtggaagtgcaaaccaagagctttgtgagagaagtcaacggcggagtggcccagaacggcgatctggtgcgggttgacctgttcgagaaggataatagatactacatgatccccatctacgtgatggataccgtgcacagcgaacttcctaacaaggccgtgaccagcagcaaaggctatgagcaatggctgaccatcgacaacagcttcaccttcaagttcagcctatacccctacgacctggtgcggctggtcaagggtaacgaggacagattcctgtacttttccaccctggatattaacagtgatagactcaacttcaaagacgtcaacaagcccagcaagcaggccgagaacagatatagcctgaagacaatcgaaaacctggaaaaatacgaggtgggcgtcctgggcgacctcagatttgtgagacaggagatcagaaagaacttc

SEQ ID NO: 128 - DNA, 3213 bp, synthetic construct
  misc_feature 1..3213: note = source = /note="Description of Artificial Sequence: Syntheticpolynucleotide"
  misc_feature 1..3213: note = source = /note="APG07433.1 mammalian codon optimized sequence"
  source 1..3213: mol_type = other DNA
atgagagagctggactacagaattggcctggacatcggcaccaacagcatcggatggggcgtgatcgagctgtcctggaacaaagaccgggagagatacgagaaggtcagaatcgtggatcaaggcgtgagaatgttcgacagagccgagatgcccaagacaggcgccagcttagctgaacccagaagaatcgccagatccagcagacggagactgaatcgcaagtcccagagaaagaaaaacatccggaacctgctggtgcaacacggcgtgatcacacaggaggaactggatagcctgtaccccctgagcaaaaagagcatggacatctggggcattcggctcgacggcctggacagactcctcaatcatttcgagtgggccagactgctgatccacctggctcagagacggggctttaagtccaacagaaagagtgaactgaaagatacagagacaggcaaggtgctgagcagcatccaactgaacgagaaacggctgagcttgtatagaaccgtgggcgagatgtggatgaaggaccccgacttctctaaatacgataggaagagaaatagccccaacgaatacgtgttcagcgtgtctagagccgagctggaaaaggaaatcgtgaccctgttcgccgcccagcggagattccagagcccttacgccagcaaagatctgcaggagacatatctgcagatctggacccaccaactgcctttcgccagcggcaatgccatcctgaacaaggtcggatactgctccctgttgaaaggcaaagaaagaaggattcccaaggctacatacaccttccaatacttctctgctctggaccaggtgaatcggaccagactgggacctgatttccagcccttcaccaaggagcaacgggaaattatcttgaacaacatgttccagaggacagattactacaagaagaaaaccatccccgaggtgacctactatgacatacggaagtggctggaattggacgagacaattcagttcaagggcctgaactacgaccctaacgaggaactgaagaagatcgagaagaagccttttatcaatctgaaggccttctacgagatcaacaaggtggtggccaactacagcgaaagaaccaacgagaccttctccaccctggactacgacggcatcggctacgccctgaccgtgtacaaaaccgacaaggatatccgcagctacctgaagagcagtcacaacctacctaagagatgctacgacgaccaactgatcgaggaactgctgagcctgagctacacaaagttcggccacctgtccctgaaagccatcaaccacgtgctgtctatcatgcagaagggcaatacctacaaggaagccgtggaccaactgggctacgacaccagcggccttaagaaggagaagaggtccaagttcctgccacctatttctgatgaaatcacgaatccaatcgtgaaaagggccctgacccaggccagaaaagtggtgaacgccataattagaagacacggatctcctcactccgtgcacatcgagctggccagagagctgagcaagaaccacgacgagcggacaaagatcgtcagcgcccaggatgaaaactacaagaaaaacaagggcgctatcagcatcctgtctgagcacggcatcctgaaccctacaggctacgacatcgtgagatacaaactgtggaaggagcagggcgaacggtgcgcctacagcctgaaggaaatccctgccgatacatttttcaacgagctgaagaaggaacgcaacggcgcccctatccttgaagtggaccacatcctgccctacagccagtccttcatcgactcctaccacaacaaggtcctggtgtacagcgacgaaaaccggaaaaagggcaacagaatcccttatacctacttcctggaaaccaacaaggattgggaggcctttgagcggtacgtgcggagcaacaaattcttctccaagaaaaagcgagagtaccttctgaagcgggcttatctgcctagagaatctgagctgatcaaagaacgccacctgaacgacaccagatacgcctctaccttcctgaagaacttcatcgagcagaacctgcagttcaaggaagccgaggacaaccccagaaaaagacgggtgcaaaccgtgaacggcgttatcaccgcccacttcagaaagcggtggggcctggagaaggaccggcaggagacatacctccatcacgctatggacgccatcatcgtggcttgtacagaccaccacatggtcaccagagtgaccgagtactatcagatcaaggaaagcaacaagagcgtgaagaagccctattttcctatgccttgggaaggcttccgggacgagctgctgagccacttggcttctcagcctatcgccaagaaaatcagcgaggaactgaaggccggctaccagagcctggactacatcttcgtgtccagaatgcctaagagaagcattacaggcgctgctcataagcagaccatcatgcggaagggaggaattgacaagaagggcaaaacaatcatcatcgaacggctgcacctgaaggatatcaagttcgacgagaacggagatttcaagatggtgggcaaggaacaggacatggccacatacgaagctattaaacagagatacctggagcacggcaagaatagcaagaaggccttcgagacccctctgtacaagcccagcaaaaagggcacaggtaacctgatcaagcgggtgaaggtggaaggacaggccaagagctttgtgagggaagtgaacggcggagtggcccaaaatggcgatctggttagagttgatttgtttgagaaggatgataagtactacatggtccccatctacgtgccagacaccgtgtgtagcgagctgcccaaaaaggtggtcgccagctctaagggctatgagcagtggctgacactggataacagcttcacctttaagttcagcctgtacccttatgatctggtgcggctggtcaagggagatgaggatcggttcctgtactttggcaccctggacatcgacagcgacagacttaacttcaaggacgtgaacaagccaagcaagaagaacgagtaccggtacagcttgaaaaccatcgaggacttggagaagtacgaggtgggcgtgctgggcgatctaagactggtccggaaggaaactcgaagaaacttccac

SEQ ID NO: 129 - DNA, 3213 bp, synthetic construct
  misc_feature 1..3213: note = source = /note="Description of Artificial Sequence: Syntheticpolynucleotide"
  misc_feature 1..3213: note = source = /note="APG07513.1 mammalian codon optimized sequence"
  source 1..3213: mol_type = other DNA
atgcgggaactggattacagaattggactggacatcgggaccaactcaattggctggggagtgatcgagctgagctggaacaaggacagagagcagtacgagaagaccagaatcgtggacaagggcgttaggatgttcgacaaggccgagatccccaagaccggagccagcctggccgagcctagacgtatcgccagatcctccagacgtagactgaacagaaagtcccagaggaagaaggacattcggaacctgctggtgcaacacgagatcattagccaaaaagaactgaccagcctgtaccctctgtctaaatccagcatggacatctgggacatccggctggacggcctggacagactgcttgatagattcgagtgggcccggctcctgatccacctggctcagcggcggggctttaaaagcaaccggaagtctgagcttaaagacgtggaaacaggaaaagttctgtccagcatccaggtgaatgaaaagcggctgagcctgtacagaaccgtgggcgagatgtggatgaagaacgccgattgcagcaagtacggcaaacgtagaaatagccccaacgagtacgtgttcagcgtgtctagagccgacttggagaaagaaattgtgacacttttcgaggcccagcgaaaattccacagcagctacgccagtgtggacctgcagaagacatacatccaaatctgggctcatcaactgccatttgcctctggcaatgccattgtgaacaaggtgggatactgctctctgctgaagggcaaagaaaagagagtgcctaaggccacatacacctttcagtattttaacaccctggaccagatcaaccggacaagactgggccctaacttccaacctttcaccaaggagcagagagatatcatcctagacaaaatgttccagagaaccgactactacaagaaaaaaacaatccctgaggtgacatactacgatatcagaaagtggctggccctggacgaaaccatccagttcaagggcctgacctacgatcctaatgaagaactgaagaagatcgagatgaagccattcatcaacctgaaacctttctacgagatcaagaaggtggtgaccaactacgccaagaagacaaacgaggtgttctctgccctggactatgacaccgtggcttatgccctcaccgtgtacaaaacagacaaggatatcagaagctaccttaagcggtccaacaacctgagcaagagatgttacgacgaccaactgatcgaggaactgctgacactgagctacaccaagttcggccacctgtccttcaaggccatcaatcacgtgctgcccatcatgcaggagggcagaacctaccaagaggctattcaccaactgggctatgacacgaccaacctgaagaaggaaaatagaagcatgttcctgcccatcatccctgacgagatcaccaaccctatcgtgaagcgggccctgacgcaggcccggaaagtggtgaatgccatcatccgcagatacggctctcctaattctgtccacatcgagctggccagagagctgagcaaaagccacgacgagcggaagaagatcatgaccgcccacgacgagaactacaagaaaaacaagggcgccgtgtccatcctgatcgataacggcatcctgaatcctacaggatacgatatcgtgcggtacaagctgtggaaggaacagggcgaaagatgcgcctatagcctgaaaaaaatccccgccaacaccttcttcaacgagctaaaaaaggaacggagcggcccacctgtgctggaagtggaccacatcctgccctacagccagagcttcatcgacagctaccacaacaaggtgctggtgtacggcgacgagaaccagaagaagggcaatagaatcccttacacatttttcagcgaagaagataaggaatgggagagcttcgagagctacgttcggagcaacagcttcttcagcaagaaaaagcgcggctacctgctgaagaaggcctacctgcccagagaaagcaacctgatcaaggaacggcacctcaacgacacacggtacgccagcagttacctgaagaatttcatcgagaagaacctgaagttcaaggaagccgtggggatcacccggaagaagtacgtgcaaaccgtgaacggcgtgatcaccgcccacctgcggaagcggtggggccttgagaaggaccggcaggagacctacctgcaccacgctatggacgccatcatcgtggcctgcaccgatcaccacatggtgacgaaggtgaccgagtactaccagatcaaagaaggcaataagagcatcaagaagccttattttcctctgccctggatgggcttcagagaagaaatcctgtctcacctggagtctcaacctatcgccagaaaaatttctgaagagctgaaaattggataccagtcccccgattacatcctggtcagccggatgcctaagagaagcgtgaccggcagcgcccacgatcagaccgtgatgaagaagggcgatatcgataagaagggcaagacaatcatcatcaagcgggtgcacctgaaggatatcaagttcgacgaaaatggcgacttcaaaatggtgggcaaggagcaggacctggctacatacgaagctatcaaacagagatacctggagtaccggaaggaaagcaagaaggccttcgagacccctctgtacaagccatctaaaaaaggaaaaggtaacctgatcaaaaagatcaaggtggaagtgcaaaccaaatctttcgtgagagagattaacggcggagtggcccagaacggcgacctggttagagtggatctgttcgagaaggacaacagatattacatggtccccatctacgtggtggacaccgtgcggtctgaactccccaacaaagcagtgacaagctccaaaggctatgaacagtggctgagcatcgacaatagtttcaccttcaagtttagtctgtacccctacgacctcgtgcggctggtcaagggcgacgaggatcgcttcctgtacttctccaccctggatatcaacagcgacagactgaacttcaaggacgtgaacaagcctagcaagcaggctgaataccggtacagcctgaagacgatcgagaacctggaaaaatacgagatcggtgttctgggagatctgagactcgttagacaggagacaagaaaaattttcaag

SEQ ID NO: 130 - DNA, 3216 bp, synthetic construct
  misc_feature 1..3216: note = source = /note="Description of Artificial Sequence: Syntheticpolynucleotide"
  misc_feature 1..3216: note = source = /note="APG08290.1 mammalian codon optimized sequence"
  source 1..3216: mol_type = other DNA
atgagcgagctggattatagaatcggcctggatatcggcaccaattctatcggatggggagtgatcgagctgttctggaacaaggatcgggaacggtacgagaaggtccgcatcgtggacaaaggggtcagaatgtttgacaaggccgagatccccaacaagggcgccagcctggccgagcctcggagaatcgcccggagcagcagaagacgcctgaacaggaagtcccaaaggaagaaggagatccggaacctgctggtgcaacacggcatgatcacccaagaggaactggacctcctgtaccctctgagcaaaaagtccatcgatatctgggacatcagactagacggcctggacagactgctgaaccacttagagtgggctagactgctgattcacctggcccagcggcggggcttcaagagcaaccgcaaaagcgagctgaaggacgctgaaacaggcaaggtgttgtctagcatccaggtgaacgagaagcggctgttcctgtacagaaccgtgggggaaatgtggatcaaggacgcagagttcagcaagtacgacaggcgtagaaactccccaaacgaatacgtgttcagcgtgtcaagagccgacctggaaaaggaaatcgtgacactgtttgaggctcagagaaagttccagagcagctatgcctctaaaaatctgcaggagacctacctgcagatctgggcccaccaactgcctttcgccagcggcaatgccatcctgaacaaagtgggctactgcagcctgctgaaagggaaggaaaggcggattcccaaggccacatacaccttccagtacttctccgccctggatcaggttaaccggacccggctcggccctgacttccaacctttcacccaggaacaaaaggagatcatcctggataagatgttccagagaacagactactacaagaagaagaccatccccgaggtttcttattatgatatcagaaagtggctggaactcgacgagacaatccagtttaagggactgaattacgaccccaacgaagagctgaagaagatcgaaaaaaaacctttcatcaacctgaaggccttctacgagatcaagaaggtggttgctaactacgccgaaagaacaaatgaagccttttctaccctggactacgacgctatcgcctatgccctgacagtctacaagaccgacaaggacatccggtcctacctgaagaaatccaacaacctgtccaagcggtgctacgatgatcaactgatcgaggaactgtttaccctgagctacaccaaattcggccacctgtctttcaaggccattaaccacgtgctgcctatcatgcaggagggcagaacctaccaggaggccatacaccaactgggctacgacaccaccaacctgaagaaagagaatagaagcatgttcctgcctctgatccctgacgagatcacaaaccccattgtgaagcgggccatcacccaggccagaaaggtggtgaacgccatcatcagaagatacggctctcctaacagtgtgcacatcgaactggccagagagctgagcaagagccacgatgagcggaaaaagatcatgaccgcccacgacgagaactacaagaaaaataagggcgctatttctatcctgatcgagaacggcattctgaaccctaccggctacgacatcgtgagatacaagctgtggaaggaacaaggcgagagatgcgcctacagcctgaaagaaattccacctgatacttttttcaacgagctgaagaaagaaagaaacggcagccccattctggaagtggaccacatcctgccttacagccagtccttcatcgacagctaccacaacaaagtgctggtgtactctgacgagaacagaaacaagggcaacagaatcccttacacctacttcctggaaacaaacaaggactgggaggcctttgaaagatacgtgcggagtaacaaactgttttctaagaaaaagagagaatatctgctgaagaagacttacctgcctagagagtctgaactaatcaaggaacggcacctgaacgacactcggtatgcttctacattcctgaagaatttcatcgagcagaatctgcagttcaaagaggtcgaggtcaatctgagaaaaaagagagtgcaaaccgtgaatggcgtgatcaccgcccacctcagaaagcgttggggcctggagaagaaccggcaggagacgtacctgcaccacgctatggacgccatcatcgtggcttgtacagaccaccacatggtgacccggataacagagtactaccagatcaaggaatccaacaagagcgtgaagaaaccttacttccccatgccttgggagggctttagagatgagctgttgtctcacctcgctagccagcctatcgcaaagaagatcagcgaggaactgaaggccggctaccagagcagcgactacatcttcgtgtccagaatgcccaagagaagcgtgaccggcgccgcccatgatcagaccatcagaagaaagggcggcatcgacaagaagggcaagacaatcatcattaagcgggtgcggctgaaagatatcaagttcgacgaaaacggcgatttcaagatggtgggcaaagagcaggacctggcaacctacgaggccatcaagcagagatacctggagcacagaaagaacagtaagaaggccttcgagacccctctgtacaaacctagcaagaagggaacaggaaacctgattaagcgggtgaaaatcgagggacagactaaggccttcgtgcgggaagtgaacggaggcgtggcccaaaatagcgacctggtcagagtggacctgttcgagaaggacgacaagtactatatggtgcctatctacgtgccagacaccgtgtgtagcgagctgcctaagaaggtggttaagagcggaaaaggctatgagcagtggctgaccctggacaacagcttcaccttcaagtctagcctgtacccctacgatctggtgcggctcgtgaagggcaacgaggacagattcctgtacttcggcacactggacattgactccgatagactgaatttcaaggatgtgaacaagcccagcaagcagaacgagtaccggtacagcctgaaaacaatcgagaacctggaaaaatacgaggttggagtgctgggagatctgcggctggtgaaacaggagaccaggaggatctttaacaga

SEQ ID NO: 131 - DNA, 4011 bp, synthetic construct
  misc_feature 1..4011: note = source = /note="Description of Artificial Sequence: Syntheticpolynucleotide"
  misc_feature 1..4011: note = source = /note="APG05459.1 mammalian codon optimized sequence"
  source 1..4011: mol_type = other DNA
atgaaaaaagattacgtgatcggcctggacattggcaccaacagcgtcggctgggccgtgatgacagaggactaccaactggtgaagaagaagatgccaatctatggcaatactgagaagaaaaagataaaaaagaatttctggggcgtgcggctgtttgaggagggccacaccgccgaggatcggcggctgaaacggaccgctagaagacggatctctcgccgcagaaatagactgagatatctgcaggccttcttcgaggaagccatgaccgccctggacgagaacttcttcgccagactgcaggagagcttcctggtgcctgaggacaagaaatggcaccggcaccctatcttcgccaagctggaggatgaggtggcctatcacgaaacctaccctacaatctaccacctgagaaagaaactggctgattcctctgaacaggctgacctgagactgatctatctggccctggcccatatcgtgaagtacagaggccacttcctgatcgaaggaaagctgagtaccgagaacatcagcgtcaaggagcagttccagcagttcatgattatctataaccagacatttgtgaacggcgaaagccggctggtgtctgcccctctgcctgagagcgtgctgatcgaagaagagctgaccgaaaaggccagccggacaaagaaatctgagaaggtgctgcaacagtttcctcaggagaaagccaatggactgttcggccagttcctgaaacttatggtaggcaacaaagccgatttcaagaaagtctttggcctggaagaagaggccaaaataacatacgccagcgagtcctacgaggaggatctggaaggcattctggccaaggtgggcgacgagtacagcgatgttttcctggccgctaagaacgtctacgacgccgtggaactgtctaccatcctggccgactccgacaagaagagccacgccaagttgtctagtagcatgatcgttagattcaccgagcaccaggaggacctgaagaagttcaagcggtttatcagagagaattgccccgacgagtacgataacctgttcaagaacgagcaaaaagacggctacgccggctacatcgcccacgccggcaaggtgtcccaactgaagttctaccagtacgtgaagaagataatccaggacatcgccggcgccgaatacttcctggagaaaatcgcccaggagaacttcctgcgaaaacagaggaccttcgacaacggcgtgatcccccaccagatccacctggccgagctgcaggccatcatccacagacaggctgcttactaccctttcctgaaggaaaatcaggaaaagattgagcaactggtgacattcagaatcccctactacgtcggccctctgagcaaaggagatgccagcaccttcgcctggctgaaaagacaaagcgaggaacctatccggccttggaacctgcaagagacagtggacctggaccagtctgctaccgcattcatcgagagaatgaccaacttcgatacctacctgccttctgagaaggtgctgcccaagcacagcctgctctacgaaaaatttatggtgttcaacgagctgaccaagatttcttacactgatgacagaggcatcaaggccaacttcagcggcaaggagaaggagaagatattcgactacctgtttaagacccgccggaaggtgaagaaaaaggatatcatccagttctaccggaacgagtacaacaccgagatcgtcaccctgtctggcctggaagaggaccagttcaatgccagcttcagcacctaccaagatctgctgaagtgcggcctaactagagccgaactggaccaccctgataatgccgagaaactggaggacatcattaagatcctaaccatcttcgaggatagacagcggatcagaacacagctcagcaccttcaagggacagttcagcgccgaggtgctgaagaagctggaacggaagcactacaccggctggggcagactgtccaagaaactcatcaacggcatctacgacaaggaatccggaaagacaatcctggactacctgatcaaagatgacggcgtttctaagcactacaacagaaacttcatgcagctaatcaacgacagccaactgagcttcaagaacgccatccagaaggcccagagcagcgagcatgaggaaaccctgtctgagaccgtgaacgagctggccggcagccctgccatcaagaaaggcatctaccagagcttaaaaatcgttgatgagctggttgccatcatgggctacgcccctaagagaatcgtggtggagatggctagagagaaccagacaacaagcaccggaaagagaagaagcatccagaggctgaagatcgtggaaaaagctatggccgagattggaagcaacctgctcaaggaacagcctaccacaaacgagcaactgagagatacaagactcttcctgtattatatgcagaacggtaaggacatgtacaccggcgacgagctgagcctgcacagactgtctcactacgacatcgaccacatcattcctcagtccttcatgaaggacgattccctggataacctggtgctggtgggcagcaccgagaaccggggcaagtctgacgacgtgcccagcaaggaagtggtgaaggacatgaaagcctactgggagaagctctacgctgctggcttgatcagccagcggaaattccagagactgacgaagggcgagcagggcggcctgaccctggaggacaaagctcatttcatccagagacaactggtggagacaagacagatcaccaagaacgtggctggaattctggatcagagatacaacgccaacagcaaagaaaaaaaggtgcaaatcataacactgaaagcctctctgaccagccagttccggagcatcttcggcctgtataaggtcagagaagtgaacgactatcaccacggccaggatgcgtacctgaactgcgtggtggccactacactcctgaaagtgtaccccaacctggctcctgagttcgtgtacggcgagtaccccaagtttcaggcctttaaggaaaacaaggctacagccaagaccatcatctacaccaatctgatgcggtttttcaccgaggatgaacccagattcatgaaggacggcgagatcctgtggagcaacagctacctgaagaacattaagaaagaactcaactaccatcagatgaacatagtgaaaaaggtggaagtgcaaaagggtggcttctccaaggaaagcatcaagcctaagggccccagcaacaagctgatccctgtgaagaacgggttggacccccagaaatacggcggatttgactctccagtggtcgcttacacagtgctgtttacccacgagaagggcaaaaaaccactgatcaagcaggagatcctgggaatcaccatcatggaaaagaccagattcgagcagaatcctatcctgttcctggaagaaaaaggcttcctgaggcctagagtgctgatgaagctgcctaaatacaccctgtacgagttccctgagggaagacggcggctgctggccagcgccaaagaagcccagaaagggaatcagatggtgctccccgaacacctgctgaccctgctgtaccacgccaaacagtgtctgctgcctaaccagagcgaatctctcgcttacgtggaacaacaccaaccggagttccaagagatcctggagagagtggtggacttcgctgaggtgcacacactggccaagagcaaggtgcaacagatcgtgaaactgtttgaggcaaaccagaccgcagatgtgaaggaaatcgccgcctccttcatccaactgatgcagttcaacgcaatgggagccccatctacctttaaattcttccagaaggacatcgagcgggcccggtacactagcatcaaggaaatcttcgacgccaccatcatctaccagagcacaaccggcctctacgagacacggagaaaggtggtggac

SEQ ID NO: 132 - DNA, 4308 bp, synthetic construct
  misc_feature 1..4308: note = source = /note="Description of Artificial Sequence: Syntheticpolynucleotide"
  misc_feature 1..4308: note = source = /note="APG04583.1 mammalian codon optimized sequence"
  source 1..4308: mol_type = other DNA
atggccaagaatatcctgggcctggatctgggtacaaacagcatcggctgggctctggtccagcaggacttcgagaacaaggagggaaacatcctgggcatgggatctagaatcatccctatgagccaggacatcctgggcgaattcggtaagggcaacagcatcagccagaccgccgagagaacaggataccggggcgtgcgtagactgagagagagacacctcctgagaagagagagactgcatagagtgctgcacctgctcggctttctgcctaagcactacgatgaaaaaatcgactttacacaaagattcggcaagttcatcaaccaggctgagcctaagctggcttttgatagcgagttcctgttcaaggacagcttccacgagatgctggccgacttcaagcagaatcaacctgaattcctgaaagacaagaacggagaggactgcctggttccttacgactggaccatctactacctgagaaagaaggctctgacccagaagatcgagaaatacgaactagcttggctgatcctgaacttcaaccaaaagcggggctactaccagcttagaggcgaagaagagaaggaaaaccctaacaaactggtgggcttccactctctgaagattgttgatgtgatccctgatgccgagaccaacaaaaagggcgagacatggtacagcctgcacctggaaaacggctgggtgtacaggcggagcagcaagatcagcctggccgactggaaggacaaggttcgggacttcatcgtcactacagatctgaacgacgatggcagcgagaagctggacaaggacggcatcgtgaagaggtccttcagagccccttctgccgacgattggacactcctgaaaaaaaagactgagaaggacatcgacaactccaacaagactgtgggcacctacatctacgacaacttgctgctgaaccctaaacagaagatcaaggggaaaatggtcagaaccatcgaacgaaagttctacaagcaggagctggagcaaatcctgaaaacccagaaagaatttcactccgagctgcagtctgaaaacctgctgcaggactgcgtgagggagctgtaccggaacaacgagcagcaccagcagatgctggaagccaaggattttgtgcacctgttcctgaacgatatcatcttctaccagagacccctgcgcagccagaaaagctctattagcaactgcaccctggaattccggaagagcaagaacgagaatggagaagaagtgatccaccggctgaaggtgatcgccaagtccaacccctactatcaagagtttcggctgctgcagtgggtgcaaaacctggctatctacactaaggacgacgacaaaaacgtcacaaacgaattcctgaagagcacccaggactgggaggatctgctgagatggctgcacagcaagaaagaaatcaagcaggatgccctgatcaaattcctgatcgaaaagaaaggcctgaagggtaaggccctgaccattgaagtggctaagtacagatggaattacgtgcaagacaaggattatcctggcaacgagacaagatacctgattcagagccggctggacaaggtggaatacgcccctaaggacttcctcacgtacgaaaatgagatggccctgtggcacatcatctactctatcaacgataagatcgaatacgaaaaggccctaaagagcttcgccaacaagaaaggtctcgacgaggtgaccttcgtggaagcctttaagaaattcccacctttcaagagcgattacggcagcttcagcgagaaggccatcaagaagctgctgcctctcatgcggttcggcacacaatggaactgggacaacatcgaccagaactcgaaggaaaggattggaaaaatcctgaccggcgagtacgacgagaatatcaagggtagagtgcgggaaaaagctagacacctgaacagcgagacggactttcaggcgctccctctgtggctggcccagtacgtggtgtacggcagacatagcgaagctgacatcgccggcaagtggaatagtgtggacgacctgaagcagttcttggacgacttcaagcaacacagcctgagaaatcccattgtagaacaggtgattactgagacactgcgggccgtgaaggatatctggaacttctacggcaagggcgccaaggactttttctctgagatccacatcgagctgggaagagagatgaaaaacaccgccgacgagagaaaaaggattaccacaatggtgacagataatgagaataccaatctgagaatcaaagctctgctggctgagatggccctggatcagaacgtggacaatgtgcggccttacagccccatgcagcaagaaatcctgaaaatctatgaggaaggcgtgctgaacgccgaagagaacatcgacgatgacatcctgaagatctctaaaactgctcagcctagcgctaccgatctgaagagatacaagctgtggctggaacagaagtacagaagcccctataccggacagatgatccctctgaacaagctgttcacccctgagtatgaaatcgagcacattatcccccagagcagatacttcgacgattctatgagcaacaaggtgatctgcgaggccgccgtgaacaagctgaaggataaccagatcggcctggtgttcatcaagaaccaccacggcgaagtggtggactttggcatgggcaagcaggtgaaaatcctggaggtgtctgattacgaagatttcgtgaagcagaactacaacaaaaacagaggcaaacggaacaagctgctcctcgaagatatccccgagaaaatgattgaacggcaactgaacgataccagatatatcagcaagtacatcactcaggtgctgtctaatatcgtcagagacgacaaggaaggctctaaggatgacggagtgaacagcaagaacattgtgcccggcaacggcaagattacaaccagactcaagcaggattggggcctgaacgatgtgtggaacgacctggtgctgcctagattcgagagaatgaacaccctcacaaactccaatgattttacaagcaagaacacacacggcaagaccattcctaccgtgcctatcgagctgagcaaggggttcagcaaaaagcggatcgatcacagacaccacgctatggacgccctggtgatcgcctgcgccacccgggaccacgtgaacctgctgaataacgaatccagcaagtctgacacaaagcggtacgacctgaatagaaaactgagaaaatacgagaaggtggcctacaacgaccccaaaaccggcgagaggattgaaaaagaagtgccaaaagacttcatcaagccttgggaaacttttaccgaagataccagaacactgctggagaatatcgtgatctcattcaaacagaacctgagagtgatcaacaaggccaccaactactacgagaagatcgagaacggcaaaaaggtgaaggtggaacaaaagggaatcaactgggccgtgagaaaggctctccataaggagaccgtgtccggccaggtccaccttgacagaatcaaggtggccaagggcaagatcctgaccgccacaagaaagaccctggacgcttcttttaacgagaaaaccatagagagcatcaccgataccggcatccagaagatcctgctgaactacctgaagtcaaaggacaacaaccctgaggtggccttctccccagaaggaattgaggaactaaacaagaatatcagactgtacaatgacggcaaggcccaccagccaatcctaaaagtgcgggtgttcgagcagggcagcaagttcacactgggcgagacaggcaacaagacaaccaagttcgtggaagctgccaagggcaccaatcttttcttcggaatctacgaggacaagagcggaaaacgtagctacgagaccatccccctgaatatcgttattgaaagacagaaacagggcctccaggccgtgcccgagaccaacgagaagggcaagcaactgctgttcaccctgagccccaacgacctggtctacgtgccagaggaaggcgtattcgacgagaacaacatcaaggtggatagaatctacaaggtcgtgtctttcagcacataccaatgttttttcgtgcggaacgacgtatctaccagcgtggtcaacaaggtggagtacagcgccctgaacaagatggaaaaatccatcgacaatatcatgatcaaggaaaactgtgtgaagctgaatgtggaccggctgggtaagatcagcaaggcc

SEQ ID NO: 133 - DNA, 4368 bp, synthetic construct
  misc_feature 1..4368: note = source = /note="Description of Artificial Sequence: Syntheticpolynucleotide"
  misc_feature 1..4368: note = source = /note="APG01688.1 mammalian codon optimized sequence"
  source 1..4368: mol_type = other DNA
atgatgatcaagaacattctgggactggatttgggcacaaacagcattggatgggctctgatcaagcaggacttcgagaacaaacacggcgagatcctgggcatgggcagcagaatcatccctatgagccaggatatcctgggcgatttcggcaaagggaactcgataagccagactgctgacagaaccaagtacagaagcgtgcggagactgagagaaagattcttgctgcggcgggaacgcctgcaccgggtgctccacctgctgaacttcctccctcaacactacgcctctcagatcgacttcgagaagaagttcggcaagtttaagtctgagaccgaacctaagctggcctgggaaaactggggaggtaaattctctttcctcttccagaacagcttcaacgagatgctggaagacttcaaggccgccggccagggactcaagattccctatgactggaccatctactacctacggaagaaagctctgtcccagaagatcgagaaagaggagcttgcctggattctgctgaacttcaaccagaagcgaggctactaccagctcagaggcgaggaagaagaagagaacccgaacaagctggtggagttctactccctgaagatcgtggatgtggtcgccgacgagcctcagaaggggaagagcgacatctggtattctctgatcctggaaaacggctgggtctaccgcagagccagtaagatcccactgtttgattggaaggataagaccagagatttcatcgtcaccacagaccttaatgacgacagaagcgtgaagaccgacaaagagggcaacgagaagcggtcctttcgggcccctagcgaaaatgattggaccctggtcaagaagaagacagagcaggagattgaccagagccacaagaccgtgggcacctacatctacgagacactgctgctcaatccaaagcagaaaatcaagggcaagctggtgcgaacaatcgagagaaaattttataaggacgagctgaagcaaatcctggaaaagcagaaggaatttcaccaggagctgaaaaacgacgatctgtacaacgactgcatcagagagctgtaccggaacaacgaggcccaccaactgaccctgagcaagaaagatttcgtgcacctgctgatggacgatctgatcttctaccaaagacccctgcgaagccagaagtccagcatctctaactgcaccctggaattcagaaaatacaaggacgaaaacggcatcgaacacacccagtacctgaaggccatcccaaagagtaatccgtactaccaagagtttagactgtggcagtggatgtacaacctgaacatctacagaaaggacgacgaggctaatgtgaccaaggaattcctgaacaccaataaggacttcgagagcctgtttgagttcctgaataatagaaaggaaatcgaacagaagcctctgatcaagttcctcctggaacagaaagatattaacaagaagctgctgaacgccgaggccgaaaagtatcggtggaattacgtggaagataagaagtatccttgcaacgaaacgaagaccatgatctccagcagactggacaaagtggagaatatctctgacgacttcctgaccagagacattgagcagaagatctggcacatcatctacagcgtcaacgacaagatagaatacgagaaggccctgaagtccttcgccaccagaaacgatctcgacgagaacagctttatcgaagcctttaagaagttcagcccattcaagagcgagtacggatctttttctgagaaagccatcaagaaactgctgcccctgatgagactgggaaagtactggtacgaggacgagattgtgaagcactctgatatttactttaagaacatcgaaaatcttctgggcgacttctccaacagagataaaaaaatatctgaggaagacaaagagaaatggaacaagtctatcaacctgaaactgcaggaggaactgaaggactttcagaccgccgagatcgacctgttccagggccttagactccatatcgcccagtacctggtgtacggccggcacagcgaagccagcatgatcggaaagtggaacagcgccgaggatctggaagaattcctgaaggacttcaagcagcacagcctgagaaaccccatcgtcgagcaagtgatcaccgagaccctgagggtggttaaggatatatggctgaaatacggcaacggcgccaaggacttcttcaacgagattcacatcgagctgggcagagaaatgaagctgcctgccgacgaccggaaaaagctgaccaaccagatcagcgagaacgaaaacacaaatttcagaatcaaggccctgctggctgaaatgatgaacgacagcagtgtggaaaatgtgcggcctttcagccctatgcagcaggagatcctgaagatctatgaagatgacgtcctgaagtctgacattgaaatcgaggatgacatccttaagatctctaaaaccgcccagccttctcctagcgatctcaagcgatacaagctctggctggagcagaagtacaagtctccatacaccggccagatcatacctctgaacaagctgtttacccctgaatacgagatcgagcacattatccctcagtctagatatttcgacgacagcttcagcaacaaagtgatctgcgagagcgccgtgaacaagctgaaagacaactacattggcctggaattcatcaagcaatttggaggcaccatcatcgaactgggctttggcaagtccatcaaagtgttcgagaccaaagagtacgaggacttcgtgaagaaacactacgccaacaatcagggcaagagaaacaagctgctcatggaagacatccccgagaaaatgatcgagagacagatgaacgacaccagatacatctctaagtacatcagcggagtgctgagcaacatcgtcagagtggaagatggatctgatgagggcgtgaacagcaagaacatcgtgcccggcaacggcaagattacaacacaactgaagcaggactggggcctgaatgacgtgtggaacgaccttatcctgccgcggttcgagagaatgaatcaactgaccaacagcaaggtgttcacagcctggaacgaaaactaccagaagttcctgcctacagtgccaatcgaatacagcaaaggcttcagcaagaagcgtatcgatcacagacaccacgccctggatgctctggttatcgcctgcgccaccaaggaccacgtgaacctgctgaacaaccagtctgccaagagcgacaccaagagatacgacttaaagaaaaaaagtatgaaattcgagaaagtggtgtacaatgatgccaagaccggcgaaaagatcgagcgggaagtgcccaagcagttcctgaagccttgggagaacttcaccctggatgtgaagcacaacctggaaacgatcatcgtctcttttaaacaaaatctgcgggtgatcaataaagccacgaactactacgagaagtacgtcgagaaggacggcaccaaaaacaaagagcgcgtggagcagacagggacaaactgggccattagaaagcccatgcacaaggatacagtgtccggcaaagtggaccttccttgggtgaaggtgcctaagggaaagatcctgaccgccacacggaagagcttggatagcagcttcgacctcaagagcatcggctctataacagatacaggcattcagaagatcctaaagaattacctggcattcaaggacggcaaccctgagctggctttcagccctgagggcatcgacgatctgaacaagaacatagagaagtacaacgatggcaaacctcaccagcctatcaacaaggtgcgagtgtttgagctgggcagcaagttccaggtgggacaatctggtaacaagaaggacaagtatgtggaagctgctaagggcaccaatctcttcttcgccgtgtacgaggatgaaaaaggaaagcggaactacgagaccatccctctcaacgaggtgatcgagagacagaagcagggcctgagcgtggtggacctgaagggtacaaacgacttctacctgtgccctaacgactttgtgtacatcccaagcggcgacgagctggaaaacatcaacaacgtggacttcaaggacatcaagaaggagattaacgagcgcatctacaaagtggtgtcttttacaggcaatagactttcttgtatcccttacatggtggccacaacgatcgtcaacaaactcgaattcactcaactgaacaaaatcgagtttaccaaggaaaaagaaatttgtatcaagctcaacgtggacagactgggcaatatctccaaggcc

SEQ ID NO: 134 - DNA, 21 bp, synthetic construct
  misc_feature 1..21: note = source = /note="Description of Artificial Sequence: Syntheticoligonucleotide"
  misc_feature 1..21: note = source = /note="SV40 large T-antigen nuclear localization sequence"
  source 1..21: mol_type = other DNA
cctaagaagaaaagaaaggtg

SEQ ID NO: 135 - DNA, 66 bp, synthetic construct
  misc_feature 1..66: note = source = /note="Description of Artificial Sequence: Syntheticoligonucleotide"
  misc_feature 1..66: note = source = /note="Triple FLAG epitope tag"
  source 1..66: mol_type = other DNA
gactacaaggaccacgacggcgactacaaagatcacgatatcgactacaaggacgacgatgataag

SEQ ID NO: 136 - DNA, 48 bp, synthetic construct
  misc_feature 1..48: note = source = /note="Description of Artificial Sequence: Syntheticoligonucleotide"
  misc_feature 1..48: note = source = /note="Nucleoplasmin nuclear localization sequence"
  source 1..48: mol_type = other DNA
aaaagacctgccgctacaaagaaggccggccaggccaagaaaaagaag

SEQ ID NO: 137 - DNA, 203 bp, synthetic construct
  misc_feature 1..203: note = source = /note="Description of Artificial Sequence: Syntheticpolynucleotide"
  misc_feature 1..203: note = source = /note="Cytomegalovirus mammalian transcription promoter"
  source 1..203: mol_type = other DNA
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 138 - DNA, 304 bp, synthetic construct
  misc_feature 1..304: note = source = /note="Description of Artificial Sequence: Syntheticpolynucleotide"
  misc_feature 1..304: note = source = /note="Cytomegalovirus transcription enhancer"
  source 1..304: mol_type = other DNA
cgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 139 - DNA, 318 bp, synthetic construct
  misc_feature 1..318: note = source = /note="Description of Artificial Sequence: Syntheticpolynucleotide"
  misc_feature 1..318: note = source = /note="Human U6 RNA promoter"
  source 1..318: mol_type = other DNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 140 - DNA, 20 bp, synthetic construct
  misc_feature 1..20: note = source = /note="Description of Artificial Sequence: Syntheticoligonucleotide"
  misc_feature 1..20: note = source = /note="target for Guide 189"
  source 1..20: mol_type = other DNA
cctgaatgctgtgcggctct

SEQ ID NO: 141 - DNA, 20 bp, synthetic construct
  misc_feature 1..20: note = source = /note="Description of Artificial Sequence: Syntheticoligonucleotide"
  misc_feature 1..20: note = source = /note="target for Guide 185"
  source 1..20: mol_type = other DNA
ggacagtgcgcatctccctg

SEQ ID NO: 142 - DNA, 20 bp, synthetic construct
  misc_feature 1..20: note = source = /note="Description of Artificial Sequence: Syntheticoligonucleotide"
  misc_feature 1..20: note = source = /note="target for Guide 168"
  source 1..20: mol_type = other DNA
cacatctcgagcaagacgtt

SEQ ID NO: 143 - DNA, 20 bp, synthetic construct
  misc_feature 1..20: note = source = /note="Description of Artificial Sequence: Syntheticoligonucleotide"
  misc_feature 1..20: note = source = /note="target for Guide 135"
  source 1..20: mol_type = other DNA
gggccattaaaacctctcca

SEQ ID NO: 144 - DNA, 20 bp, synthetic construct
  misc_feature 1..20: note = source = /note="Description of Artificial Sequence: Syntheticoligonucleotide"
  misc_feature 1..20: note = source = /note="target for Guide 139"
  source 1..20: mol_type = other DNA
aggttttaatggcccagcct

SEQ ID NO: 145 - DNA, 20 bp, synthetic construct
  misc_feature 1..20: note = source = /note="Description of Artificial Sequence: Syntheticoligonucleotide"
  misc_feature 1..20: note = source = /note="target for Guide 143"
  source 1..20: mol_type = other DNA
catggcagtacattagagca

SEQ ID NO: 146 - DNA, 20 bp, synthetic construct
  misc_feature 1..20: note = source = /note="Description of Artificial Sequence: Syntheticoligonucleotide"
  misc_feature 1..20: note = source = /note="target for Guide 190"
  source 1..20: mol_type = other DNA
cctgaatgctgtgcggctct

SEQ ID NO: 147 - DNA, 20 bp, synthetic construct
  misc_feature 1..20: note = source = /note="Description of Artificial Sequence: Syntheticoligonucleotide"
  misc_feature 1..20: note = source = /note="target for Guide 194"
  source 1..20: mol_type = other DNA
gccgcacagcattcaggtcg

SEQ ID NO: 148 - DNA, 20 bp, synthetic construct
  misc_feature 1..20: note = source = /note="Description of Artificial Sequence: Syntheticoligonucleotide"
  misc_feature 1..20: note = source = /note="target for Guide 165"
  source 1..20: mol_type = other DNA
atggggaatgtagcaagacc

SEQ ID NO: 149 - DNA, 20 bp, synthetic construct
  misc_feature 1..20: note = source = /note="Description of Artificial Sequence: Syntheticoligonucleotide"
  misc_feature 1..20: note = source = /note="target for Guide 169"
  source 1..20: mol_type = other DNA
cacatctcgagcaagacgtt

SEQ ID NO: 150 - DNA, 20 bp, synthetic construct
  misc_feature 1..20: note = source = /note="Description of Artificial Sequence: Syntheticoligonucleotide"
  misc_feature 1..20: note = source = /note="target for Guide 173"
  source 1..20: mol_type = other DNA
cttctatagcctccttcccc

SEQ ID NO: 151 - DNA, 20 bp, synthetic construct
  misc_feature 1..20: note = source = /note="Description of Artificial Sequence: Syntheticoligonucleotide"
  misc_feature 1..20: note = source = /note="target for Guide 144"
  source 1..20: mol_type = other DNA
catggcagtacattagagca

SEQ ID NO: 152 - DNA, 20 bp, synthetic construct
  misc_feature 1..20: note = source = /note="Description of Artificial Sequence: Syntheticoligonucleotide"
  misc_feature 1..20: note = source = /note="target for Guide 136"
  source 1..20: mol_type = other DNA
gggccattaaaacctctcca

SEQ ID NO: 153 - DNA, 20 bp, synthetic construct
  misc_feature 1..20: note = source = /note="Description of Artificial Sequence: Syntheticoligonucleotide"
  misc_feature 1..20: note = source = /note="target for Guide 145"
  source 1..20: mol_type = other DNA
catggcagtacattagagca

SEQ ID NO: 154 - DNA, 20 bp, synthetic construct
  misc_feature 1..20: note = source = /note="Description of Artificial Sequence: Syntheticoligonucleotide"
  misc_feature 1..20: note = source = /note="target for Guide 188"
  source 1..20: mol_type = other DNA
ggacagtgcgcatctccctg

SEQ ID NO: 155 - DNA, 20 bp, synthetic construct
  misc_feature 1..20: note = source = /note="Description of Artificial Sequence: Syntheticoligonucleotide"
  misc_feature 1..20: note = source = /note="target for Guide 192"
  source 1..20: mol_type = other DNA
cctgaatgctgtgcggctct

SEQ ID NO: 156 - DNA, 20 bp, synthetic construct
  misc_feature 1..20: note = source = /note="Description of Artificial Sequence: Syntheticoligonucleotide"
  misc_feature 1..20: note = source = /note="target for Guide 196"
  source 1..20: mol_type = other DNA
gccgcacagcattcaggtcg

SEQ ID NO: 157 - DNA, 20 bp, synthetic construct
  misc_feature 1..20: note = source = /note="Description of Artificial Sequence: Syntheticoligonucleotide"
  misc_feature 1..20: note = source = /note="target for Guide 167"
  source 1..20: mol_type = other DNA
atggggaatgtagcaagacc

SEQ ID NO: 158 - DNA, 20 bp, synthetic construct
  misc_feature 1..20: note = source = /note="Description of Artificial Sequence: Syntheticoligonucleotide"
  misc_feature 1..20: note = source = /note="target for Guide 171"
  source 1..20: mol_type = other DNA
cacatctcgagcaagacgtt

SEQ ID NO: 159 - DNA, 20 bp, synthetic construct
  misc_feature 1..20: note = source = /note="Description of Artificial Sequence: Syntheticoligonucleotide"
  misc_feature 1..20: note = source = /note="target for Guide 175"
  source 1..20: mol_type = other DNA
cttctatagcctccttcccc

SEQ ID NO: 160 - DNA, 25 bp, synthetic construct
  misc_feature 1..25: note = source = /note="Description of Artificial Sequence: Syntheticoligonucleotide"
  misc_feature 1..25: note = source = /note="target for Guide 197"
  source 1..25: mol_type = other DNA
cctcacccccacgagcttgtaggaa

SEQ ID NO: 161 - DNA, 25 bp, synthetic construct
  misc_feature 1..25: note = source = /note="Description of Artificial Sequence: Syntheticoligonucleotide"
  misc_feature 1..25: note = source = /note="target for Guide 199"
  source 1..25: mol_type = other DNA
acagtgcgcatctccctggtcacca

SEQ ID NO: 162 - DNA, 25 bp, synthetic construct
  misc_feature 1..25: note = source = /note="Description of Artificial Sequence: Syntheticoligonucleotide"
  misc_feature 1..25: note = source = /note="target for Guide 146"
  source 1..25: mol_type = other DNA
ggctgggccattaaaacctctccag

SEQ ID NO: 163 - DNA, 25 bp, synthetic construct
  misc_feature 1..25: note = source = /note="Description of Artificial Sequence: Syntheticoligonucleotide"
  misc_feature 1..25: note = source = /note="target for Guide 148"
  source 1..25: mol_type = other DNA
gaggctgggccattaaaacctctcc

SEQ ID NO: 164 - DNA, 25 bp, synthetic construct
  misc_feature 1..25: note = source = /note="Description of Artificial Sequence: Syntheticoligonucleotide"
  misc_feature 1..25: note = source = /note="target for Guide 176"
  source 1..25: mol_type = other DNA
gaggctgagacaggagagttgcttg

SEQ ID NO: 165 - DNA, 25 bp, synthetic construct
  misc_feature 1..25: note = source = /note="Description of Artificial Sequence: Syntheticoligonucleotide"
  misc_feature 1..25: note = source = /note="target for Guide 177"
  source 1..25: mol_type = other DNA
cccacaaaccgatgtagctcaagag

SEQ ID NO: 166 - DNA, 25 bp, synthetic construct
  misc_feature 1..25: note = source = /note="Description of Artificial Sequence: Syntheticoligonucleotide"
  misc_feature 1..25: note = source = /note="target for Guide 209"
  source 1..25: mol_type = other DNA
tagggtcttactctgttgtccacgc

SEQ ID NO: 167 - DNA, 25 bp, synthetic construct
  misc_feature 1..25: note = source = /note="Description of Artificial Sequence: Syntheticoligonucleotide"
  misc_feature 1..25: note = source = /note="target for Guide 151"
  source 1..25: mol_type = other DNA
ttctcccgagccaagtacacgtttc

SEQ ID NO: 168 - DNA, 25 bp, synthetic construct
  misc_feature 1..25: note = source = /note="Description of Artificial Sequence: Syntheticoligonucleotide"
  misc_feature 1..25: note = source = /note="target for Guide 152"
  source 1..25: mol_type = other DNA
gaaaatctgctcttagggctcaagg

SEQ ID NO: 169 - DNA, 3210 bp, synthetic construct
  misc_feature 1..3210: note = source = /note="Description of Artificial Sequence: Syntheticpolynucleotide"
  misc_feature 1..3210: note = source = /note="APG05083.1 Soy optimized"
  source 1..3210: mol_type = other DNA
atgagagagctagattacaggatcggactcgatatcggaactaattcaatcgggtggggaataattgagctgtcctggaataaagatcgcgagcaatatgaaaaggctaggattgtggataagggagtgaggatgttcgataaggctgaaattcctaagactggagcttctcttgctgaaccaagaagaattgctagatctagcaggaggagattaaacaggaagtctcagcgcaagaaggatataaggaaccttctcgttcagcatgaaatcatttctcagaaggagcttgcttctctctaccctctaaccaaatcaagcatggacatctgggatataaggcttgatggactcgataggcttcttgatagatttgagtggaccaggcttctaatacaccttgctcaaagaaggggcttcaaatcaaaccgcaagtccgaattaaaggatgttgagactggaaaggtgctttcaagcatccaagctaatgaaaagaggctttctttgtacaggactgttggggagatgtggatgaaaaacgaggacttctcaaaatacgataaaaggaggaactcctccaacgagtacgtgttctctgtttctagggctgatcttgagaaggagattgtgactctttttgaggctcaacgcaagttccaatcaagctacgcttctgctgatcttcaaaagacctaccttcaaatttgggctcatcagcttccttttgcttctggaaacgccattgtgaataaagtgggatactgctccctattaaagggaaaggagaagagagtgcctaaggctacctatacattccagtacttctctaccctcgatcaaatcaatagaactaggcttggacctaacttccagccttttactaaggagcagagggatgtgatcctcgatgaaatgttcaacaggaccgactactataaaaagaagaccattcccgaggtgacctactacgatataagaaagtggcttgctctcgatgaaaccattcagttcaagggactaacttatgaccccaacgaggagctgaagaaaatagaactcaagtccttcataaacctcaagcccttctacgaaatcaagaaggtggtaaccaactacgccaagaagaccaacgaagcattctctaccctcgattacgatacattcgcttacgctcttaccgtgtataaaaccgacaaggatataaggtcctacctcaagaaatcaaacaacctctccaagtgctgctacgacgatcaattaattgaggagcttctcaccctctcctatactaagttcggacacctctctttcaaggcaatcaaccatgtgcttccaataatgcaagagggaagaacttaccaagaggcaatacaccaacttggatacgatgctaccaaccttaagaaggagaacaggtctatgttccttcctctcttccctgatgagattactaaccctattgtgaagagagctttgactcaagctaggaaggtggtgaatgctattattaggaggtacggatcacctaactctgtgcatattgagcttgctagggagctttctaagtctcatgatgagaggaccaagattatgaaggctcacgatgagaactacaagaagaacaagggggctatttccattctcattgagaacggaattcttaaccctaccggatacgatattgtgaggtacaagttgtggaaggagcaaggagagagatgcgcttactctcttaagcaaattcctgctaacaccttcttcaacgagatgaagaaggaaagatcaggatctcctgtgcttgagattgatcacattctcccttactcccagtccttcattgattcttaccacaacaaggtgcttgtttacggagatgagaaccaaaagaaggggaacaggattccttacacttacttccttgagggaaacaaggattgggagtctttcgagtcttacgtgaggcttaactccttcttctctaagaagaagaggggataccttcttaagaaggcttatcttcctagggagtccaatatgattaaggagaggcacctaaacgataccaggtacgcttcttcttacctcaagaacttcatcgagaagaaccttaagttcaaggaggttgagggatctactaggaagaagcatgttcagaccgtgaacggaattattactgctcatcttagaaagaggtggggacttgagaaggataggcaagagacttaccttcatcacgctatggatgctattattgtggcttgcactgatcaccatatggtgactaaggttaccgagtactaccagattaaggagtcaaacaagtccatcaggaagccttattttcctcttccttgggttggattcagggaggagattctttctcatcttgctagacaacctatcgctagaaagatttccgaggagcttaagattggataccagtccttcgattacattcttgtgtctaggatgcctaagaggtctgttactggagctgctcatgagcagactattatgaagaagggaggaattgataagaaggggaagaccatcatcattaagagggtgtacctaaaggacatcaagttcgatgagaacggggatttcaagatggttggaaaggagcaggatcttgctacttacgaggctattaagcagaggtacattgagtatggaaaggagtctaagaaggctttcgagactcctctttacaagccttctaagaagggaaaggggaacctcatcaagaagattaaggttgaggttcagactaagtctttcgttagggaggttaatggaggagttgctcagaatggagatcttgtgagggttgatctattcgagaaggacaacaggtactacatgatccctatctacgtgatggatactgttcattccgagcttcctaacaaggctgttacttcttctaagggatacgagcagtggcttaccattgataactctttcaccttcaagttctctctctacccttacgatcttgttaggcttgttaagggaaacgaggataggttcctctacttctccaccctcgatattaactctgataggctcaacttcaaggatgtgaacaagccttcaaagcaagctgagaacaggtactctcttaagaccattgagaaccttgagaagtacgaagtgggagttcttggagatcttaggttcgttaggcaagagattaggaagaacttc

SEQ ID NO: 170 - DNA, 3210 bp, synthetic construct
  misc_feature 1..3210: note = source = /note="Description of Artificial Sequence: Syntheticpolynucleotide"
  misc_feature 1..3210: note = source = /note="APG05083.1 Corn optimized"
  source 1..3210: mol_type = other DNA
atgcgcgagctagattacaggatcggcctggatatcggcactaactcaataggctggggcatcatcgagctgagctggaataaagaccgcgagcagtatgaaaaggccaggatcgtggacaagggcgtgaggatgttcgataaggccgagatcccaaagacaggcgcctctctggccgaaccaaggaggatcgccaggtcttctaggaggagattaaacaggaagtctcagcgcaagaaggatataaggaacctgctggtccagcatgaaatcatctctcagaaggagctggccagcctgtacccgctgaccaagtcaagcatggacatctgggatataaggctggatggcctggataggctgctggataggttcgagtggacaaggctgctgatacatctggcccagaggaggggcttcaaatcaaataggaagagcgaattaaaggacgtggagacaggcaaggtgctgtcaagcattcaggccaatgaaaagcgcctgagcctgtacaggacagtgggcgagatgtggatgaaaaacgaggacttctcaaaatacgacaagcgccgcaacagcagcaacgagtacgtgttcagcgtgagcagggccgacctggagaaggagatcgtgacactgttcgaggcccagaggaagttccaatcaagctacgcctctgccgatctgcagaagacatacctgcagatctgggcccaccagctgccattcgctagcggcaatgccatcgtgaataaagtgggctactgctctctattaaagggcaaggagaagagggtgccaaaggccacctatacattccagtacttcagcaccctggaccaaatcaataggacacgcctgggcccaaacttccagccattcacaaaggagcagcgcgacgtgatcctggatgaaatgttcaaccgcaccgactactataaaaagaagaccatcccggaggtgacatactacgatataaggaagtggctggccctggatgaaaccatccagttcaagggcctaacctacgacccgaacgaggagctgaagaaaatagaactgaagagcttcataaacctgaagccgttctacgaaatcaagaaggtggtaaccaactacgccaagaaaaccaacgaagcattcagcaccctggactacgatacattcgcctacgccctgaccgtgtataaaaccgacaaggatataaggagctacctgaagaaatcaaacaacctgagcaagtgctgctacgacgaccaattaatcgaggagctgctgaccctgagctatactaagttcggccacctgagcttcaaggcaatcaaccacgtgctgccaataatgcaggagggccgcacctaccaggaggcaatacaccagctgggctacgacgcaaccaacctgaagaaggagaaccgaagcatgttcctgccgctgttcccggacgagatcaccaacccgatcgtgaagcgcgccctgacccaggcccgcaaggtggtgaacgccatcatccgccgctacggctcaccgaacagcgtgcacattgagctggcccgcgagctgagcaagagccacgacgagcgcacaaagatcatgaaggcccacgacgagaactacaagaagaacaagggcgccatcagcatcctgatcgagaacggcatcctgaacccaacaggctacgacatcgtgaggtacaagctgtggaaggagcagggcgagaggtgcgcctactctctgaagcagatcccagccaatacattcttcaacgagatgaagaaggagaggtcaggctctccagtgctggagattgatcacatcctgccatacagccagagcttcatcgacagctaccacaacaaggtgctggtgtacggcgatgagaaccagaagaagggcaatcgcatcccatacacatacttcctggagggcaacaaggattgggagagcttcgagagctacgtgaggctgaacagcttcttcagcaagaagaagaggggctacctgctgaagaaggcctacctgccaagggagagcaacatgatcaaggagaggcacctaaacgatacaaggtacgccagcagctacctgaagaacttcatcgagaagaacctgaagttcaaggaggtggagggcagcacccgtaagaagcacgtccagaccgtgaacggcatcatcacagcccacctgaggaagaggtggggcctggagaaggataggcaggagacatacctgcatcacgccatggatgccatcatcgtggcttgcacagatcaccacatggtgacaaaggtgacagagtactaccagatcaaggagagcaacaagtcaatccgcaagccatacttcccactgccatgggtgggcttcagggaggagatcctgtctcacctggccaggcagccaatcgccaggaagatctctgaggagctgaagatcggctaccagtctttcgattacatcctggtgtctaggatgccaaagaggtctgtgacaggcgccgcccacgagcagacaatcatgaagaagggcggcatcgataagaagggcaagacaatcatcatcaagagggtgtacctgaaggatatcaagttcgatgagaacggcgatttcaagatggtgggcaaggagcaggatctagccacatacgaggccatcaagcagaggtacatcgagtacggcaaggagtctaagaaggccttcgagacaccactgtacaagccatctaagaagggcaagggcaacctgatcaagaagatcaaggtggaggttcagaccaagagcttcgtgagggaggtgaatggcggcgtggcccagaatggcgatctggtgagggtggatctgttcgagaaggataacaggtactacatgatcccgatctacgtgatggatacagtgcacagcgagctgccaaacaaggccgtgacatcttctaagggctacgagcagtggctaaccatcgacaacagcttcaccttcaagttcagcctgtacccatacgacctggtgaggctggtgaagggcaatgaggacaggttcctgtacttcagcaccctggacatcaacagcgacaggctgaacttcaaggacgtgaacaagccatctaagcaggccgagaacaggtacagcctgaagaccatcgagaacctggagaagtacgaggtgggcgtgctgggcgatctgaggttcgtgaggcaggagatcaggaagaatttc

SEQ ID NO: 171 - DNA, 3213 bp, synthetic construct
  misc_feature 1..3213: note = source = /note="Description of Artificial Sequence: Syntheticpolynucleotide"
  misc_feature 1..3213: note = source = /note="APG07433.1 Soy optimized"
  source 1..3213: mol_type = other DNA
atgagagagctagattacaggatcggactcgatatcggaactaattcaattggatggggcgtgattgagctttcctggaataaagatagggagcgttatgaaaaggttaggattgtggatcagggagttaggatgtttgatagagctgagatgcctaagactggagcttcattagctgaaccaagaaggattgctagatctagcaggaggagattaaacaggaagtctcagcgcaagaagaatataaggaaccttcttgttcagcacggagttattactcaggaggagcttgattctctctaccctctctccaagaaatcaatggatatctggggaattaggcttgatggactcgatcgcctattaaaccattttgagtgggctaggcttctaatacaccttgctcaaagaaggggcttcaaatcaaaccgcaagtccgaattaaaggatactgagactggaaaggtgctttcaagcatccagctcaatgaaaagaggctttctttgtataggactgttggggagatgtggatgaaagatcccgacttctcaaaatacgataggaagaggaactctcctaacgagtacgtgttttctgtttctagagctgagcttgagaaggagattgtgactttgtttgctgctcaaaggaggttccaatctccttacgcttctaaggatcttcaagagacctaccttcagatttggactcatcagcttccttttgcttccgggaacgctattctcaataaagtgggatactgctccttattaaagggaaaggagagaaggattcctaaggccacctatacattccagtacttctctgctcttgatcaagtgaatagaactaggcttggacctgatttccagccttttactaaggagcagcgcgagatcatcctcaataatatgttccagaggaccgactactataaaaagaagaccattcccgaggtgacctactacgatataagaaagtggcttgagcttgatgaaaccattcagttcaagggattaaactacgaccccaacgaggagctgaagaaaatagagaagaagcccttcataaacctcaaggccttctacgaaatcaacaaggtggtggctaactactctgagcgaaccaatgaaaccttctctaccctcgattacgatggaattggatacgctcttaccgtgtataaaaccgacaaggatataaggtcctacctcaaatcaagccataatctccctaagaggtgctacgacgatcaattaattgaggagcttctctccctctcctatactaagttcgggcacctctctcttaaggcaatcaaccatgttctaagcattatgcagaaggggaacacatataaggaggctgttgatcagcttggatacgatacttctggacttaagaaggagaagaggtctaagttcctccctcctatttctgatgagattaccaaccctattgtgaagagagctttgactcaagctaggaaggtggtgaatgctattattaggaggcatggatctcctcattctgtgcatattgagcttgctagggagctttcaaagaatcacgatgagaggactaagattgtttctgctcaggacgagaactacaagaagaacaagggggctatttctattctctctgagcacggaattcttaaccctaccggatacgatattgtgaggtataagttgtggaaggagcaaggagagagatgcgcttactctcttaaggaaattcctgctgataccttcttcaacgagcttaagaaggaaaggaacggtgctcctattctagaggtggatcacattcttccttactcccagtccttcattgattcttaccataacaaggtgcttgtgtattccgatgagaacaggaagaagggaaacaggattccttacacctacttccttgagactaataaggattgggaggcttttgagaggtacgtgaggtctaacaagttcttctctaagaagaagagggagtaccttcttaagagggcttatcttcctagggagtcagagcttattaaggagaggcatcttaacgataccaggtacgcttctaccttcctcaagaacttcattgagcagaaccttcaattcaaggaagctgaggataatcctagaaagaggagggttcagactgtgaacggagttattactgctcattttagaaagaggtggggacttgagaaggataggcaagagacttaccttcatcacgctatggatgctattattgtggcttgcactgatcatcatatggtgactagggttaccgagtactaccagatcaaggagtcaaacaagtctgtgaagaagccttattttcctatgccttgggaaggattcagggatgagcttctttctcatcttgcttctcagcctatcgctaagaagatttctgaagagcttaaggctggataccagtcccttgattacattttcgtgtctagaatgcctaagaggtctattactggagctgctcacaagcagactattatgaggaagggaggaattgataagaaggggaagaccatcattattgagaggctccacctcaaggatatcaagttcgatgagaacggagatttcaagatggttggaaaggagcaggatatggctacttacgaggctattaagcagagatatctagagcacggaaagaactctaagaaggctttcgagactcctctttacaagccttctaagaagggaaccggaaaccttattaagagagttaaggttgagggacaggctaagtctttcgttagagaggttaatggaggagttgctcagaatggagatcttgtgagggttgatcttttcgagaaggacgataagtactacatggtgcctatctacgttcctgatactgtttgttccgagcttcctaagaaggttgttgcttcttctaagggatacgagcaatggcttacccttgataactctttcaccttcaagttctctctctacccttacgatcttgttaggctagttaagggagatgaggataggttcctttacttcgggaccctcgatattgattctgataggctcaacttcaaggatgtgaacaagccttctaagaagaacgagtacaggtactctctcaagaccattgaggatcttgagaagtacgaagtgggagttcttggagatcttagacttgttagaaaggagactaggaggaacttccac

SEQ ID NO: 172 - DNA, 3213 bp, synthetic construct
  misc_feature 1..3213: note = source = /note="Description of Artificial Sequence: Syntheticpolynucleotide"
  misc_feature 1..3213: note = source = /note="APG07433.1 Corn optimized"
  source 1..3213: mol_type = other DNA
atgcgcgagctagattacaggatcggcctggatatcggcactaactcaatcggctggggcgtgatcgagctgagctggaataaagatcgcgagcgctatgaaaaggtgcgcatcgtggatcagggcgttaggatgttcgatagggccgagatgccaaagacaggcgcctcactggccgaaccaaggaggatagccaggtcttctaggaggagattaaacaggaagtctcagcgcaagaaaaatatcaggaacctgctggttcagcacggcgtgatcacacaggaggagctggattctctgtacccactgagcaagaaaagcatggacatctggggcattaggctagatggcctggaccgcctattaaaccacttcgagtgggcccgcctattaattcacctggcccagaggaggggcttcaaatcaaataggaagagcgaattaaaggacaccgagacaggcaaggtgctgtcaagcatccagctgaatgaaaagcgcctgagcctgtacaggacagtgggcgagatgtggatgaaagacccggacttctcaaaatacgaccgcaagcgcaacagcccgaacgagtacgtgttctctgtgtctagggccgagctggagaaggagatcgtgacactgttcgccgctcagaggaggttccagtctccatacgcctctaaggatctgcaagagacatacctgcagatctggacacaccagctgccattcgcctctggcaatgccatcctgaataaagtgggctactgctctctattaaagggcaaggagaggaggattccaaaggccacctatacattccagtacttcagcgccctggaccaggtgaataggacaaggctgggcccagatttccagccattcacaaaggagcagcgcgagatcatcctgaataatatgttccagcgcaccgactactataaaaagaagaccatcccggaggtgacatactacgatataaggaagtggctggagctggatgaaaccatccagttcaagggattaaactacgacccgaacgaggagctgaagaaaatagagaagaagccgttcataaacctgaaggccttctacgaaatcaacaaggtggtggccaactacagcgagcgaaccaacgagaccttcagcaccctggactacgacggcatcggctacgccctgaccgtgtataaaaccgacaaggatataaggagctacctgaaatcaagccacaacctgccgaagcgctgctacgacgaccaattaatcgaggagctgctgagcctgagctatactaagttcggccacctgagcctgaaggcaatcaaccacgtgctaagcatcatgcagaagggcaatacatacaaggaggccgtggaccagctgggctacgacaccagcggcctgaagaaggagaagcgcagcaagttcctgccgccgatcagcgacgagatcaccaacccgatcgtgaagcgcgccctgacccaggccaggaaggtggtgaacgccatcatcaggaggcacggctctccacatagcgtgcacatcgagctggccagggagctgtctaagaatcacgatgagaggacaaagatcgtgtcagcccaggacgagaactacaagaagaataagggcgccatctctatcctgtctgagcacggcatcctgaacccaacaggctacgacatcgtgaggtacaagctgtggaaggagcagggcgagaggtgcgcctactctctgaaggagatcccagccgatacattcttcaatgagctgaagaaggagaggaatggcgccccaatcctggaggtggatcacatcctgccatactctcagagcttcatcgattcataccacaacaaggtgctggtgtacagcgacgagaacaggaagaagggcaatcgcatcccatacacatacttcctggagaccaacaaggattgggaggccttcgagaggtacgtgcgcagcaacaagttcttcagcaagaagaagcgcgagtacctgctgaagagggcctacctgcctagggagtctgagctgatcaaggagaggcacctgaatgatacaaggtacgccagcaccttcctgaagaacttcatcgagcagaacctacagttcaaggaggccgaggataacccaaggaagaggagggttcagacagtgaatggcgtgatcacagcccacttcaggaagaggtggggcctggagaaggataggcaggagacatacctgcatcacgccatggatgccatcatcgtggcttgcacagatcaccacatggtgacaagggtgacagagtactaccagatcaaggagagcaacaagagcgtgaagaagccatacttcccaatgccatgggagggcttcagggatgagctactgtctcacctggcctctcagccaatcgccaagaagatctctgaggagctgaaggccggctaccagtctctggattacatcttcgtgtctaggatgccaaagaggtctatcacaggcgccgcccacaagcagacaatcatgaggaagggcggcatcgataagaagggcaagacaatcatcatcgagaggctgcatctgaaggacatcaagttcgatgagaacggcgatttcaagatggtgggcaaggagcaggatatggccacatacgaggccatcaagcagcgctacctagagcacggcaagaactctaagaaggccttcgagacaccactgtacaagccatctaagaagggcaccggcaatctgatcaagagggtgaaggtggagggccaggccaagtctttcgtgagggaggtgaatggcggcgtggcccagaatggcgatctggtgagggtggatctgttcgagaaggatgataagtactacatggtgccaatctacgtgccagatacagtgtgctctgagctgccaaagaaggtggtggcctcttctaagggctacgagcagtggctaacactggataacagcttcacattcaagttcagcctgtacccatacgatctggtgaggctggtgaagggcgatgaggataggttcctgtacttcggcacactggatatcgatagcgacaggctgaacttcaaggacgtgaacaagccgagcaagaagaacgagtaccgctacagcctgaagacaatcgaggacctggagaagtacgaggtgggcgtgctgggcgatctgaggctggtgaggaaggagacaaggaggaatttccac

SEQ ID NO: 173 - DNA, 3213 bp, synthetic construct
  misc_feature 1..3213: note = source = /note="Description of Artificial Sequence: Syntheticpolynucleotide"
  misc_feature 1..3213: note = source = /note="APG07513.1 Soy optimized"
  source 1..3213: mol_type = other DNA
atgagagagctagattacaggatcggactcgatatcggaactaattcaattggatggggcgtgattgagctttcctggaataaagatcgcgagcaatatgaaaagaccaggattgtggataagggagtgaggatgttcgataaggctgaaattcctaagactggagcttctcttgctgaaccaagaagaattgctagatctagcaggaggagattaaacaggaagtctcagcgcaagaaggatataaggaaccttctcgttcagcatgaaatcatttcccagaaggaattaacctctctctaccctctctccaaatcaagcatggacatctgggatataaggcttgatggactcgataggcttcttgatagatttgagtgggctaggcttctaatacaccttgctcaaagaaggggcttcaaatcaaaccgcaagtccgaattaaaggatgttgagactggaaaggtgctttcaagcatccaggtgaatgaaaagaggctttctttgtataggactgttggggagatgtggatgaaaaacgccgattgctcaaaatatggaaagaggaggaactctcctaacgagtacgtgttttctgtttctagggctgatcttgagaaggagattgtgactctttttgaggctcagaggaagttccactcttcttacgcttctgtggatctccaaaagacatacattcagatttgggctcatcagcttccttttgcttctggaaacgccattgtgaataaagtgggatactgctccctattaaagggaaaggagaagagagtgcctaaggctacctatacattccagtacttcaataccctcgatcaaatcaacagaactaggcttggacctaacttccagccttttactaaggagcagagggatataatcctcgataaaatgttccagcgcaccgactactataaaaagaagaccatccccgaggtgacctactacgatataagaaagtggcttgctctcgatgaaaccattcagttcaagggactaacttatgaccccaacgaggagctgaagaaaatagagatgaaacccttcataaacctcaagcccttctacgaaatcaagaaggtggtaaccaactacgccaagaaaaccaacgaggtgttctctgctcttgattacgatactgtggcttacgctcttaccgtgtataaaaccgacaaggatataaggtcttacctcaagcgctccaataatctctctaagaggtgctacgacgatcaattaattgaggagcttctcaccctctcctatactaagttcggacacctctctttcaaggcaatcaaccatgtgcttccaataatgcaagagggaagaacttaccaagaggcaatacaccagcttggatacgatacaaccaacctcaagaaggagaatagaagcatgttcctcccaatcatccctgatgaaataaccaaccctattgtgaagagagctttgactcaagctaggaaggtggtgaatgcaataattaggaggtacggatcacctaactctgtgcatattgagcttgccagggaattaagcaagtctcatgatgagaggaagaagattatgactgctcacgacgagaactacaagaagaacaagggagctgtgtccattctcattgataacggaattcttaaccctaccggatacgatattgtgaggtacaagttgtggaaggagcaaggagagagatgcgcttactctcttaagaagattcctgctaacaccttcttcaacgagcttaagaaggaaagatctggacctcctgttcttgaggtggatcacattctaccttactcccagtccttcattgattcttaccataacaaggtgcttgtttacggagatgagaaccaaaagaaggggaacaggattccttacactttcttctctgaagaagataaggagtgggagtctttcgagtcttacgtgaggtccaactccttcttctctaagaagaagaggggataccttcttaagaaggcttatcttcctagggagtctaaccttattaaggagaggcaccttaacgataccaggtacgcttcatcttacctcaagaacttcatcgagaagaaccttaagttcaaggaggctgtgggaattactaggaagaagtacgttcagactgtgaacggagtgattactgctcatcttagaaagagatggggacttgagaaggataggcaagagacttaccttcatcacgctatggatgctattattgtggcttgcactgatcaccatatggtgactaaggttaccgagtactaccaaattaaggaggggaacaagtccatcaagaagccttattttcctctaccttggatgggattcagggaggagattctttctcatcttgagtctcaacctatcgctagaaagatttccgaggagcttaagattggataccagtcccctgattacattcttgtgtctagaatgcctaagaggtctgttactggatctgctcatgatcagactgtgatgaagaagggggatattgataagaaggggaagaccatcattattaagagggtgcacctcaaggacatcaagttcgatgagaacggagatttcaagatggttggaaaggagcaggatctagctacttacgaggctattaagcagaggtatcttgagtataggaaggagtctaagaaggctttcgagactcctctttacaagccttctaagaagggaaaggggaacctcatcaagaagattaaggttgaggttcagactaagtctttcgtgagggagattaatggaggagttgctcagaatggagatcttgtgagggttgatcttttcgagaaggacaacaggtactacatggtgcctatctacgttgttgatactgttaggtctgagctacctaacaaggctgttacttcttctaagggatacgagcagtggctctctattgataactctttcaccttcaagttctctctctacccttacgatcttgttaggcttgttaagggagatgaggataggttcctctacttctccaccctcgatattaactctgataggctcaacttcaaggatgtgaacaagccttctaagcaagctgagtacaggtactctcttaagaccattgagaaccttgagaagtacgagattggagttcttggagatctaaggcttgttaggcaggagaccaggaagattttcaag

SEQ ID NO: 174 - DNA, 3213 bp, synthetic construct
  misc_feature 1..3213: note = source = /note="Description of Artificial Sequence: Syntheticpolynucleotide"
  misc_feature 1..3213: note = source = /note="APG07513.1 Corn optimized"
  source 1..3213: mol_type = other DNA
atgcgcgagctagattacaggatcggcctggatatcggcactaactcaatcggctggggcgtgatcgagctgagctggaataaagatcgcgagcagtatgaaaagaccaggatcgtggacaagggcgtgaggatgttcgataaggccgagatcccaaagacaggcgcctctctggccgaaccaaggaggatcgccaggtcttctaggaggagattaaacaggaagtctcagcgcaagaaggatataaggaacctgctggtccagcatgaaatcatcagccagaaggagctgacaagcctgtacccgctgagcaagtcaagcatggacatctgggatataaggctggatggcctggataggctgctggataggttcgagtgggccaggctgctgatacatctggcccagaggaggggcttcaaatcaaatcgcaagagcgaattaaaggacgtggagacaggcaaggtgctgtcaagcattcaggtgaatgaaaagaggctgagcctgtacaggacagtgggcgagatgtggatgaaaaacgccgactgctcaaaatacggcaagcgccgcaacagcccaaacgagtacgtgttctctgtgtctagggccgatctggagaaggagatcgtgacactgttcgaggcccagaggaagttccactcttcttacgcctctgtggacctgcagaagacatacatccagatctgggcccaccagctgccattcgcctctggcaatgccatcgtgaataaagtgggctactgctctctattaaagggcaaggagaagagggtgccaaaggccacctatacattccagtacttcaataccctggaccaaatcaacaggacccgcctgggcccaaacttccagccattcacaaaggagcagcgcgatataatcctggataaaatgttccagcgcaccgactactataaaaagaagaccatcccggaggtgacatactacgatataaggaagtggctggccctggatgaaaccatccagttcaagggcctaacctacgacccgaacgaggagctgaagaagatcgagatgaaaccgttcataaacctgaagccgttctacgaaatcaagaaggtggtaaccaactacgccaagaaaaccaacgaggtgttcagcgccctggactacgacaccgtggcctacgccctgaccgtgtataaaaccgacaaggatataaggagctacctgaagcgcagcaataatctgagcaagcgctgctacgacgaccaattaatcgaggagctgctgaccctgagctatactaagttcggccacctgagcttcaaggcaatcaaccacgtgctgccaataatgcaggagggccgcacctaccaggaggcaatacaccagctgggctacgacacaaccaacctgaagaaggagaaccgaagcatgttcctgccaataatcccggacgagataaccaacccgatcgtgaagcgcgccctgacccaggcccgcaaggtggtgaacgcaataatccgccgctacggctcaccgaacagcgtgcacattgagctggcccgcgaattaagcaagagccacgacgagcgcaagaagatcatgaccgcccacgatgaaaactacaagaaaaataagggcgccgtgagcatcctgatcgacaacggcatcctgaacccgaccggctacgacatcgtgcgctacaagctgtggaaggagcagggcgagaggtgcgcctactctctgaagaagatcccagccaacaccttcttcaacgagctgaagaaggagaggtctggcccaccagtgctggaggtggatcacatcctgccatactcacagagcttcatcgatagctaccacaacaaggtgctggtgtacggcgatgagaaccagaagaagggcaatcgcatcccatacacattcttcagcgaggaggataaggagtgggagagcttcgagagctacgtgaggagcaacagcttcttcagcaagaagaagaggggctacctgctgaagaaggcctacctgccaagggagtctaacctgatcaaggagaggcacctgaacgatacaaggtacgccagcagctacctaaagaacttcatcgagaagaacctgaagttcaaggaggccgtgggcatcacccgcaagaagtacgtccagacagtgaacggcgtgatcacagcccacctgaggaagaggtggggcctggagaaggataggcaggagacatacctgcatcacgccatggatgccatcatcgtggcttgcacagatcaccacatggtgacaaaggtgacagagtactaccagatcaaggagggcaacaagagcatcaagaagccatacttcccactaccatggatgggcttcagggaggagatcctgtctcacctggagtctcagccaatcgccaggaagatctctgaggagctgaagatcggctaccagtctccagattacatcctggtgtctaggatgccaaagaggtctgtgacaggctctgcccacgatcagacagtgatgaagaagggcgatatcgacaagaagggcaagaccatcatcatcaagagggtgcacctgaaggacatcaagttcgacgagaacggcgacttcaagatggtgggcaaggagcaggatctagccacatacgaggccatcaagcagcgctacctggagtacaggaaggagtctaagaaggccttcgagacaccactgtacaagccatctaagaagggcaagggcaacctgatcaagaagatcaaggtggaggtccagaccaagagcttcgtgagggagatcaatggcggcgtggcccagaatggcgatctggtgagggtggatctgttcgagaaggataacaggtactacatggtgccaatctacgtggtggatacagtgaggagcgagctaccaaacaaggccgtgacatcttctaagggctacgagcagtggctgagcatcgacaacagcttcaccttcaagttcagcctgtacccatacgatctggtgaggctggtgaagggcgatgaggacaggttcctgtacttcagcacactggacatcaacagcgacaggctgaacttcaaggacgtgaacaagccatctaagcaggccgagtacaggtacagcctgaagaccatcgagaacctggagaagtacgagatcggcgtgctgggcgatctaaggctggtgaggcaggagacaaggaagatcttcaag

SEQ ID NO: 175 - DNA, 3216 bp, synthetic construct
  misc_feature 1..3216: note = source = /note="Description of Artificial Sequence: Syntheticpolynucleotide"
  misc_feature 1..3216: note = source = /note="APG08290.1 Soy optimized"
  source 1..3216: mol_type = other DNA
atgtctgagctagattacaggatcggactcgatatcggaactaattcaattggatggggagtgattgagcttttctggaataaagatcgcgagcgctatgaaaaggttaggattgtggataagggagtgaggatgttcgataaggccgagattcctaataaaggagcttcacttgctgaaccaagaaggattgctagatctagcaggaggagattaaacaggaagtctcagaggaagaaggagattaggaaccttcttgttcagcacggaatgattactcaggaggagcttgatcttctctaccctctctccaagaaatcaatcgatatctgggatataaggcttgatggactcgataggctattaaaccatcttgagtgggctaggcttctaatacaccttgctcaaagaaggggcttcaaatcaaaccgcaagtccgaattaaaggatgctgagactggaaaggttctttcaagcatccaggtgaatgaaaagaggcttttcttgtataggactgtgggcgagatgtggataaaagatgccgagttctcaaaatatgataggaggaggaactctcctaacgagtacgtgttttctgtttctagggctgatcttgagaaggagattgtgactttgtttgaggctcaacgcaagttccaatcaagctacgcttctaagaaccttcaagagacctaccttcaaatttgggctcatcagcttccttttgcttccgggaacgctattctcaataaagtgggatactgctccttattaaagggaaaggagagaaggattcctaaggccacctatacattccagtacttctctgctcttgatcaagtgaatagaactaggcttggacctgatttccaacctttcactcaggagcagaaggaaataatcctcgataaaatgttccagcgcaccgactactataaaaagaagaccatccccgaggtgtcctactacgatataagaaagtggcttgagcttgatgaaaccattcagttcaagggattaaactacgaccccaacgaggagctgaagaaaatagagaagaagcccttcataaacctcaaggccttctacgaaatcaagaaggtggttgctaactacgctgaacgaaccaacgaagcattctctaccctcgattacgatgctattgcttacgctcttaccgtgtataaaaccgacaaggatataaggtcctacctcaagaaatcaaacaacctctccaagaggtgctacgacgatcaattaatcgaggagcttttcaccctctcctatactaagttcggacacctctctttcaaggcaatcaaccatgtgcttcctattatgcaagagggaagaacttaccaagaggctattcatcagcttggatacgataccaccaaccttaagaaggagaacaggtctatgttcctccctctcattcctgatgagattactaaccctattgtgaagagggctattactcaagctaggaaggtggtgaatgctattattaggaggtacggatcacctaactctgtgcatattgagcttgctagggagctttctaagtctcatgatgagaggaagaagattatgactgctcacgacgagaactacaagaagaacaagggggctatttccattctcattgagaacggaattcttaaccctaccggatacgatattgtgaggtacaagttgtggaaggagcaaggagagagatgcgcttactctcttaaggaaattcctcctgataccttcttcaacgagcttaagaaggagaggaacggatcacctattcttgaggtggatcacattcttccttactcccagtccttcattgattcttaccataacaaggtgcttgtgtattccgatgagaacaggaacaagggaaacaggattccttacacctacttccttgagactaataaggattgggaggcttttgagaggtatgtgaggtctaacaagctcttctctaagaagaagagggagtacctccttaagaagacttaccttcctagggagtctgagcttattaaggagaggcatctaaacgataccaggtacgcttctaccttcctcaagaacttcattgagcagaaccttcaattcaaggaggttgaggttaaccttaggaagaagagggttcagactgtgaacggagttattactgctcatcttagaaagagatggggacttgagaagaacaggcaagagacttaccttcatcacgctatggatgctattattgtggcttgcactgatcatcacatggtgactaggattaccgagtactaccagattaaggagtcaaacaagtctgtgaagaagccttattttcctatgccttgggagggattcagggatgagcttctttctcatcttgcttctcagcctatcgctaagaagatttctgaagagcttaaggctggataccagtcctctgattacattttcgtgtctagaatgcctaagaggtctgttactggagctgctcatgatcaaactattaggaggaagggaggaattgataagaaggggaagaccatcattattaagagggtgaggctaaaggacatcaagttcgatgagaacggggatttcaagatggttggaaaggagcaggatcttgctacttacgaggctattaagcagagatatcttgagcacaggaagaactctaagaaggctttcgagactcctctttacaagccttctaagaagggaaccggaaaccttatcaagagagttaagattgagggacagactaaggctttcgttagagaggttaatggaggagttgctcagaactctgatcttgtgagggttgatctattcgagaaggacgataagtactacatggtgcctatctacgttcctgatactgtttgttccgagcttcctaagaaggttgttaagtctggaaagggatacgagcaatggcttacccttgataactctttcaccttcaagtcttctctctacccttacgatcttgttaggcttgttaagggaaacgaggataggttcctttacttcgggaccctcgatattgattctgataggctcaacttcaaggatgtgaacaagccttcaaagcaaaacgagtacaggtactctctcaagaccattgagaaccttgagaagtacgaagtgggagttcttggagatcttaggcttgttaagcaagagaccaggaggattttcaatagg

SEQ ID NO: 176 - DNA, 3216 bp, synthetic construct
  misc_feature 1..3216: note = source = /note="Description of Artificial Sequence: Syntheticpolynucleotide"
  misc_feature 1..3216: note = source = /note="APG08290.1 Corn optimized"
  source 1..3216: mol_type = other DNA
atgtcagagctggactacaggatcggcctggacataggcactaattcaatcggctggggcgtgatcgagctgttctggaataaagaccgcgagcgctatgaaaaggtgcgcatcgtggacaagggcgttaggatgttcgataaggccgagatcccgaataaaggcgcctcactagccgaaccaaggaggatcgccaggtcttctaggaggagattaaacaggaagtctcagaggaagaaggagatcaggaacctgctggtccagcacggcatgatcacacaggaggagctggatctgctgtacccactgagcaagaaaagcattgacatctgggacatcaggctagatggcctggacaggctgctgaatcacctggagtgggccaggctgctgatacatctggcccagaggaggggcttcaaatcaaatcgcaagtctgaattaaaggatgccgagacaggcaaggtgctgtcaagcatccaggtgaatgaaaagaggctgttcctctaccgcacagtgggcgagatgtggataaaagacgccgagttcagcaagtacgacaggaggaggaacagcccaaacgagtacgtgttctctgtgtcaagggccgatctggagaaggagatcgtgacactgtttgaggctcagcgcaagttccaatcaagctacgccagcaagaacctgcaagagacctacctgcagatctgggcccaccagctgccattcgctagcggcaatgccatcctgaataaagtgggctactgctctctattaaagggcaaggagaggaggattccaaaggccacctatacattccagtacttcagcgccctggaccaggtgaataggacaaggctgggcccagatttccagccattcacacaggagcagaaggaaataatcctggataaaatgttccagcgcaccgactactataaaaagaagaccatcccggaggtgtcatactacgatataaggaagtggctggagctggatgaaaccatccagttcaagggattaaactacgacccgaacgaggagctgaagaaaatagagaagaagccgttcataaacctgaaggccttctacgaaatcaagaaggtggtggccaactacgccgagcgaaccaacgaagcattcagcaccctggactacgacgccatcgcctacgccctgaccgtgtataaaaccgacaaggatataaggtcatacctgaagaaatcaaacaacctgagcaagcgctgctacgacgaccaattaatcgaggagctgttcaccctgagctatactaagttcggccacctgagcttcaaggcaatcaaccacgtgctgccaataatgcaggagggccgcacctaccaggaggcaatacaccagctgggctacgacaccaccaacctgaagaaggagaaccgcagcatgttcctgccgctaatcccggacgagatcaccaacccgatcgtgaagcgcgccatcacccaggctaggaaggtggtgaacgccatcatccgccgctacggcagcccgaacagcgtgcacatcgagctggcccgcgagctgagcaagagccacgacgagcgcaagaagatcatgacagcccacgacgagaactacaagaagaacaagggcgccatctcaatcctgatcgagaacggcatcctgaacccaaccggctatgacatcgtgcgctacaagctgtggaaggagcagggcgagaggtgcgcctactctctgaaggagatcccaccagatacattcttcaacgagctgaagaaggagaggaatggctctccaatcctggaggtggatcacatcctgccatacagccagtcattcatcgacagctaccacaacaaggtgctggtgtacagcgatgagaacaggaacaagggcaaccgcatcccgtacacatacttcctggagaccaacaaggattgggaggccttcgagaggtacgtgaggagcaacaagctgttcagcaagaagaagcgcgagtacctgctgaagaagacctacctgccaagggagtcagagctgatcaaggagaggcacctgaacgatacaaggtacgctagcaccttcctgaagaacttcatcgagcagaacctgcagttcaaggaggtggaggtgaacctgaggaagaagcgcgttcagacagtgaacggcgtgatcacagcccacctgaggaagaggtggggcctggagaagaataggcaggagacatacctacaccacgccatggatgccatcatcgtggcttgcacagatcaccacatggtgacaaggatcacagagtactaccagatcaaggagagcaacaagagcgtgaagaagccatacttcccaatgccatgggagggcttcagggatgagctgctgtctcacctggcctctcagccaatcgccaagaagatctcagaggagctgaaggccggctaccagtcttctgattacatcttcgtgtctaggatgccaaagaggtctgtgacaggcgccgcccacgatcagacaatcaggaggaagggcggcatcgataagaagggcaagacaatcatcatcaagagggtgaggctgaaggatatcaagttcgatgagaacggcgatttcaagatggtgggcaaggagcaggatctagccacatacgaggccatcaagcagcgctacctggagcacaggaagaactctaagaaggccttcgagacaccactgtacaagccatctaagaagggcaccggcaatctgatcaagagggtgaagatcgagggccagacaaaggccttcgtgagggaggtgaatggcggcgtggcccagaattctgatctggtgagggtggatctattcgagaaggacgataagtactacatggtgccaatctacgtgccagatacagtgtgctccgagctgccaaagaaggtggtgaagtctggcaagggctacgagcagtggctcacactggataacagcttcacattcaagagcagcctgtacccatacgatctggtgaggctggtgaagggcaatgaggacaggttcctgtacttcggcacactagacatcgatagcgacaggctgaacttcaaggacgtgaacaagccaagcaagcagaacgagtaccgctacagcctgaagaccatcgagaacctggagaagtacgaggtgggcgtgctgggcgatctgaggctggtgaagcaggagacaaggaggatcttcaatagg

SEQ ID NO: 177 - DNA, 4011 bp, synthetic construct
  misc_feature 1..4011: note = source = /note="Description of Artificial Sequence: Syntheticpolynucleotide"
  misc_feature 1..4011: note = source = /note="APG05459.1 Soy optimized"
  source 1..4011: mol_type = other DNA
atgaagaaggactacgtgatcggactagatattggaactaactctgtgggatgggctgttatgactgaggattaccagctagttaagaagaagatgcccatctacgggaatacagagaagaagaagatcaagaagaacttctggggagtgagactatttgaagagggtcatactgctgaggataggagattaaagagaactgctaggagaaggatttctaggaggaggaacaggcttcgttaccttcaagcattctttgaagaggctatgaccgctctagatgaaaatttcttcgctaggcttcaggagtcttttcttgtgcctgaggataaaaagtggcacaggcatcctatttttgctaagttagaggatgaggtggcttaccatgaaacctaccctactatctaccaccttaggaagaagttggctgattcttcagagcaagccgacttgagattaatctaccttgctcttgcccacattgtaaaatataggggacacttccttatcgagggaaaactctccaccgaaaatatttcagtgaaggagcagtttcagcagttcatgatcatctataaccaaaccttcgtgaacggagaatcaaggcttgtgtctgctcctcttcctgagtccgtattaattgaggaggaacttactgagaaagcatccagaaccaagaagtctgagaaggttcttcaacagtttcctcaagagaaggctaacggacttttcggacagttcctcaaattaatggtggggaataaagccgacttcaagaaggtttttggacttgaggaggaggccaaaataacttacgcttctgagtcttatgaggaggatcttgagggaatactagctaaggtgggagatgaatacagcgatgttttccttgctgctaagaacgtttacgacgctgtggaattaagcaccatactagctgactccgataaaaagtctcacgccaaactctcttcaagcatgattgttaggttcactgagcatcaggaggatcttaagaagttcaagaggttcattagggagaactgccctgatgagtacgataaccttttcaagaacgagcaaaaggatgggtacgctggatacattgctcatgctggaaaggtgtcacaattaaagttctaccagtacgtgaagaaaataatccaggatatcgctggagccgagtacttccttgagaaaatagctcaggagaacttccttaggaagcagaggactttcgataacggagtgattcctcaccaaatacaccttgctgagctacaggcaataattcataggcaagctgcatactatcctttcctcaaggagaatcaagagaaaatagagcagctcgtgaccttcagaattccatactacgtgggacctctttctaagggagatgcttctacttttgcttggcttaagagacagtcagaggaaccaattaggccttggaatcttcaagagactgtggatcttgatcaatctgctactgctttcatagagaggatgaccaacttcgatacataccttccttctgagaaggtgctccctaagcattctctcctctatgaaaagttcatggtgttcaacgagctaaccaaaatatcctacaccgacgataggggaatcaaggctaatttctctggaaaggagaaggagaaaatattcgactacctcttcaagaccaggaggaaggtgaagaagaaggacataatacagttctaccgcaacgaatacaacaccgagattgtgaccttgtcaggacttgaggaggatcaattcaacgcttctttctctacctaccaggatctattaaagtgcggacttactagggctgagcttgatcatcctgataatgctgagaagctggaggacataatcaagatactaaccatcttcgaggataggcaaaggattaggacccagctttctactttcaagggacaattctctgctgaggttcttaagaagttggagaggaagcattatactggatggggaaggctctctaagaagctcattaacggaatctacgataaggagtcagggaagaccatcctagattacctcattaaggatgatggagtgtctaagcactacaacaggaacttcatgcagctcattaacgattcccagctctccttcaagaacgctattcaaaaggctcaatcttctgagcatgaagagactctttctgagactgttaacgaattagctgggtcccctgctattaagaagggaatctaccagtctctcaagattgtggatgagcttgtggctattatgggatatgctcctaagaggattgttgttgagatggctagggagaaccaaactacttctactggaaagaggaggtctattcagaggctaaagattgttgaaaaggctatggctgagattggatctaaccttcttaaggagcagcctactactaacgagcaacttagggataccaggctcttcctttactacatgcagaacggaaaggatatgtacactggagatgagctttctcttcataggctctcacactacgacatagatcacattatccctcagtccttcatgaaggatgattctctcgataaccttgtgcttgtgggatctactgagaataggggaaagtctgatgatgttccttctaaggaggtggttaaggatatgaaggcttactgggagaaactatacgctgctggacttatttctcaaaggaagttccagagacttactaagggagagcaaggaggacttactcttgaggataaggctcatttcattcaaaggcagcttgttgagaccaggcagattactaagaacgtggctggaattctagatcaaaggtacaacgctaactctaaggagaagaaggttcagatcattaccctcaaggcttctcttacctcccagttcaggtctattttcggactttacaaggttagggaggtgaacgattaccatcatggacaagatgcttaccttaactgcgtggttgctactactctacttaaggtttatcctaaccttgctcctgagttcgtttacggagagtatcctaagttccaggctttcaaggagaataaggctaccgctaagaccattatctacaccaaccttatgaggttctttactgaggatgagcctaggtttatgaaggatggagagattctctggtcaaactcctacctcaagaacatcaagaaggagcttaactaccaccagatgaacattgtgaagaaggtggaggttcaaaagggaggattctccaaggagtctattaagcctaagggaccttctaacaagctcattcctgtgaagaacggacttgatcctcaaaagtacggaggattcgattcacctgttgttgcttacactgttcttttcactcacgaaaaggggaagaagcctctcattaagcaggagattctcggaattaccattatggagaagactaggttcgagcagaaccctattctttttctagaggagaagggatttcttaggcctagggttcttatgaagctccctaagtacactctttatgagtttcctgagggaagaaggagacttcttgcttctgctaaggaggctcaaaagggaaaccaaatggttcttcctgagcaccttcttactcttctttaccatgctaagcaatgcctacttcctaaccaatctgagtctcttgcttacgtagaacaacatcaacctgagttccaagagattcttgagagggttgtggatttcgctgaggttcatacccttgctaagtctaaggttcagcagattgttaagttgttcgaggctaaccagactgctgatgttaaggagatcgctgcttcattcattcagcttatgcaattcaatgctatgggtgctccttctaccttcaagttcttccagaaggatatagagagggctaggtacacctccatcaaggagattttcgatgctaccattatctaccagtctactactggactttatgagactaggaggaaggtggttgat

SEQ ID NO: 178 - DNA, 4011 bp, synthetic construct
  misc_feature 1..4011: note = source = /note="Description of Artificial Sequence: Syntheticpolynucleotide"
  misc_feature 1..4011: note = source = /note="APG05459.1 Corn optimized"
  source 1..4011: mol_type = other DNA
atgaaaaaggactacgtgatcggcctggacatcggcactaattcagtgggctgggccgtgatgacagaggactaccagctggtgaagaagaagatgcctatctacggcaacactgagaagaagaagatcaagaagaacttctggggcgtgcgcctattcgaggagggccacacagccgaggaccgcagattaaagaggacagccaggaggaggatctctaggaggaggaataggctgcgctacctgcaagcattcttcgaggaggccatgacagccctggatgaaaatttcttcgccaggctgcaggagtctttcctagtgccggaggataaaaagtggcacaggcaccctatcttcgccaagctggaggatgaggtggcctaccatgaaacatacccaacaatctaccacctgaggaagaagctggccgattcttctgagcaggccgacctgagattaatctacctggccctggcccacatcgtcaaatacaggggccacttcctgatcgagggcaaactgagcacagaaaatatcagcgtgaaggagcagttccagcagttcatgatcatctataaccaaaccttcgtgaacggcgaatcaaggctggtgagcgccccactgccggagagcgtattaatcgaggaggaactgaccgagaaagcatcccgaaccaagaagagcgagaaggtgctgcagcagttcccgcaagagaaggctaatggcctgttcggtcagttcctgaaactgatggtgggcaataaagccgacttcaagaaggtgttcggcctagaggaggaggccaaaataacctacgccagcgagagctacgaggaggacctggagggcatactagccaaggtgggcgatgaatacagcgatgtgttcctggccgccaagaatgtgtacgatgccgtggagctgtctacaatactagccgacagcgataaaaagagccacgccaagctctcttcaagcatgatcgtgaggttcacagagcatcaggaggacctgaagaagttcaagcgcttcatcagggagaactgcccggacgagtacgacaacctgttcaagaacgagcagaaggatggctacgccggatacatcgcccacgccggtaaggtgtcccaattaaagttctaccagtacgtgaagaaaataatccaggacatcgccggcgccgagtacttcctggagaaaatagcccaggagaacttcctgcgcaagcagaggacattcgacaacggcgtcatcccgcaccagatacatctggccgagctgcaggccatcatacataggcaggccgcatactacccattcctaaaggagaaccaagagaaaatagagcagctggtgaccttccgcatcccatactacgtgggcccactgtctaagggcgatgcctctacattcgcctggctgaagaggcagtctgaggaaccaatcaggccatggaatctgcaagagacagtggatctggatcagtctgccacagccttcatcgagaggatgaccaacttcgatacatacctaccaagcgagaaggtgctgccaaagcatagcctgctgtatgaaaagttcatggtgttcaacgagctaaccaagatcagctacaccgacgaccgcggaatcaaggccaacttcagcggcaaggagaaggagaaaatattcgactacctgttcaagacccgccgcaaggtgaagaagaaggacataatacagttctaccgcaacgaatacaacaccgagatcgtgaccctgagcggccttgaggaggaccagttcaacgccagcttctcaacctaccaggacctattaaagtgcggcctgacccgcgccgagctggaccacccggataacgccgagaagctggaggacataatcaagatactaaccatcttcgaggaccgccagcgcatccgcacccaattaagcaccttcaagggccagttcagcgccgaggtattaaagaagctggagcgcaagcattacacaggctggggcaggctgagcaagaagctaatcaacggcatctacgacaaggagagcggcaagaccatccttgactacctgatcaaggacgatggcgtgagcaagcactacaaccgcaacttcatgcagctgatcaacgacagccagctgagcttcaagaacgccatccagaaggcccagtcttctgagcacgaggagacactgtctgagacagtgaatgagctggccggctcaccagccatcaagaagggcatctaccagtctctgaagatcgtggatgagctggtggccatcatgggctacgccccaaagaggatcgtggtggagatggccagggagaatcagacaacatcaacaggcaagaggaggtctatccagaggctgaagatcgtggagaaggccatggccgagatcggctctaatctgctgaaggagcagccaacaacaaatgagcagctgagggatacaaggctgttcctgtactacatgcagaatggcaaggatatgtacacaggcgatgagctgtctctgcataggctgagccactacgacatcgatcacatcatcccgcagtcattcatgaaggacgactctctggataatctggtgctggtgggctctacagagaacaggggcaagagcgatgatgtgccatctaaggaggtggtgaaggatatgaaggcctactgggagaagctgtacgccgccggcctgatctctcagaggaagttccagaggctgacaaagggcgagcagggcggcctgacactggaggataaggcccacttcatccagaggcagctagtggagacaaggcagatcacaaagaatgtggccggcatcctggatcagaggtacaatgccaattctaaggagaagaaggttcagatcatcacactgaaggcctctctgacatctcagttcaggtctatcttcggcctgtacaaggtgcgcgaggtgaatgattaccaccacggccaggatgcctacctgaattgcgtggtggccacaacactactgaaggtgtacccaaatctggccccagagttcgtgtacggcgagtacccaaagttccaggccttcaaggagaataaggccacagccaagacaatcatctacacaaacctgatgaggttcttcacagaggacgagccaaggttcatgaaggatggcgagatcctgtggagcaacagctacctgaagaacatcaagaaggagctgaactaccaccagatgaacatcgtgaagaaggtggaggtccagaagggcggcttctcaaaggagagcatcaagccgaagggcccgagcaacaagctgatcccagtgaagaatggcctggacccacagaagtacggcggctttgattctccagtggtggcctacacagtgctgttcacacacgagaagggcaagaagccactgatcaagcaggagatcctgggcatcacaatcatggagaagacaaggttcgagcagaacccaatcctgttcctggaggagaagggcttcctgaggccaagggtgctgatgaagctaccaaagtacacactgtacgagttcccagagggcaggaggaggctgctggcctctgccaaggaggcccagaagggcaatcagatggtgctgcctgagcacctgctgacactgctgtaccacgccaagcagtgcctgctgccaaatcagtctgagtctctggcctacgtggagcagcaccagccagagttccaggagatcctggagagggtggtggatttcgccgaggtgcacacactggctaagtctaaggttcagcagatcgtgaagctgttcgaggccaatcagacagccgatgtgaaggagatcgccgcctcattcatccagctgatgcagttcaatgccatgggcgccccatctacattcaagttcttccagaaggatatcgagagggccaggtacacatctatcaaggagatcttcgatgccacaatcatctaccagtctacaacaggcctgtacgagacaaggaggaaggtggtggat

SEQ ID NO: 179 - DNA, 4308 bp, synthetic construct
  misc_feature 1..4308: note = source = /note="Description of Artificial Sequence: Syntheticpolynucleotide"
  misc_feature 1..4308: note = source = /note="APG04583.1 Soy optimized"
  source 1..4308: mol_type = other DNA
atggccaaaaatattctaggactcgatctcggaaccaactctattggatgggctcttgttcagcaggactttgaaaataaggaggggaacatactaggaatggggtccagaataattcctatgtctcaggatatactaggagagttcgggaagggtaactctatttctcaaactgctgagagaactggatataggggagttagaaggttaagggagagacatcttcttagaagggagaggcttcatagggtgcttcatcttcttggattcctacctaagcattacgacgagaaaatagacttcactcagaggttcgggaagttcatcaatcaagctgaaccaaagttggctttcgattcagagttcctcttcaaggactccttccatgaaatgctagctgacttcaagcagaatcaacctgagttcctcaaggataaaaacggagaggattgccttgttccttacgactggactatatactacctaaggaagaaagcattgacccagaagatcgaaaaatatgagctggcctggctcatcctcaacttcaatcaaaagaggggatactatcaattaaggggagaggaggagaaggagaaccctaataaactcgtggggttccactctctcaaaatagtggatgttattcctgacgctgaaaccaataaaaagggagagacctggtactcacttcatcttgagaacggatgggtttataggaggtcctccaaaatatctctcgctgattggaaggacaaagtgagggatttcattgtgaccaccgatcttaatgatgatggatcagagaagctcgataaggatggaattgtgaagaggtcttttagggctccttctgctgatgactggaccctattaaagaagaagaccgagaaggatatcgacaactctaataaaaccgtggggacctacatctacgacaacctcctattaaaccccaagcagaaaatcaagggaaagatggttaggaccattgagaggaagttctacaagcaagagctggagcagatattaaagactcagaaggagttccattctgagcttcagtctgagaacttgcttcaagattgcgttagggagctgtacaggaataatgagcaacatcaacagatgcttgaggctaaggatttcgtgcacctcttcctcaacgatataattttctaccagaggcctcttaggtcccagaaatcaagcatttctaattgcacccttgagttccgcaagtccaagaatgaaaacggagaggaggtaatacacagattaaaggtgatcgccaaatcaaacccatactaccaggagttcaggcttcttcaatgggttcagaacctcgctatctatactaaggacgacgataagaacgtaaccaacgagttcctcaaatcaactcaggattgggaggatcttcttaggtggcttcactctaagaaggaaatcaagcaggatgctctaatcaagttcctcattgagaagaagggactcaaggggaaagcattgactattgaggtggcaaaatataggtggaactacgttcaggataaggattaccctggaaatgaaacccgctacctcattcaatcaaggcttgataaggttgagtacgctcctaaggatttcctcacctatgaaaatgaaatggccctctggcatatcatctactcaatcaacgacaagatcgagtatgaaaaggcattaaagtccttcgccaataaaaaggggctcgatgaggtgaccttcgtggaagcattcaagaagttccctcctttcaagtccgattacggatctttctctgagaaggcaatcaagaagctccttccattaatgaggttcggaactcaatggaactgggataacatcgaccaaaatagcaaggagaggattggaaaaatactaaccggggagtacgatgaaaatattaaagggagggttagggagaaggctaggcaccttaactctgagaccgattttcaagcattacctctttggcttgctcagtacgttgtttatggaaggcactctgaagctgatattgctggaaagtggaactctgtggatgatctcaagcagttccttgatgatttcaagcagcattccttgaggaaccctattgtggagcaggttattactgagactcttagggctgttaaggatatttggaacttctacggaaagggggctaaggatttcttctcagaaatacacattgagcttgggagggagatgaaaaataccgctgatgaaaggaagaggattaccaccatggtgaccgacaatgaaaataccaacctcagaatcaaagcattgcttgctgagatggctcttgatcagaacgttgataacgttaggccttactctcctatgcagcaggagattcttaagatctatgaggagggagttctaaacgctgaggagaacatcgatgatgatatcctcaagatttctaagactgctcagccttctgctaccgatcttaagaggtataagttgtggcttgagcaaaagtacaggtctccttacactggacagatgattcctcttaacaagttgttcacccctgagtacgagatcgagcacattattcctcagtctaggtacttcgacgattcaatgtccaacaaggttatttgcgaggctgctgtgaacaagttgaaggataaccagattggactcgtgttcattaagaaccatcatggagaggtggttgatttcggaatgggaaagcaggtgaagattcttgaggtttctgattacgaggatttcgtgaagcagaactataacaagaacaggggaaagaggaacaagctcctcttagaggatatccctgagaagatgattgagaggcagcttaacgataccaggtacatctctaagtacattacccaggtgctttccaacattgttagggatgataaggagggatctaaggatgatggagtgaactccaagaacattgttcctggaaacggaaagattaccaccaggcttaaacaggattggggacttaacgatgtgtggaacgatcttgttcttcctaggttcgagaggatgaacactcttaccaactctaacgatttcacctctaagaacactcacggaaagactattcctactgtgcctattgagctttcaaagggattctccaagaagaggattgatcacaggcatcatgctatggatgctcttgttattgcttgcgctactagggatcatgtgaaccttctcaacaacgagtcttctaagtctgataccaagaggtacgatcttaacaggaagttgaggaagtacgagaaggttgcttacaacgatcctaagactggagagagaattgagaaggaggtgcctaaggattttattaagccttgggagacctttactgaggatactaggacccttcttgagaacatcgtgatttctttcaagcagaacctcagggtgattaacaaggctaccaactactacgagaagattgagaacggaaagaaggtgaaggttgagcagaagggaattaattgggctgttaggaaggctctacataaggagactgtttctggacaggtgcaccttgataggattaaagtggctaagggaaagattcttaccgctactaggaagactcttgatgcttctttcaacgagaagaccattgagtctattaccgataccgggattcagaagattctcctcaactacctcaagtccaaggataacaatcctgaggttgctttctctcctgaaggaattgaggagcttaacaagaacatcaggctctataacgatggaaaggctcaccagcctattcttaaggttagggttttcgagcagggatctaagtttactcttggagagactggaaacaagactactaagtttgttgaggctgctaagggaaccaaccttttcttcggaatctacgaggataagtctggaaagaggtcttacgagaccatccctttgaacattgttattgagaggcaaaagcaaggactacaagctgttcctgagactaacgagaagggaaagcagcttcttttcaccctttctcctaacgatcttgtttatgttcctgaggagggagttttcgatgagaacaacatcaaggtggataggatctacaaggtggtgtctttctctacttaccaatgcttcttcgtgaggaacgatgtgtctacttctgtggttaacaaggttgagtactctgctctcaacaagatggagaagtccatcgacaacatcatgattaaggagaactgcgtgaagttgaacgttgataggctcggaaagatttccaaggct

SEQ ID NO: 180 - DNA, 4308 bp, synthetic construct
  misc_feature 1..4308: note = source = /note="Description of Artificial Sequence: Syntheticpolynucleotide"
  misc_feature 1..4308: note = source = /note="APG04583.1 Corn optimized"
  source 1..4308: mol_type = other DNA
atggccaagaacatactaggcctggacctcggaaccaattctatcggctgggccctggttcagcaggactttgagaacaaggagggcaacatactaggcatgggcagccgaataatccctatgtctcaggacatactgggcgagttcggtaagggcaacagcatctcacagacagccgagaggacaggctaccgcggcgtgaggaggctcagggagaggcacctgcttaggagggagaggctacacagggtgctgcatctgctgggcttcctgccaaagcattacgatgaaaagatagatttcacacagaggttcggcaagttcatcaatcaagccgaaccaaagctagccttcgattctgagttcctgttcaaggacagcttccatgaaatgctggccgacttcaagcagaatcaaccggagttcctgaaggataaaaacggcgaggactgcctagtgccgtacgactggaccatatactacctgcgcaagaaggccctcacccagaagatcgaaaaatacgagctggcctggctgatccttaacttcaaccaaaagcgcggatactaccagctacgcggcgaggaggagaaggagaacccgaataaactggtgggcttccacagcctgaaaatagtggacgtgatcccggacgccgagactaataaaaagggcgagacctggtactcacttcacctggagaacggctgggtgtaccgcaggagcagcaaaatatctctggccgactggaaggataaagtgagggacttcatcgtgaccacagacctgaacgacgatggctcagagaagctggacaaggatggcatcgtgaagaggtcattcagggccccatctgccgatgactggaccctattaaagaagaagaccgagaaggacatcgacaacagcaataaaaccgtgggcacctacatctacgacaacctgctattaaacccgaagcagaaaatcaagggcaagatggtgcgcaccatcgagcgcaagttctacaagcaggagctggagcagatactaaagacccagaaggagttccacagcgagctgcagagcgagaacctgctgcaggactgcgtgcgcgagctgtaccgcaataatgagcagcaccagcagatgctggaggccaaggacttcgtgcacctgttcctgaacgatataatcttctaccagaggccgctcaggagccagaagtcaagcatcagcaattgcaccctggagttccgcaagagcaagaatgaaaacggcgaggaggtgatacatcgattaaaggtgatcgccaagagcaacccatactaccaggagttccgcctgctgcagtgggtccagaacctggccatctatactaaggacgacgacaagaacgtaaccaacgagttcctgaaatcaacccaggactgggaggacctgctgcgctggctccacagcaagaaggaaatcaagcaggacgccctaatcaagttcctgatcgagaagaagggattaaagggcaaagcattgaccatcgaggtggcaaaatacaggtggaactacgtccaggacaaggactacccgggcaatgaaacccgctacctaatacagagccgcctggacaaggtggagtacgccccgaaggacttcctgacctacgagaatgaaatggccctgtggcatatcatctactcaatcaacgacaagatcgagtatgaaaaggcattaaagagcttcgccaataaaaagggcctggacgaggtgaccttcgtggaagcattcaagaagttcccgccgttcaagagcgactacggcagcttcagcgagaaggcaatcaagaagctgctgccattaatgcgcttcggcacccagtggaactgggacaacatcgaccaaaatagcaaggagcgcatcggcaaaatactaaccggcgagtacgacgagaacataaagggccgcgtgcgcgagaaggcccgccacctgaacagcgagaccgacttccaagcattgccgctgtggctggcccagtacgtggtctacggcaggcacagcgaggccgatatcgccggcaagtggaacagcgtggatgatctcaagcagttcctggacgatttcaagcagcacagcctgaggaatccaatcgtggagcaggtgatcacagagacactaagggccgtgaaggacatctggaatttctacggcaagggcgccaaggacttcttcagcgagatacatatcgagctgggccgcgagatgaaaaacaccgccgatgaaaggaagaggatcacaaccatggtgaccgacaatgaaaacaccaacctgcgaatcaaggccctgctggccgagatggccctggatcagaacgtggataacgtgaggccatactctccaatgcagcaggagatcctgaaaatatacgaggagggcgtattaaacgccgaggaaaatatcgacgacgacatcctgaaaataagcaagaccgcccaaccaagcgccaccgacctgaagcgatataagctgtggctggagcaaaaatacaggagcccgtacaccggccagatgatcccgctgaataaactgttcaccccggagtatgaaatcgagcatataatccctcagagccgctacttcgacgacagcatgagcaacaaggtgatctgcgaggccgccgtgaacaagctaaaggacaaccagatcggcctggtgttcatcaagaaccaccacggcgaggtggtggacttcggcatgggcaagcaggtgaagatcctggaggtgagcgactacgaggacttcgtgaagcagaactacaacaagaaccgcggcaagaggaacaagctgctgctggaggacatccctgagaagatgatcgagaggcagctgaacgacacccgctacatcagcaagtacatcacacaggtgctgagcaacatcgtgagggacgataaggagggctcaaaggatgatggcgtgaacagcaagaacatcgtgccaggcaacggcaagatcacaacaaggctgaagcaggattggggcctgaacgatgtgtggaatgatctggtgctgccaaggttcgagaggatgaacaccctgaccaacagcaacgacttcacctctaagaacacccacggcaagacaatcccaacagtgccgatcgagctgagcaagggcttcagcaagaagaggatcgatcacaggcaccacgccatggatgccctggtgatcgcctgcgccacaagggatcacgtgaatctactgaataacgagtctagcaagagcgatacaaagaggtacgatctgaacaggaagctgcgcaagtacgagaaggtggcctacaacgatccaaagacaggcgagaggatcgagaaggaggtgccgaaggatttcatcaagccatgggagacattcacagaggacacaaggaccctgctggagaacatcgtgatcagcttcaagcagaacctgcgcgtgatcaacaaggccaccaactactacgagaagatcgagaacggcaagaaggtgaaggtggagcagaagggcatcaattgggccgtgaggaaggccctacataaggagacagtgtctggccaggtgcacctggataggatcaaggtggccaagggcaagatcctgacagccacaaggaagacactggatgcctctttcaacgagaagacaatcgagagcatcacagacaccggcatccagaagatcctgctgaactacctgaagagcaaggacaacaacccagaggtggccttctctccagagggcatcgaggagctgaacaagaacatcaggctgtacaatgatggcaaggcccaccagccaatcctgaaggtgcgcgtgttcgagcagggctcaaagttcacactgggcgagacaggcaataagacaacaaagttcgtggaggccgccaagggcacaaatctgttcttcggcatctacgaggataagtctggcaagaggtcttacgagacaatcccgctgaatatcgtgatcgagaggcagaagcagggactgcaggccgtgccagagacaaatgagaagggcaagcagctgctgttcacactgtctccaaatgatctggtgtacgtgccagaggagggcgtgttcgatgagaacaatatcaaggtggacaggatctacaaggtggtgagcttctcaacctaccagtgcttcttcgtgaggaacgacgtgagcacaagcgtggtgaacaaggtggagtacagcgccctgaacaagatggagaagagcatcgacaacatcatgatcaaggagaactgcgtgaagctgaacgtggaccgcctgggcaagatctctaaggcc

SEQ ID NO: 181 - DNA, 4368 bp, synthetic construct
  misc_feature 1..4368: note = source = /note="Description of Artificial Sequence: Syntheticpolynucleotide"
  misc_feature 1..4368: note = source = /note="APG01688.1 Soy optimized"
  source 1..4368: mol_type = other DNA
atgatgatcaagaacatactaggactcgatctcggaaccaactctatcggatgggctctaatcaagcaggatttcgagaacaagcatggagaaatactagggatgggctccagaataattcctatgtcccaggatatactaggagatttcggaaagggtaactctatttctcagaccgctgatagaaccaagtataggtctgttagaaggcttagggagagatttcttcttaggagagagaggcttcatagggtgctccacctattaaacttccttcctcaacactacgcttctcagatcgatttcgagaagaagttcggaaagtttaagtctgagaccgaaccaaagttagcttgggaaaattggggaggaaagttctctttcctcttccaaaatagcttcaatgaaatgctggaggatttcaaggctgctggacaggggctcaaaataccttacgactggaccatatactatctcaggaagaaagcattgtcccagaaaatagagaaggaggagctagcttggatactcctcaacttcaatcaaaagaggggatactatcaattaagaggtgaggaggaggaggagaaccctaataaactcgtggagttctactccctcaaaatagtggatgttgttgctgatgagcctcagaagggaaagtccgatatctggtactcattaattcttgagaacggatgggtttatcgcagggcttccaaaatacctctcttcgactggaaggataaaaccagggatttcattgtgaccaccgatcttaacgatgataggtctgtgaagactgataaggagggaaatgaaaagaggtcttttagggctccttcagagaacgattggactcttgtgaagaagaagactgagcaggagattgatcagtctcataaaaccgtgggaacctacatctatgaaaccctcctattaaaccccaagcagaaaatcaagggaaagttggtgaggaccattgagaggaagttctacaaggacgagcttaagcagatactagagaagcaaaaggagttccaccaggaattaaagaacgacgacctatataacgactgcattagggagctgtacaggaataatgaggctcaccaattaaccctctccaagaaggatttcgtgcacctattaatggacgatctcattttctaccagaggcctcttaggtcccagaaatcaagcatttctaattgcaccctggagttcagaaaatacaaggatgaaaacgggattgagcacacccagtacctcaaggctatcccaaaatcaaacccatactaccaagagtttaggctttggcagtggatgtacaacttgaacatctacagaaaggatgatgaggctaacgtaaccaaggagttcctcaacaccaataaagacttcgagtctctcttcgagttcctcaataatcgcaaggagattgagcagaagcccctaatcaagttccttcttgagcagaaggacataaacaagaagctattaaacgccgaggctgagaaatacaggtggaactacgtggaggataaaaagtacccctgcaatgaaaccaagaccatgatttcctctaggcttgacaaggtggaaaatatctccgatgatttcctcaccagggacatcgagcagaaaatatggcatataatctactccgtgaacgacaagatcgagtatgaaaaggcattaaagtccttcgctaccaggaacgacctcgatgaaaatagcttcatcgaagcattcaagaagttctcccctttcaagtccgagtacggatctttctctgagaaggcaatcaagaagctccttccattaatgaggctcggaaaatactggtacgaggatgaaatcgtgaagcattccgacatctacttcaaaaatattgagaacctcctcggggatttctccaaccgcgacaagaaaatatctgaggaagacaaggagaagtggaacaagtcaatcaaccttaagctccaggaggaattaaaggatttccagaccgctgagattgatttgttccagggacttaggcttcacattgctcagtaccttgtttatggaaggcactccgaagcatctatgattggaaagtggaattctgctgaggatcttgaggagttccttaaggatttcaagcagcattcccttaggaaccctattgtggagcaggttattactgagactcttagggtggttaaggatatttggctaaagtacggaaacggggctaaggatttcttcaacgagattcatattgagcttggaagggaaatgaagttgcctgctgatgataggaagaagctcaccaaccaaatttctgagaacgagaacaccaacttcaggattaaggctctccttgctgagatgatgaacgattcttctgttgagaacgttaggcctttctctcctatgcagcaggagattctaaagatctacgaggatgatgtgctcaagtccgatatcgagatcgaggatgatatcctcaagatttctaagactgctcaaccttctccttctgatcttaagaggtacaagttgtggcttgagcagaagtacaagtctccttacaccggacagattattcctcttaacaagttgttcacccctgagtacgagatcgagcacattattcctcagtcaaggtacttcgatgattccttctccaataaggtgatttgcgagtctgctgtgaacaagctcaaggataactacatcgggcttgagttcattaagcagttcggaggaactattattgagcttggattcgggaagtctattaaggtgttcgagactaaggagtacgaggatttcgtgaagaagcattacgctaacaaccagggaaagaggaacaagctccttatggaagatatccctgagaagatgattgagaggcagatgaacgataccaggtacatctcaaagtacatttctggggtgctttctaacattgttagggtggaagatggatctgatgagggagtgaactctaagaacattgttcctggaaacggaaagattaccacccaacttaagcaggattggggacttaacgatgtgtggaacgatctcattcttcctaggttcgagaggatgaatcagcttactaactctaaggttttcactgcttggaacgagaactaccagaagttccttcctactgtgcctattgagtactccaagggattctcaaagaagaggattgatcacaggcatcatgctcttgatgctcttgttattgcttgcgctactaaggatcacgtgaaccttcttaacaaccagtctgctaagtctgataccaagaggtacgatctcaagaagaagtccatgaagttcgagaaggtggtttacaacgatgctaagactggagaaaagattgagagggaggttcctaagcaatttcttaagccttgggagaacttcactcttgatgtgaagcacaaccttgagaccatcattgtgtcattcaagcagaacctcagggtgattaacaaggctaccaactactacgagaagtacgttgagaaggatggaactaagaataaggagagggttgagcaaactggaactaactgggctattaggaagcctatgcacaaggatactgtgtctggaaaagttgatcttccttgggttaaggtgcctaagggaaagattcttactgctaccaggaagtctctcgattcttctttcgatctcaagtccattggatctattaccgataccgggattcagaagatcctaaagaactacctcgctttcaaggatggaaatcctgagcttgctttctctcctgagggaattgatgacctcaacaagaacatcgagaagtataacgatggaaagcctcaccagcctattaacaaggttagggttttcgagcttggatctaagttccaagttggacagtctggaaacaagaaggataagtatgttgaggctgctaagggaaccaaccttttcttcgctgtttatgaggatgagaagggaaagaggaactacgagactattcctctaaacgaggttattgagaggcaaaagcagggactttctgtggttgatcttaaggggaccaacgatttctatctttgccctaacgatttcgtgtacattccttctggagatgagctggagaacatcaacaacgtggacttcaaggatatcaagaaggagattaacgagaggatctacaaggtggtgtctttcactggaaacaggctttcttgcattccttacatggtggctaccactattgtgaacaagttggagttcacccagctaaacaagattgagttcaccaaggagaaggagatctgcattaagttgaacgtggataggctcggaaacatttctaaggct

SEQ ID NO: 182 - DNA, 4368 bp, synthetic construct
  misc_feature 1..4368: note = source = /note="Description of Artificial Sequence: Syntheticpolynucleotide"
  misc_feature 1..4368: note = source = /note="APG01688.1 Corn optimized"
  source 1..4368: mol_type = other DNA
atgatgatcaagaacatcctaggcctggacctcggaaccaattctatcggctgggccctgatcaagcaggacttcgagaacaagcatggcgagatactaggcatgggcagccgaataatcccgatgagccaggacatactaggcgatttcggcaagggcaatagcatctctcagaccgccgacagaaccaagtacaggtctgttaggaggctgagggagaggttccttcttaggagggagaggctgcatagggtgctccacctattaaacttcctgccacagcactacgccagccagatcgacttcgagaagaagttcggcaagttcaagagcgagacagaaccaaagctggcttgggaaaactggggcggcaagttcagcttcctgttccaaaatagcttcaatgaaatgctggaggacttcaaggccgccggccagggattaaagatcccctacgactggaccatatactacctgcgcaagaaagcattgagccagaaaatagagaaggaggagctggcctggatactgctgaacttcaaccaaaagcgcggatactaccagctgcgcggtgaggaggaggaggagaacccgaataaactggtggagttctactcactgaaaatagtggacgtggtggccgacgagccgcagaagggcaagagcgacatctggtacagcctgatactagagaacggctgggtgtaccgccgcgctagcaaaataccgctgttcgactggaaggataaaacccgcgacttcatcgtgaccaccgacctgaacgacgacaggagcgtgaagacagataaggagggcaatgaaaagaggagcttcagggccccaagcgagaacgattggaccctggtgaagaagaagacagagcaggagatcgatcagagccataaaaccgtgggcacatacatctatgaaaccctgctattaaacccgaagcagaaaatcaagggcaagctggtgcgcaccatcgagcgcaagttctacaaggacgagctgaagcagatactagagaagcagaaggagttccaccaggaattaaagaacgacgacctatataacgactgcatccgcgagctgtaccgcaataatgaggcccaccaattaaccctgagcaagaaggacttcgtgcacctattaatggacgacctgatcttctaccagcgcccgctgcgcagccagaagtcaagcatcagcaattgcaccctggagttccgcaaatacaaggatgaaaacggcatcgagcacacccagtacctgaaggccatcccgaagagcaacccatactaccaggagttccgcctgtggcagtggatgtacaacctgaacatctaccgcaaggacgacgaggccaacgtaaccaaggagttcctgaatacaaataaagacttcgagagcctgttcgagttcctgaataatcgcaaggagatcgagcagaagccgctaatcaagttcctgctggagcagaaggacataaacaagaagctattaaacgccgaggccgagaaatacaggtggaactacgtggaggataaaaagtacccgtgcaacgaaaccaagaccatgatcagcagccgcctggacaaggtggaaaatatcagcgacgacttcctgacccgcgacatcgagcagaaaatatggcatataatctacagcgtgaacgacaagatcgagtatgaaaaggcattaaagagcttcgccacccgcaacgacctggatgaaaacagcttcatcgaagcattcaagaagttcagcccgttcaagagcgagtacggcagcttcagcgagaaggcaatcaagaagctgctgccattaatgcgcctgggaaaatactggtacgaggacgagatcgtgaagcatagcgacatctacttcaaaaatatcgagaacctgctgggcgacttcagcaaccgcgacaagaaaataagcgaggaggacaaggagaagtggaacaaaagcatcaacctgaagctgcaggaggaattaaaggacttccagaccgccgagatcgacctgttccagggcctgcgcctccacatcgcccagtacctggtgtacggccgccacagcgaggcaagcatgatcggcaagtggaacagcgccgaggacctggaggagttcctgaaggacttcaagcagcatagcctgcgcaacccgatcgtggagcaggtgatcaccgagaccctgcgcgtggtgaaggacatctggctaaaatacggcaacggcgccaaggacttcttcaatgaaattcacatcgagctgggccgcgagatgaaactgccggccgacgaccgcaagaagctgaccaaccaaatcagcgagaatgaaaacaccaacttccgaatcaaggccctgctggccgagatgatgaacgacagcagcgtggagaacgtgcgcccgttcagcccgatgcagcaggagatcctgaaaatatacgaggacgacgtgctgaagagcgacatcgagatcgaggacgacatcctgaagatcagcaagaccgcccagccgagcccgagcgacctgaagcgctacaagctgtggctggagcagaagtacaagagcccgtacaccggccagatcatcccgctaaacaagctgttcaccccggagtacgagatcgagcacatcatcccgcagagccgctacttcgacgacagcttcagcaacaaggtgatctgcgagagcgccgtgaacaagctgaaggataactacatcggcctggagttcatcaagcagttcggcggcaccatcatcgagctgggcttcggcaagagcatcaaggtgttcgagaccaaggagtacgaggacttcgtgaagaagcactacgccaacaaccagggcaagcgcaacaagctgctgatggaggacatcccggagaagatgatcgagcgccagatgaacgacacccgctacatcagcaagtacatcagcggcgtgctgagcaacatcgtgcgcgtggaggatggctcagatgagggcgtgaacagcaagaacattgtgccgggcaacggcaagatcacaacacagcttaagcaggactggggcctgaacgatgtgtggaacgatctgattctgccaaggttcgagaggatgaaccagctgaccaacagcaaggtgttcaccgcctggaacgagaactaccagaagttcctgccaaccgtgccaatcgagtacagcaagggcttcagcaagaagaggatcgatcacaggcatcatgctctggatgctctggtgatcgcctgcgctacaaaggatcacgtgaacctgctgaacaaccagagcgccaagagcgacacaaagaggtacgacctgaagaagaagagcatgaagttcgagaaggtggtgtacaacgacgccaagaccggcgagaagattgagagggaggtgccgaagcagttcctaaagccatgggagaacttcacactggacgtgaagcacaacctggagaccatcatcgtgagcttcaagcagaacctgagggtgatcaacaaggccaccaactactacgagaagtacgtggagaaggacggcaccaagaacaaggagagggtggagcagacaggcacaaattgggccatccgcaagccaatgcacaaggatacagtgtctggcaaggtggatctgccatgggttaaggtgccaaagggcaagatcctgacagccacaaggaagagcctggatagcagcttcgatctgaagagcatcggctctatcacagacaccggcatccagaagatcctgaagaactacctggccttcaaggatggcaatccagagctggctttctctccagagggcatcgacgacctgaacaagaacatcgagaagtacaacgacggcaagccacaccagccaatcaataaggtgcgcgtgttcgagctaggctctaagttccaggttggccagagcggcaacaagaaggataagtacgttgaggccgccaagggcacaaatctgttcttcgctgtgtacgaggatgagaagggcaagcgcaactacgagacaatcccactgaacgaggtgatcgaaaggcagaagcagggcctgtctgttgtggatctgaagggcaccaacgatttctacctgtgcccgaacgacttcgtgtacattccatctggcgacgagctggagaacatcaacaacgtggacttcaaggacatcaagaaggagatcaacgagcgcatctacaaggtggtgagcttcaccggcaacaggctgagctgcatcccatacatggtggccaccaccatcgtgaacaagctggagttcacccagctgaacaagatcgagttcaccaaggagaaggagatctgcatcaagctgaacgtggacaggctgggcaacatcagcaaggcc

SEQ ID NO: 183 - DNA, 3210 bp, Bacillus sp.
  misc_feature 1..3210: note = source = /note="APG05083.1 Native Seq"
  source 1..3210: mol_type = genomic DNA
atgagagagttggattatcgcataggattagatattggaacgaattctattggctgggggatcattgaattatcttggaacaaagatagagaacaatatgagaaagcaagaattgtcgacaagggtgttcgtatgtttgataaggctgaaatacctaagactggtgcttctcttgctgaaccgaggcgtatagcacgctcatcacgtagaagattaaatcgcaaaagccagagaaaaaaagatatacgtaatttactcgttcaacatgaaattattagtcaaaaggaattggcttcgttatatcccctgacaaaaagttcaatggatatttgggatattcgcttggatggattagatcgcttgttagaccgttttgaatggactcgattattaattcatttagcgcaaagacgcggttttaaatcaaatcgaaagtctgaactgaaagatgtggagacaggaaaagtattatcaagtattcaagcaaatgaaaaacgattatcactgtaccgtacagtgggagagatgtggatgaagaatgaagattttagtaaatatgacaaaaggcgtaattcttctaatgagtatgtattttcggttagccgtgcagatttagaaaaagaaattgtgacactatttgaagcgcaaagaaaatttcagtcatcatatgcatcggctgatttacaaaaaacatacttacaaatttgggcacaccaacttccttttgcttctgggaatgcaattgtaaataaagtaggatattgttcattattaaaagggaaagaaaagagagttccgaaagcaacatatacttttcaatatttcagtacactagatcaaataaatcgaacacgattaggtcctaatttccaaccatttacgaaggaacagagagacgttattttagatgaaatgtttaatcgaacagattattataaaaaaaagacaatacccgaagtcacttactatgatattcggaaatggttagcattagatgaaacaattcaatttaaaggacttacctatgacccaaatgaagagctgaaaaaaatagaattgaaatcctttattaatttaaagccattctatgaaattaaaaaggtagttactaattacgccaaaaaaacaaatgaggcattctcaacattagactatgatacatttgcatatgctttaacagtttataaaacggacaaagatattagatcttatttaaagaaatctaataatttatcaaaatgttgctacgatgatcaattaatagaagagctattaactctctcctatacaaagtttggtcatttatcatttaaagcaattaatcatgtactgccaattatgcaagagggaaggacttatcaggaagcaatacaccaattaggatatgatgccactaatctaaaaaaagaaaacagaagtatgttcttgcccctttttcccgatgagataacaaatccaattgttaaaagagcactaactcaagcacgtaaagttgtaaatgctattattagaagatatggttcccccaattctgttcatattgaactagctcgtgagctttctaaaagtcatgatgagagaacgaaaataatgaaagctcatgatgaaaattataagaaaaataaaggagccatatcaattttgattgagaatggaattttaaatccgacaggatatgatattgtacgttataagttatggaaagagcaaggagaacgatgtgcttattcgctaaaacagattcctgctaatacgttttttaatgaaatgaaaaaagagcgaagtggctccccagttctagagatagatcacattttaccgtatagtcagagttttattgatagttatcataataaagtactagtttatggagatgagaatcaaaaaaagggaaatcgaattccatatacttattttttagaaggaaataaggactgggaaagctttgaaagctacgtacgattgaatagttttttttctaaaaagaagcgcggatatttattgaaaaaagcttacttgccaagagagagtaacatgattaaggagcgtcatttaaatgatactcgatatgctagtagctatttgaaaaacttcattgagaaaaatttgaaatttaaagaagttgaaggtagtacacgaaaaaaacatgtacagacggttaacggtataattacagcccatcttcgaaaaagatggggattagaaaaagataggcaggaaacatatttgcatcatgcaatggacgctattattgttgcttgtacagaccatcatatggtcactaaagtaacggagtactatcaaataaaagaaagtaataagtcaataaggaaaccatactttcctttgccatgggtgggctttagagaggaaattttatcacatttagcaaggcagccaattgctagaaaaattagtgaggaacttaaaattggatatcaatcatttgattatatacttgtatcgcgaatgccaaaaagatctgtcactggagcagctcatgaacagacaatcatgaaaaaaggtggtatcgacaaaaaaggaaaaactattatcataaagcgtgtgtacttaaaggatattaagtttgatgagaatggcgattttaaaatggttggaaaagaacaggatttagcaacttatgaagcgataaagcaaagatatatagagtatggaaaagaatcgaaaaaagcatttgaaacacctttatacaagcctagtaaaaaaggaaaaggaaacctcattaaaaagatcaaagtagaagtgcaaaccaaatcttttgttcgagaagttaatgggggcgtagctcaaaatggtgatttagtaagagtagatttgtttgaaaaggataatagatattatatgatacctatttatgtaatggatactgttcattccgaactaccaaataaagctgtaacaagcagtaaaggctatgagcaatggttaacaatagataacagctttacgttcaaattcagtttatatccttatgatttagtacgacttgtaaaaggtaatgaagatcgtttcctatattttagtacccttgatattaattcggatcgtcttaatttcaaagatgtaaacaagccatcaaagcaggctgaaaatcgttatagccttaaaacaattgagaatttagaaaaatatgaggttggtgttttaggtgatttaaggtttgtgagacaagaaatacgtaaaaatttt

SEQ ID NO: 184 - DNA, 3210 bp, synthetic construct
  misc_feature 1..3210: note = source = /note="Description of Artificial Sequence: Syntheticpolynucleotide"
  misc_feature 1..3210: note = source = /note="APG05083.1 E. coli optimized"
  source 1..3210: mol_type = other DNA
atgcgcgagctagattatcgcatcggcctggatattggcactaactcaataggctggggcatcatcgaactgagctggaataaagaccgcgagcagtatgaaaaggcgcgcatcgtggataaaggcgtgcgcatgttcgacaaagcggaaattccgaaaaccggtgcaagtctggcggaaccaagacgtattgcacgtagtagtcgccgccgattaaaccgcaaaagtcagcgcaagaaggatataaggaacctgctggtccagcatgaaatcatcagccagaaggaactggcgagcctgtacccgctgaccaaatcaagtatggacatctgggatataaggctggacggcctggatcgtctgctggatcgctttgaatggacccgcctattaatccatctggcacagcgccgtggcttcaaatcaaaccgcaagagcgaattaaaggacgtggagaccggtaaagtgctgtcaagcattcaggcgaatgaaaagcgcctgagcctgtatcgtaccgtgggcgagatgtggatgaaaaacgaggacttctcaaaatacgataaaaggcgcaacagcagcaacgagtacgtgttcagcgtgagccgcgcggacctggagaaagaaatcgtgaccctgtttgaggcgcagcgcaaattccaatcaagctatgcgagcgcggatctgcagaaaacctacctgcagatttgggcacatcagctgccgtttgcaagcggtaacgcgatcgtgaataaagtgggctattgcagcctattaaaaggcaaagaaaaacgcgtgccgaaagcgacctatacattccagtacttcagcaccctggaccaaatcaaccgcacccgcctgggcccgaacttccagccgttcaccaaagaacagcgcgacgtgatcctggatgaaatgttcaaccgcaccgactactataaaaagaaaaccatcccggaggtgacatactacgatataaggaaatggctggcgctggatgaaaccatccagttcaaaggcttaacctacgatccgaacgaggaattaaagaagatcgaattaaaatcgttcattaatctgaaaccgttctatgaaattaaaaaggtggtaaccaactacgcgaagaaaaccaacgaagcattcagcaccctggactacgatacattcgcgtacgcgctgaccgtgtataaaaccgacaaggatataaggagctacctgaagaaatcaaacaacctgagcaaatgctgctacgacgaccaattaatcgaagaactgctgaccctgagctacaccaaattcggccacctgagcttcaaagcgatcaaccacgtgctgccgatcatgcaggaaggccgcacctaccaggaagcgatccaccagctgggctacgatgcgaccaacctgaaaaaggaaaaccgcagcatgttcctgccgctgttccctgatgaaatcaccaacccgatcgtgaaacgcgcgctgacccaggcgcgcaaggtggtgaacgcgatcatccgccgctacggctcaccgaacagcgtgcacatcgaactggcgcgcgaactgagcaaaagccacgatgaacgcaccaaaatcatgaaagcgcacgatgaaaactacaaaaagaacaaaggcgcgatcagcatcctgatcgaaaacggcatcctgaacccgaccggctacgatatcgtgcgctacaaactgtggaaagaacagggcgaacgctgcgcgtacagcctgaaacagatcccggcgaacaccttcttcaacgaaatgaaaaaggaacgctcaggcagcccggtgctggaaatcgatcacatcctgccgtacagccagagcttcatcgatagctaccacaacaaagtgctggtgtacggcgatgaaaaccagaaaaagggcaaccgcatcccgtacacctacttcctggaaggcaacaaagattgggagagcttcgaaagctacgtgcgcctgaacagcttcttcagcaaaaagaaacgcggctacctgctgaaaaaggcgtacctgccgcgcgaaagcaacatgatcaaagaacgccacttaaacgatacccgctacgcgagcagctacctgaaaaacttcatcgagaaaaacctgaaattcaaagaagtggaaggcagcacccgtaaaaagcatgtccagaccgtgaacggcatcatcaccgcgcatctgcgcaaacgctggggcctggaaaaagatcgccaggaaacctatctgcatcatgcgatggatgcgatcatcgtggcgtgcaccgatcatcatatggtgaccaaagtgaccgaatattatcagatcaaagaaagcaacaaatcaatccgcaaaccgtattttccgctgccgtgggtgggctttcgcgaagaaatcctgagccatctggcgcgtcagccgatcgcgcgtaaaatcagcgaagaactgaaaatcggctatcagagctttgattatatcctggtgagccgcatgccgaaacgcagcgtgaccggcgcggcgcatgaacagaccatcatgaaaaagggcggcatcgataaaaagggcaaaaccatcatcatcaaacgcgtgtatctgaaagatatcaaatttgatgaaaacggcgattttaaaatggtgggcaaggaacaggacttagcgacctacgaagcgatcaaacagcgctacatcgagtacggcaaagagagcaaaaaggcgtttgaaaccccgctgtacaaaccgagcaaaaagggcaagggcaacctgatcaaaaagatcaaagtggaagtccagaccaaatcgtttgtgcgcgaagtgaacggcggcgtggcgcagaacggcgatctggtgcgcgtggatctgtttgaaaaagataaccgctactatatgatcccgatctacgtgatggataccgtgcatagcgagctgccgaacaaagcggtgaccagcagcaaaggctatgaacagtggttaaccatcgataacagctttaccttcaaattcagcctgtatccgtacgacctggtgcgcctggtgaaaggcaacgaagatcgcttcctgtatttcagcaccctggacatcaacagcgatcgcctgaacttcaaggacgtgaacaaaccgagcaaacaggcggaaaaccgctatagcctgaagaccatcgagaacctggagaaatatgaagtgggcgtgctgggcgatctgcgctttgtgcgccaggaaattcgcaaaaacttc

SEQ ID NO: 185 - DNA, 3213 bp, Bacillus sp.
  misc_feature 1..3213: note = source = /note="APG07433.1 Native Seq"
  source 1..3213: mol_type = genomic DNA
atgagggagttggactatcgcatagggttagatattgggacgaattctattggatggggtgttattgaattatcctggaacaaggacagagaacgatatgaaaaagtaaggattgtcgaccagggtgttcgtatgtttgatagggctgaaatgccaaaaacaggtgcttctcttgctgaaccgaggcgtatagcacgttcatcacgtagaaggttaaatcgtaaaagtcagaggaagaaaaatatacgtaatttacttgttcaacatggggtgattactcaagaagagctggattcgttatatcctctttcaaaaaaatcaatggatatttggggtattcgattggatggattagatcgtctgctaaatcattttgaatgggctcgattattaattcatttagctcaaagacgtggttttaaatcgaatcgtaagtctgaactgaaagatacggagacggggaaggtattatcgagtattcaattaaatgaaaaacgattatcactgtaccgtacagtgggagaaatgtggatgaaagatcctgattttagtaagtatgatagaaaacggaattctcctaatgagtatgtattttcggttagtcgtgcggaactagaaaaggaaatagttactttatttgcagcacaaagaaggtttcagtcaccatacgcatcgaaggatttacaagaaacatatttacaaatttggacacaccaacttccctttgcttctgggaatgcaattttaaataaagtgggatattgttcattattgaaagggaaagaaagaagaattccgaaagcaacatatacttttcagtattttagtgcactagatcaagtgaatcgaacacgattaggacctgattttcagccatttacgaaggaacaaagagaaattattttaaataacatgtttcaacgtacagattattataaaaaaaagactatacctgaagttacatactatgatatccggaaatggctagaactagatgaaactattcaatttaaaggacttaattatgatccaaacgaagaattgaaaaaaatagaaaagaaaccatttattaatttaaaggcattctatgaaattaataaggtagtcgcaaattattctgagagaacaaatgagacattttcaacgttagactatgatgggattggatatgctttaacagtttataaaacggacaaagatattaggtcttatttgaagagctctcataatttacctaaacgttgttacgatgatcaattaatagaggaactattaagtctttcttatacaaagtttggtcacttatcactgaaagcaatcaatcacgtactatcgattatgcaaaaaggcaatacttataaagaagcagtagaccagttagggtatgatacaagcggtttaaagaaagaaaaaagaagtaagttcttgcctcctatttcagatgagataacgaatccaattgttaaaagagcgttaacacaagcgcgtaaagttgtgaatgctataattagaagacatggttctccacattcagttcatattgaactggctcgtgaactttctaaaaatcatgacgagagaacaaaaatagtgtcagctcaagatgaaaattataagaagaataaaggggctatatcaattttaagtgagcatggaattttgaatccaacaggctatgatattgttcgttataagttatggaaagaacaaggagaacgatgtgcttattcgttaaaggagattcctgcggatacgttttttaatgaattaaaaaaagaacgaaatggtgctccaattctagaggtagatcacattttaccgtacagtcaaagttttattgatagttatcataataaagtattagtttacagtgatgaaaatcgaaaaaaggggaatcgaattccatatacctattttttagagacaaataaggattgggaagcttttgaaagatatgtaagatcaaataaatttttttctaaaaagaagcgtgagtatttattgaaaagagcatatttgccaagagaaagtgaactgataaaggagcgacatttaaatgatacgcgatatgctagtacctttttgaaaaacttcattgagcagaatttgcaatttaaagaagctgaagataatccgcgtaaaagacgtgtacaaacagttaacggtgtaattacagcacattttcgaaaaagatggggattagaaaaagatagacaagaaacatacttacatcatgcaatggacgctatcattgttgcttgtacagaccatcatatggttactagagtaacagagtattatcaaataaaagaaagtaataaatcagtaaagaaaccgtattttcctatgccatgggagggctttagagatgaactcttatcacatttagcgagtcagccaatagctaaaaaaattagtgaggagcttaaagctggatatcaatcattagattatatatttgtgtctcgaatgccaaaaagatctatcactggagcagcacataaacaaacgatcatgagaaaaggcggtattgataaaaaaggaaaaactattattatagagcgtttgcatttaaaggatattaagtttgatgagaatggtgattttaaaatggttggcaaagaacaagatatggcaacttacgaggcgataaagcaaagatatttggagcatggaaaaaactcgaaaaaagcatttgaaacacctctatacaaacctagtaaaaaaggaacaggaaatcttattaaaagagttaaagttgaaggacaagctaaatcttttgttcgagaagtaaatgggggcgtagcccaaaatggtgatttagtgagagttgatttatttgaaaaagatgataaatattacatggtgcctatttatgtaccagataccgtttgttcagaattacccaaaaaagttgtggcaagtagtaagggttatgagcaatggttaacactagataacagctttacgtttaaatttagtttatacccttatgatttagtacggcttgtaaaaggggacgaagatcgtttcttatactttggtactctcgatatcgattcagatcgtcttaattttaaagatgtaaataagccatcaaaaaagaatgaatatcgttatagccttaaaacaattgaggatttagaaaaatatgaggtgggtgttttaggagatttaaggttagtaagaaaagaaacacgtagaaattttcac

SEQ ID NO: 186 - DNA, 3213 bp, synthetic construct
  misc_feature 1..3213: note = source = /note="Description of Artificial Sequence: Syntheticpolynucleotide"
  misc_feature 1..3213: note = source = /note="APG07433.1 E. coli optimized"
  source 1..3213: mol_type = other DNA
atgcgcgagctagattatcgcatcggcctggatattggcactaactcaataggctggggcgtgatcgaactgagctggaataaagaccgcgagcgctatgaaaaggtgcgcattgtggatcagggcgtcagaatgtttgatcgtgcggaaatgccgaaaaccggtgcatcactggcagaaccaagacgtatagcacgtagtagtcgccgccgattaaaccgtaaaagccagcgcaagaaaaatatccgcaacctgctggttcagcatggcgtcatcacccaggaagaactggatagcctgtacccgctgagcaagaaatcaatggacatctggggcattcgtctggatggtctggaccgcctattaaaccactttgaatgggcccgcctattaattcatctggcgcagcgccgcggcttcaaatcaaaccgcaagagcgaattaaaggacaccgagaccggtaaagtgctgtcatcgatccagctgaatgaaaaacgcctgagcctgtatcgcaccgtgggcgagatgtggatgaaagacccggacttctcaaaatacgaccgcaaacgcaacagcccgaacgaatacgtgtttagcgttagccgtgcggaactggagaaagaaatcgtgaccctgtttgcagctcagcgccgttttcagagcccgtatgcgagcaaagatctgcaagaaacctacctgcagatctggacccatcagctgccgtttgcaagcggtaacgcgatattaaacaaagtgggctattgcagcctattaaaaggcaaagaacgccgcatcccgaaagcgacctatacattccagtacttcagcgcgctggaccaggtgaaccgtacccgtctgggtccggattttcagccgttcaccaaagaacagcgcgagatcatattaaacaacatgttccagcgcaccgactactataaaaagaaaaccatcccggaggtgacatattacgatataaggaaatggctggaactggatgaaaccatccagttcaaaggcctgaactacgatccgaacgaggagctgaaaaaaatcgagaaaaaaccgttcattaatctgaaagcgttctatgaaatcaacaaagtggtggcgaactacagcgaacgcaccaatgaaaccttctcaaccctggattacgatggcatcggctacgcgctgaccgtgtataaaaccgacaaggatataaggagctacctgaaatcaagccacaacctgccgaaacgctgctacgacgaccaattaatcgaagaactgctgagcctgagctatactaagttcggccacctgtcattaaaagcgattaatcacgtgttaagcatcatgcagaaaggcaacacatataaagaagcggtggatcagctgggctacgataccagcggattaaaaaaagaaaaacgcagcaaattcctgccgccgatcagcgatgaaatcaccaacccgatcgtgaaacgcgcgctgacccaggcgcgcaaagtggtgaacgcgatcatccgccgccacggcagcccgcacagcgtgcacatcgaactggcgcgcgaactgagcaaaaaccacgatgaacgcaccaaaatcgtgagcgcgcaggatgaaaactacaaaaagaacaaaggcgcgatcagcatcctgtcagaacacggcatcctgaacccgaccggctacgatatcgtgcgctacaaactgtggaaagagcagggcgaacgctgcgcgtacagcctgaaagaaatcccggcggataccttcttcaacgagctgaaaaaagaacgcaacggcgccccgatcctggaagtggatcacatcctgccgtacagccagagcttcatcgatagctaccacaacaaagtgctggtgtacagcgatgaaaaccgcaaaaaaggcaaccgcatcccgtacacctacttcttagagaccaacaaagattgggaagcgttcgaacgctacgtgcgcagcaacaagttcttcagcaaaaagaagcgcgagtacctgctgaaacgcgcgtacctgccgcgcgaaagcgaactgatcaaagaacgccatctgaacgatacccgctacgcgagcaccttcctgaaaaacttcatcgaacagaacctgcagttcaaagaagcggaagataacccgcgcaaacgccgcgtccagaccgtgaacggcgtgatcaccgcgcattttcgcaaacgctggggcttagaaaaagatcgccaggaaacctatctgcatcatgcgatggatgcgatcatcgtggcgtgcaccgatcatcatatggtgacccgcgtgaccgaatattatcagatcaaagaaagcaacaaaagcgtgaaaaaaccgtattttccgatgccgtgggaaggctttcgcgatgaactgctgagccatctggcgagccagccgatcgcgaaaaaaatcagcgaagaactgaaagcgggctatcagagcctggattatatctttgtgagccgcatgccgaaacgctcaatcaccggcgcggcgcataaacagaccatcatgcgcaaaggcggcatcgataaaaaaggcaaaaccatcatcatcgaacgcctgcatctgaaagatatcaaatttgatgaaaacggcgattttaaaatggtgggcaaagaacaggacatggcgacctacgaagcgatcaaacagcgctatctggagcatggcaagaacagcaaaaaagcgtttgagaccccgctgtacaaaccgagcaaaaaaggcaccggcaacctgatcaaacgcgtgaaagtggaaggccaggcgaaatcatttgtgcgcgaagtgaacggcggcgtggcgcagaacggcgatctggtgcgcgtggatctgtttgaaaaagatgacaaatattatatggtgccgatctatgtgccggataccgtgtgtagcgaactgccgaaaaaagtggtggcgagcagcaaaggctatgaacagtggctgaccctggataacagctttaccttcaaattcagcctgtatccgtatgatctggtgcgcctggtgaaaggcgatgaagatcgctttctgtattttggcaccctggacatcgattcagatcgcctgaacttcaaggacgtgaacaaaccgagcaaaaagaacgaatatcgctatagcctgaaaaccattgaagatctggaaaaatatgaagtgggcgtgctgggcgatctgcgcctggtgcgcaaagagacccgccgcaactttcat

SEQ ID NO: 187 - DNA, 3213 bp, Bacillus sp.
  misc_feature 1..3213: note = source = /note="APG07513.1 Native Seq"
  source 1..3213: mol_type = genomic DNA
atgagagagttggattatcgcataggattagatattggaacgaattctattggctggggtgttattgaattatcttggaacaaagatagagaacaatatgagaaaacgagaattgtcgacaagggtgttcgtatgtttgataaggctgaaatacctaagactggtgcttctcttgctgaaccgagacgtatagcgcgctcatcacgtagaaggttaaatcgtaaaagccagagaaaaaaagatatacgtaatttacttgttcaacatgaaattattagccaaaaggaattgacttcgttatatcccctgtcaaaaagttcaatggatatttgggatattcgcttagatggattagatcgcttgttagaccgttttgaatgggctcgattattaattcatttagcacaaagacgcggttttaaatcaaatcgaaagtctgaactgaaagatgtggagacaggaaaagtattatcaagtattcaagtaaatgaaaaacgattatctctgtaccgtacagtgggagagatgtggatgaagaatgcggattgtagtaaatatggcaaaaggcgtaattctcctaatgagtatgtattttcggttagccgtgcagatttagaaaaggaaattgtgactctatttgaggcgcaaagaaaattccattcatcatatgcatcggttgatttacaaaaaacatatatacaaatttgggcacaccaacttccttttgcttctgggaatgcaattgtaaataaagtaggatattgttcattattaaaaggtaaagaaaagagagttccaaaagcaacatatacttttcaatatttcaatacactagatcaaataaaccgaacacgattagggcccaatttccaaccatttacgaaggaacagagagacataattttagataaaatgtttcaacggacagattattataaaaaaaagacaatacccgaagttacttactatgatattcggaaatggttagcactagatgaaacaattcaatttaaaggacttacctatgacccaaacgaagagctgaaaaaaatagaaatgaaaccctttattaatttaaagccattctatgaaattaaaaaggtagttactaattacgcaaaaaaaacaaatgaggtattctcagcattagattatgatacagttgcatacgctttaacagtttataaaacggacaaagatattagatcttatttgaagagatctaataatttatcaaaacgttgctacgatgatcaattaatagaagagctattaactctctcctatacaaagtttggtcatttatcatttaaagcaattaatcatgtactgccaattatgcaagagggaaggacttatcaggaagcaatacaccaattagggtatgataccactaatcttaaaaaagaaaacagaagtatgttcttgcctattattccagatgagataacaaatccaattgttaaaagagcgttaactcaagcacgtaaagttgtaaatgctattattagaagatatggttctccaaattctgttcatattgaactagctcgtgagctttcgaaaagtcatgacgagagaaaaaaaataatgacagctcatgatgaaaattataagaaaaataaaggagctgtatcaattttgatcgataatggaattttaaatccgacaggatatgatattgtacgttataagttatggaaagagcaaggagaacgatgtgcttattcgttaaaaaagattcctgctaatacgttttttaatgaattgaaaaaagagcgaagtggccctccggttctagaggtagatcacattttaccgtatagtcagagttttattgatagttaccataataaagtattagtttatggggatgaaaatcaaaaaaagggaaatcgaattccatatacttttttttcagaagaagataaggagtgggaaagctttgaaagctacgtaagatcgaatagttttttttctaaaaagaagcgcggatatttattgaaaaaagcttacttgccaagagagagtaacttgattaaggagcgtcatttaaatgatacccgatatgctagtagctatttgaaaaacttcattgagaaaaatttaaaatttaaagaagctgtaggtattacacgaaaaaaatatgtacagacggttaacggtgtaattacagcccatctgcgaaaaaggtggggtttagaaaaagataggcaggaaacatatttgcatcatgcaatggacgctattattgttgcttgtacagatcatcatatggtcactaaagtaacggagtactatcaaataaaagaaggtaataagtcaataaagaaaccgtattttcctttgccatggatgggatttagagaggaaattttatcacatttagaaagtcagccaattgctagaaaaattagtgaggaacttaaaattggatatcaatcacctgattatatacttgtatcgcgaatgccaaaaagatctgtcactggatcagctcatgatcagacagtcatgaaaaaaggcgatatcgataaaaaaggaaagactattatcataaagcgtgtgcacttaaaggatattaagtttgatgagaatggcgattttaaaatggttggcaaagaacaagatttagcaacttacgaagcgataaagcaaagatatttagagtatagaaaagaatcgaaaaaagcatttgaaacacctttatacaagcctagtaaaaaaggaaaaggaaacctcattaaaaaaattaaagtagaagtgcaaaccaaatcttttgttcgagaaattaatgggggcgtagctcaaaatggtgatttagtaagagtagatttgtttgaaaaggataataggtattatatggtgcctatttacgtagtagatactgttcgttccgaactaccaaataaagctgtaacaagcagtaaaggctatgagcaatggttatcaatagataacagctttacgttcaaattcagtttatatccttatgatttagtacgacttgtaaaaggcgatgaagatcgttttctatactttagtactcttgatattaattcggaccgtcttaattttaaagatgtaaataagccatcaaagcaagctgaatatcgttatagtcttaaaacaattgagaatttagaaaaatatgagattggtgttttaggtgatttaaggttagtgagacaagaaacacgtaaaatttttaaa

SEQ ID NO: 188 - DNA, 3213 bp, synthetic construct
  misc_feature 1..3213: note = source = /note="Description of Artificial Sequence: Syntheticpolynucleotide"
  misc_feature 1..3213: note = source = /note="APG07513.1 E. coli optimized"
  source 1..3213: mol_type = other DNA
atgcgcgagctagattatcgcatcggcctggatattggcactaactcaataggctggggcgtgatcgaactgagctggaataaagaccgcgagcagtatgaaaagacccgcatcgtggataaaggcgtgcgcatgttcgacaaagcggaaattccgaaaaccggtgcaagtctggcggaaccaagacgtattgcacgtagtagtcgccgccgattaaaccgcaaaagtcagcgcaagaaggatatcaggaacctgctggtccagcatgaaatcatcagccagaaggaattaaccagcctgtacccgctgagcaaatcaagtatggacatctgggatataaggctggacggcctggaccgcctgctggatcgttttgaatgggcccgcctattaatccatctggcacagcgccgtggcttcaaatcaaaccgcaagagcgaattaaaggacgtggagaccggtaaagtgctgtcaagcattcaggtgaatgaaaagcgcctgagcctgtatcgcaccgtgggcgagatgtggatgaaaaacgcggactgctcaaaatatggcaaacgccgcaacagcccgaacgaatacgtgtttagcgttagccgtgcggacctggaaaaagaaatcgtgaccctgtttgaggcgcagcgcaaattccatagcagctatgcgagcgtggatctgcagaaaacatacatccagatctgggcacatcagctgccgtttgcgagcggtaacgcgatcgtgaataaagtgggctattgcagcctattaaaaggcaaagaaaaacgcgtgccgaaagcgacctatacattccagtacttcaatacactggaccaaatcaaccgcacccgcctgggcccgaacttccagccgttcaccaaagaacagcgcgatataatcctggataaaatgttccagcgcaccgactactataaaaagaaaaccatcccggaggtgacatactacgatataaggaaatggctggcgctggatgaaaccatccagttcaagggcttaacctacgatccgaacgaggagctgaaaaagatcgagatgaaaccgttcattaatctgaaaccgttctatgaaattaaaaaggtggtaaccaactacgcgaagaaaaccaacgaagtgttcagcgcgctggattacgataccgtggcgtacgcgctgaccgtgtataaaaccgacaaggatataaggagctacctgaaacgcagcaataatctgagcaaacgctgctacgacgaccaattaatcgaagaactgctgaccctgagctatactaagttcggccacctgagcttcaaagcgattaatcacgtgctgccaataatgcaggaaggccgcacctaccaggaagcaatacaccagctgggctacgatacaaccaacctgaaaaaggaaaataggagcatgttcctgccaataatcccggatgaaatcaccaacccgatcgtgaaacgcgcgctgacccaggcgcgcaaagtggtgaacgcaataatccgccgctacggctcaccgaacagcgtgcacatcgaactggcgcgcgaactgagcaaaagccacgatgaacgcaaaaagatcatgaccgcgcacgatgaaaactacaaaaagaacaaaggcgcggtgagcatcctgatcgataacggcatcctgaacccgaccggctacgatatcgtgcgctacaaactgtggaaagaacagggcgaacgctgcgcgtacagcctgaaaaagatcccggcgaacaccttcttcaacgaactgaaaaaggaacgctcaggcccgccggtgctggaagtggatcacatcctgccgtacagccagagcttcatcgatagctaccacaacaaagtgctggtgtacggcgacgaaaaccagaaaaagggcaaccgcatcccgtacaccttcttcagcgaagaagacaaagaatgggagagcttcgaaagctacgtgcgcagcaacagcttcttcagcaaaaagaaacgcggctacctgctgaaaaaggcgtacctgccgcgcgaaagcaacttaatcaaagaacgccatctgaacgatacccgctacgcgagcagctacctgaaaaacttcattgagaaaaacctgaaattcaaggaagcggtgggcatcacccgcaagaagtacgtccagaccgtgaacggcgtgatcaccgcgcatctgcgcaaacgctggggcctggaaaaagatcgccaggaaacctatctgcatcatgcgatggatgcgatcatcgtggcgtgcaccgatcatcatatggtgaccaaagtgaccgaatattatcagatcaaagaaggcaacaaatcaatcaaaaagccgtattttccgctgccgtggatgggcttccgcgaagaaatccttagccatctggaaagccagccgatcgcgcgcaaaatcagcgaagaactgaaaatcggctatcagagcccggattatatcctggtgagccgcatgccgaaacgcagcgtgaccggcagcgcgcatgatcagaccgtgatgaaaaagggcgatatcgataaaaagggcaaaaccatcatcatcaaacgcgtgcatctgaaagacatcaaattcgacgaaaacggcgacttcaaaatggtgggcaaggaacaggacttagcgacctacgaagcgatcaaacagcgctacctggaataccgcaaagagagcaaaaaggcgtttgaaaccccgctgtacaaaccgagcaaaaagggcaagggcaacctgatcaaaaagatcaaagtggaagtccagaccaaatcgtttgtgcgcgagatcaacggcggcgtggcgcagaacggcgatctggtgcgcgtggatctgtttgaaaaagataaccgctattatatggtgccgatctacgtggtggataccgtgcgcagcgaactgccgaacaaagcggtgaccagcagcaaaggctatgaacagtggttaagcatcgataacagcttcaccttcaaattcagcctgtacccgtatgatctggtgcgcctggtgaaaggcgatgaagatcgcttcctgtattttagcaccctggacatcaacagcgatcgcctgaacttcaaagacgtgaacaaaccgagcaaacaggcggaatatcgctacagcctgaagaccatcgagaacctggagaaatacgaaattggcgtgctgggcgatctgcgcctggtgcgccaggaaacccgcaaaatcttcaaa

SEQ ID NO: 189 - DNA, 3216 bp, Bacillus sp.
  misc_feature 1..3216: note = source = /note="APG08290.1 Native Seq"
  source 1..3216: mol_type = genomic DNA
atgagtgagttggattaccgcatagggttggatattggtacgaattccattggctggggtgttattgaattattttggaacaaggacagagaacgatatgaaaaagtaaggattgtcgacaagggcgttcgtatgtttgataaggctgaaatacctaataagggtgcttctcttgctgaaccgagacgtatagcgcgttcatcacgtagaaggttaaatcgcaaaagtcagaggaagaaagagatacgtaatttacttgttcaacacggaatgattacccaagaggaattggatttgttatatcctctttcaaaaaaatcaatagatatttgggatattcgtttggatggattagatcgtctgttgaatcatcttgaatgggctcgattattaattcatttagcacaaagacgtggttttaagtcgaatcgaaagtctgaattgaaagatgctgagacggggaaggtattatcgagtattcaagtaaatgaaaaacgactatttctgtaccgtacagtgggggaaatgtggataaaagatgctgagtttagtaagtatgatagaagacgtaattctcctaatgagtatgtattttcggttagtcgtgcggacttagaaaaggaaattgttactctatttgaagcacaaagaaagtttcagtcatcatacgcatcgaagaacttacaagaaacatatttacaaatttgggcacaccaacttccttttgcttctgggaatgcaattttaaacaaagtaggctattgttcattattgaaagggaaagaaaggagaattccgaaagcaacatatacttttcaatatttcagtgcactagatcaggtgaatcgaacacgattaggacctgattttcaaccatttacgcaggaacagaaagagattattttagataaaatgtttcaacgtacagattattataaaaaaaagactatacctgaagttagctactatgatatacggaaatggctagagttagatgaaactattcaatttaaaggacttaattatgatccaaacgaagaactgaaaaaaatagaaaaaaaaccatttattaatttaaaggcattctatgaaattaaaaaggtagttgcaaattatgctgagagaacaaatgaggcattttcaacgttagactatgatgcgattgcatatgctttaacagtttataaaacggacaaagatattagatcttatttgaagaaatctaataatttatcaaaacgttgctacgacgatcaattaatagaagagctatttactctctcctatacaaagtttggtcatttatcatttaaagcaattaatcatgtacttccaattatgcaagagggaaggacttatcaagaagcgatacaccaattaggatatgataccactaatcttaaaaaagaaaacagaagtatgttcttgcctcttattccagatgaaataacaaatccaattgttaaaagagcgataactcaagcacgtaaagttgtaaacgctattattagaagatatggttctccaaattctgttcatattgaactagctcgtgagctttctaaaagtcatgacgagagaaagaaaataatgacggctcatgatgaaaattataagaaaaataaaggagctatatccattttgatcgagaatggaattttaaatccgacaggatatgatattgtacgttataagttatggaaagagcaaggagaacgatgtgcttattcgttaaaggagattcctccggatacgttttttaatgaattaaaaaaagaacgaaatggttctccaattctagaggtagatcacattttaccgtatagccaaagttttattgatagttatcataataaagtattagtttacagtgatgaaaatcgaaacaagggaaatcgaattccatatacctattttttagagacaaataaggattgggaagcttttgaaaggtatgtaagatcaaataagcttttttctaaaaagaagcgtgagtatttattgaaaaaaacatatttgccaagagagagtgaactaataaaggagcgacatttaaatgatacgcgatatgctagtacctttttgaaaaacttcattgagcagaacttgcaatttaaagaagttgaagttaatctgcgtaaaaaacgtgtgcaaacagttaatggtgtaattacagcgcaccttcgaaaaagatggggattagaaaaaaatagacaggaaacatatctgcatcatgcaatggacgctatcattgttgcttgtacagaccatcatatggttactagaataacagagtattatcaaataaaagaaagtaataaatcagtaaagaaaccatattttccgatgccatgggagggttttagagatgaactcttatcacatttagcgagccagccaattgctaaaaaaattagtgaggagcttaaagctggatatcaatcatctgattatatatttgtgtctcgaatgccaaaaagatctgtcactggagcagctcatgatcaaacgattaggagaaaaggtggtattgataaaaaaggaaaaactattattataaagcgtgtgcgcttaaaggatattaagtttgatgagaatggcgattttaaaatggttggaaaagaacaagatttagcaacttatgaggcgataaagcaaagatatttggagcatagaaaaaactcgaaaaaagcatttgaaacacctctatacaagcctagtaaaaaagggacaggaaatctcatcaaaagagttaaaattgaaggacaaactaaagcttttgttcgagaagtaaatgggggagtagcccaaaatagtgatttagtaagagttgatttatttgaaaaagatgataaatattacatggtgcctatttatgtaccagataccgtttgttcagaattacccaaaaaagttgtgaaaagtggtaagggttatgagcaatggctaacactagataacagctttacgtttaaatctagtttatacccttatgatttagtacgtcttgtaaaaggtaatgaagatcgttttttatactttggtactctcgatatcgattctgaccgtcttaattttaaagatgtaaataagccatcaaaacagaatgaatatcgttatagtcttaaaacaattgagaatttagaaaaatatgaggttggtgttttaggtgatttaaggttagtgaaacaagaaacacgtagaatttttaataga

SEQ ID NO: 190 - DNA, 3216 bp, synthetic construct
  misc_feature 1..3216: note = source = /note="Description of Artificial Sequence: Syntheticpolynucleotide"
  misc_feature 1..3216: note = source = /note="APG08290.1 E. coli optimized"
  source 1..3216: mol_type = other DNA
atgagcgagctagattatcgcatcggcctggatatcggcactaactcaataggctggggcgtgatcgaactgttctggaataaagaccgcgagcgctatgaaaaggtgcgcatcgtggacaaaggcgtcaggatgttcgacaaagcggagatcccgaataaaggtgcatcactggcggaaccaagacgtatagcacgtagtagtcgccgccgattaaaccgcaaaagtcagcgcaagaaagagatccgtaacctgctggttcagcatggcatgatcacccaggaagaactggatctgctgtacccgctgagcaagaaatcaatcgacatctgggatataaggctggacggcctggaccgcctattaaaccatctggaatgggcccgcctattaatccatctggcgcagcgtagaggcttcaaatcaaaccgcaagagcgaattaaaggacgcggagaccggtaaagtgctgtcaagcattcaggtgaatgaaaagcgcctgttcctgtatcgcaccgtgggcgagatgtggattaaagacgcggagttctcaaaatacgaccgccgtcgcaacagcccgaacgaatacgtgtttagcgttagccgtgcggacctggagaaagaaatcgtgaccctgtttgaagcgcagcgcaaattccaatcaagctacgcgagcaagaacctgcaagagacctacctgcagatttgggcacatcagctgccgtttgcaagcggcaacgcgatattaaacaaagtgggctattgcagcctattaaaaggcaaagaacgccgcatcccgaaagcgacctatacattccagtacttcagcgcgctggaccaggtgaaccgtacccgtctgggccctgattttcagccgtttacccaggaacagaaagaaataatcctggataaaatgttccagcgcaccgactactataaaaagaaaaccatcccggaagtgtcatattacgatataaggaaatggctggagctggatgagaccatccagttcaagggcctgaactacgatccgaacgaggagctgaaaaaaatcgagaaaaaaccgttcattaatctgaaagcgttctatgaaattaaaaaagtggtggcgaactacgcggaacgaaccaacgaagcattcagcaccctggattacgatgcgatcgcgtacgcgttaaccgtgtataaaaccgacaaggatataaggagctacctgaaaaaatcaaacaacctgagcaaacgctgctacgacgaccaattaatcgaagaactgttcaccctgagctatactaagttcggccacctgagcttcaaagcgattaatcacgtgctgccaataatgcaggaaggccgcacctaccaggaagcgatccaccagctgggctacgataccaccaacctgaaaaaagaaaaccgcagcatgttcctgccgctgatcccggatgaaatcaccaacccgatcgtgaaacgcgcgatcacccaggcgcgcaaagtggtgaacgcgatcatccgccgctacggctcaccgaacagcgtgcacatcgagctggcgcgcgagctgagcaaaagccacgacgagcgcaaaaaaatcatgaccgcgcacgacgagaactacaagaaaaacaaaggcgcgatcagcatcctgatcgagaacggcatcctgaacccgaccggctacgatatcgtgcgctacaaactgtggaaagaacagggtgaacgctgcgcgtacagcctgaaagaaatcccgccggataccttcttcaacgaactgaaaaaagaacgcaacggctcaccgatcctggaagtggatcacatcctgccgtacagccagagcttcatcgatagctaccacaacaaagtgctggtgtacagcgatgaaaaccgcaacaaaggcaaccgcatcccgtacacctacttcctggaaaccaacaaagattgggaagcgttcgaacgctacgtgcgcagcaacaaactgttcagcaaaaagaaacgcgaatacctgctgaaaaaaacctacctgccgcgcgaaagcgaactgatcaaagaacgccacttaaacgatacccgctacgcgagcaccttcctgaaaaacttcatcgaacagaacctgcagttcaaagaagtggaggtgaacctgcgtaaaaagcgcgtccagaccgtgaacggcgtgatcaccgcgcatctgcgcaaacgctggggcctggaaaaaaaccgccaggaaacctatctgcatcatgcgatggatgcgatcatcgtggcgtgcaccgatcatcatatggtgacccgcatcaccgaatattatcagatcaaagaaagcaacaaatcagtgaaaaaaccgtattttccgatgccgtgggaaggctttcgcgatgaactgctgagccatctggcgagccagccgatcgcgaaaaaaatcagcgaagaactgaaagcgggctatcagagcagcgattatatctttgtgagccgcatgccgaaacgcagcgtgaccggcgcggcgcatgatcagaccatccgccgcaaaggcggcatcgataaaaaaggcaaaaccatcatcatcaaacgcgtgcgcctgaaagatatcaaatttgatgaaaacggcgattttaaaatggtgggcaaagaacaggacttagcgacctacgaagcgatcaaacagcgctatctggagcatcgcaagaacagcaaaaaagcgtttgagaccccgctgtacaaaccgagcaaaaaaggcaccggcaacctgatcaaacgcgtgaaaatcgagggccagaccaaagcgtttgtgcgcgaagtgaacggcggcgtggcgcagaacagcgatctggtgcgcgtggatctgtttgaaaaagatgacaaatattatatggtgccgatctatgtgccggataccgtgtgtagcgaactgccgaaaaaagtggtgaaaagcggcaaaggctatgaacagtggttaaccctggataacagcttcaccttcaagagcagcctgtacccgtatgatctggtgcgcctggtgaaaggcaacgaagatcgctttctgtattttggcaccctggacatcgatagcgatcgcctgaacttcaaagacgtgaacaaaccgagcaagcagaacgaataccgctacagcctgaagaccatcgagaacctggagaaatatgaagtgggcgtgctgggcgatctgcgcctggtgaaacaggaaacccgccgcatttttaaccgc

SEQ ID NO: 191 - DNA, 4011 bp, Enterococcus sp.
  misc_feature 1..4011: note = source = /note="APG05459.1 Native Seq"
  source 1..4011: mol_type = genomic DNA
atgaagaaagactacgttattggtctggatatagggactaattctgtcggctgggcagttatgacagaagactatcagttggtgaagaaaaaaatgcctatttatggaaacactgaaaaaaagaaaatcaagaaaaatttttggggtgtgcgtttatttgaagaagggcatacagccgaagatcgccgattaaaaagaacagcacgacgaagaattagtcgtcgacggaatcgtttacgctacttacaagctttttttgaagaagcgatgacagctttggatgaaaacttttttgctcgtttacaagagagttttttagtgcctgaagataagaagtggcacagacatccgatttttgctaagttggaagatgaagtagcttaccatgaaacgtatccgacaatctaccatttacgcaaaaaattagcagattcatctgagcaagcagatttacgactaatttatttggcgttggcacatattgtcaaatatcgtggacattttttaattgaaggaaaattaagtacagaaaatatttctgttaaagaacaatttcaacaatttatgatcatttataaccaaacctttgtgaatggagagagtcgcttagttagtgcgccattacctgaatctgtcttgattgaggaagagttgactgaaaaagcttctcgtactaaaaaatctgaaaaagtcttacaacaatttcctcaagaaaaagctaatggcttatttggtcagttcttaaagctaatggtagggaataaagctgactttaaaaaagtttttggtttggaagaagaagccaaaataacctacgctagtgaaagctatgaagaagatttagaaggcattttagcaaaggttggggatgaatatagtgacgtgtttttagctgctaaaaatgtgtacgatgcagttgaattatcaacgattttagcagattcggataaaaaaagtcacgcgaaattatcttctagcatgattgtccgttttacagaacatcaagaagatttaaaaaaattcaaacgatttattcgtgaaaattgtccagacgaatatgataatctctttaaaaatgaacagaaagacggctatgcaggctacattgcacacgcaggtaaggtttcacagcttaaattttaccagtatgttaagaaaatcattcaagatattgctggagcagaatattttttagaaaaaattgctcaagaaaactttttaagaaagcaacggacctttgataatggggtgattcctcatcaaattcatttggctgagttacaagcaatcattcatcgtcaagcggcctattatccatttttaaaagaaaatcaagaaaaaattgaacaactggttacatttcgaattccttattatgtcggaccattatcaaaaggagatgcaagtaccttcgcttggctaaaacgtcaaagtgaggaaccaattcgaccttggaaccttcaagaaaccgttgatttggaccaatcagcgacagcctttattgaacgaatgaccaattttgatacgtatttaccttctgaaaaagttttaccgaaacatagtttgttatatgaaaagtttatggtatttaatgaattgaccaagatttcttatacggatgatcgaggaatcaaagccaatttttcaggtaaagaaaaagaaaaaatctttgattatctgtttaagacgcgtcgaaaagttaagaaaaaggatatcattcaattctatcgaaacgaatataatacggagattgtcacgcttagtggacttgaagaagaccaatttaatgctagttttagcacgtatcaggatttactgaaatgtggtttaacaagagctgaattagaccaccctgataatgccgaaaaattggaagatatcataaaaattttaactatttttgaagatcggcaacgaattcggacgcaactttccacattcaaagggcagttctcagcagaagtactaaaaaaattagaacgcaagcactacactggctggggaagattgtcgaaaaaattaatcaatggtatctacgataaagaatcgggtaaaacaattttggactatttaattaaagatgatggggtctcaaaacactataatcgcaattttatgcaactgattaatgattcacaattatcttttaaaaatgctattcaaaaagcacagtccagtgaacatgaagaaacattaagtgaaactgtcaatgaattagctggtagtccagcaataaaaaaaggaatttatcaaagtttaaaaattgtcgatgaactagtcgcgattatgggttatgcgcccaagcggattgttgtcgaaatggcacgtgaaaatcaaacgactagcactggcaagagaagatccatccaacgcttgaaaatagttgaaaaagcaatggccgaaatcggcagcaatttattaaaagaacaaccaacgactaatgaacagttacgagatacccgtcttttcctttactatatgcaaaatggtaaagatatgtacacgggagatgaattatcgcttcatcgtttatctcactatgatattgatcatattatcccacaaagctttatgaaagacgattcattggataacctagttttagtgggctctactgaaaatcgagggaaatccgatgatgtacctagcaaggaagttgttaaggatatgaaagcttattgggagaaattatatgctgctggcttaattagtcaacggaaattccaacgtctgaccaagggggagcaaggtggcttgactctcgaagacaaagcgcattttatccaacgacaattagtagaaactcgtcaaattaccaaaaatgttgcagggatcttagatcaacgctacaatgctaattcaaaagagaaaaaagtccaaatcatcaccttgaaagctagtttaacgagccaatttcgttcgatttttggcttgtataaagttcgtgaagtgaatgattatcaccatggacaagatgcttatttaaactgtgtggtcgcaaccacgttattaaaagtttatcctaatttagcaccagaatttgtttacggagaatatcctaaattccaggcctttaaagaaaataaagccacggcgaagactataatttatacaaatttgatgcgctttttcacggaagatgaaccacggttcatgaaggatggcgaaattctttggagtaacagttatttaaaaaatatcaaaaaagaattaaattaccatcaaatgaatattgtcaaaaaagtagaagtgcaaaaaggcggtttttcaaaagagtcgattaaacccaaaggaccatcgaataaattgattcctgtcaaaaatggtttggatccccaaaaatatggcggttttgatagtcctgtagttgcttatacagtgttattcacgcatgaaaaaggtaaaaaacctcttattaaacaggagatactgggcattacgattatggaaaaaacaaggtttgaacaaaatcctattctttttttagaggagaaaggcttcctacggcctcgtgtattaatgaaattgcctaagtatacactgtatgaatttccagaggggcgcaggcgcttattagccagcgctaaagaagcgcaaaaagggaaccagatggttttacctgaacatttacttacgttactgtaccatgcgaaacaatgtttgttaccaaatcaatcagaaagtttggcttatgtagaacaacatcaaccagaatttcaagagattttagagagagtggttgactttgctgaagtgcatacattagccaagtcaaaagtccagcaaattgtgaaactttttgaagcaaatcaaacagcagatgtgaaagagattgcggcatcctttatccaacttatgcagtttaatgcgatgggcgcaccaagtacgtttaaatttttccaaaaagacattgagcgagcacgctacacgtcaatcaaagaaatttttgatgccactatcatttaccaatcgactacgggactgtatgaaacacgcagaaaggttgtcgac

SEQ ID NO: 192 - DNA, 4011 bp, synthetic construct
  misc_feature 1..4011: note = source = /note="Description of Artificial Sequence: Syntheticpolynucleotide"
  misc_feature 1..4011: note = source = /note="APG05459.1 E. coli optimized"
  source 1..4011: mol_type = other DNA
atgaaaaaggactacgtgatcggcctagacatcggcactaacagcgttggttgggcggtgatgaccgaagattaccagctagtgaagaagaagatgcctatctacggtaatacagagaagaagaagattaaaaagaacttctggggcgtgaggctattcgaggagggtcacaccgcggaggaccgccgcctgaagcgcaccgctagacgccgtatttcacgtcgtcgtaaccgtctgcgttacctgcaagcattcttcgaagaagcaatgaccgcgctggatgaaaacttcttcgctaggctgcaggagtcatttctggtgccggaggataaaaagtggcaccgtcatccgatcttcgcgaaactagaagacgaggtggcgtaccatgaaacctacccgactatctaccacctgcgcaaaaagctggcggatagcagcgaacaagcagacctgcgattaatctatctggcgctggcgcacatcgtaaaatacagaggccacttcctcatcgagggcaaattatcgaccgaaaatattagcgtgaaggagcagttccagcagttcatgatcatctacaaccaaaccttcgtgaacggcgaatcaaggctggtgagcgcgccgctgccggagagcgtattaattgaggaggaattaaccgagaaagcgagccgcactaagaaaagcgagaaagtgcttcagcagttcccgcaagaaaaagcgaacggcctgttcggccagttcctgaaattaatggtgggcaataaagcggacttcaaaaaggtgttcggcctggaagaggaggcgaaaataacctacgcgagcgaaagctacgaggaagacctggagggcatactagcgaaagtgggcgacgaatacagtgacgtgttcctggcggctaaaaacgtgtacgacgcggtggaattaagcaccatactagcggacagcgataaaaagagtcacgcgaaattaagctcaagcatgatcgtgcgcttcaccgaacaccaggaagacctgaaaaagttcaaacgcttcatccgcgaaaactgcccggacgaatacgataacctgttcaaaaacgaacagaaagatggctacgcgggatacatcgcgcacgcgggcaaagtgtcacaattaaagttctaccagtacgtgaagaaaataatccaggatatcgcgggcgcggaatacttcctggagaaaatagcgcaggagaacttccttaggaaacagcgcaccttcgacaacggcgtgatcccgcaccaaatacacctggcggaactacaggcaataattcaccgccaggcggcatactacccgttcctgaaagagaaccaggagaaaatagaacagctggtgaccttccgcatcccatactacgtgggcccgcttagcaaaggcgatgcgagcaccttcgcgtggctgaaacgccagagcgaagaaccaatccgcccgtggaacttacaagaaaccgtggatctggatcagagcgcgaccgcgttcatcgaacgcatgaccaacttcgatacatacctgccgagcgaaaaagtgctgccgaagcatagcctgctgtatgaaaaattcatggtgttcaacgaactgaccaaaataagctacaccgatgatcgcggcattaaagcgaacttcagcggcaaagaaaaagagaaaatattcgattacctattcaaaacccgccgcaaagtgaaaaagaaagacataatacagttctaccgcaacgaatacaacaccgaaatcgtgaccctgagcggcctggaagaagatcagttcaacgcgagcttcagcacctaccaggacttattaaaatgcggcctgacccgcgcggagcttgaccacccggacaacgcggaaaaactggaagatatcattaaaatactgaccatcttcgaagatcgccagcgcatccgcacccagctgagcaccttcaagggccagttcagcgcggaagtgctgaaaaagctggaacgcaaacattacaccggctggggccgcctgagcaaaaagctgatcaacggcatctacgataaagaatcaggcaaaaccatcctggactacctgatcaaagatgacggcgtgagcaaacactacaaccgcaacttcatgcagctgatcaacgatagccagctgagcttcaagaacgcgatccagaaagcgcagagcagcgaacatgaagaaaccctgagcgaaaccgtgaacgaactggcgggcagcccggcgatcaaaaagggcatctatcagagcctgaaaatcgtggatgaattagtggcgatcatgggctatgcgccgaaacgcatcgtggtggaaatggcgcgcgaaaaccagaccaccagcaccggcaaacgccgcagcatccagcgcctgaaaatcgtggaaaaagcgatggcggaaatcggcagcaacctgctgaaagaacagccgaccaccaacgaacagctgcgcgatacccgcctgtttctgtattatatgcagaacggcaaagatatgtataccggcgatgaactgtcactgcatcgcctcagccattatgatatcgatcatatcatcccgcagagctttatgaaagatgatagcctggataacctggtgctggtgggcagcaccgaaaaccgcggcaaaagcgatgatgtgccgagcaaagaagtggtgaaagatatgaaagcgtattgggaaaaactgtatgcggcgggcctgatcagccagcgcaaatttcagcgcctgaccaaaggcgaacagggcggcctgaccttagaagataaagcgcattttatccagcgccagctggtggaaacccgccagatcaccaaaaacgtggcgggcatcctggatcagcgctataacgcgaacagcaaagaaaagaaagtccagatcatcaccctgaaagcgagcctgaccagccagtttcgcagcatctttggcctgtataaagtgcgcgaagtgaacgattatcatcatggccaggatgcgtatctgaactgcgtggtggcgaccaccttactgaaagtgtatccgaacctggcgccggaatttgtgtatggcgaatatccgaaatttcaggcgtttaaagaaaacaaagcgaccgcgaaaaccatcatctataccaacctgatgcgcttctttaccgaagatgaaccgcgctttatgaaagatggcgaaattctgtggagcaacagctatctgaaaaacatcaaaaaggaactgaactaccaccagatgaacatcgtgaaaaaggtggaggtccagaaaggcggcttctcaaaggaaagcatcaaaccgaaaggcccgagcaacaaactgatcccggtgaaaaacggcctggacccgcagaaatatggcggctttgatagcccggtggtggcgtataccgtgctgtttacccatgaaaaaggcaaaaagccgctgatcaaacaggaaattctgggcatcaccatcatggagaaaacccgctttgaacagaacccgatcctgttcctggaagaaaaaggctttctgcgcccgcgcgtgctgatgaaactgccgaaatataccctctatgaatttccggaaggccgccgccgcctgctggcgagcgcgaaagaagcgcagaaaggcaaccagatggtgttaccggaacatctgctgaccctgctgtatcatgcgaaacagtgcctgctgccgaaccagagcgaaagcctggcgtatgtggaacagcatcagccggaatttcaggaaattctggaacgcgtggtggattttgcggaagtgcataccctggcgaaaagcaaagttcagcagattgttaaactgtttgaagcgaaccagaccgcggatgtgaaagaaattgcggcgagctttattcagctgatgcagtttaacgcgatgggcgccccgagcaccttcaaattctttcagaaagatattgaacgcgcgcgctataccagcattaaagaaatttttgatgcgaccattatctatcagtcaaccaccggcctgtatgaaacccgccgcaaagtggtggat

SEQ ID NO: 193 - DNA, 4308 bp, Enterococcus sp.
  misc_feature 1..4308: note = source = /note="APG04583.1 Native Seq"
  source 1..4308: mol_type = genomic DNA
atggctaaaaatatacttggattagatttgggaaccaatagtattggttgggcgttggtacagcaagactttgaaaacaaagaaggaaatattcttggaatgggaagtaggattattccgatgtcgcaagatattttaggagaattcggtaaggggaattctatttctcaaactgctgaacgtactggttatcgtggtgtccgtcggttaagagaacgacatttattacgtcgtgagcgtttgcaccgagttttgcatttgttgggtttcttgccaaaacattatgatgaaaaaatagattttacacaacgttttgggaaattcattaaccaagccgaacctaaattggcttttgatagtgaatttctttttaaagattctttccatgaaatgttagctgattttaaacaaaatcaaccagagtttttgaaagataaaaatggagaagattgtttagttccttatgattggacgatttattatttacgtaaaaaagcattaacgcaaaaaattgagaaatatgaattagcttggttgattcttaattttaatcaaaaacgtggttattatcaattaagaggtgaagaagagaaagaaaatccaaataaattggtgggatttcattctttgaaaatcgttgatgttattcccgatgctgaaacaaataaaaaaggagagacttggtattctttgcatttagaaaatggttgggtatatcgccgttcttctaaaatttcattagcggattggaaagataaagttcgagattttattgttacgactgatttaaacgatgatggttctgaaaaattggataaagatggaattgtgaaacgtagctttcgtgcaccaagtgcggatgattggactttgttgaaaaagaaaacagaaaaagatattgataactctaacaaaactgttggaacttatatttacgacaatcttttattgaacccaaaacaaaaaataaaaggaaaaatggttcgtaccatcgaacgtaagttttacaagcaagaattagaacaaattttaaaaactcaaaaagaatttcattcagaattacaaagtgaaaatttgctacaagattgtgttcgagaattgtatcgaaataatgaacaacatcaacaaatgttagaagctaaagattttgtgcatttgttcctaaatgatattattttctatcaacgtcctttgagaagtcaaaaatccagtatatcgaattgtactttggaatttagaaaatcgaaaaatgaaaatggtgaagaagttattcatcgtttaaaagtaattgcaaaatcgaatccatattatcaggaatttagattgttacaatgggtgcaaaatttagcaatttatacaaaagatgatgataaaaatgtaacaaacgaatttctaaagtctactcaagattgggaggatttattgagatggctacattctaaaaaagaaattaaacaggatgctttaattaagtttttgattgaaaagaaaggtttaaaaggcaaagctttaactattgaagtagcaaaatatcgttggaattatgttcaggacaaagattaccctggtaatgaaacccgatatttaattcaatctcgtttggataaagttgaatatgcacctaaggattttttaacttatgaaaatgaaatggctttgtggcacatcatttattcgataaacgataaaattgagtacgaaaaagccttaaaatcttttgccaacaaaaaaggtttggacgaagtaacttttgttgaagcgtttaagaaattcccgccttttaaaagtgattacggaagtttttctgaaaaagcaatcaagaaattattgcctttgatgcgttttggaactcaatggaattgggataatatcgatcaaaattctaaagaaagaattggaaaaatattgacaggcgaatatgatgaaaacatcaaaggtcgtgttcgcgaaaaagcaagacatctcaattctgaaaccgattttcaagctttacctttgtggttggcgcaatacgtagtttacggaagacattctgaagctgatattgcaggtaaatggaattcggtggatgatttaaagcaatttttagatgactttaaacaacattcgcttcgtaatcctattgtagagcaagtgattactgaaacgttgcgtgcggtaaaagatatttggaatttttatggaaaaggtgctaaagatttcttctctgaaattcatatcgagctggggcgtgaaatgaaaaatacggctgatgaacgcaagcgtattactacaatggttacggataacgaaaacaccaatttgcgtatcaaagctttgttggctgaaatggctttggatcaaaatgtagataatgttcgtccatattctccaatgcaacaagaaattttgaaaatctatgaagaaggtgttttgaatgctgaagaaaatatcgatgatgatattctgaaaattagtaaaacggctcaaccttctgctacagatttaaagcgttacaaattgtggttagaacaaaaatatcgttcgccttatacaggtcaaatgattcctttgaataagctgtttacacctgaatatgagattgaacacataattccgcaaagtcgctattttgatgatagtatgagcaataaagtgatttgcgaagcagcggtgaataaacttaaagacaatcaaattggtttggttttcattaagaatcatcacggagaagttgtggatttcggaatgggaaaacaagtgaagattttggaagtttccgattatgaggattttgtaaaacaaaattacaataaaaacagaggaaaacgtaataagttgttattggaagatattcctgagaaaatgattgaacgtcaactaaatgatacacgttacatcagcaaatatattacgcaagtgttatcgaatattgttcgtgatgataaagaaggtagtaaagatgatggtgtaaattctaaaaatattgttcctggaaacggaaaaattaccacaagacttaaacaagattggggattgaatgacgtttggaatgatttggttttacctcgttttgaacgaatgaatacattgactaattcaaatgattttacatcaaaaaatacacatggaaaaacaattccaacagttccaattgagttatccaaaggtttttctaaaaaacgtatcgatcatcgtcatcacgcaatggatgcgttggtaattgcctgtgcaacgagagatcacgtgaatttgttgaataacgaaagttcaaaatcggatacaaaacgttatgatttgaataggaaattaagaaaatacgagaaagtagcttacaatgatcccaaaacaggcgaacgaattgagaaagaagttccaaaagacttcattaaaccttgggagacctttaccgaagatacgcgaactttgttagaaaatattgtaattagtttcaagcaaaatctacgagttatcaataaagcaaccaattattatgaaaaaattgagaatgggaaaaaagtaaaagttgaacaaaagggaataaattgggctgtaagaaaagctttgcataaagaaaccgtttctggacaagtgcatttggatagaataaaagttgcaaaaggtaaaatattaacggctactcgtaaaactttggatgcctcttttaatgaaaaaacaattgagtcaattacggatacagggattcaaaagattttattgaattatttaaaatcaaaagataacaatccagaggttgcattttcaccagaaggaatagaagaattaaataaaaatattaggctatataatgatggaaaagcacatcaaccaattttgaaagttcgtgtttttgagcaaggaagcaaatttactttaggtgagacgggaaataaaaccactaagtttgtagaagcagcaaagggtactaatctatttttcggaatttatgaagacaaatcaggtaagagaagctacgaaaccattccattaaatattgtcatcgaaagacaaaaacaaggtctgcaagctgttcccgaaaccaatgagaaagggaaacaattgttgtttacgttatctccaaatgatttggtttacgttcctgaagaaggagtttttgatgagaataatatcaaggtagataggatttataaggtagtgagtttttctacctatcaatgtttttttgtaagaaatgatgtttctacttctgtagttaataaggttgaatattccgctttaaataaaatggaaaaatctattgacaacataatgataaaagaaaactgcgtcaaactgaatgtagaccgtttaggaaagatttcaaaagct

SEQ ID NO: 194 - DNA, 4308 bp, synthetic construct
  misc_feature 1..4308: note = source = /note="Description of Artificial Sequence: Syntheticpolynucleotide"
  misc_feature 1..4308: note = source = /note="APG04583.1 E. coli optimized"
  source 1..4308: mol_type = other DNA
atggcgaagaacatactaggcctggatctcggaaccaacagcattggttgggcactggtccagcaggacttcgagaataaagagggcaacatactaggcatgggcagccgaataatccctatgagtcaggacatactaggcgagttcggcaagggcaacagcattagccagaccgcagaacgtactggttatcgtggtgtgcgccgtttacgtgaacgtcatctgttacgtcgtgaacgtctgcatcgtgttctgcatctgttaggcttcctgccgaagcattacgatgaaaagatcgacttcacccagcgcttcggcaagttcattaatcaagcggaaccaaagctggcgttcgacagcgagttcctgttcaaggacagcttccatgaaatgctagcggacttcaagcagaatcaaccggagttcctgaaggataaaaacggcgaggactgcctggtgccgtacgactggaccatatactacctgcgcaagaaagcactgacccagaagatcgaaaaatacgagctagcgtggctcatcctgaacttcaatcaaaagcgcggatactaccagcttaggggcgaggaggagaaagagaacccgaataaactggtgggcttccacagcctgaaaatagtggacgtgatcccggacgcggaaaccaataaaaagggcgagacctggtactcactgcatctggaaaacggctgggtgtaccgccgcagcagcaaaataagcctggcggactggaaagacaaagtgcgcgacttcatcgtgaccaccgacctgaacgatgacggcagcgaaaaactggacaaagacggcatcgtgaaacgcagctttcgtgcaccgagcgcagatgattggaccctattaaagaaaaagaccgaaaaagacatcgacaacagcaataaaaccgtgggcacctacatctacgacaacctgttattaaacccgaagcagaaaatcaaaggcaaaatggtgcgcaccatcgaacgcaaattctacaaacaggagctggagcagatcctgaaaacccagaaggaattccacagcgaactgcagagcgaaaacctgctgcaggattgcgtgcgcgaactgtaccgcaataatgaacagcaccagcagatgctggaagcgaaagatttcgtgcacctgttcctgaacgatataatcttctaccagcgcccgctgcgcagccagaaatcaagcatcagcaattgcaccctggaattccgcaaaagcaagaatgaaaacggcgaagaagtgatccaccgcctgaaagtgatcgcgaaaagcaacccatactaccaggaattccgcctgctgcagtgggtccagaacctggcgatctatactaaagacgacgacaagaacgtaaccaacgagttcctgaaatcaacccaggactgggaagatctgctgcgctggctgcatagcaagaaggagattaaacaggacgcgctgatcaagttcctgatcgagaaaaagggcctgaagggcaaagcattgaccatcgaggtggcaaaataccgctggaactacgtccaggacaaagattacccgggcaatgaaacccgctacctgatccaatcaaggctggacaaagtggaatacgcgccgaaagatttcctgacctacgagaatgaaatggcgctgtggcacatcatctacagcattaatgataaaatcgaatatgaaaaagcattaaaatcgttcgcgaataaaaagggcctggatgaagtgaccttcgtggaagcattcaaaaagttcccgccgttcaaaagcgattacggcagcttcagcgaaaaagcgattaaaaagctgctgccattaatgcgcttcggcacccagtggaactgggataacatcgatcaaaatagcaaagaacgcatcggcaaaatactaaccggcgaatacgatgaaaatattaaaggccgcgtgcgcgaaaaagcgcgccatctgaacagcgaaaccgatttccaagcattgccgctgtggctggcgcagtacgtggtgtacggccgccatagcgaagcggatatcgcgggcaaatggaacagcgtggatgatctgaaacagttcctggatgatttcaaacagcatagcctgcgcaacccgatcgtggaacaggtgatcaccgaaaccctgcgcgcggtgaaagatatctggaacttctatggcaaaggcgcgaaagatttcttcagcgaaatacacatcgaactgggccgcgaaatgaaaaataccgcggatgaaagaaaacgcatcaccaccatggtgaccgataatgaaaataccaacttacgcattaaagcattgctggcggaaatggcgctggatcagaacgtggataacgtgcgcccgtatagcccgatgcagcaggaaatcctgaaaatatatgaagaaggcgtattaaacgcggaagaaaatatcgacgatgatatcctgaaaataagcaaaaccgcgcaaccaagcgcgaccgatctgaaacgctataaactgtggctggaacagaaatatcgcagcccgtataccggccagatgatcccgctgaacaaactgtttaccccggaatatgaaatcgaacatatcatcccgcagagccgctattttgatgatagcatgagcaacaaagtgatctgcgaagcggcggtgaacaaactgaaagataaccagatcggcctggtgtttatcaaaaaccatcatggcgaagtggtggattttggcatgggcaaacaggtgaaaatcctggaagtgagcgattatgaagattttgtgaaacagaactataacaaaaaccgcggcaaacgcaacaaattactgctggaagatatcccggagaaaatgatcgaacgccagctgaacgatacccgctatatcagcaaatatatcacccaggtgctgagcaacatcgtgcgcgatgataaagaaggcagcaaagatgatggcgtgaacagcaaaaacatcgtgccgggcaacggcaaaatcaccacccgcctgaaacaggattggggcctgaacgatgtgtggaacgatctggtgctgccgcgctttgaacgcatgaacaccctgaccaacagcaacgattttaccagcaaaaacacccatggcaaaaccattccgaccgtgccgattgaactgagcaaaggctttagcaaaaagcgcattgatcatcgccatcatgcgatggatgcgctggtgattgcgtgcgcgacccgcgatcatgtgaacttactgaacaacgaaagcagcaaaagcgataccaaacgctatgatctgaaccgcaaactgcgcaaatatgaaaaagtggcgtataacgatccgaaaaccggcgaacgcattgaaaaagaagtgccgaaagattttattaaaccgtgggaaacctttaccgaagatacccgcaccctgctggaaaacattgtgattagctttaaacagaacctgcgcgtgattaacaaagcgaccaactattatgagaaaattgaaaacggcaaaaaggtgaaagtggaacagaaaggcattaactgggcggtgcgcaaagcgctgcataaagaaaccgtgagcggccaggtgcatctggatcgcattaaagtggcgaaaggcaaaattctgaccgcgacccgcaaaaccctggatgcgtcattcaacgagaaaaccattgaaagcattaccgataccggcattcagaaaattctgctgaactatctgaaaagcaaagataacaacccggaagtggcgtttagcccggaaggcattgaagaactgaacaaaaacattcgcctgtataacgatggcaaagcgcatcagccgattctgaaagtgcgcgtgtttgaacagggcagcaaattcaccctgggcgaaaccggcaacaaaaccaccaaatttgtggaagcggcgaaaggcaccaacctgttctttggcatctatgaagataaaagcggcaaacgcagctatgaaaccattccgctgaacattgtgattgaacgccagaaacagggcctgcaagcggtgccggaaaccaacgaaaaaggcaaacagctgctgtttaccctgtcaccgaacgatctggtgtatgtgccggaagaaggcgtgtttgatgaaaacaacattaaagtggatcgcatctataaagtggtgagctttagcacctatcagtgcttctttgtgcgcaacgatgtgagcaccagcgtggtgaacaaagtggaatatagcgcgctgaacaaaatggaaaaatcgattgataacattatgattaaagaaaactgcgtgaaactgaacgtggatcgcctgggcaaaattagcaaagcg

SEQ ID NO: 195 - DNA, 4368 bp, Empedobacter sp.
  misc_feature 1..4368: note = source = /note="APG01688.1 Native Seq"
  source 1..4368: mol_type = genomic DNA
atgatgattaaaaatatacttggattagatttggggactaactctattgggtgggcattgataaaacaagattttgaaaataagcatggcgaaattcttggaatgggtagccggattattccgatgtcacaggatattctgggcgattttgggaaaggaaattctatttcgcaaaccgctgatcgtaccaaatacagaagcgtgagaagattacgtgaacgatttttattgaggagagaacgactacacagagttttacatcttttaaattttcttccacagcattatgcttcacaaattgattttgaaaaaaaattcgggaagtttaaatctgaaactgaacccaaattggcatgggaaaattggggtggaaagttttcattccttttccaaaactctttcaatgaaatgcttgaagattttaaagcagctggacagggtttaaaaattccttacgactggacaatttattatctccgtaaaaaagcactttcacaaaaaattgaaaaggaggaactggcctggattcttttaaactttaatcagaaacgaggatattatcaattgcgtggtgaggaagaagaagagaatcctaataagctggttgaattttattctttaaaaattgtagatgttgtagcagatgaacctcaaaaaggaaaatctgatatttggtattctttgattttagagaatggatgggtttacagacgagcaagcaaaatacccctatttgactggaaagataaaacaagagattttattgtaacaactgatttgaatgatgacagaagtgttaaaacagacaaagaaggaaatgaaaaaagaagtttcagagcaccaagcgaaaacgattggacattggtaaaaaagaaaaccgaacaggaaatcgaccaatctcacaaaaccgttggaacctatatctacgaaacacttcttctaaatccgaaacaaaaaattaaaggaaaattggttcggacgattgaaagaaaattctataaagatgagctaaaacaaattttagaaaaacaaaaggaatttcatcaggaacttaaaaatgatgatttgtataatgattgcattcgtgagttgtacagaaacaacgaagcacatcagctgactttgagcaagaaagattttgttcatcttttgatggatgatcttattttctaccaaagacctttgagaagccagaaatcatctatttccaactgtacgttagagtttagaaaatataaagatgaaaatggaatagagcatacacaatatttaaaagccattccaaaatccaatccgtattatcaggaatttcgtctttggcaatggatgtataatctgaatatttacagaaaggacgatgaagcgaatgttaccaaagaatttttaaatacgaacaaagattttgaaagtctgtttgaatttttaaataatagaaaagaaattgagcaaaagccattgattaaatttcttttggaacaaaaagatatcaataaaaaattgcttaacgctgaagcagaaaaatatcgctggaactatgtagaagacaagaaatatccttgcaatgaaaccaaaacgatgatttcttctcgtttggataaagtcgaaaacatttctgatgatttcctgacaagggacattgagcagaaaatttggcacatcatctattccgtcaatgataaaatagaatatgaaaaagctttgaaatcttttgcaactagaaacgatttggatgaaaactcttttatcgaagcgtttaagaaattctcgccttttaaaagtgaatatggttctttttcggaaaaagcaattaaaaagttactgcctttaatgcgattgggtaaatattggtatgaagatgaaattgtaaagcatagtgatatttatttcaaaaatattgagaatcttttgggtgatttttcaaatagagacaaaaaaatttctgaagaagacaaagagaaatggaataaatctataaatctaaagttacaggaagagttaaaagattttcaaacagctgaaatagatttatttcaaggattacgattgcatattgctcaataccttgtttatggaagacattcagaagcttcaatgattggaaaatggaattccgccgaagatttagaagaatttttaaaggattttaaacagcattcgcttcgcaacccgattgtagaacaagtgattacagaaactttgcgtgttgtaaaagatatttggttgaaatacggaaatggagcaaaggatttcttcaatgaaattcatattgagttaggaagagaaatgaaacttcccgcagatgatcgaaaaaaactaacgaaccagatttctgaaaacgaaaataccaatttccgcatcaaagctctattggctgaaatgatgaatgacagttcggtagaaaatgtccgtccgttttcgccgatgcagcaagaaattttaaaaatttatgaagacgatgttttaaaatcagatatagaaattgaagatgatattctgaaaatcagcaaaaccgctcaaccttctccttccgatttgaaacgatataaactttggttggaacagaaatacaaatcgccttacacggggcaaattattcctttgaataaattgtttacaccagaatacgaaattgagcacattattccgcagagccgatattttgatgacagttttagcaataaagtaatttgtgagtctgcggttaataaattgaaagataactacatcggacttgaatttattaagcagttcggaggaacgattattgaacttggttttggtaaaagcataaaagtttttgaaacaaaagaatacgaagatttcgtcaaaaaacattacgccaacaatcaaggtaaaagaaacaaacttttgatggaagatattccagagaaaatgattgaacgtcaaatgaacgatacacgatatatcagcaaatatatttcgggcgttttgtctaatattgttcgggtagaagatggttcagatgaaggggtaaattctaaaaatattgttcccggaaacggaaaaatcaccacacagctgaaacaagattggggattgaatgatgtttggaatgatttgattttaccacgctttgaaagaatgaaccaactcaccaattcaaaagtttttactgcctggaatgagaattatcaaaagtttttaccaactgttcctattgaatattccaaagggttttcaaagaaaagaatagaccaccgccatcacgctttagatgctttggtgattgcctgtgctacaaaagatcacgtgaatttattaaataatcaatcggcaaaatcggataccaaacgatacgatttgaaaaagaaatcgatgaagtttgaaaaagtagtttacaatgatgccaaaacaggagagaaaatcgaaagggaagtgccaaaacaatttttaaaaccttgggaaaattttacgctagatgttaaacataatttggaaactattattgtaagttttaagcaaaatcttcgtgttattaataaagcgactaattattacgaaaagtatgttgagaaagacggtacaaaaaataaggaaagagtagagcagaccggaacaaactgggcgattaggaaaccaatgcataaagacacggtttccggtaaagtagatttaccttgggtaaaagtcccaaaagggaaaattttaacagcaacaaggaaaagccttgatagttcgtttgacttaaagtcaataggctctattacggatacaggaattcagaaaatactcaaaaattatttagcatttaaagacggaaatcctgaactggctttttcaccagaaggaattgacgatttgaataaaaatattgaaaaatacaatgatggaaaaccgcatcaacccatcaataaagtaagggtttttgaattgggaagtaaatttcaggtaggacaaagtggaaataaaaaagataaatatgtagaagctgcaaaaggaactaatctattctttgctgtttatgaagatgaaaaaggaaagagaaattatgaaaccattcctttgaatgaagtgattgaaaggcaaaagcaaggtttatctgtggttgatttaaaaggtacaaatgatttctacttatgtccgaatgattttgtatatattccatcaggcgacgaacttgaaaatataaataatgttgattttaaagacattaaaaaagagataaacgaaagaatttacaaagtagtaagttttacaggtaatagactttcctgtattccatatatggttgcaacaacgattgttaataaattagagtttacgcaacttaataaaattgaattcacaaaagaaaaagaaatttgtataaaactaaatgtcgatcgtttaggtaacatttcaaaagcg

SEQ ID NO: 196 - DNA, 4368 bp, synthetic construct
  misc_feature 1..4368: note = source = /note="Description of Artificial Sequence: Syntheticpolynucleotide"
  misc_feature 1..4368: note = source = /note="APG01688.1 E. coli optimized"
  source 1..4368: mol_type = other DNA
atgatgattaaaaatatcctaggcctggacctgggaaccaacagcatcggctgggcgctgattaaacaggacttcgaaaataagcatggcgagatactaggcatgggcagccgaataatcccgatgagtcaggacatactaggcgacttcggcaagggcaacagcatcagccagaccgcggaccgcacaaaatatcgcagcgtgcgtagactgcgcgaacgctttctgttacgtcgtgaacgtctgcatcgtgttcttcacctattaaacttcctgccgcagcattacgctagccagatcgacttcgagaagaagttcggcaagttcaagagcgagaccgaaccaaaactggcgtgggaaaactggggcggcaaattcagcttcctattccaaaatagcttcaacgagatgctagaggacttcaaggcggcgggtcagggattaaaaataccgtacgactggaccatatactacctgcgcaagaaagcgctgagccagaagatcgagaaggaggagctggcgtggatactactgaactttaatcaaaagcgcggatactaccagctgcgcggcgaggaggaggaggagaacccgaataaactggtggagttctacagcctgaaaatagtggacgtggtggcggacgagccgcagaaaggcaaaagcgacatctggtactcactcatactagaaaacggctgggtgtatcgccgcgcgagcaaaataccgctgttcgactggaaagataaaacccgcgacttcatcgtgaccaccgacctgaacgacgaccgcagcgtgaaaaccgacaaagaaggcaatgaaaaacgcagctttcgcgcgccgagcgaaaacgattggaccctggtgaaaaagaaaaccgaacaggaaatcgatcagagccataaaaccgtgggcacatatatctatgaaaccctgttattaaacccgaagcagaagattaaaggcaaactggtgcgcaccatcgaacgcaaattctacaaagacgaattaaaacagatactagaaaaacagaaagaattccaccaggaattaaagaacgacgacttatataacgattgcatccgcgaactgtaccgcaataatgaagcgcaccaattaaccctgagcaaaaaggatttcgtgcacttattaatggatgatctgatcttctaccagcgcccgctgcgcagccagaaatcaagcatcagcaattgcaccctggaattccgcaaatacaaagatgaaaacggcatcgaacacacccagtacctgaaagcgatcccgaaatcaaacccatactaccaggaattccgcctgtggcagtggatgtacaacctgaacatctaccgcaaagatgatgaagcgaacgtaaccaaagaattcctgaacaccaataaagatttcgaaagcctgttcgaattcctgaataatcgcaaagaaatcgaacagaaaccattaatcaaattcctgctggaacagaaagatattaataaaaagttattaaacgcggaagcggaaaaatacagatggaactacgtggaagataaaaagtacccgtgcaatgaaaccaaaaccatgatcagcagccgcctggataaagtggaaaatatcagcgatgatttcctgacccgcgatatcgaacagaaaatatggcatataatctacagcgtgaacgataaaatcgaatatgaaaaagcattaaaatcgttcgcgacccgcaacgatctggatgaaaatagcttcatcgaagcattcaaaaagttcagcccgttcaaaagcgaatacggcagcttcagcgaaaaagcgattaaaaagctgctgccattaatgcgcctgggaaaatactggtacgaagatgaaatcgtgaaacacagcgatatctacttcaaaaacatcgaaaacctgctgggcgatttcagcaaccgcgataaaaagatcagcgaagaagataaagaaaaatggaacaaaagcatcaacctgaaactgcaggaagaactgaaagatttccagaccgcggaaatcgatctgttccagggcctgcgcctgcatatcgcgcagtacctggtgtacggccgccatagcgaagcgagcatgatcggcaaatggaactcagcggaagatctggaagaattcctgaaagatttcaaacagcatagcctgcgcaacccgatcgtggaacaggtgatcaccgaaaccctgcgcgtggtgaaagatatctggctgaaatatggcaacggcgcgaaagatttctttaacgaaattcatatcgaactgggccgcgaaatgaaactgccggcggatgatcgcaaaaagctgaccaaccagatcagcgaaaacgaaaacaccaactttcgcatcaaagcgctgctggcggaaatgatgaacgatagcagcgtggaaaacgtgcgcccgtttagcccgatgcagcaggaaatcctgaaaatctatgaagatgatgtgctgaaaagcgatatcgaaatcgaagatgatatcttaaaaatcagcaaaaccgcgcagccgagcccgagcgatctgaaacgctataaactgtggctggaacagaaatataaaagcccgtataccggccagatcatcccgctgaacaaactgtttaccccggaatatgaaatcgaacatatcatcccgcagagccgctattttgatgatagctttagcaacaaagtgatctgcgaaagcgcggtgaacaaactgaaagataactatatcggcctggaattcatcaaacagtttggcggcaccatcatcgaactgggctttggcaaaagcatcaaagtgtttgaaaccaaagaatatgaagattttgtgaaaaagcattatgcgaacaaccagggcaaacgcaacaaactgttaatggaagatatcccggagaaaatgatcgaacgccagatgaacgatacccgctatatcagcaaatatatcagcggcgtgctgagcaacatcgtgcgcgtggaagatggcagcgatgaaggcgtgaacagcaaaaacatcgtgccgggcaacggcaaaatcaccacccagctgaaacaggattggggcctgaacgatgtgtggaacgatctgatcctgccgcgctttgaacgcatgaaccagctgaccaacagcaaagtgtttaccgcgtggaacgaaaactatcagaaatttctgccgaccgtgccgattgaatatagcaaaggctttagcaaaaagcgcattgatcatcgccatcatgcgctggatgcgttagtgattgcgtgcgcgaccaaagatcatgtgaacctgctgaacaaccagagcgcgaaaagcgataccaaacgctatgatctgaaaaagaaaagcatgaaatttgaaaaagtggtgtataacgatgcgaaaaccggcgagaaaattgaacgcgaagtgccgaaacagtttctgaaaccgtgggaaaactttaccctggatgtgaaacataacctggaaaccattattgtgagctttaaacagaacctgcgcgtgattaacaaagcgaccaactattatgaaaaatatgtggaaaaagatggcaccaaaaacaaagaacgcgtggaacagaccggcaccaactgggcgattcgcaaaccgatgcataaagataccgtgtcaggcaaagtggatctgccgtgggtgaaagtgccgaaaggcaaaattctgaccgcgacccgcaaaagcctggatagcagctttgatctgaaaagcattggcagcattaccgataccggcattcagaaaattctgaaaaactatctggcgtttaaagatggcaacccggaactggcgtttagcccggaaggcattgatgatctgaacaaaaacattgaaaaatataacgatggcaaaccgcatcagccgattaacaaagtgcgcgtgtttgaactgggcagcaaatttcaggtgggccagagcggcaacaaaaaggacaaatatgtggaagcggcgaaaggcaccaacctgttctttgcggtgtatgaagatgaaaaaggcaaacgcaactatgaaaccatcccgttaaacgaagtgattgaacgccagaaacagggcctgagcgtggtggatctgaaaggcaccaacgatttttatctgtgcccgaacgattttgtgtatattccgagcggcgatgaactggaaaacattaacaacgtggattttaaagatattaaaaaggaaattaacgaacgcatctataaagtggtgagctttaccggcaaccgcctgagctgcattccgtatatggtggcgaccaccattgtgaacaaactggaattcacccagctgaacaaaattgaattcaccaaagaaaaagaaatttgcattaaactgaacgtggatcgcctgggcaacattagcaaagcg

SEQ ID NO: 197 - DNA, 25 bp, Homo sapiens
  misc_feature 1..25: note = source = /note="target seq for Ataxia-telangiectasia syndrome"
  source 1..25: mol_type = genomic DNA
gaattattccagaaagccaaagtag

SEQ ID NO: 198 - DNA, 20 bp, Homo sapiens
  misc_feature 1..20: note = source = /note="target seq for Very long chain acyl-CoAdehydrogenase deficiency"
  source 1..20: mol_type = genomic DNA
ggcttcatgaaggtacagga

SEQ ID NO: 199 - DNA, 25 bp, Homo sapiens
  misc_feature 1..25: note = source = /note="target seq for Abnormality of T cell physiology"
  source 1..25: mol_type = genomic DNA
ggtgtgctcaccagaatggagtaca

SEQ ID NO: 200 - DNA, 25 bp, Homo sapiens
  misc_feature 1..25: note = source = /note="target seq for Cardiovascular phenotype"
  source 1..25: mol_type = genomic DNA
gcagatcattggggtggatcccgaa

SEQ ID NO: 201 - DNA, 25 bp, Homo sapiens
  misc_feature 1..25: note = source = /note="target seq for 3-Oxo-5 alpha-steroid delta4-dehydrogenase deficiency"
  source 1..25: mol_type = genomic DNA
ggctgcaagcttttcaccaccayag

SEQ ID NO: 202 - DNA, 25 bp, Homo sapiens
  misc_feature 1..25: note = source = /note="target seq for Acute myeloid leukemia"
  source 1..25: mol_type = genomic DNA
gggcggcatgaaccggaggcccatc

SEQ ID NO: 203 - DNA, 20 bp, Homo sapiens
  misc_feature 1..20: note = source = /note="target seq for Acute myeloid leukemia"
  source 1..20: mol_type = genomic DNA
gcatgggcggcatgaaccgg

SEQ ID NO: 204 - DNA, 20 bp, Homo sapiens
  misc_feature 1..20: note = source = /note="target seq for Cutaneous malignant melanoma 3"
  source 1..20: mol_type = genomic DNA
gtggccactgtggggatcac

SEQ ID NO: 205 - DNA, 20 bp, Homo sapiens
  misc_feature 1..20: note = source = /note="target seq for Alpha-1-antitrypsin deficiency"
  source 1..20: mol_type = genomic DNA
gtgctgaccatcgacgagaa

SEQ ID NO: 206 - DNA, 20 bp, Homo sapiens
  misc_feature 1..20: note = source = /note="target seq for Charcot-Marie-Tooth disease, type 2"
  source 1..20: mol_type = genomic DNA
ggcgagctgcatgatctgcg

SEQ ID NO: 207 - DNA, 25 bp, Homo sapiens
  misc_feature 1..25: note = source = /note="target seq for Hereditary cancer-predisposing syndrome"
  source 1..25: mol_type = genomic DNA
gcacatgacggaggttgtgaggcgc

SEQ ID NO: 208 - DNA, 25 bp, Homo sapiens
  misc_feature 1..25: note = source = /note="target seq for Hereditary cancer-predisposing syndrome"
  source 1..25: mol_type = genomic DNA
gaggttgtgaggcactgcccccacc

SEQ ID NO: 209 - DNA, 20 bp, Homo sapiens
  misc_feature 1..20: note = source = /note="target seq for Hereditary cancer-predisposing syndrome"
  source 1..20: mol_type = genomic DNA
gcagcatccggctgcaggta

SEQ ID NO: 210 - DNA, 25 bp, Homo sapiens
  misc_feature 1..25: note = source = /note="target seq for Brugada syndrome"
  source 1..25: mol_type = genomic DNA
ggccaaggggatccgcacgctgctc

SEQ ID NO: 211 - DNA, 20 bp, Homo sapiens
  misc_feature 1..20: note = source = /note="target seq for Brugada syndrome"
  source 1..20: mol_type = genomic DNA
gcctgatgacgcaggactgc

SEQ ID NO: 212 - DNA, 20 bp, Homo sapiens
  misc_feature 1..20: note = source = /note="target seq for GRACILE syndrome"
  source 1..20: mol_type = genomic DNA
ggtacgaagtctcgacactg

SEQ ID NO: 213 - DNA, 20 bp, Homo sapiens
  misc_feature 1..20: note = source = /note="target seq for Enhanced s-cone syndrome"
  source 1..20: mol_type = genomic DNA
ggcattggcggtggacccca

SEQ ID NO: 214 - DNA, 25 bp, Homo sapiens
  misc_feature 1..25: note = source = /note="target seq for Charcot-Marie-Tooth disease, type 2"
  source 1..25: mol_type = genomic DNA
gaagaacaggttctggacgtcaaag

SEQ ID NO: 215 - DNA, 20 bp, Homo sapiens
  misc_feature 1..20: note = source = /note="target seq for Charcot-Marie-Tooth disease, type 2"
  source 1..20: mol_type = genomic DNA
gaacaggttctggacgtcaa

SEQ ID NO: 216 - DNA, 20 bp, Homo sapiens
  misc_feature 1..20: note = source = /note="target seq for Arylsulfatase a, allele a"
  source 1..20: mol_type = genomic DNA
gacaggtcatagagcagcgg

SEQ ID NO: 217 - DNA, 20 bp, Homo sapiens
  misc_feature 1..20: note = source = /note="target seq for Familial hypercholesterolemia"
  source 1..20: mol_type = genomic DNA
gaacgtggtcgctctggaca

SEQ ID NO: 218 - DNA, 20 bp, Homo sapiens
  misc_feature 1..20: note = source = /note="target seq for Familial hypercholesterolemia"
  source 1..20: mol_type = genomic DNA
gaacgtggtcgctctggaca

SEQ ID NO: 219 - DNA, 25 bp, Homo sapiens
  misc_feature 1..25: note = source = /note="target seq for HEMOGLOBIN ARLINGTON PARK"
  source 1..25: mol_type = genomic DNA
ggtgcatctgactcctgaggagaag

SEQ ID NO: 220 - DNA, 20 bp, Homo sapiens
  misc_feature 1..20: note = source = /note="target seq for Familial hypertrophic cardiomyopathy 1"
  source 1..20: mol_type = genomic DNA
gaagtccgaggctcgccgca

SEQ ID NO: 221 - DNA, 20 bp, Homo sapiens
  misc_feature 1..20: note = source = /note="target seq for Cardiovascular phenotype"
  source 1..20: mol_type = genomic DNA
gtcaaggtcatcggtgaggc

SEQ ID NO: 222 - DNA, 25 bp, Homo sapiens
  misc_feature 1..25: note = source = /note="target seq for Cardiovascular phenotype"
  source 1..25: mol_type = genomic DNA
ggtcatcagtgaggccggccggggt

SEQ ID NO: 223 - DNA, 25 bp, Homo sapiens
  misc_feature 1..25: note = source = /note="target seq for Brugada syndrome"
  source 1..25: mol_type = genomic DNA
gtctcagccttacgcaccttccgag

SEQ ID NO: 224 - DNA, 25 bp, Homo sapiens
  misc_feature 1..25: note = source = /note="target seq for Brugada syndrome"
  source 1..25: mol_type = genomic DNA
gcaccttccaagtcctccgggccct

SEQ ID NO: 225 - DNA, 26 bp, Homo sapiens
  misc_feature 1..26: note = source = /note="target seq for Hereditary cancer-predisposing syndrome"
  source 1..26: mol_type = genomic DNA
gaagccagcccctcagggcaactgac

SEQ ID NO: 226 - DNA, 20 bp, Homo sapiens
  misc_feature 1..20: note = source = /note="target seq for Deficiency of butyryl-CoA dehydrogenase"
  source 1..20: mol_type = genomic DNA
ggcgactcatgggttctgaa

SEQ ID NO: 227 - DNA, 25 bp, Homo sapiens
  misc_feature 1..25: note = source = /note="target seq for Deficiency of butyryl-CoA dehydrogenase"
  source 1..25: mol_type = genomic DNA
gcgactcacgggttctgaatggaac

SEQ ID NO: 228 - DNA, 20 bp, Homo sapiens
  misc_feature 1..20: note = source = /note="target seq for Benign scapuloperoneal musculardystrophy with cardiomyopathy"
  source 1..20: mol_type = genomic DNA
ggaactgcgggcccagcatg

SEQ ID NO: 229 - DNA, 20 bp, Homo sapiens
  misc_feature 1..20: note = source = /note="target seq for Benign scapuloperoneal musculardystrophy with cardiomyopathy"
  source 1..20: mol_type = genomic DNA
ggacgagtaccaggagcttc

SEQ ID NO: 230 - DNA, 20 bp, Homo sapiens
  misc_feature 1..20: note = source = /note="target seq for Cone-rod dystrophy 6"
  source 1..20: mol_type = genomic DNA
ggatctgatccgggagcgca

SEQ ID NO: 231 - DNA, 25 bp, Homo sapiens
  misc_feature 1..25: note = source = /note="target seq for Cone-rod dystrophy 6"
  source 1..25: mol_type = genomic DNA
gaatgcactatgttctattccatcc

SEQ ID NO: 232 - DNA, 25 bp, Homo sapiens
  misc_feature 1..25: note = source = /note="target seq for Stargardt disease 1"
  source 1..25: mol_type = genomic DNA
gagctgctcacaggacgagaacatc

SEQ ID NO: 233 - DNA, 20 bp, Homo sapiens
  misc_feature 1..20: note = source = /note="target seq for Leber congenital amaurosis 2"
  source 1..20: mol_type = genomic DNA
gtctatccagtaagtatctc

SEQ ID NO: 234 - DNA, 20 bp, Homo sapiens
  misc_feature 1..20: note = source = /note="target seq for Cone-rod dystrophy 3"
  source 1..20: mol_type = genomic DNA
ggtacgtccatgccacaccc

SEQ ID NO: 235 - DNA, 20 bp, Homo sapiens
  misc_feature 1..20: note = source = /note="target seq for Nonsyndromic Oculocutaneous Albinism"
  source 1..20: mol_type = genomic DNA
ggacctttacggcgtaatcc

SEQ ID NO: 236 - DNA, 20 bp, Homo sapiens
  misc_feature 1..20: note = source = /note="target seq for Phenylketonuria"
  source 1..20: mol_type = genomic DNA
gtcatcctttggtgaattac

SEQ ID NO: 237 - DNA, 25 bp, Homo sapiens
  misc_feature 1..25: note = source = /note="target seq for Phenylketonuria"
  source 1..25: mol_type = genomic DNA
gtattacgtggcagagagttttaat

SEQ ID NO: 238 - DNA, 20 bp, Homo sapiens
  misc_feature 1..20: note = source = /note="target seq for Breast-ovarian cancer, familial 1"
  source 1..20: mol_type = genomic DNA
ggacgctcttgtattatctg

SEQ ID NO: 239 - DNA, 25 bp, Homo sapiens
  misc_feature 1..25: note = source = /note="target seq for Hyperphenylalaninemia, non-pku"
  source 1..25: mol_type = genomic DNA
gcccttctcagttcgctacgaccca

SEQ ID NO: 240 - DNA, 20 bp, Homo sapiens
  misc_feature 1..20: note = source = /note="target seq for Hyperphenylalaninemia, non-pku"
  source 1..20: mol_type = genomic DNA
gctacgacccatacacccaa

SEQ ID NO: 241 - DNA, 20 bp, Homo sapiens
  misc_feature 1..20: note = source = /note="target seq for Hereditary cancer-predisposing syndrome"
  source 1..20: mol_type = genomic DNA
gcgctatgttctattccatc

SEQ ID NO: 242 - DNA, 25 bp, Homo sapiens
  misc_feature 1..25: note = source = /note="target seq for Hereditary cancer-predisposing syndrome"
  source 1..25: mol_type = genomic DNA
ggggctggtattcatgaaaggcaac

SEQ ID NO: 243 - DNA, 20 bp, Homo sapiens
  misc_feature 1..20: note = source = /note="target seq for Hereditary cancer-predisposing syndrome"
  source 1..20: mol_type = genomic DNA
gcgctatgttctattccatc

SEQ ID NO: 244 - DNA, 25 bp, Homo sapiens
  misc_feature 1..25: note = source = /note="target seq for Hereditary cancer-predisposing syndrome"
  source 1..25: mol_type = genomic DNA
gtgttctcaggttatcggtaagttt

SEQ ID NO: 245 - DNA, 25 bp, Homo sapiens
  misc_feature 1..25: note = source = /note="target seq for Hereditary cancer-predisposing syndrome"
  source 1..25: mol_type = genomic DNA
ggaaatgctgttagtcggtatgtcg

SEQ ID NO: 246 - DNA, 25 bp, Homo sapiens
  misc_feature 1..25: note = source = /note="target seq for Hereditary cancer-predisposing syndrome"
  source 1..25: mol_type = genomic DNA
ggaaatgctgttagtcagtatgtcg

SEQ ID NO: 247 - DNA, 20 bp, Homo sapiens
  misc_feature 1..20: note = source = /note="target seq for Anterior segment dysgenesis 6"
  source 1..20: mol_type = genomic DNA
gtggccactgatcggaaacg

SEQ ID NO: 248 - DNA, 25 bp, Homo sapiens
  misc_feature 1..25: note = source = /note="target seq for Brugada syndrome"
  source 1..25: mol_type = genomic DNA
gaacctgatcctggccgtggtcgca

SEQ ID NO: 249 - DNA, 20 bp, Homo sapiens
  misc_feature 1..20: note = source = /note="target seq for Brugada syndrome"
  source 1..20: mol_type = genomic DNA
gatcctggccgtggtcgcaa

SEQ ID NO: 250 - DNA, 25 bp, Homo sapiens
  misc_feature 1..25: note = source = /note="target seq for Ornithine carbamoyltransferasedeficiency"
  source 1..25: mol_type = genomic DNA
gaagggccatgaccttctcactcta

SEQ ID NO: 251 - DNA, 25 bp, Homo sapiens
  misc_feature 1..25: note = source = /note="target seq for Osteogenesis imperfecta type I"
  source 1..25: mol_type = genomic DNA
ggtgctcgaggattgcccggaacag

SEQ ID NO: 252 - DNA, 25 bp, Homo sapiens
  misc_feature 1..25: note = source = /note="target seq for Osteogenesis imperfecta type I"
  source 1..25: mol_type = genomic DNA
gctcgaggattgcccggaacagctg

SEQ ID NO: 253 - DNA, 25 bp, Homo sapiens
  misc_feature 1..25: note = source = /note="target seq for Constipation"
  source 1..25: mol_type = genomic DNA
gattccagttaaatggatggcaatt

SEQ ID NO: 254 - DNA, 25 bp, Homo sapiens
  misc_feature 1..25: note = source = /note="target seq for Dopamine beta hydroxylase deficiency"
  source 1..25: mol_type = genomic DNA
ggacactgcctattttgcggtgagt

SEQ ID NO: 255 - DNA, 25 bp, Homo sapiens
  misc_feature 1..25: note = source = /note="target seq for Cystic fibrosis"
  source 1..25: mol_type = genomic DNA
gacttcatccaggtatgtaaaaata

SEQ ID NO: 256 - DNA, 25 bp, Homo sapiens
  misc_feature 1..25: note = source = /note="target seq for Phenylketonuria"
  source 1..25: mol_type = genomic DNA
gatgtaaacctgacccacactgaat

SEQ ID NO: 257 - DNA, 20 bp, Homo sapiens
  misc_feature 1..20: note = source = /note="target seq for Deficiency ofUDPglucose-hexose-1-phosphate uridylyltransferase"
  source 1..20: mol_type = genomic DNA
gagcccctgacacccttacc

SEQ ID NO: 258 - DNA, 20 bp, Homo sapiens
  misc_feature 1..20: note = source = /note="target seq for Amyloid Cardiomyopathy,Transthyretin-related"
  source 1..20: mol_type = genomic DNA
gtcaccaatcccaaggaatg

SEQ ID NO: 259 - DNA, 25 bp, Homo sapiens
  misc_feature 1..25: note = source = /note="target seq for Carbohydrate-deficientglycoprotein syndrome type I"
  source 1..25: mol_type = genomic DNA
ggctactccatgacagcgcctgagg

SEQ ID NO: 260 - DNA, 25 bp, Homo sapiens
  misc_feature 1..25: note = source = /note="target seq for Metachromatic leukodystrophy"
  source 1..25: mol_type = genomic DNA
gcatcccgtactcccacgaccagat

SEQ ID NO: 261 - DNA, 25 bp, Homo sapiens
  misc_feature 1..25: note = source = /note="target seq for Smith-Lemli-Opitz syndrome"
  source 1..25: mol_type = genomic DNA
gttcttcaatgggcgccccgggatc

SEQ ID NO: 262 - DNA, 20 bp, Homo sapiens
  misc_feature 1..20: note = source = /note="target seq for Deafness, autosomal recessive 1A"
  source 1..20: mol_type = genomic DNA
gcaggtgagcccgccggccc

SEQ ID NO: 263 - DNA, 25 bp, Homo sapiens
  misc_feature 1..25: note = source = /note="target seq for Congenital omphalocele"
  source 1..25: mol_type = genomic DNA
gccagcgctcctagtggccatgcac

SEQ ID NO: 264 - DNA, 25 bp, Homo sapiens
  misc_feature 1..25: note = source = /note="target seq for Congenital omphalocele"
  source 1..25: mol_type = genomic DNA
gcgctcctagtggccatgcacgtgg

SEQ ID NO: 265 - DNA, 20 bp, Homo sapiens
  misc_feature 1..20: note = source = /note="target seq for Congenital omphalocele"
  source 1..20: mol_type = genomic DNA
gtccacgccagcgctcctag

SEQ ID NO: 266 - DNA, 20 bp, Homo sapiens
  misc_feature 1..20: note = source = /note="target seq for Congenital omphalocele"
  source 1..20: mol_type = genomic DNA
gctcctagtggccatgcacg

SEQ ID NO: 267 - DNA, 25 bp, Homo sapiens
  misc_feature 1..25: note = source = /note="target seq for Congenital myotonia, autosomaldominant form"
  source 1..25: mol_type = genomic DNA
gagaggattctttgcagccacgttc

SEQ ID NO: 268 - DNA, 20 bp, Homo sapiens
  misc_feature 1..20: note = source = /note="target seq for Breast-ovarian cancer, familial 1"
  source 1..20: mol_type = genomic DNA
ggtgacccgagagtgggtgt

SEQ ID NO: 269 - DNA, 20 bp, Homo sapiens
  misc_feature 1..20: note = source = /note="target seq for Breast and/or ovarian cancer"
  source 1..20: mol_type = genomic DNA
gatgcctggacagaggacaa

SEQ ID NO: 270 - DNA, 20 bp, Homo sapiens
  misc_feature 1..20: note = source = /note="target seq for Breast-ovarian cancer, familial 1"
  source 1..20: mol_type = genomic DNA
ggacaatggcttccatggta

SEQ ID NO: 271 - DNA, 20 bp, Homo sapiens
  misc_feature 1..20: note = source = /note="target seq for Breast-ovarian cancer, familial 1"
  source 1..20: mol_type = genomic DNA
gggctagaaatctgttgcta

SEQ ID NO: 272 - DNA, 20 bp, Homo sapiens
  misc_feature 1..20: note = source = /note="target seq for Breast-ovarian cancer, familial 1"
  source 1..20: mol_type = genomic DNA
ggtgacccgagagtgggtgt

SEQ ID NO: 273 - DNA, 20 bp, Homo sapiens
  misc_feature 1..20: note = source = /note="target seq for Breast-ovarian cancer, familial 1"
  source 1..20: mol_type = genomic DNA
gattctgcaactttcaattg

SEQ ID NO: 274 - DNA, 20 bp, Homo sapiens
  misc_feature 1..20: note = source = /note="target seq for Breast-ovarian cancer, familial 1"
  source 1..20: mol_type = genomic DNA
ggacaatggcttccatggta

SEQ ID NO: 275 - DNA, 25 bp, Homo sapiens
  misc_feature 1..25: note = source = /note="target seq for Inborn genetic diseases"
  source 1..25: mol_type = genomic DNA
gaaagcccgacttatagccagtaat

SEQ ID NO: 276 - DNA, 20 bp, Homo sapiens
  misc_feature 1..20: note = source = /note="target seq for Breast-ovarian cancer, familial 2"
  source 1..20: mol_type = genomic DNA
gattacatgaacaaatgggc

SEQ ID NO: 277 - DNA, 20 bp, Homo sapiens
  misc_feature 1..20: note = source = /note="target seq for Breast-ovarian cancer, familial 2"
  source 1..20: mol_type = genomic DNA
gattacatgaacaaatgggc

SEQ ID NO: 278 - DNA, 20 bp, Homo sapiens
  misc_feature 1..20: note = source = /note="target seq for Breast-ovarian cancer, familial 2"
  source 1..20: mol_type = genomic DNA
gactggaaaaggaatacagt

SEQ ID NO: 279 - DNA, 25 bp, Homo sapiens
  misc_feature 1..25: note = source = /note="target seq for Breast and/or ovarian cancer"
  source 1..25: mol_type = genomic DNA
gttggctgatggtggatggctcata

SEQ ID NO: 280 - DNA, 25 bp, Homo sapiens
  misc_feature 1..25: note = source = /note="target seq for Breast-ovarian cancer, familial 2"
  source 1..25: mol_type = genomic DNA
gttggctgatggtggatggctcata

SEQ ID NO: 281 - DNA, 20 bp, Homo sapiens
  misc_feature 1..20: note = source = /note="target seq for Breast-ovarian cancer, familial 2"
  source 1..20: mol_type = genomic DNA
ggtcagaagattattcttca

SEQ ID NO: 282 - DNA, 20 bp, Homo sapiens
  misc_feature 1..20: note = source = /note="target seq for Breast-ovarian cancer, familial 2"
  source 1..20: mol_type = genomic DNA
ggcttctagtctcttttgtt

SEQ ID NO: 283 - DNA, 20 bp, Homo sapiens
  misc_feature 1..20: note = source = /note="target seq for Breast-ovarian cancer, familial 2"
  source 1..20: mol_type = genomic DNA
ggcttctagtctcttttgtt

SEQ ID NO: 284 - DNA, 20 bp, Homo sapiens
  misc_feature 1..20: note = source = /note="target seq for Smith-Lemli-Opitz syndrome"
  source 1..20: mol_type = genomic DNA
gttgcgattttgcagccatt

SEQ ID NO: 285 - DNA, 25 bp, Homo sapiens
  misc_feature 1..25: note = source = /note="target seq for Alport syndrome 1, X-linked recessive"
  source 1..25: mol_type = genomic DNA
ggcctatgggtccccctggtttcgg

SEQ ID NO: 286 - DNA, 20 bp, Homo sapiens
  misc_feature 1..20: note = source = /note="target seq for Acute neuronopathic Gaucher's disease"
  source 1..20: mol_type = genomic DNA
ggcatcaggtgagtgagtca

SEQ ID NO: 287 - DNA, 20 bp, Homo sapiens
  misc_feature 1..20: note = source = /note="target seq for Gonadotropin deficiency"
  source 1..20: mol_type = genomic DNA
gtaactctccagcataccat

SEQ ID NO: 288 - DNA, 20 bp, Homo sapiens
  misc_feature 1..20: note = source = /note="target seq for Distal arthrogryposis type 1A"
  source 1..20: mol_type = genomic DNA
gaagctggaggaggccgaga

SEQ ID NO: 289 - DNA, 20 bp, Homo sapiens
  misc_feature 1..20: note = source = /note="target seq for Distal arthrogryposis type 1A"
  source 1..20: mol_type = genomic DNA
gctggaggaggccgagaagg

SEQ ID NO: 290 - DNA, 20 bp, Homo sapiens
  misc_feature 1..20: note = source = /note="target seq for Hereditary cancer-predisposing syndrome"
  source 1..20: mol_type = genomic DNA
gctgccaccttcagggcctc

SEQ ID NO: 291 - DNA, 20 bp, Homo sapiens
  misc_feature 1..20: note = source = /note="target seq for Inborn genetic diseases"
  source 1..20: mol_type = genomic DNA
gataaacttcttgaagagga

SEQ ID NO: 292 - DNA, 25 bp, Homo sapiens
  misc_feature 1..25: note = source = /note="target seq for Death in early adulthood"
  source 1..25: mol_type = genomic DNA
gtgttctccatgttcgaacagaccc

SEQ ID NO: 293 - DNA, 20 bp, Homo sapiens
  misc_feature 1..20: note = source = /note="target seq for Death in early adulthood"
  source 1..20: mol_type = genomic DNA
gttcgaacagacccaaatcc

SEQ ID NO: 294 - DNA, 20 bp, Homo sapiens
  misc_feature 1..20: note = source = /note="target seq for Severe autosomal recessive musculardystrophy of childhood - North African type"
  source 1..20: mol_type = genomic DNA
gaaatctgtgtgtgtccaga

SEQ ID NO: 295 - DNA, 20 bp, Homo sapiens
  misc_feature 1..20: note = source = /note="target seq for Cardiovascular phenotype"
  source 1..20: mol_type = genomic DNA
ggtcatcattgagagcgacc

SEQ ID NO: 296 - DNA, 20 bp, Homo sapiens
  misc_feature 1..20: note = source = /note="target seq for Carbohydrate-deficient glycoproteinsyndrome type I"
  source 1..20: mol_type = genomic DNA
gaatgctgaagatgatcgac

SEQ ID NO: 297 - DNA, 20 bp, Homo sapiens
  misc_feature 1..20: note = source = /note="target seq for Charcot-Marie-Tooth disease, type I"
  source 1..20: mol_type = genomic DNA
gaatgctgaagatgatcgac

SEQ ID NO: 298 - DNA, 25 bp, Homo sapiens
  misc_feature 1..25: note = source = /note="target seq for Inborn genetic diseases"
  source 1..25: mol_type = genomic DNA
gctcagtacaccaccgtcggccgca

SEQ ID NO: 299 - DNA, 20 bp, Homo sapiens
  misc_feature 1..20: note = source = /note="target seq for Inborn genetic diseases"
  source 1..20: mol_type = genomic DNA
gtacaccaccgtcggccgca

SEQ ID NO: 300 - DNA, 25 bp, Homo sapiens
  misc_feature 1..25: note = source = /note="target seq for Familial Mediterranean fever"
  source 1..25: mol_type = genomic DNA
ggacacgtgatggagggaagaacac

SEQ ID NO: 301 - DNA, 25 bp, Homo sapiens
  misc_feature 1..25: note = source = /note="target seq for Deafness, autosomal recessive 2"
  source 1..25: mol_type = genomic DNA
ggacctttgtcaatgggacacggac

SEQ ID NO: 302 - DNA, 25 bp, Homo sapiens
  misc_feature 1..25: note = source = /note="target seq for Deafness, autosomal recessive 2"
  source 1..25: mol_type = genomic DNA
ggacctttgtcaatgggacacagac

SEQ ID NO: 303 - DNA, 25 bp, Homo sapiens
  misc_feature 1..25: note = source = /note="target seq for Deafness, X-linked 2"
  source 1..25: mol_type = genomic DNA
gcgctcctagtggccatgcacgtgg

SEQ ID NO: 304 - DNA, 25 bp, Homo sapiens
  misc_feature 1..25: note = source = /note="target seq for Enlarged vestibular aqueduct"
  source 1..25: mol_type = genomic DNA
gccaccactgctctttcccgcacgg

SEQ ID NO: 305 - DNA, 20 bp, Homo sapiens
  misc_feature 1..20: note = source = /note="target seq for Congenital sensorineuralhearing impairment"
  source 1..20: mol_type = genomic DNA
gtggtcattaacacaaactc

SEQ ID NO: 306 - DNA, 20 bp, Homo sapiens
  misc_feature 1..20: note = source = /note="target seq for Deficiency ofUDPglucose-hexose-1-phosphate uridylyltransferase"
  source 1..20: mol_type = genomic DNA
gccagatattgcccagcgtg

SEQ ID NO: 307 - DNA, 20 bp, Homo sapiens
  misc_feature 1..20: note = source = /note="target seq for Very long chain acyl-CoAdehydrogenase deficiency"
  source 1..20: mol_type = genomic DNA
gccactaatcgtacccagtt

SEQ ID NO: 308 - DNA, 20 bp, Homo sapiens
  misc_feature 1..20: note = source = /note="target seq for Cardiovascular phenotype"
  source 1..20: mol_type = genomic DNA
gatattccactggtggtcga

SEQ ID NO: 309 - DNA, 20 bp, Homo sapiens
  misc_feature 1..20: note = source = /note="target seq for Pyruvate kinase deficiency of red cells"
  source 1..20: mol_type = genomic DNA
ggaagactcctgggcataag

SEQ ID NO: 310 - DNA, 25 bp, Homo sapiens
  misc_feature 1..25: note = source = /note="target seq for Autosomal dominant progressiveexternal ophthalmoplegia with mitochondrial DNAdeletions 1"
  source 1..25: mol_type = genomic DNA
gaagtcgttgatggatctggccaat

SEQ ID NO: 311 - DNA, 20 bp, Homo sapiens
  misc_feature 1..20: note = source = /note="target seq for Very long chain acyl-CoAdehydrogenase deficiency"
  source 1..20: mol_type = genomic DNA
gaagatcacagcttttgtgg

SEQ ID NO: 312 - DNA, 20 bp, Homo sapiens
  misc_feature 1..20: note = source = /note="target seq for Cystinosis, ocular nonnephropathic"
  source 1..20: mol_type = genomic DNA
gtccatctccttctaccctc

SEQ ID NO: 313 - DNA, 20 bp, Homo sapiens
  misc_feature 1..20: note = source = /note="target seq for Pyruvate kinase deficiency of redcells"
  source 1..20: mol_type = genomic DNA
gctcagcccagcttctgtct

SEQ ID NO: 314 - DNA, 25 bp, Homo sapiens
  misc_feature 1..25: note = source = /note="target seq for Distal myopathy, Tateyama type"
  source 1..25: mol_type = genomic DNA
gattgacctggtgaaccaagacccc

SEQ ID NO: 315 - DNA, 25 bp, Homo sapiens
  misc_feature 1..25: note = source = /note="target seq for Malignant hyperthermia,susceptibility to, 1"
  source 1..25: mol_type = genomic DNA
ggccctgcggatccgcgccatcctc

SEQ ID NO: 316 - DNA, 25 bp, Homo sapiens
  misc_feature 1..25: note = source = /note="target seq for Malignant hyperthermia,susceptibility to, 1"
  source 1..25: mol_type = genomic DNA
ggatccgcgccatcctccgctccct

SEQ ID NO: 317 - DNA, 25 bp, Homo sapiens
  misc_feature 1..25: note = source = /note="target seq for Myopathy, Central Core"
  source 1..25: mol_type = genomic DNA
ggtctgatcatcgacgcttttggtg

SEQ ID NO: 318 - DNA, 20 bp, Homo sapiens
  misc_feature 1..20: note = source = /note="target seq for Myopathy, Central Core"
  source 1..20: mol_type = genomic DNA
ggtctgatcatcgacgcttt

SEQ ID NO: 319 - DNA, 25 bp, Homo sapiens
  misc_feature 1..25: note = source = /note="target seq for Myopathy, Central Core"
  source 1..25: mol_type = genomic DNA
gatcatcgacacttttggtgagctc

SEQ ID NO: 320 - DNA, 25 bp, Homo sapiens
  misc_feature 1..25: note = source = /note="target seq for Malignant hyperthermia,susceptibility to, 1"
  source 1..25: mol_type = genomic DNA
ggcggaggcattggggacgagatcg

SEQ ID NO: 321 - DNA, 20 bp, Homo sapiens
  misc_feature 1..20: note = source = /note="target seq for Ceroid lipofuscinosis neuronal 2"
  source 1..20: mol_type = genomic DNA
gtgacagtggggccgggtgt

SEQ ID NO: 322 - DNA, 25 bp, Homo sapiens
  misc_feature 1..25: note = source = /note="target seq for Ceroid lipofuscinosis neuronal 2"
  source 1..25: mol_type = genomic DNA
ggggccgggtattggtctgtctctg

SEQ ID NO: 323 - DNA, 25 bp, Homo sapiens
  misc_feature 1..25: note = source = /note="target seq for Niemann-Pick disease type C1"
  source 1..25: mol_type = genomic DNA
ggatcgacgattatttcgactgggt

SEQ ID NO: 324 - DNA, 25 bp, Homo sapiens
  misc_feature 1..25: note = source = /note="target seq for Glutaric aciduria, type 1"
  source 1..25: mol_type = genomic DNA
gagtatcacgtgatccggcacgcca

SEQ ID NO: 325 - DNA, 25 bp, Homo sapiens
  misc_feature 1..25: note = source = /note="target seq for CAPN3-Related Disorders"
  source 1..25: mol_type = genomic DNA
gccctgatgcagaagaaccggcgga

SEQ ID NO: 326 - DNA, 20 bp, Homo sapiens
  misc_feature 1..20: note = source = /note="target seq for CAPN3-Related Disorders"
  source 1..20: mol_type = genomic DNA
gatgcagaagaaccggcgga

SEQ ID NO: 327 - DNA, 20 bp, Homo sapiens
  misc_feature 1..20: note = source = /note="target seq for Glycogen storage disease, type II"
  source 1..20: mol_type = genomic DNA
gtcccagaacagctcccctc

SEQ ID NO: 328 - DNA, 25 bp, Homo sapiens
  misc_feature 1..25: note = source = /note="target seq for Nonsyndromic Oculocutaneous Albinism"
  source 1..25: mol_type = genomic DNA
gtacagggatctgccaacgatccta

SEQ ID NO: 329 - DNA, 20 bp, Homo sapiens
  misc_feature 1..20: note = source = /note="target seq for Familial hypercholesterolemia"
  source 1..20: mol_type = genomic DNA
gcgaagatggctcggatgag

SEQ ID NO: 330 - DNA, 20 bp, Homo sapiens
  misc_feature 1..20: note = source = /note="target seq for Familial hypercholesterolemia"
  source 1..20: mol_type = genomic DNA
gagtggccgcagcgctgtag

SEQ ID NO: 331 - DNA, 20 bp, Homo sapiens
  misc_feature 1..20: note = source = /note="target seq for Deafness, autosomal recessive 7"
  source 1..20: mol_type = genomic DNA
ggtctcaagctctctcttcg

SEQ ID NO: 332 - DNA, 20 bp, Homo sapiens
  misc_feature 1..20: note = source = /note="target seq for Chronic infantile neurological,cutaneous and articular syndrome"
  source 1..20: mol_type = genomic DNA
gacggcttcgatgagctgca

SEQ ID NO: 333 - DNA, 20 bp, Homo sapiens
  misc_feature 1..20: note = source = /note="target seq for Eichsfeld type congenitalmuscular dystrophy"
  source 1..20: mol_type = genomic DNA
gactctccgggagactgtcc

SEQ ID NO: 334 - DNA, 20 bp, Homo sapiens
  misc_feature 1..20: note = source = /note="target seq for Inborn genetic diseases"
  source 1..20: mol_type = genomic DNA
gctcaaaagtggatgagcgc

SEQ ID NO: 335 - DNA, 20 bp, Homo sapiens
  misc_feature 1..20: note = source = /note="target seq for Hyperkalemic Periodic ParalysisType 1"
  source 1..20: mol_type = genomic DNA
gcagctgcgggtcttcaagc

SEQ ID NO: 336 - DNA, 20 bp, Homo sapiens
  misc_feature 1..20: note = source = /note="target seq for Inclusion body myopathy 2"
  source 1..20: mol_type = genomic DNA
ggacgtggatgtggtggttt

SEQ ID NO: 337 - DNA, 25 bp, Homo sapiens
  misc_feature 1..25: note = source = /note="target seq for Severe APG05083.1, APG07433.1,APG07513.1, APG08290.1 immunodeficiency due to ADADeficiency"
  source 1..25: mol_type = genomic DNA
gaagagcggcattcaccgtactgtc

SEQ ID NO: 338 - DNA, 25 bp, Homo sapiens
  misc_feature 1..25: note = source = /note="target seq for Severe APG05083.1, APG07433.1,APG07513.1, APG08290.1 immunodeficiency due to ADADeficiency"
  source 1..25: mol_type = genomic DNA
gcggtacagtccgcacctgctggcc

SEQ ID NO: 339 - DNA, 20 bp, Homo sapiens
  misc_feature 1..20: note = source = /note="target seq for Cardiovascular phenotype"
  source 1..20: mol_type = genomic DNA
gtgtccgagcagcgccactg

SEQ ID NO: 340 - DNA, 20 bp, Homo sapiens
  misc_feature 1..20: note = source = /note="target seq for Cystic fibrosis"
  source 1..20: mol_type = genomic DNA
ggacacttcgtgccttcgga

SEQ ID NO: 341 - DNA, 20 bp, Homo sapiens
  misc_feature 1..20: note = source = /note="target seq for Cystic fibrosis"
  source 1..20: mol_type = genomic DNA
ggacacttcgtgccttcgga

SEQ ID NO: 342 - DNA, 25 bp, Homo sapiens
  misc_feature 1..25: note = source = /note="target seq for Adrenocortical carcinoma, pediatric"
  source 1..25: mol_type = genomic DNA
gggcgtgagcacttcgagatgttcc

SEQ ID NO: 343 - DNA, 20 bp, Homo sapiens
  misc_feature 1..20: note = source = /note="target seq for Fumarase deficiency"
  source 1..20: mol_type = genomic DNA
gattggacgtactcatactc

SEQ ID NO: 344 - DNA, 20 bp, Homo sapiens
  misc_feature 1..20: note = source = /note="target seq for Adenocarcinoma of prostate"
  source 1..20: mol_type = genomic DNA
gaacactgtccattggcatg

SEQ ID NO: 345 - DNA, 20 bp, Homo sapiens
  misc_feature 1..20: note = source = /note="target seq for Familial hypertrophic cardiomyopathy 1"
  source 1..20: mol_type = genomic DNA
gcatcctctacggggacttc

SEQ ID NO: 346 - DNA, 25 bp, Homo sapiens
  misc_feature 1..25: note = source = /note="target seq for Adult hypophosphatasia"
  source 1..25: mol_type = genomic DNA
gacaacgagatgccccctgaggcct

SEQ ID NO: 347 - DNA, 25 bp, Homo sapiens
  misc_feature 1..25: note = source = /note="target seq for Adult hypophosphatasia"
  source 1..25: mol_type = genomic DNA
ggtactcagacaacaagatgccccc

SEQ ID NO: 348 - DNA, 25 bp, Homo sapiens
  misc_feature 1..25: note = source = /note="target seq for Adult hypophosphatasia"
  source 1..25: mol_type = genomic DNA
gacaacaagatgccccctgaggcct

SEQ ID NO: 349 - DNA, 25 bp, Homo sapiens
  misc_feature 1..25: note = source = /note="target seq for Inborn genetic diseases"
  source 1..25: mol_type = genomic DNA
gccttcttggacaacgtcaccacca

SEQ ID NO: 350 - DNA, 25 bp, Homo sapiens
  misc_feature 1..25: note = source = /note="target seq for Inborn genetic diseases"
  source 1..25: mol_type = genomic DNA
ggtttgccatccgaggggtagtgct

SEQ ID NO: 351 - DNA, 20 bp, Homo sapiens
  misc_feature 1..20: note = source = /note="target seq for Crouzon syndrome"
  source 1..20: mol_type = genomic DNA
gcaaatgcctccacagtggt

SEQ ID NO: 352 - DNA, 20 bp, Homo sapiens
  misc_feature 1..20: note = source = /note="target seq for Propionyl-CoA carboxylase deficiency"
  source 1..20: mol_type = genomic DNA
gacatcataggcacctccat

SEQ ID NO: 353 - DNA, 25 bp, Homo sapiens
  misc_feature 1..25: note = source = /note="target seq for Cardiovascular phenotype"
  source 1..25: mol_type = genomic DNA
ggtggaagggatcggctacgacttc

SEQ ID NO: 354 - DNA, 25 bp, Homo sapiens
  misc_feature 1..25: note = source = /note="target seq for Dysostosis multiplex"
  source 1..25: mol_type = genomic DNA
ggagcagctctgggccgaagtgtcg

SEQ ID NO: 355 - DNA, 20 bp, Homo sapiens
  misc_feature 1..20: note = source = /note="target seq for Multiple sulfatase deficiency"
  source 1..20: mol_type = genomic DNA
ggcgactcctttgtctttga

SEQ ID NO: 356 - DNA, 20 bp, Homo sapiens
  misc_feature 1..20: note = source = /note="target seq for Bifunctional peroxisomal enzymedeficiency"
  source 1..20: mol_type = genomic DNA
ggtggtactggtcaccggcg

SEQ ID NO: 357 - DNA, 25 bp, Homo sapiens
  misc_feature 1..25: note = source = /note="target seq for Bifunctional peroxisomal enzymedeficiency"
  source 1..25: mol_type = genomic DNA
gtactggtcaccagcgcgggggcag

SEQ ID NO: 358 - DNA, 20 bp, Homo sapiens
  misc_feature 1..20: note = source = /note="target seq for Hereditary cancer-predisposingsyndrome"
  source 1..20: mol_type = genomic DNA
ggaaacttgtaagggcttcg

SEQ ID NO: 359 - DNA, 25 bp, Homo sapiens
  misc_feature 1..25: note = source = /note="target seq for Cardiovascular phenotype"
  source 1..25: mol_type = genomic DNA
gtgcagacattgacgaatgccgcat

SEQ ID NO: 360 - DNA, 25 bp, Homo sapiens
  misc_feature 1..25: note = source = /note="target seq for Cardiovascular phenotype"
  source 1..25: mol_type = genomic DNA
gacattgacgaatgccgcatatctc

SEQ ID NO: 361 - DNA, 20 bp, Homo sapiens
  misc_feature 1..20: note = source = /note="target seq for Limb-girdle muscular dystrophy,type 2L"
  source 1..20: mol_type = genomic DNA
gcatagtagtaaactagacg

SEQ ID NO: 362 - DNA, 25 bp, Homo sapiens
  misc_feature 1..25: note = source = /note="target seq for Familial hypercholesterolemia"
  source 1..25: mol_type = genomic DNA
gcatcgatgtcaacgagggcaaccg

SEQ ID NO: 363 - DNA, 20 bp, Homo sapiens
  misc_feature 1..20: note = source = /note="target seq for Spastic Paraplegia, Recessive"
  source 1..20: mol_type = genomic DNA
gcactgctgctcggcccccc

SEQ ID NO: 364 - DNA, 20 bp, Homo sapiens
  misc_feature 1..20: note = source = /note="target seq for Isovaleryl-CoA dehydrogenasedeficiency"
  source 1..20: mol_type = genomic DNA
ggagcagtggggctcagtta

SEQ ID NO: 365 - DNA, 20 bp, Homo sapiens
  misc_feature 1..20: note = source = /note="target seq for Familial hypercholesterolemia"
  source 1..20: mol_type = genomic DNA
gaggagctgcctcacaggtg

SEQ ID NO: 366 - DNA, 20 bp, Homo sapiens
  misc_feature 1..20: note = source = /note="target seq for Biotinidase deficiency"
  source 1..20: mol_type = genomic DNA
gctccagcgcctgagttgta

SEQ ID NO: 367 - DNA, 20 bp, Homo sapiens
  misc_feature 1..20: note = source = /note="target seq for Biotinidase deficiency"
  source 1..20: mol_type = genomic DNA
gcctgagttgtatggccatc

SEQ ID NO: 368 - DNA, 25 bp, Homo sapiens
  misc_feature 1..25: note = source = /note="target seq for Leber congenital amaurosis"
  source 1..25: mol_type = genomic DNA
gcataatttgtacagctctaagagt

SEQ ID NO: 369 - DNA, 25 bp, Homo sapiens
  misc_feature 1..25: note = source = /note="target seq for Familial hyperinsulinism"
  source 1..25: mol_type = genomic DNA
ggtggctgagcccagcccggccccc

SEQ ID NO: 370 - DNA, 25 bp, Homo sapiens
  misc_feature 1..25: note = source = /note="target seq for Familial cancer of breast"
  source 1..25: mol_type = genomic DNA
ggatgcttggcagtgggaaaaactt

SEQ ID NO: 371 - DNA, 20 bp, Homo sapiens
  misc_feature 1..20: note = source = /note="target seq for Cohen syndrome"
  source 1..20: mol_type = genomic DNA
ggaggtctactacaggtctg

SEQ ID NO: 372 - DNA, 25 bp, Homo sapiens
  misc_feature 1..25: note = source = /note="target seq for Cardiovascular phenotype"
  source 1..25: mol_type = genomic DNA
gctccctctgtactgtgcaggagtc

SEQ ID NO: 373 - DNA, 20 bp, Homo sapiens
  misc_feature 1..20: note = source = /note="target seq for Wilson disease"
  source 1..20: mol_type = genomic DNA
ggcaatgaacacaaagagca

SEQ ID NO: 374 - DNA, 25 bp, Homo sapiens
  misc_feature 1..25: note = source = /note="target seq for Wilson disease"
  source 1..25: mol_type = genomic DNA
gccctggatgggctcagcggccatg

SEQ ID NO: 375 - DNA, 20 bp, Homo sapiens
  misc_feature 1..20: note = source = /note="target seq for Wilson disease"
  source 1..20: mol_type = genomic DNA
gcagccctggatgggctcag

SEQ ID NO: 376 - DNA, 20 bp, Homo sapiens
  misc_feature 1..20: note = source = /note="target seq for Familial hypercholesterolemia"
  source 1..20: mol_type = genomic DNA
gtggcccagcgaagatgcga

SEQ ID NO: 377 - DNA, 20 bp, Homo sapiens
  misc_feature 1..20: note = source = /note="target seq for Familial hypercholesterolemia"
  source 1..20: mol_type = genomic DNA
gaagatgcgaaggtgatttc

SEQ ID NO: 378 - DNA, 20 bp, Homo sapiens
  misc_feature 1..20: note = source = /note="target seq for Floating-Harbor syndrome"
  source 1..20: mol_type = genomic DNA
gctgaagctggagtgggtcg

SEQ ID NO: 379 - DNA, 25 bp, Homo sapiens
  misc_feature 1..25: note = source = /note="target seq for Congenital long QT syndrome"
  source 1..25: mol_type = genomic DNA
gatgctacacgtcgaccgccaggga

SEQ ID NO: 380 - DNA, 20 bp, Homo sapiens
  misc_feature 1..20: note = source = /note="target seq for Congenital long QT syndrome"
  source 1..20: mol_type = genomic DNA
gatgctacacgtcgaccgcc

SEQ ID NO: 381 - DNA, 25 bp, Homo sapiens
  misc_feature 1..25: note = source = /note="target seq for Andersen Tawil syndrome"
  source 1..25: mol_type = genomic DNA
gaggaagagatcaaggtcctttcca

SEQ ID NO: 382 - DNA, 25 bp, Homo sapiens
  misc_feature 1..25: note = source = /note="target seq for Cardiovascular phenotype"
  source 1..25: mol_type = genomic DNA
gagctgataaccaccctgtacatcg

SEQ ID NO: 383 - DNA, 20 bp, Homo sapiens
  misc_feature 1..20: note = source = /note="target seq for Familial hypercholesterolemia"
  source 1..20: mol_type = genomic DNA
ggaaaactgcggtatgggcg

SEQ ID NO: 384 - DNA, 25 bp, Homo sapiens
  misc_feature 1..25: note = source = /note="target seq for Cardiovascular phenotype"
  source 1..25: mol_type = genomic DNA
gtatttgaaggtctcctccggggtc

SEQ ID NO: 385 - DNA, 20 bp, Homo sapiens
  misc_feature 1..20: note = source = /note="target seq for Gastrointestinal stroma tumor"
  source 1..20: mol_type = genomic DNA
gagggctaaagtgggctcgg

SEQ ID NO: 386 - DNA, 20 bp, Homo sapiens
  misc_feature 1..20: note = source = /note="target seq for Dyskeratosis congenita"
  source 1..20: mol_type = genomic DNA
gctgccaagcctgctggcaa

SEQ ID NO: 387 - DNA, 25 bp, Homo sapiens
  misc_feature 1..25: note = source = /note="target seq for Dyskeratosis congenita"
  source 1..25: mol_type = genomic DNA
gccctgcctgtgacttccagcactg

SEQ ID NO: 388 - DNA, 25 bp, Homo sapiens
  misc_feature 1..25: note = source = /note="target seq for Glycogen storage disease IIIa"
  source 1..25: mol_type = genomic DNA
gatgagtagaacagaaaaatacaag

SEQ ID NO: 389 - DNA, 25 bp, Homo sapiens
  misc_feature 1..25: note = source = /note="target seq for Dilated cardiomyopathy 1DD"
  source 1..25: mol_type = genomic DNA
gtctcgtagtccggtgagccggtca

SEQ ID NO: 390 - DNA, 20 bp, Homo sapiens
  misc_feature 1..20: note = source = /note="target seq for Renal carnitine transport defect"
  source 1..20: mol_type = genomic DNA
gtggccatggaatagcgccg

SEQ ID NO: 391 - DNA, 25 bp, Homo sapiens
  misc_feature 1..25: note = source = /note="target seq for Baraitser-Winter syndrome 1"
  source 1..25: mol_type = genomic DNA
gatcctcaccgagcgcggctacagc

SEQ ID NO: 392 - DNA, 25 bp, Homo sapiens
  misc_feature 1..25: note = source = /note="target seq for Very long chain acyl-CoAdehydrogenase deficiency"
  source 1..25: mol_type = genomic DNA
gggcatgcatgaccttggcgtgggc

SEQ ID NO: 393 - DNA, 20 bp, Homo sapiens
  misc_feature 1..20: note = source = /note="target seq for Familial hypercholesterolemia"
  source 1..20: mol_type = genomic DNA
gtcagatttgtccttgcagt

SEQ ID NO: 394 - DNA, 20 bp, Homo sapiens
  misc_feature 1..20: note = source = /note="target seq for Limb-girdle muscular dystrophy,type 2A"
  source 1..20: mol_type = genomic DNA
gtacggggttgctctgccgg

SEQ ID NO: 395 - DNA, 25 bp, Homo sapiens
  misc_feature 1..25: note = source = /note="target seq for Familial hypercholesterolemia"
  source 1..25: mol_type = genomic DNA
gacagtagcccctgctcggccttcg

SEQ ID NO: 396 - DNA, 20 bp, Homo sapiens
  misc_feature 1..20: note = source = /note="target seq for Aortic aneurysm, familial thoracic 6"
  source 1..20: mol_type = genomic DNA
gccccatgccatcatgcgtc

SEQ ID NO: 397 - DNA, 25 bp, Homo sapiens
  misc_feature 1..25: note = source = /note="target seq for Acromicric dysplasia"
  source 1..25: mol_type = genomic DNA
gctttgtcatcgacatttataccgg

SEQ ID NO: 398 - DNA, 25 bp, Homo sapiens
  misc_feature 1..25: note = source = /note="target seq for Charcot-Marie-Tooth disease type 2C"
  source 1..25: mol_type = genomic DNA
ggcggacatgcggcaccaggactcg

SEQ ID NO: 399 - DNA, 25 bp, Homo sapiens
  misc_feature 1..25: note = source = /note="target seq for Breast-ovarian cancer, familial 2"
  source 1..25: mol_type = genomic DNA
gtgtaactgtatacgtatggcgttt

SEQ ID NO: 400 - DNA, 20 bp, Homo sapiens
  misc_feature 1..20: note = source = /note="target seq for Breast-ovarian cancer, familial 1"
  source 1..20: mol_type = genomic DNA
gatgcctggacagaggacaa

SEQ ID NO: 401 - DNA, 25 bp, Homo sapiens
  misc_feature 1..25: note = source = /note="target seq for Charcot-Marie-Tooth disease type 2C"
  source 1..25: mol_type = genomic DNA
gcctaactgatgaggagtttcgagg

SEQ ID NO: 402 - DNA, 25 bp, Homo sapiens
  misc_feature 1..25: note = source = /note="target seq for Charcot-Marie-Tooth disease type 2C"
  source 1..25: mol_type = genomic DNA
gcctaactgatgaggagtttcaagg

SEQ ID NO: 403 - DNA, 20 bp, Homo sapiens
  misc_feature 1..20: note = source = /note="target seq for Hereditary cancer-predisposingsyndrome"
  source 1..20: mol_type = genomic DNA
gagcgctgctcagatagcga

SEQ ID NO: 404 - DNA, 25 bp, Homo sapiens
  misc_feature 1..25: note = source = /note="target seq for Early infantile epilepticencephalopathy"
  source 1..25: mol_type = genomic DNA
gatgatccgcatggaccggcgggga

SEQ ID NO: 405 - DNA, 20 bp, Homo sapiens
  misc_feature 1..20: note = source = /note="target seq for Early infantile epilepticencephalopathy"
  source 1..20: mol_type = genomic DNA
ggatgatccgcatggaccgg

SEQ ID NO: 406 - DNA, 25 bp, Homo sapiens
  misc_feature 1..25: note = source = /note="target seq for Early infantile epilepticencephalopathy"
  source 1..25: mol_type = genomic DNA
gcggatgatccgcatggaccagcgg

SEQ ID NO: 407 - DNA, 20 bp, Homo sapiens
  misc_feature 1..20: note = source = /note="target seq for Acromicric dysplasia"
  source 1..20: mol_type = genomic DNA
gtacgtgatccatcctaggt

SEQ ID NO: 408 - DNA, 25 bp, Homo sapiens
  misc_feature 1..25: note = source = /note="target seq for Hypertrophic cardiomyopathy"
  source 1..25: mol_type = genomic DNA
gacaccacggtctccctcaagtggc

SEQ ID NO: 409 - DNA, 25 bp, Homo sapiens
  misc_feature 1..25: note = source = /note="target seq for Cardiovascular phenotype"
  source 1..25: mol_type = genomic DNA
gctgtccggggtagccccaagatag

SEQ ID NO: 410 - DNA, 25 bp, Homo sapiens
  misc_feature 1..25: note = source = /note="target seq for Cardiovascular phenotype"
  source 1..25: mol_type = genomic DNA
gtccacggtgagggggccctggtgt

SEQ ID NO: 411 - DNA, 25 bp, Homo sapiens
  misc_feature 1..25: note = source = /note="target seq for Cardiovascular phenotype"
  source 1..25: mol_type = genomic DNA
gcctagactgcaggacacagggact

SEQ ID NO: 412 - DNA, 25 bp, Homo sapiens
  misc_feature 1..25: note = source = /note="target seq for Familial hypertrophiccardiomyopathy 1"
  source 1..25: mol_type = genomic DNA
gcatctgcagacatagagacctgtg

SEQ ID NO: 413 - DNA, 20 bp, Homo sapiens
  misc_feature 1..20: note = source = /note="target seq for Benign scapuloperoneal musculardystrophy with cardiomyopathy"
  source 1..20: mol_type = genomic DNA
gccctccaagagcttgcggt

SEQ ID NO: 414 - DNA, 25 bp, Homo sapiens
  misc_feature 1..25: note = source = /note="target seq for Glycogen storage disease, type II"
  source 1..25: mol_type = genomic DNA
ggccctcaccctgcgctacgcactc

SEQ ID NO: 415 - DNA, 25 bp, Homo sapiens
  misc_feature 1..25: note = source = /note="target seq for Diffuse mesangial sclerosis"
  source 1..25: mol_type = genomic DNA
gtttaggcacttgttttacctgtat

SEQ ID NO: 416 - DNA, 20 bp, Homo sapiens
  misc_feature 1..20: note = source = /note="target seq for Colobomatous microphthalmia"
  source 1..20: mol_type = genomic DNA
gggtcgaggcggacgccata

SEQ ID NO: 417 - DNA, 20 bp, Homo sapiens
  misc_feature 1..20: note = source = /note="target seq for Ataxia-telangiectasia syndrome"
  source 1..20: mol_type = genomic DNA
gtaaatacatatttactact

SEQ ID NO: 418 - DNA, 20 bp, Homo sapiens
  misc_feature 1..20: note = source = /note="target seq for Familial cancer of breast"
  source 1..20: mol_type = genomic DNA
ggctacagcacacagctcgt

SEQ ID NO: 419 - DNA, 25 bp, Homo sapiens
  misc_feature 1..25: note = source = /note="target seq for Limb-girdle muscular dystrophy,type 2A"
  source 1..25: mol_type = genomic DNA
gctacgagatgcaaaatgcagtcaa

SEQ ID NO: 420 - DNA, 20 bp, Homo sapiens
  misc_feature 1..20: note = source = /note="target seq for Hereditary cancer-predisposingsyndrome"
  source 1..20: mol_type = genomic DNA
gccgcttaccaaaaggagta

SEQ ID NO: 421 - DNA, 25 bp, Homo sapiens
  misc_feature 1..25: note = source = /note="target seq for Asymmetric septal hypertrophy"
  source 1..25: mol_type = genomic DNA
gtgctgcaggttgttggtgcatggc

SEQ ID NO: 422 - DNA, 20 bp, Homo sapiens
  misc_feature 1..20: note = source = /note="target seq for Hereditary cancer-predisposingsyndrome"
  source 1..20: mol_type = genomic DNA
gtaattctataactccttag

SEQ ID NO: 423 - DNA, 25 bp, Homo sapiens
  misc_feature 1..25: note = source = /note="target seq for Familial hypertrophiccardiomyopathy 2"
  source 1..25: mol_type = genomic DNA
gctaaagtcaccgggcgctggaaat

SEQ ID NO: 424 - DNA, 25 bp, Homo sapiens
  misc_feature 1..25: note = source = /note="target seq for Familial hypertrophiccardiomyopathy 2"
  source 1..25: mol_type = genomic DNA
ggctaaagtcaccgggcgctagaaa

SEQ ID NO: 425 - DNA, 25 bp, Homo sapiens
  misc_feature 1..25: note = source = /note="target seq for Familial hypertrophiccardiomyopathy 2"
  source 1..25: mol_type = genomic DNA
gctaaagtcaccgggcgctagaaat

SEQ ID NO: 426 - DNA, 25 bp, Homo sapiens
  misc_feature 1..25: note = source = /note="target seq for Erythrocytosis, familial, 2"
  source 1..25: mol_type = genomic DNA
gaaagagcaatgcctccaggttgtc

SEQ ID NO: 427 - DNA, 25 bp, Homo sapiens
  misc_feature 1..25: note = source = /note="target seq for Death in infancy"
  source 1..25: mol_type = genomic DNA
gtattggaatccggagcatccacgt

SEQ ID NO: 428 - DNA, 20 bp, Homo sapiens
  misc_feature 1..20: note = source = /note="target seq for Muscular Diseases"
  source 1..20: mol_type = genomic DNA
gcatcttccggatctttgag

SEQ ID NO: 429 - DNA, 25 bp, Homo sapiens
  misc_feature 1..25: note = source = /note="target seq for Familial hypercholesterolemia"
  source 1..25: mol_type = genomic DNA
gctgcgagcatggggccctgaggct

SEQ ID NO: 430 - DNA, 20 bp, Homo sapiens
  misc_feature 1..20: note = source = /note="target seq for Familial cancer of breast"
  source 1..20: mol_type = genomic DNA
ggcagcgttacccagtccga

SEQ ID NO: 431 - DNA, 25 bp, Homo sapiens
  misc_feature 1..25: note = source = /note="target seq for Hereditary cancer-predisposingsyndrome"
  source 1..25: mol_type = genomic DNA
gtgtaggcaacttctatagccacag

SEQ ID NO: 432 - DNA, 25 bp, Homo sapiens
  misc_feature 1..25: note = source = /note="target seq for Familial hypercholesterolemia"
  source 1..25: mol_type = genomic DNA
ggaactcccgccaagatcaagaaag

SEQ ID NO: 433 - DNA, 25 bp, Homo sapiens
  misc_feature 1..25: note = source = /note="target seq for Familial hypercholesterolemia"
  source 1..25: mol_type = genomic DNA
gcggctgccagtatctgtgcctccc

SEQ ID NO: 434 - DNA, 25 bp, Homo sapiens
  misc_feature 1..25: note = source = /note="target seq for Familial hypercholesterolemia"
  source 1..25: mol_type = genomic DNA
gaatgatctgcaggtgagcgtcgcc

SEQ ID NO: 435 - DNA, 25 bp, Homo sapiens
  misc_feature 1..25: note = source = /note="target seq for Inclusion body myopathy 2"
  source 1..25: mol_type = genomic DNA
gtgctgtagtgcaccaatgtaatct

SEQ ID NO: 436 - DNA, 20 bp, Homo sapiens
  misc_feature 1..20: note = source = /note="target seq for Ataxia-telangiectasia syndrome"
  source 1..20: mol_type = genomic DNA
gctttgatgttaacaatcgc

SEQ ID NO: 437 - DNA, 25 bp, Homo sapiens
  misc_feature 1..25: note = source = /note="target seq for Benign familialneonatal-infantile seizures"
  source 1..25: mol_type = genomic DNA
gtcagttctccgatcattccagctg

SEQ ID NO: 438 - DNA, 20 bp, Homo sapiens
  misc_feature 1..20: note = source = /note="target seq for Marfan Syndrome/Loeys-DietzSyndrome/Familial Thoracic Aortic Aneurysms andDissections"
  source 1..20: mol_type = genomic DNA
gtatctccattgtctcctcg

SEQ ID NO: 439 - DNA, 25 bp, Homo sapiens
  misc_feature 1..25: note = source = /note="target seq for Dilated cardiomyopathy 1G"
  source 1..25: mol_type = genomic DNA
gtttcagattcccctggcccacgtg

SEQ ID NO: 440 - DNA, 20 bp, Homo sapiens
  misc_feature 1..20: note = source = /note="target seq for Familial hypercholesterolemia"
  source 1..20: mol_type = genomic DNA
gcggtgagtctcggtgcagg

SEQ ID NO: 441 - DNA, 20 bp, Homo sapiens
  misc_feature 1..20: note = source = /note="target seq for Breast-ovarian cancer, familial 2"
  source 1..20: mol_type = genomic DNA
gatacagtattaattgactg

SEQ ID NO: 442 - DNA, 20 bp, Homo sapiens
  misc_feature 1..20: note = source = /note="target seq for Familial hypercholesterolemia"
  source 1..20: mol_type = genomic DNA
gcgagtgcatccactccagc

SEQ ID NO: 443 - DNA, 25 bp, Homo sapiens
  misc_feature 1..25: note = source = /note="target seq for Familial hypercholesterolemia"
  source 1..25: mol_type = genomic DNA
ggctacaagtgccagtgtgaggaag

SEQ ID NO: 444 - DNA, 25 bp, Homo sapiens
  misc_feature 1..25: note = source = /note="target seq for Familial hypercholesterolemia"
  source 1..25: mol_type = genomic DNA
gggtggctacaagtgccagcgtgag

SEQ ID NO: 445 - DNA, 25 bp, Homo sapiens
  misc_feature 1..25: note = source = /note="target seq for Familial hypercholesterolemia"
  source 1..25: mol_type = genomic DNA
gtacaccagcctcacccccaacctg

SEQ ID NO: 446 - DNA, 25 bp, Homo sapiens
  misc_feature 1..25: note = source = /note="target seq for Familial cancer of breast"
  source 1..25: mol_type = genomic DNA
gcctgcccgatagacgagcctcccg

SEQ ID NO: 447 - DNA, 20 bp, Homo sapiens
  misc_feature 1..20: note = source = /note="target seq for Hereditary hemorrhagictelangiectasia type 2"
  source 1..20: mol_type = genomic DNA
gatccgcagcgcggtgagtc

SEQ ID NO: 448-449 - skipped (2 entries)

SEQ ID NO: 450 - protein, 415 aa, synthetic construct
  REGION 1..415: note = source = /note="Description of Artificial Sequence: Syntheticpolypeptide"
  REGION 1..415: note = source = /note="ADAT"
MAKRPAATKKAGQAKKKKSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSS

SEQ ID NO: 451 - protein, 1494 aa, synthetic construct
  REGION 1..1494: note = source = /note="Description of Artificial Sequence: Syntheticpolypeptide"
  REGION 1..1494: note = source = /note="Nuc-ADAT-Linker-dAPG08290.1-Linker-SV40"
MAKRPAATKKAGQAKKKKSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSSMSELDYRIGLAIGTNSIGWGVIELFWNKDRERYEKVRIVDKGVRMFDKAEIPNKGASLAEPRRIARSSRRRLNRKSQRKKEIRNLLVQHGMITQEELDLLYPLSKKSIDIWDIRLDGLDRLLNHLEWARLLIHLAQRRGFKSNRKSELKDAETGKVLSSIQVNEKRLFLYRTVGEMWIKDAEFSKYDRRRNSPNEYVFSVSRADLEKEIVTLFEAQRKFQSSYASKNLQETYLQIWAHQLPFASGNAILNKVGYCSLLKGKERRIPKATYTFQYFSALDQVNRTRLGPDFQPFTQEQKEIILDKMFQRTDYYKKKTIPEVSYYDIRKWLELDETIQFKGLNYDPNEELKKIEKKPFINLKAFYEIKKVVANYAERTNEAFSTLDYDAIAYALTVYKTDKDIRSYLKKSNNLSKRCYDDQLIEELFTLSYTKFGHLSFKAINHVLPIMQEGRTYQEAIHQLGYDTTNLKKENRSMFLPLIPDEITNPIVKRAITQARKVVNAIIRRYGSPNSVHIALARELSKSHDERKKIMTAHDENYKKNKGAISILIENGILNPTGYDIVRYKLWKEQGERCAYSLKEIPPDTFFNELKKERNGSPILEVDHILPYSQSFIDSYHNKVLVYSDENRNKGNRIPYTYFLETNKDWEAFERYVRSNKLFSKKKREYLLKKTYLPRESELIKERHLNDTRYASTFLKNFIEQNLQFKEVEVNLRKKRVQTVNGVITAHLRKRWGLEKNRQETYLHHAMDAIIVACTDHHMVTRITEYYQIKESNKSVKKPYFPMPWEGFRDELLSHLASQPIAKKISEELKAGYQSSDYIFVSRMPKRSVTGAAHDQTIRRKGGIDKKGKTIIIKRVRLKDIKFDENGDFKMVGKEQDLATYEAIKQRYLEHRKNSKKAFETPLYKPSKKGTGNLIKRVKIEGQTKAFVREVNGGVAQNSDLVRVDLFEKDDKYYMVPIYVPDTVCSELPKKVVKSGKGYEQWLTLDNSFTFKSSLYPYDLVRLVKGNEDRFLYFGTLDIDSDRLNFKDVNKPSKQNEYRYSLKTIENLEKYEVGVLGDLRLVKQETRRIFNRPKKKRKV

SEQ ID NO: 452 - protein, 1494 aa, synthetic construct
  REGION 1..1494: note = source = /note="Description of Artificial Sequence: Syntheticpolypeptide"
  REGION 1..1494: note = source = /note="Nuc-ADAT-Linker-nAPG08290.1-Linker-SV40"
MAKRPAATKKAGQAKKKKSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTDSGGSSGGSSGSETPGTSESATPESSGGSSGGSSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTDSGGSSGGSSGSETPGTSESATPESSGGSSGGSSMSELDYRIGLAIGTNSIGWGVIELFWNKDRERYEKVRIVDKGVRMFDKAEIPNKGASLAEPRRIARSSRRRLNRKSQRKKEIRNLLVQHGMITQEELDLLYPLSKKSIDIWDIRLDGLDRLLNHLEWARLLIHLAQRRGFKSNRKSELKDAETGKVLSSIQVNEKRLFLYRTVGEMWIKDAEFSKYDRRRNSPNEYVFSVSRADLEKEIVTLFEAQRKFQSSYASKNLQETYLQIWAHQLPFASGNAILNKVGYCSLLKGKERRIPKATYTFQYFSALDQVNRTRLGPDFQPFTQEQKEIILDKMFQRTDYYKKKTIPEVSYYDIRKWLELDETIQFKGLNYDPNEELKKIEKKPFINLKAFYEIKKVVANYAERTNEAFSTLDYDAIAYALTVYKTDKDIRSYLKKSNNLSKRCYDDQLIEELFTLSYTKFGHLSFKAINHVLPIMQEGRTYQEAIHQLGYDTTNLKKENRSMFLPLIPDEITNPIVKRAITQARKVVNAIIRRYGSPNSVHIELARELSKSHDERKKIMTAHDENYKKNKGAISILIENGILNPTGYDIVRYKLWKEQGERCAYSLKEIPPDTFFNELKKERNGSPILEVDHILPYSQSFIDSYHNKVLVYSDENRNKGNRIPYTYFLETNKDWEAFERYVRSNKLFSKKKREYLLKKTYLPRESELIKERHLNDTRYASTFLKNFIEQNLQFKEVEVNLRKKRVQTVNGVITAHLRKRWGLEKNRQETYLHHAMDAIIVACTDHHMVTRITEYYQIKESNKSVKKPYFPMPWEGFRDELLSHLASQPIAKKISEELKAGYQSSDYIFVSRMPKRSVTGAAHDQTIRRKGGIDKKGKTIIIKRVRLKDIKFDENGDFKMVGKEQDLATYEAIKQRYLEHRKNSKKAFETPLYKPSKKGTGNLIKRVKIEGQTKAFVREVNGGVAQNSDLVRVDLFEKDDKYYMVPIYVPDTVCSELPKKVVKSGKGYEQWLTLDNSFTFKSSLYPYDLVRLVKGNEDRFLYFGTLDIDSDRLNFKDVNKPSKQNEYRYSLKTIENLEKYEVGVLGDLRLVKQETRRIFNRPKKKRKV

SEQ ID NO: 453 - DNA, 25 bp, Homo sapiens
  misc_feature 1..25: note = source = /note="Hurler target 1"
  source 1..25: mol_type = genomic DNA
ggagcagctctaggccgaagtgtcg

SEQ ID NO: 454 - DNA, 25 bp, Homo sapiens
  misc_feature 1..25: note = source = /note="Hurler target 2"
  source 1..25: mol_type = genomic DNA
taggccgaagtgtcgcaggccggga

SEQ ID NO: 455 - DNA, 25 bp, Homo sapiens
  misc_feature 1..25: note = source = /note="Hurler target 3"
  source 1..25: mol_type = genomic DNA
gctctaggccgaagtgtcgcaggcc

SEQ ID NO: 456 - DNA, 135 bp, synthetic construct
  misc_feature 1..135: note = source = /note="Description of Artificial Sequence: Syntheticpolynucleotide"
  misc_feature 1..135: note = source = /note="sgRNA Hurler target 1"
  source 1..135: mol_type = other DNA
ggagcagctctaggccgaagtgtcggtcatagttccatgaaagccaaaagtggctttgatgtttctatgataagggtttcggcccgtggcgtcggggatcgcctgcccattccgatgggcttctccccatttatt

SEQ ID NO: 457 - DNA, 135 bp, synthetic construct
  misc_feature 1..135: note = source = /note="Description of Artificial Sequence: Syntheticpolynucleotide"
  misc_feature 1..135: note = source = /note="sgRNA Hurler target 2"
  source 1..135: mol_type = other DNA
taggccgaagtgtcgcaggccgggagtcatagttccatgaaagccaaaagtggctttgatgtttctatgataagggtttcggcccgtggcgtcggggatcgcctgcccattccgatgggcttctccccatttatt

SEQ ID NO: 458 - DNA, 135 bp, synthetic construct
  misc_feature 1..135: note = source = /note="Description of Artificial Sequence: Syntheticpolynucleotide"
  misc_feature 1..135: note = source = /note="sgRNA Hurler target 3"
  source 1..135: mol_type = other DNA
ggcctgcgacacttcggcctagagcgtcatagttccatgaaagccaaaagtggctttgatgtttctatgataagggtttcggcccgtggcgtcggggatcgcctgcccattccgatgggcttctccccatttatt

SEQ ID NO: 459 - DNA, 15 bp, synthetic construct
  misc_feature 1..15: note = source = /note="Description of Artificial Sequence: Syntheticoligonucleotide"
  misc_feature 1..15: note = source = /note="Hurler Forward Amplification Primer"
  source 1..15: mol_type = other DNA
gactccttcaccaag

SEQ ID NO: 460 - DNA, 14 bp, synthetic construct
  misc_feature 1..14: note = source = /note="Description of Artificial Sequence: Syntheticoligonucleotide"
  misc_feature 1..14: note = source = /note="Hurler Reverse Amplification Primer"
  source 1..14: mol_type = other DNA
gtagatcagcaccg

SEQ ID NO: 461 - DNA, 14 bp, synthetic construct
  misc_feature 1..14: note = source = /note="Description of Artificial Sequence: Syntheticoligonucleotide"
  misc_feature 1..14: note = source = /note="Hurler Wild Type Probe"
  source 1..14: mol_type = other DNA
ctctgggccgaagt

SEQ ID NO: 462 - DNA, 14 bp, synthetic construct
  misc_feature 1..14: note = source = /note="Description of Artificial Sequence: Syntheticoligonucleotide"
  misc_feature 1..14: note = source = /note="Hurler W402X Probe-beta"
  source 1..14: mol_type = other DNA
ctctaggccgaagt

SEQ ID NO: 463 - DNA, 13 bp, synthetic construct
  misc_feature 1..13: note = source = /note="Description of Artificial Sequence: Syntheticoligonucleotide"
  misc_feature 1..13: note = source = /note="Hurler NGS forward"
  source 1..13: mol_type = other DNA
acttcctccagcc

SEQ ID NO: 464 - DNA, 13 bp, synthetic construct
  misc_feature 1..13: note = source = /note="Description of Artificial Sequence: Syntheticoligonucleotide"
  misc_feature 1..13: note = source = /note="Hurler NGS Reverse"
  source 1..13: mol_type = other DNA
gaaccccggctta

SEQ ID NO: 465 - DNA, 31 bp, Homo sapiens
  misc_feature 1..31: note = source = /note="Human W402X"
  source 1..31: mol_type = genomic DNA
ggagcagctctaggccgaagtgtcgcaggcc

SEQ ID NO: 466 - DNA, 31 bp, Mus musculus
  misc_feature 1..31: note = source = /note="Mouse W392X"
  source 1..31: mol_type = genomic DNA
agaacaactctaggcagaggtctcaaaggct

SEQ ID NO: 467 - DNA, 31 bp, synthetic construct; identical to SEQ ID NO: 465
  misc_feature 1..31: note = source = /note="Description of Artificial Sequence: Syntheticoligonucleotide"
  misc_feature 1..31: note = source = /note="humanized mouse"
  source 1..31: mol_type = other DNA

SEQ ID NO: 468 - DNA, 25 bp, Homo sapiens
  misc_feature 1..25: note = source = /note="FRDA target 1"
  source 1..25: mol_type = genomic DNA
atcacctgaggtccggagttcaaga

SEQ ID NO: 469 - DNA, 25 bp, Homo sapiens
  misc_feature 1..25: note = source = /note="FRDA target 2"
  source 1..25: mol_type = genomic DNA
gtcttgaactccggacctcaggtga

SEQ ID NO: 470 - DNA, 25 bp, Homo sapiens
  misc_feature 1..25: note = source = /note="FRDA target 3"
  source 1..25: mol_type = genomic DNA
tgaactccggacctcaggtgatcca

SEQ ID NO: 471 - DNA, 25 bp, Homo sapiens
  misc_feature 1..25: note = source = /note="FRDA target 4"
  source 1..25: mol_type = genomic DNA
gaaaagttagccgggcgtggtgtcg

SEQ ID NO: 472 - DNA, 500 bp, synthetic construct
  misc_feature 1..500: note = source = /note="Description of Artificial Sequence: Syntheticpolynucleotide"
  misc_feature 1..500: note = source = /note="BCL11A enhancer region"
  source 1..500: mol_type = other DNA
aaaaatggacaattatgaggaggggagagtgcagacaggggaagcttcacctcctttacaattttgggagtccacacggcatggcatacaaattatttcattcccattgagaaataaaatccaattctccatcaccaagagagccttccgaaagaggcccccctgggcaaacggccaccgatggagaggtctgccagtcctcttctaccccacccacgcccccaccctaatcagaggccaaacccttcctggagcctgtgataaaagcaactgttagcttgcactagactagcttcaaagttgtattgaccctggtgtgttatgtctaagagtagatgccatatctcttttctggcctatgttattacctgtatggactttgcactggaatcagctatctgctcttacttatgcacacctggggcatagagccagccctgtatcgcttttcagccatctcactacagataactcccaagtcctgtctagctgccttcctt

SEQ ID NO: 473 - DNA, 26 bp, Homo sapiens
  misc_feature 1..26: note = source = /note="SCD target seq 1"
  source 1..26: mol_type = genomic DNA
gcactagactagcttcaaagttgtag

SEQ ID NO: 474 - DNA, 26 bp, Homo sapiens
  misc_feature 1..26: note = source = /note="SCD target seq 2"
  source 1..26: mol_type = genomic DNA
cctaatcagaggccaaacccttcctg

SEQ ID NO: 475 - DNA, 26 bp, Homo sapiens
  misc_feature 1..26: note = source = /note="SCD target seq 3"
  source 1..26: mol_type = genomic DNA
caagctaacagttgcttttatcacag

SEQ ID NO: 476 - DNA, 26 bp, Homo sapiens
  misc_feature 1..26: note = source = /note="SCD target seq 4"
  source 1..26: mol_type = genomic DNA
gcactagactagcttcaaagttgtag

SEQ ID NO: 477 - DNA, 26 bp, Homo sapiens
  misc_feature 1..26: note = source = /note="SCD target seq 5"
  source 1..26: mol_type = genomic DNA
cctaatcagaggccaaacccttcctg

SEQ ID NO: 478 - DNA, 26 bp, Homo sapiens
  misc_feature 1..26: note = source = /note="SCD target seq 6"
  source 1..26: mol_type = genomic DNA
caagctaacagttgcttttatcacag

SEQ ID NO: 479 - DNA, 22 bp, synthetic construct
  misc_feature 1..22: note = source = /note="Description of Artificial Sequence: Syntheticoligonucleotide"
  misc_feature 1..22: note = source = /note="RelA FWD"
  source 1..22: mol_type = other DNA
cttagtttcaccgcaggttcta

SEQ ID NO: 480 - DNA, 22 bp, synthetic construct
  misc_feature 1..22: note = source = /note="Description of Artificial Sequence: Syntheticoligonucleotide"
  misc_feature 1..22: note = source = /note="RelA REV"
  source 1..22: mol_type = other DNA
ctgtgcactcaacactgatcta

SEQ ID NO: 481 - DNA, 21 bp, synthetic construct
  misc_feature 1..21: note = source = /note="Description of Artificial Sequence: Syntheticoligonucleotide"
  misc_feature 1..21: note = source = /note="AurkB FWD"
  source 1..21: mol_type = other DNA
cccagccctaggttgtttatt

SEQ ID NO: 482 - DNA, 23 bp, synthetic construct
  misc_feature 1..23: note = source = /note="Description of Artificial Sequence: Syntheticoligonucleotide"
  misc_feature 1..23: note = source = /note="AurkB REV"
  source 1..23: mol_type = other DNA
ctggctacatcttccttgactac

SEQ ID NO: 483 - DNA, 20 bp, synthetic construct
  misc_feature 1..20: note = source = /note="Description of Artificial Sequence: Syntheticoligonucleotide"
  misc_feature 1..20: note = source = /note="HPRT1 FWD"
  source 1..20: mol_type = other DNA
gtggcagaagcagtgagtaa

SEQ ID NO: 484 - DNA, 22 bp, synthetic construct
  misc_feature 1..22: note = source = /note="Description of Artificial Sequence: Syntheticoligonucleotide"
  misc_feature 1..22: note = source = /note="HPRT1 REV"
  source 1..22: mol_type = other DNA
tcccatctaggcactaggtaaa

SEQ ID NO: 485 - DNA, 54 bp, synthetic construct
  misc_feature 1..54: note = source = /note="Description of Artificial Sequence: Syntheticoligonucleotide"
  misc_feature 1..54: note = source = /note="FWD_Guide 134, Guide 135, Guide 136, Guide 137"
  source 1..54: mol_type = other DNA
gtctcgtgggctcggagatgtgtataagagacagacaattctcctgcctcagcc

SEQ ID NO: 486 - DNA, 53 bp, synthetic construct
  misc_feature 1..53: note = source = /note="Description of Artificial Sequence: Syntheticoligonucleotide"
  misc_feature 1..53: note = source = /note="REV_Guide 134, Guide 135, Guide 136, Guide 137"
  source 1..53: mol_type = other DNA
tcgtcggcagcgtcagatgtgtataagagacagactgccatgggaagaaggtg

SEQ ID NO: 487 - DNA, 54 bp, synthetic construct; identical to SEQ ID NO: 485
  misc_feature 1..54: note = source = /note="Description of Artificial Sequence: Syntheticoligonucleotide"
  misc_feature 1..54: note = source = /note="FWD_Guide 138, Guide 139, Guide 140, Guide 141"
  source 1..54: mol_type = other DNA

SEQ ID NO: 488 - DNA, 53 bp, synthetic construct; identical to SEQ ID NO: 486
  misc_feature 1..53: note = source = /note="Description of Artificial Sequence: Syntheticoligonucleotide"
  misc_feature 1..53: note = source = /note="REV_Guide 138, Guide 139, Guide 140, Guide 141"
  source 1..53: mol_type = other DNA

SEQ ID NO: 489 - DNA, 53 bp, synthetic construct
  misc_feature 1..53: note = source = /note="Description of Artificial Sequence: Syntheticoligonucleotide"
  misc_feature 1..53: note = source = /note="REV_Guide 142, Guide 143, Guide 144, Guide 145"
  source 1..53: mol_type = other DNA
tcgtcggcagcgtcagatgtgtataagagacaggagctgcacatttgacgagc

SEQ ID NO: 490 - DNA, 54 bp, synthetic construct
  misc_feature 1..54: note = source = /note="Description of Artificial Sequence: Syntheticoligonucleotide"
  misc_feature 1..54: note = source = /note="FWD_Guide 142, Guide 143, Guide 144, Guide 145"
  source 1..54: mol_type = other DNA
gtctcgtgggctcggagatgtgtataagagacagattacaggtgtgagccacgg

SEQ ID NO: 491 - DNA, 53 bp, synthetic construct
  misc_feature 1..53: note = source = /note="Description of Artificial Sequence: Syntheticoligonucleotide"
  misc_feature 1..53: note = source = /note="REV_Guide 164, Guide 165, Guide 166, Guide 167"
  source 1..53: mol_type = other DNA
tcgtcggcagcgtcagatgtgtataagagacagctgacctcaggtgatacgcc

SEQ ID NO: 492 - DNA, 54 bp, synthetic construct
  misc_feature 1..54: note = source = /note="Description of Artificial Sequence: Syntheticoligonucleotide"
  misc_feature 1..54: note = source = /note="FWD_Guide 164, Guide 165, Guide 166, Guide 167"
  source 1..54: mol_type = other DNA
gtctcgtgggctcggagatgtgtataagagacagctttgggaggctgagacagg

SEQ ID NO: 493 - DNA, 53 bp, synthetic construct
  misc_feature 1..53: note = source = /note="Description of Artificial Sequence: Syntheticoligonucleotide"
  misc_feature 1..53: note = source = /note="REV_Guide 168, Guide 169, Guide 170, Guide 171"
  source 1..53: mol_type = other DNA
tcgtcggcagcgtcagatgtgtataagagacagtgctctattgtccaggctgg

SEQ ID NO: 494 - DNA, 54 bp, synthetic construct
  misc_feature 1..54: note = source = /note="Description of Artificial Sequence: Syntheticoligonucleotide"
  misc_feature 1..54: note = source = /note="FWD_Guide 168, Guide 169, Guide 170, Guide 171"
  source 1..54: mol_type = other DNA
gtctcgtgggctcggagatgtgtataagagacagtccagcaggtcagcaaagaa

SEQ ID NO: 495 - DNA, 53 bp, synthetic construct
  misc_feature 1..53: note = source = /note="Description of Artificial Sequence: Syntheticoligonucleotide"
  misc_feature 1..53: note = source = /note="REV_Guide 172, Guide 173, Guide 174, Guide 175"
  source 1..53: mol_type = other DNA
tcgtcggcagcgtcagatgtgtataagagacaggcagtataactggccagcct

SEQ ID NO: 496 - DNA, 56 bp, synthetic construct
  misc_feature 1..56: note = source = /note="Description of Artificial Sequence: Syntheticoligonucleotide"
  misc_feature 1..56: note = source = /note="FWD_Guide 172, Guide 173, Guide 174, Guide 175"
  source 1..56: mol_type = other DNA
gtctcgtgggctcggagatgtgtataagagacagtcagttgaggagttcagcttaa

SEQ ID NO: 497 - DNA, 53 bp, synthetic construct
  misc_feature 1..53: note = source = /note="Description of Artificial Sequence: Syntheticoligonucleotide"
  misc_feature 1..53: note = source = /note="REV_Guide 185, Guide 186, Guide 187, Guide 188"
  source 1..53: mol_type = other DNA
tcgtcggcagcgtcagatgtgtataagagacagtggcccctatgtggagatca

SEQ ID NO: 498 - DNA, 54 bp, synthetic construct
  misc_feature 1..54: note = source = /note="Description of Artificial Sequence: Syntheticoligonucleotide"
  misc_feature 1..54: note = source = /note="FWD_Guide 185, Guide 186, Guide 187, Guide 188"
  source 1..54: mol_type = other DNA
gtctcgtgggctcggagatgtgtataagagacagggcagagctcagcctcatag

SEQ ID NO: 499 - DNA, 53 bp, synthetic construct
  misc_feature 1..53: note = source = /note="Description of Artificial Sequence: Syntheticoligonucleotide"
  misc_feature 1..53: note = source = /note="REV_Guide 189, Guide 190, Guide 191, Guide 192"
  source 1..53: mol_type = other DNA
tcgtcggcagcgtcagatgtgtataagagacagatatccccacttcccctgct

SEQ ID NO: 500 - DNA, 54 bp, synthetic construct
  misc_feature 1..54: note = source = /note="Description of Artificial Sequence: Syntheticoligonucleotide"
  misc_feature 1..54: note = source = /note="FWD_Guide 189, Guide 190, Guide 191, Guide 192"
  source 1..54: mol_type = other DNA
gtctcgtgggctcggagatgtgtataagagacagcacctcaaggacagctctgg

SEQ ID NO: 501 - DNA, 53 bp, synthetic construct; identical to SEQ ID NO: 499
  misc_feature 1..53: note = source = /note="Description of Artificial Sequence: Syntheticoligonucleotide"
  misc_feature 1..53: note = source = /note="REV_Guide 193, Guide 194, Guide 195, Guide 196"
  source 1..53: mol_type = other DNA

SEQ ID NO: 502 - DNA, 54 bp, synthetic construct; identical to SEQ ID NO: 500
  misc_feature 1..54: note = source = /note="Description of Artificial Sequence: Syntheticoligonucleotide"
  misc_feature 1..54: note = source = /note="FWD_Guide 193, Guide 194, Guide 195, Guide 196"
  source 1..54: mol_type = other DNA

SEQ ID NO: 503 - DNA, 54 bp, synthetic construct; identical to SEQ ID NO: 485
  misc_feature 1..54: note = source = /note="Description of Artificial Sequence: Syntheticoligonucleotide"
  misc_feature 1..54: note = source = /note="FWD_Guide 146"
  source 1..54: mol_type = other DNA

SEQ ID NO: 504 - DNA, 53 bp, synthetic construct; identical to SEQ ID NO: 486
  misc_feature 1..53: note = source = /note="Description of Artificial Sequence: Syntheticoligonucleotide"
  misc_feature 1..53: note = source = /note="REV_Guide 146"
  source 1..53: mol_type = other DNA

SEQ ID NO: 505 - DNA, 54 bp, synthetic construct; identical to SEQ ID NO: 485
  misc_feature 1..54: note = source = /note="Description of Artificial Sequence: Syntheticoligonucleotide"
  misc_feature 1..54: note = source = /note="FWD_Guide 147"
  source 1..54: mol_type = other DNA

SEQ ID NO: 506 - DNA, 53 bp, synthetic construct; identical to SEQ ID NO: 486
  misc_feature 1..53: note = source = /note="Description of Artificial Sequence: Syntheticoligonucleotide"
  misc_feature 1..53: note = source = /note="REV_Guide 147"
  source 1..53: mol_type = other DNA

SEQ ID NO: 507 - DNA, 53 bp, synthetic construct; identical to SEQ ID NO: 486
  misc_feature 1..53: note = source = /note="Description of Artificial Sequence: Syntheticoligonucleotide"
  misc_feature 1..53: note = source = /note="REV_Guide 148"
  source 1..53: mol_type = other DNA

SEQ ID NO: 508 - DNA, 54 bp, synthetic construct; identical to SEQ ID NO: 485
  misc_feature 1..54: note = source = /note="Description of Artificial Sequence: Syntheticoligonucleotide"
  misc_feature 1..54: note = source = /note="FWD_Guide 148"
  source 1..54: mol_type = other DNA

SEQ ID NO: 509 - DNA, 53 bp, synthetic construct
  misc_feature 1..53: note = source = /note="Description of Artificial Sequence: Syntheticoligonucleotide"
  misc_feature 1..53: note = source = /note="REV_Guide 176"
  source 1..53: mol_type = other DNA
tcgtcggcagcgtcagatgtgtataagagacaggtagctcactgcagcctcaa

SEQ ID NO: 510 - DNA, 54 bp, synthetic construct
  misc_feature 1..54: note = source = /note="Description of Artificial Sequence: Syntheticoligonucleotide"
  misc_feature 1..54: note = source = /note="FWD_Guide 176"
  source 1..54: mol_type = other DNA
gtctcgtgggctcggagatgtgtataagagacagtgggtgatgaacatacgggt

SEQ ID NO: 511 - DNA, 57 bp, synthetic construct
  misc_feature 1..57: note = source = /note="Description of Artificial Sequence: Syntheticoligonucleotide"
  misc_feature 1..57: note = source = /note="REV_Guide 177"
  source 1..57: mol_type = other DNA
tcgtcggcagcgtcagatgtgtataagagacagtcagactgaagagctattgtgtga

SEQ ID NO: 512 - DNA, 54 bp, synthetic construct
  misc_feature 1..54: note = source = /note="Description of Artificial Sequence: Syntheticoligonucleotide"
  misc_feature 1..54: note = source = /note="FWD_Guide 177"
  source 1..54: mol_type = other DNA
gtctcgtgggctcggagatgtgtataagagacagccccacaaaccgatgtagct

SEQ ID NO: 513 - DNA, 53 bp, synthetic construct
  misc_feature 1..53: note = source = /note="Description of Artificial Sequence: Syntheticoligonucleotide"
  misc_feature 1..53: note = source = /note="REV_Guide 209"
  source 1..53: mol_type = other DNA
tcgtcggcagcgtcagatgtgtataagagacagtggagtgcagtggtgtgatc

SEQ ID NO: 514 - DNA, 54 bp, synthetic construct
  misc_feature 1..54: note = source = /note="Description of Artificial Sequence: Syntheticoligonucleotide"
  misc_feature 1..54: note = source = /note="FWD_Guide 209"
  source 1..54: mol_type = other DNA
gtctcgtgggctcggagatgtgtataagagacagttgaggctgcagtgagctac

SEQ ID NO: 515 - DNA, 53 bp, synthetic construct; identical to SEQ ID NO: 497
  misc_feature 1..53: note = source = /note="Description of Artificial Sequence: Syntheticoligonucleotide"
  misc_feature 1..53: note = source = /note="REV_Guide 197"
  source 1..53: mol_type = other DNA

SEQ ID NO: 516 - DNA, 54 bp, synthetic construct; identical to SEQ ID NO: 498
  misc_feature 1..54: note = source = /note="Description of Artificial Sequence: Syntheticoligonucleotide"
  misc_feature 1..54: note = source = /note="FWD_Guide 197"
  source 1..54: mol_type = other DNA

SEQ ID NO: 517 - DNA, 53 bp, synthetic construct
  misc_feature 1..53: note = source = /note="Description of Artificial Sequence: Syntheticoligonucleotide"
  misc_feature 1..53: note = source = /note="REV_Guide 198"
  source 1..53: mol_type = other DNA
tcgtcggcagcgtcagatgtgtataagagacagccaagccgaccaaacaagtg

SEQ ID NO: 518 - DNA, 54 bp, synthetic construct
  misc_feature 1..54: note = source = /note="Description of Artificial Sequence: Syntheticoligonucleotide"
  misc_feature 1..54: note = source = /note="FWD_Guide 198"
  source 1..54: mol_type = other DNA
gtctcgtgggctcggagatgtgtataagagacaggatgcgctgactgatagcct

SEQ ID NO: 519 - DNA, 53 bp, synthetic construct; identical to SEQ ID NO: 497
  misc_feature 1..53: note = source = /note="Description of Artificial Sequence: Syntheticoligonucleotide"
  misc_feature 1..53: note = source = /note="REV_Guide 199"
  source 1..53: mol_type = other DNA

SEQ ID NO: 520 - DNA, 54 bp, synthetic construct; identical to SEQ ID NO: 498
  misc_feature 1..54: note = source = /note="Description of Artificial Sequence: Syntheticoligonucleotide"
  misc_feature 1..54: note = source = /note="FWD_Guide 199"
  source 1..54: mol_type = other DNA

SEQ ID NO: 521 - DNA, 54 bp, synthetic construct
  misc_feature 1..54: note = source = /note="Description of Artificial Sequence: Syntheticoligonucleotide"
  misc_feature 1..54: note = source = /note="FWD_Guide 149"
  source 1..54: mol_type = other DNA
gtctcgtgggctcggagatgtgtataagagacagggtaaggggaaactggaggc

SEQ ID NO: 522 - DNA, 53 bp, synthetic construct
  misc_feature 1..53: note = source = /note="Description of Artificial Sequence: Syntheticoligonucleotide"
  misc_feature 1..53: note = source = /note="REV_Guide 149"
  source 1..53: mol_type = other DNA
tcgtcggcagcgtcagatgtgtataagagacagtccactttctaagcaggcgg

SEQ ID NO: 523 - DNA, 54 bp, synthetic construct
  misc_feature 1..54: note = source = /note="Description of Artificial Sequence: Syntheticoligonucleotide"
  misc_feature 1..54: note = source = /note="FWD_Guide 150"
  source 1..54: mol_type = other DNA
gtctcgtgggctcggagatgtgtataagagacaggtgggcctggatttcgatct

SEQ ID NO: 524 - DNA, 53 bp, synthetic construct
  misc_feature 1..53: note = source = /note="Description of Artificial Sequence: Syntheticoligonucleotide"
  misc_feature 1..53: note = source = /note="REV_Guide 150"
  source 1..53: mol_type = other DNA
tcgtcggcagcgtcagatgtgtataagagacagatgaacaggcaggaagtcgg

SEQ ID NO: 525 - DNA, 53 bp, synthetic construct
  misc_feature 1..53: note = source = /note="Description of Artificial Sequence: Syntheticoligonucleotide"
  misc_feature 1..53: note = source = /note="REV_Guide 151"
  source 1..53: mol_type = other DNA
tcgtcggcagcgtcagatgtgtataagagacagcggttctgagttgccttcct

SEQ ID NO: 526 - DNA, 54 bp, synthetic construct; identical to SEQ ID NO: 521
  misc_feature 1..54: note = source = /note="Description of Artificial Sequence: Syntheticoligonucleotide"
  misc_feature 1..54: note = source = /note="FWD_Guide 151"
  source 1..54: mol_type = other DNA

SEQ ID NO: 527 - DNA, 53 bp, synthetic construct
  misc_feature 1..53: note = source = /note="Description of Artificial Sequence: Syntheticoligonucleotide"
  misc_feature 1..53: note = source = /note="REV_Guide 179"
  source 1..53: mol_type = other DNA
tcgtcggcagcgtcagatgtgtataagagacagtcagtggtccacatgcaagt

SEQ ID NO: 528 - DNA, 54 bp, synthetic construct
  misc_feature 1..54: note = source = /note="Description of Artificial Sequence: Syntheticoligonucleotide"
  misc_feature 1..54: note = source = /note="FWD_Guide 179"
  source 1..54: mol_type = other DNA
gtctcgtgggctcggagatgtgtataagagacagaaggcaagttgatcgctcga

SEQ ID NO: 529 - DNA, 53 bp, synthetic construct
  misc_feature 1..53: note = source = /note="Description of Artificial Sequence: Syntheticoligonucleotide"
  misc_feature 1..53: note = source = /note="REV_Guide 180"
  source 1..53: mol_type = other DNA
tcgtcggcagcgtcagatgtgtataagagacagagtggttctgttgcacacgt

SEQ ID NO: 530 - DNA, 54 bp, synthetic construct
  misc_feature 1..54: note = source = /note="Description of Artificial Sequence: Syntheticoligonucleotide"
  misc_feature 1..54: note = source = /note="FWD_Guide 180"
  source 1..54: mol_type = other DNA
gtctcgtgggctcggagatgtgtataagagacagtcgcttaagcccaggagttc

SEQ ID NO: 531 - DNA, 55 bp, synthetic construct
  misc_feature 1..55: note = source = /note="Description of Artificial Sequence: Syntheticoligonucleotide"
  misc_feature 1..55: note = source = /note="REV_Guide 181"
  source 1..55: mol_type = other DNA
tcgtcggcagcgtcagatgtgtataagagacagtctggctgatccgtactaatcc

SEQ ID NO: 532 - DNA, 54 bp, synthetic construct
  misc_feature 1..54: note = source = /note="Description of Artificial Sequence: Syntheticoligonucleotide"
  misc_feature 1..54: note = source = /note="FWD_Guide 181"
  source 1..54: mol_type = other DNA
gtctcgtgggctcggagatgtgtataagagacagtaggctcaagggatgctcct

SEQ ID NO: 533 - DNA, 53 bp, synthetic construct
  misc_feature 1..53: note = source = /note="Description of Artificial Sequence: Syntheticoligonucleotide"
  misc_feature 1..53: note = source = /note="REV_Guide 200"
  source 1..53: mol_type = other DNA
tcgtcggcagcgtcagatgtgtataagagacagccagagctgtccttgaggtg

SEQ ID NO: 534 - DNA, 54 bp, synthetic construct
  misc_feature 1..54: note = source = /note="Description of Artificial Sequence: Syntheticoligonucleotide"
  misc_feature 1..54: note = source = /note="FWD_Guide 200"
  source 1..54: mol_type = other DNA
gtctcgtgggctcggagatgtgtataagagacaggtccatacccaccttggcaa

SEQ ID NO: 535 - DNA, 53 bp, synthetic construct; identical to SEQ ID NO: 517
  misc_feature 1..53: note = source = /note="Description of Artificial Sequence: Syntheticoligonucleotide"
  misc_feature 1..53: note = source = /note="REV_Guide 201"
  source 1..53: mol_type = other DNA

SEQ ID NO: 536 - DNA, 54 bp, synthetic construct; identical to SEQ ID NO: 518
  misc_feature 1..54: note = source = /note="Description of Artificial Sequence: Syntheticoligonucleotide"
  misc_feature 1..54: note = source = /note="FWD_Guide 201"
  source 1..54: mol_type = other DNA

SEQ ID NO: 537 - DNA, 53 bp, synthetic construct
  misc_feature 1..53: note = source = /note="Description of Artificial Sequence: Syntheticoligonucleotide"
  misc_feature 1..53: note = source = /note="REV_Guide 202"
  source 1..53: mol_type = other DNA
tcgtcggcagcgtcagatgtgtataagagacagaggctatcagtcagcgcatc

SEQ ID NO: 538 - DNA, 54 bp, synthetic construct
  misc_feature 1..54: note = source = /note="Description of Artificial Sequence: Syntheticoligonucleotide"
  misc_feature 1..54: note = source = /note="FWD_Guide 202"
  source 1..54: mol_type = other DNA
gtctcgtgggctcggagatgtgtataagagacagagcaggggaagtggggatat

SEQ ID NO: 539 - DNA, 54 bp, synthetic construct; identical to SEQ ID NO: 490
  misc_feature 1..54: note = source = /note="Description of Artificial Sequence: Syntheticoligonucleotide"
  misc_feature 1..54: note = source = /note="FWD_Guide 152"
  source 1..54: mol_type = other DNA

SEQ ID NO: 540 - DNA, 53 bp, synthetic construct; identical to SEQ ID NO: 489
  misc_feature 1..53: note = source = /note="Description of Artificial Sequence: Syntheticoligonucleotide"
  misc_feature 1..53: note = source = /note="REV_Guide 152"
  source 1..53: mol_type = other DNA

SEQ ID NO: 541 - DNA, 54 bp, synthetic construct; identical to SEQ ID NO: 485
  misc_feature 1..54: note = source = /note="Description of Artificial Sequence: Syntheticoligonucleotide"
  misc_feature 1..54: note = source = /note="FWD_Guide 153"
  source 1..54: mol_type = other DNA

SEQ ID NO: 542 - DNA, 53 bp, synthetic construct; identical to SEQ ID NO: 486
  misc_feature 1..53: note = source = /note="Description of Artificial Sequence: Syntheticoligonucleotide"
  misc_feature 1..53: note = source = /note="REV_Guide 153"
  source 1..53: mol_type = other DNA

SEQ ID NO: 543 - DNA, 54 bp, synthetic construct; identical to SEQ ID NO: 490
  misc_feature 1..54: note = source = /note="Description of Artificial Sequence: Syntheticoligonucleotide"
  misc_feature 1..54: note = source = /note="FWD_Guide 154"
  source 1..54: mol_type = other DNA

SEQ ID NO: 544 - DNA, 53 bp, synthetic construct; identical to SEQ ID NO: 489
  misc_feature 1..53: note = source = /note="Description of Artificial Sequence: Syntheticoligonucleotide"
  misc_feature 1..53: note = source = /note="REV_Guide 154"
  source 1..53: mol_type = other DNA

SEQ ID NO: 545 - DNA, 54 bp, synthetic construct; identical to SEQ ID NO: 510
  misc_feature 1..54: note = source = /note="Description of Artificial Sequence: Syntheticoligonucleotide"
  misc_feature 1..54: note = source = /note="FWD_Guide 182"
  source 1..54: mol_type = other DNA

SEQ ID NO: 546 - DNA, 53 bp, synthetic construct; identical to SEQ ID NO: 509
  misc_feature 1..53: note = source = /note="Description of Artificial Sequence: Syntheticoligonucleotide"
  misc_feature 1..53: note = source = /note="REV_Guide 182"
  source 1..53: mol_type = other DNA

SEQ ID NO: 547 - DNA, 54 bp, synthetic construct
  misc_feature 1..54: note = source = /note="Description of Artificial Sequence: Syntheticoligonucleotide"
  misc_feature 1..54: note = source = /note="FWD_Guide 183"
  source 1..54: mol_type = other DNA
gtctcgtgggctcggagatgtgtataagagacagtgcctgtagtcccagctact

SEQ ID NO: 548 - DNA, 53 bp, synthetic construct
  misc_feature 1..53: note = source = /note="Description of Artificial Sequence: Syntheticoligonucleotide"
  misc_feature 1..53: note = source = /note="REV_Guide 183"
  source 1..53: mol_type = other DNA
tcgtcggcagcgtcagatgtgtataagagacagtcagctcagtgcaacctctg

SEQ ID NO: 549 - DNA, 54 bp, synthetic construct; identical to SEQ ID NO: 492
  misc_feature 1..54: note = source = /note="Description of Artificial Sequence: Syntheticoligonucleotide"
  misc_feature 1..54: note = source = /note="FWD_Guide 184"
  source 1..54: mol_type = other DNA

SEQ ID NO: 550 - DNA, 53 bp, synthetic construct; identical to SEQ ID NO: 491
  misc_feature 1..53: note = source = /note="Description of Artificial Sequence: Syntheticoligonucleotide"
  misc_feature 1..53: note = source = /note="REV_Guide 184"
  source 1..53: mol_type = other DNA

SEQ ID NO: 551 - DNA, 54 bp, synthetic construct; identical to SEQ ID NO: 518
  misc_feature 1..54: note = source = /note="Description of Artificial Sequence: Syntheticoligonucleotide"
  misc_feature 1..54: note = source = /note="FWD_Guide 203"
  source 1..54: mol_type = other DNA

SEQ ID NO: 552 - DNA, 53 bp, synthetic construct; identical to SEQ ID NO: 517
  misc_feature 1..53: note = source = /note="Description of Artificial Sequence: Syntheticoligonucleotide"
  misc_feature 1..53: note = source = /note="REV_Guide 203"
  source 1..53: mol_type = other DNA

SEQ ID NO: 553 - DNA, 54 bp, synthetic construct; identical to SEQ ID NO: 500
  misc_feature 1..54: note = source = /note="Description of Artificial Sequence: Syntheticoligonucleotide"
  misc_feature 1..54: note = source = /note="FWD_Guide 204"
  source 1..54: mol_type = other DNA

SEQ ID NO: 554 - DNA, 53 bp, synthetic construct; identical to SEQ ID NO: 499
  misc_feature 1..53: note = source = /note="Description of Artificial Sequence: Syntheticoligonucleotide"
  misc_feature 1..53: note = source = /note="REV_Guide 204"
  source 1..53: mol_type = other DNA

SEQ ID NO: 555 - DNA, 54 bp, synthetic construct; identical to SEQ ID NO: 498
  misc_feature 1..54: note = source = /note="Description of Artificial Sequence: Syntheticoligonucleotide"
  misc_feature 1..54: note = source = /note="FWD_Guide 205"
  source 1..54: mol_type = other DNA

SEQ ID NO: 556 - DNA, 53 bp, synthetic construct; identical to SEQ ID NO: 497
  misc_feature 1..53: note = source = /note="Description of Artificial Sequence: Syntheticoligonucleotide"
  misc_feature 1..53: note = source = /note="REV_Guide 205"
  source 1..53: mol_type = other DNA

SEQ ID NO: 557-558 - skipped (2 entries)

SEQ ID NO: 559 - DNA, 30 bp, synthetic construct
  misc_feature 1..30: note = source = /note="Description of Artificial Sequence: Syntheticoligonucleotide"
  misc_feature 1..30: note = source = /note="sequence 2 for cleavage site assay"
  source 1..30: mol_type = other DNA
ccatgatatagacgttgtggctgttgtagt

SEQ ID NO: 560 - protein, 6 aa, synthetic construct
  REGION 1..6: note = source = /note="Description of Artificial Sequence: Synthetic6xHis tag"
HHHHHH

SEQ ID NO: 561 - protein, 10 aa, synthetic construct
  REGION 1..10: note = source = /note="Description of Artificial Sequence: Synthetic10xHis tag"
HHHHHHHHHH

SEQ ID NO: 562 - DNA, 34 bp, synthetic construct
  misc_feature 1..34: note = source = /note="Description of Artificial Sequence: Syntheticoligonucleotide"
  source 1..34: mol_type = other DNA
cctgggtgtgaggctgggccattaaaacctctcc

SEQ ID NO: 563 - DNA, 15 bp, synthetic construct
  misc_feature 1..15: note = source = /note="Description of Artificial Sequence: Syntheticoligonucleotide"
  source 1..15: mol_type = other DNA
cctgaaaacctctcc

SEQ ID NO: 564 - DNA, 33 bp, synthetic construct
  misc_feature 1..33: note = source = /note="Description of Artificial Sequence: Syntheticoligonucleotide"
  source 1..33: mol_type = other DNA
cctgggtgtgagctgggccattaaaacctctcc

SEQ ID NO: 565 - DNA, 22 bp, synthetic construct
  misc_feature 1..22: note = source = /note="Description of Artificial Sequence: Syntheticoligonucleotide"
  source 1..22: mol_type = other DNA
cctgggccattaaaacctctcc

SEQ ID NO: 566 - DNA, 11 bp, synthetic construct
  misc_feature 1..11: note = source = /note="Description of Artificial Sequence: Syntheticoligonucleotide"
  source 1..11: mol_type = other DNA
cctggctctcc

SEQ ID NO: 567 - DNA, 20 bp, synthetic construct
  misc_feature 1..20: note = source = /note="Description of Artificial Sequence: Syntheticoligonucleotide"
  source 1..20: mol_type = other DNA
cgggccattaaaacctctcc

SEQ ID NO: 568 - DNA, 40 bp, synthetic construct
  misc_feature 1..40: note = source = /note="Description of Artificial Sequence: Syntheticoligonucleotide"
  source 1..40: mol_type = other DNA
cctgggtgtgaggccagacctgggccattaaaacctctcc

SEQ ID NO: 569 - DNA, 79 bp, synthetic construct
  misc_feature 1..79: note = source = /note="Description of Artificial Sequence: Syntheticoligonucleotide"
  source 1..79: mol_type = other DNA
cctgggtgtgaggggaagctgacgtcctttccatggctgctcgcctgtgttgccaccgctgggccattaaaacctctcc

SEQ ID NO: 570 - DNA, 33 bp, synthetic construct
  misc_feature 1..33: note = source = /note="Description of Artificial Sequence: Syntheticoligonucleotide"
  source 1..33: mol_type = other DNA
cctgggtgtgacctgggccattaaaacctctcc

SEQ ID NO: 571 - DNA, 35 bp, synthetic construct
  misc_feature 1..35: note = source = /note="Description of Artificial Sequence: Syntheticoligonucleotide"
  source 1..35: mol_type = other DNA
cctgggtgtgaggactgggccattaaaacctctcc

SEQ ID NO: 572 - DNA, 34 bp, synthetic construct
  misc_feature 1..34: note = source = /note="Description of Artificial Sequence: Syntheticoligonucleotide"
  source 1..34: mol_type = other DNA
agttggcagatgctctaatgtactgccatgggaa

SEQ ID NO: 573 - DNA, 31 bp, synthetic construct
  misc_feature 1..31: note = source = /note="Description of Artificial Sequence: Syntheticoligonucleotide"
  source 1..31: mol_type = other DNA
agttggcagatgcaatgtactgccatgggaa

SEQ ID NO: 574 - DNA, 30 bp, synthetic construct
  misc_feature 1..30: note = source = /note="Description of Artificial Sequence: Syntheticoligonucleotide"
  source 1..30: mol_type = other DNA
agttggcagatgcatgtactgccatgggaa

SEQ ID NO: 575 - DNA, 31 bp, synthetic construct
  misc_feature 1..31: note = source = /note="Description of Artificial Sequence: Syntheticoligonucleotide"
  source 1..31: mol_type = other DNA
agttggcagatgctatgtactgccatgggaa

SEQ ID NO: 576 - DNA, 32 bp, synthetic construct
  misc_feature 1..32: note = source = /note="Description of Artificial Sequence: Syntheticoligonucleotide"
  source 1..32: mol_type = other DNA
agttggcagatgctaatgtactgccatgggaa

SEQ ID NO: 577 - DNA, 31 bp, synthetic construct
  misc_feature 1..31: note = source = /note="Description of Artificial Sequence: Syntheticoligonucleotide"
  source 1..31: mol_type = other DNA
agttggcagatgctatgtaatgccatgggaa

SEQ ID NO: 578 - DNA, 33 bp, synthetic construct
  misc_feature 1..33: note = source = /note="Description of Artificial Sequence: Syntheticoligonucleotide"
  source 1..33: mol_type = other DNA
agttggcagatgctctatgtactgccatgggaa

SEQ ID NO: 579 - DNA, 39 bp, synthetic construct
  misc_feature 1..39: note = source = /note="Description of Artificial Sequence: Syntheticoligonucleotide"
  source 1..39: mol_type = other DNA
cgacctgaatgctgtgcggcgctctggcttcattcaatc

SEQ ID NO: 580 - DNA, 35 bp, synthetic construct
  misc_feature 1..35: note = source = /note="Description of Artificial Sequence: Syntheticoligonucleotide"
  source 1..35: mol_type = other DNA
cgacctgaatgctgtgcggctctgcttccaggtga

SEQ ID NO: 581 - DNA, 36 bp, synthetic construct
  misc_feature 1..36: note = source = /note="Description of Artificial Sequence: Syntheticoligonucleotide"
  source 1..36: mol_type = other DNA
cgacctgaatgctgtgcggcatctgcttccaggtga

SEQ ID NO: 582 - DNA, 25 bp, synthetic construct
  misc_feature 1..25: note = source = /note="Description of Artificial Sequence: Syntheticoligonucleotide"
  source 1..25: mol_type = other DNA
cgacctgaatgctgcttccaggtga

SEQ ID NO: 583 - DNA, 28 bp, synthetic construct
  misc_feature 1..28: note = source = /note="Description of Artificial Sequence: Syntheticoligonucleotide"
  source 1..28: mol_type = other DNA
cgacctgaatgcttctgcttccaggtga

SEQ ID NO: 584 - DNA, 22 bp, synthetic construct
  misc_feature 1..22: note = source = /note="Description of Artificial Sequence: Syntheticoligonucleotide"
  source 1..22: mol_type = other DNA
cgacctgaatgcttccaggtga

SEQ ID NO: 585 - DNA, 35 bp, synthetic construct
  misc_feature 1..35: note = source = /note="Description of Artificial Sequence: Syntheticoligonucleotide"
  source 1..35: mol_type = other DNA
gcgtggggactacgacctgaatgctgtgcggctct

SEQ ID NO: 586 - DNA, 24 bp, synthetic construct
  misc_feature 1..24: note = source = /note="Description of Artificial Sequence: Syntheticoligonucleotide"
  source 1..24: mol_type = other DNA
gcgacctgaatgctgtgcggctct

SEQ ID NO: 587 - DNA, 28 bp, synthetic construct
  misc_feature 1..28: note = source = /note="Description of Artificial Sequence: Syntheticoligonucleotide"
  source 1..28: mol_type = other DNA
gcgtggggactacgagctgtgcggctct

SEQ ID NO: 588 - DNA, 28 bp, synthetic construct
  misc_feature 1..28: note = source = /note="Description of Artificial Sequence: Syntheticoligonucleotide"
  source 1..28: mol_type = other DNA
gcgtggggactgaatgctgtgcggctct

SEQ ID NO: 589 - DNA, 31 bp, synthetic construct
  misc_feature 1..31: note = source = /note="Description of Artificial Sequence: Syntheticoligonucleotide"
  source 1..31: mol_type = other DNA
gcgtggggactcctgaatgctgtgcggctct

SEQ ID NO: 590 - DNA, 36 bp, synthetic construct
  misc_feature 1..36: note = source = /note="Description of Artificial Sequence: Syntheticoligonucleotide"
  source 1..36: mol_type = other DNA
gcgtggggactacgaacctgaatgctgtgcggctct

SEQ ID NO: 591 - DNA, 30 bp, synthetic construct
  misc_feature 1..30: note = source = /note="Description of Artificial Sequence: Syntheticoligonucleotide"
  source 1..30: mol_type = other DNA
gcgtggggactacgaatgctgtgcggctct

SEQ ID NO: 592 - DNA, 32 bp, synthetic construct
  misc_feature 1..32: note = source = /note="Description of Artificial Sequence: Syntheticoligonucleotide"
  source 1..32: mol_type = other DNA
gcgtggggactacctgaatgctgtgcggctct

SEQ ID NO: 593 - DNA, 21 bp, synthetic construct
  misc_feature 1..21: note = source = /note="Description of Artificial Sequence: Syntheticoligonucleotide"
  source 1..21: mol_type = other DNA
gcgtgaatgctgtgcggctct

SEQ ID NO: 594 - DNA, 24 bp, synthetic construct
  misc_feature 1..24: note = source = /note="Description of Artificial Sequence: Syntheticoligonucleotide"
  source 1..24: mol_type = other DNA
gcgacctgactgctgtgcggctct

SEQ ID NO: 595 - DNA, 34 bp, synthetic construct
  misc_feature 1..34: note = source = /note="Description of Artificial Sequence: Syntheticoligonucleotide"
  source 1..34: mol_type = other DNA
gcgtggggactacgcctgaatgctgtgcggctct

SEQ ID NO: 596 - DNA, 42 bp, synthetic construct
  misc_feature 1..42: note = source = /note="Description of Artificial Sequence: Syntheticoligonucleotide"
  source 1..42: mol_type = other DNA
atggaggagttggcagatgctctaatgtactgccatgggaag

SEQ ID NO: 597 - DNA, 40 bp, synthetic construct
  misc_feature 1..40: note = source = /note="Description of Artificial Sequence: Syntheticoligonucleotide"
  source 1..40: mol_type = other DNA
atggaggagttggcagatgctaatgtactgccatgggaag

SEQ ID NO: 598 - DNA, 34 bp, synthetic construct
  misc_feature 1..34: note = source = /note="Description of Artificial Sequence: Syntheticoligonucleotide"
  source 1..34: mol_type = other DNA
atggaggagttggcagatgtactgccatgggaag

SEQ ID NO: 599 - DNA, 18 bp, synthetic construct
  misc_feature 1..18: note = source = /note="Description of Artificial Sequence: Syntheticoligonucleotide"
  source 1..18: mol_type = other DNA
atgtactgccatgggaag

SEQ ID NO: 600 - DNA, 30 bp, synthetic construct
  misc_feature 1..30: note = source = /note="Description of Artificial Sequence: Syntheticoligonucleotide"
  source 1..30: mol_type = other DNA
atggaggagttggtgtactgccatgggaag

SEQ ID NO: 601 - DNA, 43 bp, synthetic construct
  misc_feature 1..43: note = source = /note="Description of Artificial Sequence: Syntheticoligonucleotide"
  source 1..43: mol_type = other DNA
atggaggagttggcagatgctctaaatgtactgccatgggaag

SEQ ID NO: 602 - DNA, 45 bp, synthetic construct
  misc_feature 1..45: note = source = /note="Description of Artificial Sequence: Syntheticoligonucleotide"
  source 1..45: mol_type = other DNA
atggaggagttggcagatgctcttctaatgtactgccatgggaag

SEQ ID NO: 603 - DNA, 44 bp, synthetic construct
  misc_feature 1..44: note = source = /note="Description of Artificial Sequence: Syntheticoligonucleotide"
  source 1..44: mol_type = other DNA
atggaggagttggcagatgccctctaatgtactgccatgggaag

SEQ ID NO: 604 - DNA, 40 bp, synthetic construct
  misc_feature 1..40: note = source = /note="Description of Artificial Sequence: Syntheticoligonucleotide"
  source 1..40: mol_type = other DNA
atggaggagttggcagataatgtactgccatgggaagaag

SEQ ID NO: 605 - DNA, 24 bp, synthetic construct
  misc_feature 1..24: note = source = /note="Description of Artificial Sequence: Syntheticoligonucleotide"
  source 1..24: mol_type = other DNA
atggcgtactgccatgggaagaag

SEQ ID NO: 606 - DNA, 46 bp, synthetic construct
  misc_feature 1..46: note = source = /note="Description of Artificial Sequence: Syntheticoligonucleotide"
  source 1..46: mol_type = other DNA
atggaggagttggcagatgcttctaatgtactgccatgggaagaag

SEQ ID NO: 607 - DNA, 32 bp, synthetic construct
  misc_feature 1..32: note = source = /note="Description of Artificial Sequence: Syntheticoligonucleotide"
  source 1..32: mol_type = other DNA
atggaggagttggcatctgccatgggaagaag

SEQ ID NO: 608 - DNA, 42 bp, synthetic construct
  misc_feature 1..42: note = source = /note="Description of Artificial Sequence: Syntheticoligonucleotide"
  source 1..42: mol_type = other DNA
atggaggagttggcagatgcaatgtactgccatgggaagaag

SEQ ID NO: 609 - DNA, 124 bp, synthetic construct
  misc_feature 1..124: note = source = /note="Description of Artificial Sequence: Syntheticpolynucleotide"
  source 1..124: mol_type = other DNA
atggaggagttggcagatgccaaactgaaaaacaaatcaaagcactcttattgagtgctggcgatccccgacgccacgggccgaaacccttatcatagaaactctaatgtactgccatgggaag

SEQ ID NO: 610 - DNA, 84 bp, synthetic construct
  misc_feature 1..84: note = source = /note="Description of Artificial Sequence: Syntheticoligonucleotide"
  source 1..84: mol_type = other DNA
atggaggagttggcagatgctgcttatatagacctcccaccgtacacgcctaccgcccattttctaatgtactgccatgggaag

SEQ ID NO: 611 - DNA, 34 bp, synthetic construct
  misc_feature 1..34: note = source = /note="Description of Artificial Sequence: Syntheticoligonucleotide"
  source 1..34: mol_type = other DNA
atggaggagttgtctaatgtactgccatgggaag

SEQ ID NO: 612 - DNA, 16 bp, synthetic construct
  misc_feature 1..16: note = source = /note="Description of Artificial Sequence: Syntheticoligonucleotide"
  source 1..16: mol_type = other DNA
ctgccatgggaagaag

SEQ ID NO: 613 - DNA, 90 bp, synthetic construct
  misc_feature 1..90: note = source = /note="Description of Artificial Sequence: Syntheticoligonucleotide"
  source 1..90: mol_type = other DNA
atggaggagttggcagatgcgcggctgttcctgtacagaaccgtgggcgagatgtggatcaaggatgctctaatgtactgccatgggaag

SEQ ID NO: 614 - DNA, 41 bp, synthetic construct
  misc_feature 1..41: note = source = /note="Description of Artificial Sequence: Syntheticoligonucleotide"
  source 1..41: mol_type = other DNA
atggaggagttggcagatgcctaatgtactgccatgggaag

SEQ ID NO: 615 - DNA, 97 bp, synthetic construct
  misc_feature 1..97: note = source = /note="Description of Artificial Sequence: Syntheticoligonucleotide"
  source 1..97: mol_type = other DNA
atggaggagttggcagatgctgtcatgatctttttccgctcgtcgtgggacttgctcagttctctggccagctcgtctaatgtactgccatgggaag

SEQ ID NO: 616 - DNA, 41 bp, synthetic construct
  misc_feature 1..41: note = source = /note="Description of Artificial Sequence: Syntheticoligonucleotide"
  source 1..41: mol_type = other DNA
atggaggagttggcagatgctctatgtactgccatgggaag

SEQ ID NO: 617 - DNA, 32 bp, synthetic construct
  misc_feature 1..32: note = source = /note="Description of Artificial Sequence: Syntheticoligonucleotide"
  source 1..32: mol_type = other DNA
cagggacagtgcgcatctccctggtcaccaag

SEQ ID NO: 618 - DNA, 17 bp, synthetic construct
  misc_feature 1..17: note = source = /note="Description of Artificial Sequence: Syntheticoligonucleotide"
  source 1..17: mol_type = other DNA
cagggacagtcaccaag

SEQ ID NO: 619 - DNA, 31 bp, synthetic construct
  misc_feature 1..31: note = source = /note="Description of Artificial Sequence: Syntheticoligonucleotide"
  source 1..31: mol_type = other DNA
cagggacagtgcgcatctcctggtcaccaag

SEQ ID NO: 620 - DNA, 30 bp, synthetic construct
  misc_feature 1..30: note = source = /note="Description of Artificial Sequence: Syntheticoligonucleotide"
  source 1..30: mol_type = other DNA
cagggacagtgcgcatctctggtcaccaag

SEQ ID NO: 621 - DNA, 33 bp, synthetic construct
  misc_feature 1..33: note = source = /note="Description of Artificial Sequence: Syntheticoligonucleotide"
  source 1..33: mol_type = other DNA
cagggacagtgcgcatctcctctggtcaccaag

SEQ ID NO: 622 - DNA, 29 bp, synthetic construct
  misc_feature 1..29: note = source = /note="Description of Artificial Sequence: Syntheticoligonucleotide"
  source 1..29: mol_type = other DNA
cagggacagtgcgcatcctggtcaccaag

SEQ ID NO: 623 - DNA, 17 bp, synthetic construct
  misc_feature 1..17: note = source = /note="Description of Artificial Sequence: Syntheticoligonucleotide"
  source 1..17: mol_type = other DNA
cagggacggtcaccaag

SEQ ID NO: 624 - DNA, 31 bp, synthetic construct
  misc_feature 1..31: note = source = /note="Description of Artificial Sequence: Syntheticoligonucleotide"
  source 1..31: mol_type = other DNA
cggggacagggcgcatctcctggtcaccaag

SEQ ID NO: 625 - DNA, 32 bp, synthetic construct
  misc_feature 1..32: note = source = /note="Description of Artificial Sequence: Syntheticoligonucleotide"
  source 1..32: mol_type = other DNA
cgacctgaatgctgtgcggctctgcttccagg

SEQ ID NO: 626 - DNA, 33 bp, synthetic construct
  misc_feature 1..33: note = source = /note="Description of Artificial Sequence: Syntheticoligonucleotide"
  source 1..33: mol_type = other DNA
cgacctgaatgctgtgcggcatctgcttccagg

SEQ ID NO: 627 - DNA, 33 bp, synthetic construct
  misc_feature 1..33: note = source = /note="Description of Artificial Sequence: Syntheticoligonucleotide"
  source 1..33: mol_type = other DNA
cgacctgaatgctgtgcggcttctgcttccagg

SEQ ID NO: 628 - DNA, 28 bp, synthetic construct
  misc_feature 1..28: note = source = /note="Description of Artificial Sequence: Syntheticoligonucleotide"
  source 1..28: mol_type = other DNA
cgacctgaatgctgtgtctgcttccagg

SEQ ID NO: 629 - DNA, 33 bp, synthetic construct
  misc_feature 1..33: note = source = /note="Description of Artificial Sequence: Syntheticoligonucleotide"
  source 1..33: mol_type = other DNA
cgacctgcatgctgtgcggcatctgcttccagg

SEQ ID NO: 630 - DNA, 33 bp, synthetic construct
  misc_feature 1..33: note = source = /note="Description of Artificial Sequence: Syntheticoligonucleotide"
  source 1..33: mol_type = other DNA
cgacctgcatgctgtgcggcttctgcttccagg

SEQ ID NO: 631 - DNA, 28 bp, synthetic construct
  misc_feature 1..28: note = source = /note="Description of Artificial Sequence: Syntheticoligonucleotide"
  source 1..28: mol_type = other DNA
cgacctgcatgctgtgtctgcttccagg

SEQ ID NO: 632 - DNA, 33 bp, synthetic construct
  misc_feature 1..33: note = source = /note="Description of Artificial Sequence: Syntheticoligonucleotide"
  source 1..33: mol_type = other DNA
cgacctgaatgctgtgcgacatctgcttccagg

SEQ ID NO: 633 - DNA, 32 bp, synthetic construct
  misc_feature 1..32: note = source = /note="Description of Artificial Sequence: Syntheticoligonucleotide"
  source 1..32: mol_type = other DNA
tggggactacgacctgaatgctgtgcggctct

SEQ ID NO: 634 - DNA, 27 bp, synthetic construct
  misc_feature 1..27: note = source = /note="Description of Artificial Sequence: Syntheticoligonucleotide"
  source 1..27: mol_type = other DNA
tggggactacgaatgctgtgcggctct

SEQ ID NO: 635 - DNA, 66 bp, synthetic construct
  misc_feature 1..66: note = source = /note="Description of Artificial Sequence: Syntheticoligonucleotide"
  source 1..66: mol_type = other DNA
tggggactacgagcaggcagaagtatgcaaagcatgcatctcaattcctgaatgctgtgcggctct

SEQ ID NO: 636 - DNA, 72 bp, synthetic construct
  misc_feature 1..72: note = source = /note="Description of Artificial Sequence: Syntheticoligonucleotide"
  source 1..72: mol_type = other DNA
tggggactacgaagaaggcgatagaaggccatgcgctgcgaatcgggagcggcctgaatgctgtgcggctct

SEQ ID NO: 637 - DNA, 99 bp, synthetic construct
  misc_feature 1..99: note = source = /note="Description of Artificial Sequence: Syntheticoligonucleotide"
  source 1..99: mol_type = other DNA
tggggactacgatgactcgctgcgctcggtcgttcggctgcggcgagcggtatcagctcactcaaaggcggtaatacggcctgaatgctgtgcggctct

SEQ ID NO: 638 - DNA, 14 bp, synthetic construct
  misc_feature 1..14: note = source = /note="Description of Artificial Sequence: Syntheticoligonucleotide"
  source 1..14: mol_type = other DNA
tggggtgcggctct

SEQ ID NO: 639 - DNA, 27 bp, synthetic construct
  misc_feature 1..27: note = source = /note="Description of Artificial Sequence: Syntheticoligonucleotide"
  source 1..27: mol_type = other DNA
tggggactacgactgctgtgcggctct

SEQ ID NO: 640 - DNA, 39 bp, synthetic construct; identical to SEQ ID NO: 579
  misc_feature 1..39: note = source = /note="Description of Artificial Sequence: Syntheticoligonucleotide"
  source 1..39: mol_type = other DNA

SEQ ID NO: 641 - DNA, 35 bp, synthetic construct; identical to SEQ ID NO: 580
  misc_feature 1..35: note = source = /note="Description of Artificial Sequence: Syntheticoligonucleotide"
  source 1..35: mol_type = other DNA

SEQ ID NO: 642 - DNA, 36 bp, synthetic construct; identical to SEQ ID NO: 581
  misc_feature 1..36: note = source = /note="Description of Artificial Sequence: Syntheticoligonucleotide"
  source 1..36: mol_type = other DNA

SEQ ID NO: 643 - DNA, 25 bp, synthetic construct
  misc_feature 1..25: note = source = /note="Description of Artificial Sequence: Syntheticoligonucleotide"
  source 1..25: mol_type = other DNA
cgacctgaatgctgcttccaggtga

SEQ ID NO: 644 - DNA, 28 bp, synthetic construct
  misc_feature 1..28: note = source = /note="Description of Artificial Sequence: Syntheticoligonucleotide"
  source 1..28: mol_type = other DNA
cgacctgaatgcttctgcttccaggtga

SEQ ID NO: 645 - DNA, 22 bp, synthetic construct
  misc_feature 1..22: note = source = /note="Description of Artificial Sequence: Syntheticoligonucleotide"
  source 1..22: mol_type = other DNA
cgacctgaatgcttccaggtga

SEQ ID NO: 646 - protein, 4 aa, Homo sapiens
EEQL

SEQ ID NO: 647 - protein, 6 aa, Homo sapiens
AEVSQA

SEQ ID NO: 648 - protein, 4 aa, Mus musculus
GEQL

SEQ ID NO: 649 - protein, 6 aa, Mus musculus
AEVSKA

SEQ ID NO: 650 - protein, 4 aa, synthetic construct
  REGION 1..4: note = source = /note="Description of Artificial Sequence: Syntheticpeptide"
GEQL

SEQ ID NO: 651 - protein, 6 aa, synthetic construct
  REGION 1..6: note = source = /note="Description of Artificial Sequence: Syntheticpeptide"
AEVSQA

(19) United States
(12) Patent Application Publication
(10) Pub. No.: US 2025/0283117 A1
(43) Pub. Date: Sep. 11, 2025

(54) RNA-GUIDED NUCLEASES AND ACTIVE FRAGMENTS AND VARIANTS THEREOF AND METHODS OF USE

(71) Applicant: Life Edit Therapeutics, Inc., Durham, NC (US)

(72) Inventors: Alexandra Briner Crawley, Cary, NC (US); Rodolphe Barrangou, Raleigh, NC (US); Tyson D. Bowen, Morrisville, NC (US); Michael Coyle, Chapel Hill, NC (US); Tedd D. Elich, Durham, NC (US)

(73) Assignee: Life Edit Therapeutics, Inc., Durham, NC (US)

(21) Appl. No.: 19/216,399

(22) Filed: May 22, 2025

Related U.S. Application Data
(63) Continuation of application No. 18/431,662, filed on Feb. 2, 2024, now Pat. No. 12,338,455.
Application No. 18/431,662 is a continuation of application No. 17/478,374, filed on Sep. 17, 2021, now Pat. No. 11,926,843.
Application No. 17/478,374 is a continuation of application No. 16/432,321, filed on Jun. 5, 2019, now Pat. No. 11,162,114.
(60) Provisional application No. 62/680,845, filed on Jun. 5, 2018.
(60) Provisional application No. 62/680,846, filed on Jun. 5, 2018.
(60) Provisional application No. 62/680,853, filed on Jun. 5, 2018.
(60) Provisional application No. 62/680,859, filed on Jun. 5, 2018.
(60) Provisional application No. 62/680,862, filed on Jun. 5, 2018.
(60) Provisional application No. 62/680,863, filed on Jun. 5, 2018.
(60) Provisional application No. 62/686,901, filed on Jun. 19, 2018.
(60) Provisional application No. 62/805,041, filed on Feb. 13, 2019.
(60) Provisional application No. 62/805,045, filed on Feb. 13, 2019.

(51) Int. Cl. C12N 15/90; C12N 9/22; C12N 15/11

(52) U.S. Cl. CPC C12N 15/907; C12N 9/22; C12N 15/11; C12N 15/902; C12N 2310/20; C12N 2800/80

(57) ABSTRACT

Compositions and methods for binding to a target sequence of interest are provided. The compositions find use in cleaving or modifying a target sequence of interest, visualization of a target sequence of interest, and modifying the expression of a sequence of interest. Compositions comprise RNA-guided nuclease polypeptides, CRISPR RNAs, trans-activating CRISPR RNAs, guide RNAs, and nucleic acid molecules encoding the same. Vectors and host cells comprising the nucleic acid molecules are also provided. Further provided are CRISPR systems for binding a target sequence of interest, wherein the CRISPR system comprises an RNA-guided nuclease polypeptide and one or more guide RNAs.

### CROSS-REFERENCE TO RELATED APPLICATIONS

This application is a continuation of U.S. application Ser. No. 18/431,662, filed Feb. 2, 2024, which is a continuation of U.S. application Ser. No. 17/478,374, filed Sep. 17, 2021, now U.S. Pat. No. 11,926,843, which is a continuation of U.S. application Ser. No. 16/432,321, filed Jun. 5, 2019, now U.S. Pat. No. 11,162,114, which claims the benefit of U.S. Provisional Application Nos. 62/805,041, filed Feb. 13, 2019, 62/805,045, filed Feb. 13, 2019, 62/686,901, filed Jun. 19, 2018, 62/680,845, filed Jun. 5, 2018, 62/680,846, filed Jun. 5, 2018, 62/680,853, filed Jun. 5, 2018, 62/680,859, filed Jun. 5, 2018, 62/680,862, filed Jun. 5, 2018, and 62/680,863, filed Jun. 5, 2018, each of which is incorporated by reference herein in its entirety.

### REFERENCE TO A SEQUENCE LISTING SUBMITTED ELECTRONICALLY AS AN XML FILE

The instant application contains a Sequence Listing which has been submitted in ST.26 XML format via USPTO Patent Center and is hereby incorporated by reference in its entirety. Said ST.26 XML copy, created on Aug. 6, 2024 is named L103438_1160US_C2_SL.XML, and is 1,099,916 bytes in size.

### FIELD OF THE INVENTION

The present invention relates to the field of molecular biology and gene editing.

### BACKGROUND OF THE INVENTION

Targeted genome editing or modification is rapidly becoming an important tool for basic and applied research. Initial methods involved engineering nucleases such as meganucleases, zinc finger fusion proteins or TALENs, requiring the generation of chimeric nucleases with engineered, programmable, sequence-specific DNA-binding domains specific for each particular target sequence. RNA-guided nucleases, such as the Clustered Regularly Interspaced Short Palindromic Repeats (CRISPR)-associated (cas) proteins of the CRISPR-cas bacterial system, allow for the targeting of specific sequences by complexing the nucleases with guide RNA that specifically hybridizes with a particular target sequence. Producing target-specific guide RNAs is less costly and more efficient than generating chimeric nucleases for each target sequence. Such RNA-guided nucleases can be used to edit genomes through the introduction of a sequence-specific, double-stranded break that is repaired via error-prone non-homologous end-joining (NHEJ) to introduce a mutation at a specific genomic location. Alternatively, heterologous DNA may be introduced into the genomic site via homology-directed repair.

### BRIEF SUMMARY OF THE INVENTION

Compositions and methods for binding a target sequence of interest are provided. The compositions find use in cleaving or modifying a target sequence of interest, detection of a target sequence of interest, and modifying the expression of a sequence of interest. Compositions comprise RNA-guided nuclease (RGN) polypeptides, CRISPR RNAs (crRNAs), trans-activating CRISPR RNAs (tracrRNAs), guide RNAs (gRNAs), nucleic acid molecules encoding the same, and vectors and host cells comprising the nucleic acid molecules. Also provided are CRISPR systems for binding a target sequence of interest, wherein the CRISPR system comprises an RNA-guided nuclease polypeptide and one or more guide RNAs. Thus, methods disclosed herein are drawn to binding a target sequence of interest, and in some embodiments, cleaving or modifying the target sequence of interest. The target sequence of interest can be modified, for example, as a result of non-homologous end joining or homology-directed repair with an introduced donor sequence.

### DETAILED DESCRIPTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

### I. Overview

RNA-guided nucleases (RGNs) allow for the targeted manipulation of a single site within a genome and are useful in the context of gene targeting for therapeutic and research applications. In a variety of organisms, including mammals, RNA-guided nucleases have been used for genome engineering by stimulating non-homologous end joining and homologous recombination, for example. The compositions and methods described herein are useful for creating single- or double-stranded breaks in polynucleotides, modifying polynucleotides, detecting a particular site within a polynucleotide, or modifying the expression of a particular gene.

The RNA-guided nucleases disclosed herein can alter gene expression by modifying a target sequence. In specific embodiments, the RNA-guided nucleases are directed to the target sequence by a guide RNA (gRNA) as part of a Clustered Regularly Interspaced Short Palindromic Repeats (CRISPR) RNA-guided nuclease system. Guide RNAs form a complex with the RNA-guided nucleases to direct the RNA-guided nuclease to bind to a target sequence and in some embodiments, introduce a single-stranded or double-stranded break at the target sequence. After the target sequence has been cleaved, the break can be repaired such that the DNA sequence of the target sequence is modified during the repair process. Thus, provided herein are methods for using the RNA-guided nucleases to modify a target sequence in the DNA of host cells. For example, RNA-guided nucleases can be used to modify a target sequence at a genomic locus of eukaryotic cells or prokaryotic cells.

### II. RNA-Guided Nucleases

Provided herein are RNA-guided nucleases. The term RNA-guided nuclease (RGN) refers to a polypeptide that binds to a particular target nucleotide sequence in a sequence-specific manner and is directed to the target nucleotide sequence by a guide RNA molecule that is complexed with the polypeptide and hybridizes with the target sequence. Although an RNA-guided nuclease can be capable of cleaving the target sequence upon binding, the term RNA-guided nuclease also encompasses nuclease-dead RNA-guided nucleases that are capable of binding to, but not cleaving, a target sequence. Cleavage of a target sequence by an RNA-guided nuclease can result in a single- or double-stranded break. RNA-guided nucleases only capable of cleaving a single strand of a double-stranded nucleic acid molecule are referred to herein as nickases.

The RNA-guided nucleases disclosed herein include the APG05083.1, APG07433.1, APG07513.1, APG08290.1, APG05459.1, APG04583.1, and APG1688.1 RNA-guided nucleases, the amino acid sequences of which are set forth, respectively, as SEQ ID NOs: 1, 11, 19, 27, 36, 45, or 54, and active fragments or variants thereof that retain the ability to bind to a target nucleotide sequence in an RNA-guided sequence-specific manner. In some of these embodiments, the active fragment or variant of the APG05083.1, APG07433.1, APG07513.1, APG08290.1, APG05459.1, APG04583.1, and APG1688.1 RGN is capable of cleaving a single- or double-stranded target sequence. In some embodiments, an active variant of the APG05083.1, APG07433.1, APG07513.1, APG08290.1, APG05459.1, APG04583.1, or APG1688.1 RGN comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth as SEQ ID NOs: 1, 11, 19, 27, 36, 45, or 54. In certain embodiments, an active fragment of the APG05083.1, APG07433.1, APG07513.1, APG08290.1, APG05459.1, APG04583.1, or APG1688.1 RGN comprises at least 50, 100, 150, 200, 250, 300, 350, 400, 450, 500, 550, 600, 650, 700, 750, 800, 850, 900, 950, 1000, 1050 or more contiguous amino acid residues of the amino acid sequence set forth as SEQ ID NOs: 1, 11, 19, 27, 36, 45, or 54. RNA-guided nucleases provided herein can comprise at least one nuclease domain (e.g., DNase, RNase domain) and at least one RNA recognition and/or RNA binding domain to interact with guide RNAs. Further domains that can be found in RNA-guided nucleases provided herein include, but are not limited to: DNA binding domains, helicase domains, protein-protein interaction domains, and dimerization domains. In specific embodiments, the RNA-guided nucleases provided herein can comprise at least 70%, 75%, 80%, 85%, 90%, 91%, 92%, 93%, 94%, 95%, 96%, 97%, 98%, 99% to one or more of a DNA binding domains, helicase domains, protein-protein interaction domains, and dimerization domains.

A target nucleotide sequence is bound by an RNA-guided nuclease provided herein and hybridizes with the guide RNA associated with the RNA-guided nuclease. The target sequence can then be subsequently cleaved by the RNA-guided nuclease if the polypeptide possesses nuclease activity. The terms “cleave” or “cleavage” refer to the hydrolysis of at least one phosphodiester bond within the backbone of a target nucleotide sequence that can result in either single-stranded or double-stranded breaks within the target sequence. The presently disclosed RGNs can cleave nucleotides within a polynucleotide, functioning as an endonuclease or can be an exonuclease, removing successive nucleotides from the end (the 5′ and/or the 3′ end) of a polynucleotide. In other embodiments, the disclosed RGNs can cleave nucleotides of a target sequence within any position of a polynucleotide and thus function as both an endonuclease and exonuclease. The cleavage of a target polynucleotide by the presently disclosed RGNs can result in staggered breaks or blunt ends.

The presently disclosed RNA-guided nucleases can be wild-type sequences derived from bacterial or archaeal species. Alternatively, the RNA-guided nucleases can be variants or fragments of wild-type polypeptides. The wild-type RGN can be modified to alter nuclease activity or alter PAM specificity, for example. In some embodiments, the RNA-guided nuclease is not naturally-occurring.

In certain embodiments, the RNA-guided nuclease functions as a nickase, only cleaving a single strand of the target nucleotide sequence. Such RNA-guided nucleases have a single functioning nuclease domain. In some of these embodiments, additional nuclease domains have been mutated such that the nuclease activity is reduced or eliminated.

In other embodiments, the RNA-guided nuclease lacks nuclease activity altogether or exhibits reduced nuclease activity, and is referred to herein as nuclease-dead. Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated by reference in its entirety.

RNA-guided nucleases that lack nuclease activity can be used to deliver a fused polypeptide, polynucleotide, or small molecule payload to a particular genomic location. In some of these embodiments, the RGN polypeptide or guide RNA can be fused to a detectable label to allow for detection of a particular sequence. As a non-limiting example, a nuclease-dead RGN can be fused to a detectable label (e.g., fluorescent protein) and targeted to a particular sequence associated with a disease to allow for detection of the disease-associated sequence.

Alternatively, nuclease-dead RGNs can be targeted to particular genomic locations to alter the expression of a desired sequence. In some embodiments, the binding of a nuclease-dead RNA-guided nuclease to a target sequence results in the repression of expression of the target sequence or a gene under transcriptional control by the target sequence by interfering with the binding of RNA polymerase or transcription factors within the targeted genomic region. In other embodiments, the RGN (e.g., a nuclease-dead RGN) or its complexed guide RNA further comprises an expression modulator that, upon binding to a target sequence, serves to either repress or activate the expression of the target sequence or a gene under transcriptional control by the target sequence. In some of these embodiments, the expression modulator modulates the expression of the target sequence or regulated gene through epigenetic mechanisms.

In other embodiments, the nuclease-dead RGNs or a RGN with only nickase activity can be targeted to particular genomic locations to modify the sequence of a target polynucleotide through fusion to a base-editing polypeptide, for example a deaminase polypeptide or active variant or fragment thereof that deaminates a nucleotide base, resulting in conversion from one nucleotide base to another. The base-editing polypeptide can be fused to the RGN at its N-terminal or C-terminal end. Additionally, the base-editing polypeptide may be fused to the RGN via a peptide linker. A non-limiting example of a deaminase polypeptide that is useful for such compositions and methods include cytidine deaminase or the adenosine deaminase base editor described in Gaudelli et al. (2017) Nature 551:464-471, U.S. Publ. Nos. 2017/0121693 and 2018/0073012, and International Publ. No. WO/2018/027078, each of which is herein incorporated by reference in its entirety.

RNA-guided nucleases that are fused to a polypeptide or domain can be separated or joined by a linker. The term “linker,” as used herein, refers to a chemical group or a molecule linking two molecules or moieties, e.g., a binding domain and a cleavage domain of a nuclease. In some embodiments, a linker joins a gRNA binding domain of an RNA guided nuclease and a base-editing polypeptide, such as a deaminase. In some embodiments, a linker joins a nuclease-dead RGN and a deaminase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

The presently disclosed RNA-guided nucleases can comprise at least one nuclear localization signal (NLS) to enhance transport of the RGN to the nucleus of a cell. Nuclear localization signals are known in the art and generally comprise a stretch of basic amino acids (see, e.g., Lange et al., J. Biol. Chem. (2007) 282:5101-5105). In particular embodiments, the RGN comprises 2, 3, 4, 5, 6 or more nuclear localization signals. The nuclear localization signal(s) can be a heterologous NLS. Non-limiting examples of nuclear localization signals useful for the presently disclosed RGNs are the nuclear localization signals of SV40 Large T-antigen, nucleopasmin, and c-Myc (see, e.g., Ray et al. (2015) Bioconjug Chem 26 (6): 1004-7). In particular embodiments, the RGN comprises the NLS sequence set forth as SEQ ID NO: 67. The RGN can comprise one or more NLS sequences at its N-terminus, C-terminus, or both the N-terminus and C-terminus. For example, the RGN can comprise two NLS sequences at the N-terminal region and four NLS sequences at the C-terminal region.

Other localization signal sequences known in the art that localize polypeptides to particular subcellular location(s) can also be used to target the RGNs, including, but not limited to, plastid localization sequences, mitochondrial localization sequences, and dual-targeting signal sequences that target to both the plastid and mitochondria (see, e.g., Nassoury and Morse (2005) Biochim Biophys Acta 1743:5-19; Kunze and Berger (2015) Front Physiol dx.doi.org/10.3389/fphys.2015.00259; Herrmann and Neupert (2003) IUBMB Life 55:219-225; Soll (2002) Curr Opin Plant Biol 5:529-535; Carrie and Small (2013) Biochim Biophys Acta 1833:253-259; Carrie et al. (2009) FEBS J 276:1187-1195; Silva-Filho (2003) Curr Opin Plant Biol 6:589-595; Peeters and Small (2001) Biochim Biophys Acta 1541:54-63; Murcha et al. (2014) J Exp Bot 65:6301-6335; Mackenzie (2005) Trends Cell Biol 15:548-554; Glaser et 5 al. (1998) Plant Mol Biol 38:311-338).

In certain embodiments, the presently disclosed RNA-guided nucleases comprise at least one cell-penetrating domain that facilitates cellular uptake of the RGN. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

The nuclear localization signal, plastid localization signal, mitochondrial localization signal, dual-targeting localization signal, and/or cell-penetrating domain can be located at the amino-terminus (N-terminus), the carboxyl-terminus (C-terminus), or in an internal location of the RNA-guided nuclease. The presently disclosed RGNs can be fused to an effector domain, such as a cleavage domain, a deaminase domain, or an expression modulator domain, either directly or indirectly via a linker peptide. Such a domain can be located at the N-terminus, the C-terminus, or an internal location of the RNA-guided nuclease. In some of these embodiments, the RGN component of the fusion protein is a nuclease-dead RGN.

In some embodiments, the RGN fusion protein comprises a cleavage domain, which is any domain that is capable of cleaving a polynucleotide (i.e., RNA, DNA, or RNA/DNA hybrid) and includes, but is not limited to, restriction endonucleases and homing endonucleases, such as Type IIS endonucleases (e.g., FokI) (see, e.g., Belfort et al. (1997) Nucleic Acids Res. 25:3379-3388; Linn et al. (eds.) Nucleases, Cold Spring Harbor Laboratory Press, 1993).

In other embodiments, the RGN fusion protein comprises a deaminase domain that deaminates a nucleotide base, resulting in conversion from one nucleotide base to another, and includes, but is not limited to, a cytidine deaminase or an adenosine deaminase base editor (see, e.g., Gaudelli et al. (2017) Nature 551:464-471, U.S. Publ. Nos. 2017/0121693 and 2018/0073012, U.S. Pat. No. 9,840,699, and International Publ. No. WO/2018/027078).

In some embodiments, the effector domain of the RGN fusion protein can be an expression modulator domain, which is a domain that either serves to upregulate or downregulate transcription. The expression modulator domain can be an epigenetic modification domain, a transcriptional repressor domain or a transcriptional activation domain.

In some of these embodiments, the expression modulator of the RGN fusion protein comprises an epigenetic modification domain that covalently modifies DNA or histone proteins to alter histone structure and/or chromosomal structure without altering the DNA sequence, leading to changes in gene expression (i.e., upregulation or downregulation). Non-limiting examples of epigenetic modifications include acetylation or methylation of lysine residues, arginine methylation, serine and threonine phosphorylation, and lysine ubiquitination and sumoylation of histone proteins, and methylation and hydroxymethylation of cytosine residues in DNA. Non-limiting examples of epigenetic modification domains include histone acetyltransferase domains, histone deacetylase domains, histone methyltransferase domains, histone demethylase domains, DNA methyltransferase domains, and DNA demethylase domains.

In other embodiments, the expression modulator of the fusion protein comprises a transcriptional repressor domain, which interacts with transcriptional control elements and/or transcriptional regulatory proteins, such as RNA polymerases and transcription factors, to reduce or terminate transcription of at least one gene. Transcriptional repressor domains are known in the art and include, but are not limited to, Sp1-like repressors, IκB, and Krüppel associated box (KRAB) domains.

In yet other embodiments, the expression modulator of the fusion protein comprises a transcriptional activation domain, which interacts with transcriptional control elements and/or transcriptional regulatory proteins, such as RNA polymerases and transcription factors, to increase or activate transcription of at least one gene. Transcriptional activation domains are known in the art and include, but are not limited to, a herpes simplex virus VP16 activation domain and an NFAT activation domain.

The presently disclosed RGN polypeptides can comprise a detectable label or a purification tag. The detectable label or purification tag can be located at the N-terminus, the C-terminus, or an internal location of the RNA-guided nuclease, either directly or indirectly via a linker peptide. In some of these embodiments, the RGN component of the fusion protein is a nuclease-dead RGN. In other embodiments, the RGN component of the fusion protein is a RGN with nickase activity.

A detectable label is a molecule that can be visualized or otherwise observed. The detectable label may be fused to the RGN as a fusion protein (e.g., fluorescent protein) or may be a small molecule conjugated to the RGN polypeptide that can be detected visually or by other means. Detectable labels that can be fused to the presently disclosed RGNs as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as 3H and 35S.

RGN polypeptides can also comprise a purification tag, which is any molecule that can be utilized to isolate a protein or fused protein from a mixture (e.g., biological sample, culture medium). Non-limiting examples of purification tags include biotin, myc, maltose binding protein (MBP), and glutathione-S-transferase (GST).

### II. Guide RNA

The present disclosure provides guide RNAs and polynucleotides encoding the same. The term “guide RNA” refers to a nucleotide sequence having sufficient complementarity with a target nucleotide sequence to hybridize with the target sequence and direct sequence-specific binding of an associated RNA-guided nuclease to the target nucleotide sequence. Thus, a RGN's respective guide RNA is one or more RNA molecules (generally, one or two), that can bind to the RGN and guide the RGN to bind to a particular target nucleotide sequence, and in those instances wherein the RGN has nickase or nuclease activity, also cleave the target nucleotide sequence. In general, a guide RNA comprises a CRISPR RNA (crRNA) and a trans-activating CRISPR RNA (tracrRNA). Native guide RNAs that comprise both a crRNA and a tracrRNA generally comprise two separate RNA molecules that hybridize to each other through the repeat sequence of the crRNA and the anti-repeat sequence of the tracrRNA.

Native direct repeat sequences within a CRISPR array generally range in length from 28 to 37 base pairs, although the length can vary between about 23 bp to about 55 bp. Spacer sequences within a CRISPR array generally range from about 32 to about 38 bp in length, although the length can be between about 21 bp to about 72 bp. Each CRISPR array generally comprises less than 50 units of the CRISPR repeat-spacer sequence. The CRISPRs are transcribed as part of a long transcript termed the primary

CRISPR transcript, which comprises much of the CRISPR array. The primary CRISPR transcript is cleaved by Cas proteins to produce crRNAs or in some cases, to produce pre-crRNAs that are further processed by additional Cas proteins into mature crRNAs. Mature crRNAs comprise a spacer sequence and a CRISPR repeat sequence. In some embodiments in which pre-crRNAs are processed into mature (or processed) crRNAs, maturation involves the removal of about one to about six or more 5′, 3′, or 5′ and 3′ nucleotides. For the purposes of genome editing or targeting a particular target nucleotide sequence of interest, these nucleotides that are removed during maturation of the pre-crRNA molecule are not necessary for generating or designing a guide RNA.

A CRISPR RNA (crRNA) comprises a spacer sequence and a CRISPR repeat sequence. The “spacer sequence” is the nucleotide sequence that directly hybridizes with the target nucleotide sequence of interest. The spacer sequence is engineered to be fully or partially complementary with the target sequence of interest. In various embodiments, the spacer sequence can comprise from about 8 nucleotides to about 30 nucleotides, or more. For example, the spacer sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer sequence is about 10 to about 26 nucleotides in length, or about 12 to about 30 nucleotides in length. In particular embodiments, the spacer sequence is about 30 nucleotides in length. In some embodiments, the degree of complementarity between a spacer sequence and its corresponding target sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the spacer sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106 (1): 23-24).

RGN proteins can have varying sensitivity to mismatches between a spacer sequence in a gRNA and its target sequence that affects the efficiency of cleavage. As discussed in Example 5, APG05459.1 RGN has an unusual sensitivity to mismatches between the spacer sequence and target sequence, extending at least 15 nucleotides 5′ of the PAM site. Thus, APG05459.1 has the potential to more finely (i.e., specifically) target particular sequences with greater precision than other RGNs with less sensitivity to mismatches between the spacer sequence and target sequence.

The CRISPR RNA repeat sequence comprises a nucleotide sequence that comprises a region with sufficient complementarity to hybridize to a tracrRNA. In various embodiments, the CRISPR RNA repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the CRISPR repeat sequence is about 21 nucleotides in length. In some embodiments, the degree of complementarity between a CRISPR repeat sequence and its corresponding tracrRNA sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the CRISPR repeat sequence comprises the nucleotide sequence of SEQ ID NOs: 2, 12, 20, 28, 37, 46, or 55, or an active variant or fragment thereof that when comprised within a guide RNA, is capable of directing the sequence-specific binding of an associated RNA-guided nuclease provided herein to a target sequence of interest. In certain embodiments, an active CRISPR repeat sequence variant of a wild-type sequence comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth as SEQ ID NOs: 2, 12, 20, 28, 37, 46, or 55. In certain embodiments, an active CRISPR repeat sequence fragment of a wild-type sequence comprises at least 5, 6, 7, 8, 9, 10, 11, 12, 13, 14, 15, 16, 17, 18, 19, or 20 contiguous nucleotides of the nucleotide sequence set forth as SEQ ID NOs: 2, 12, 20, 28, 37, 46, or 55.

In certain embodiments, the crRNA is not naturally-occurring. In some of these embodiments, the specific CRISPR repeat sequence is not linked to the engineered spacer sequence in nature and the CRISPR repeat sequence is considered heterologous to the spacer sequence. In certain embodiments, the spacer sequence is an engineered sequence that is not naturally occurring.

A trans-activating CRISPR RNA or tracrRNA molecule comprises a nucleotide sequence comprising a region that has sufficient complementarity to hybridize to a CRISPR repeat sequence of a crRNA, which is referred to herein as the anti-repeat region. In some embodiments, the tracrRNA molecule further comprises a region with secondary structure (e.g., stem-loop) or forms secondary structure upon hybridizing with its corresponding crRNA. In particular embodiments, the region of the tracrRNA that is fully or partially complementary to a CRISPR repeat sequence is at the 5′ end of the molecule and the 3′ end of the tracrRNA comprises secondary structure. This region of secondary structure generally comprises several hairpin structures, including the nexus hairpin, which is found adjacent to the anti-repeat sequence. The nexus hairpin often has a conserved nucleotide sequence in the base of the hairpin stem, with the motif UNANNG, UNANNU, or UNANNA (SEQ ID NOs: 68, 557, and 558, respectively) found in many nexus hairpins in tracrRNAs. There are often terminal hairpins at the 3′ end of the tracrRNA that can vary in structure and number, but often comprise a GC-rich Rho-independent transcriptional terminator hairpin followed by a string of U's at the 3′ end. See, for example, Briner et al. (2014) Molecular Cell 56:333-339, Briner and Barrangou (2016) Cold Spring Harb Protoc; doi: 10.1101/pdb.top090902, and U.S. Publication No. 2017/0275648, each of which is herein incorporated by reference in its entirety.

In various embodiments, the anti-repeat region of the tracrRNA that is fully or partially complementary to the CRISPR repeat sequence comprises from about 8 nucleotides to about 30 nucleotides, or more. For example, the region of base pairing between the tracrRNA anti-repeat sequence and the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In particular embodiments, the anti-repeat region of the tracrRNA that is fully or partially complementary to a CRISPR repeat sequence is about 20 nucleotides in length. In some embodiments, the degree of complementarity between a CRISPR repeat sequence and its corresponding tracrRNA anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA can comprise from about 60 nucleotides to more than about 140 nucleotides. For example, the tracrRNA can be about 60, about 65, about 70, about 75, about 80, about 85, about 90, about 95, about 100, about 105, about 110, about 115, about 120, about 125, about 130, about 135, about 140, or more nucleotides in length. In particular embodiments, the tracrRNA is about 80 to about 90 nucleotides in length, including about 80, about 81, about 82, about 83, about 84, about 85, about 86, about 87, about 88, about 89, and about 90 nucleotides in length. In certain embodiments, the tracrRNA is about 85 nucleotides in length.

In particular embodiments, the tracrRNA comprises the nucleotide sequence of SEQ ID NOs: 3, 13, 21, 29, 38, 47, or 56, or an active variant or fragment thereof that when comprised within a guide RNA is capable of directing the sequence-specific binding of an associated RNA-guided nuclease provided herein to a target sequence of interest. In certain embodiments, an active tracrRNA sequence variant of a wild-type sequence comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth as SEQ ID NOs: 3, 13, 21, 29, 38, 47, or 56. In certain embodiments, an active tracrRNA sequence fragment of a wild-type sequence comprises at least 5, 10, 15, 20, 25, 30, 35, 40, 45, 50, 55, 60, 65, 70, 75, 80, or more contiguous nucleotides of the nucleotide sequence set forth as SEQ ID NOs: 3, 13, 21, 29, 38, 47, or 56.

Two polynucleotide sequences can be considered to be substantially complementary when the two sequences hybridize to each other under stringent conditions. Likewise, an RGN is considered to bind to a particular target sequence within a sequence-specific manner if the guide RNA bound to the RGN binds to the target sequence under stringent conditions. By “stringent conditions” or “stringent hybridization conditions” is intended conditions under which the two polynucleotide sequences will hybridize to each other to a detectably greater degree than to other sequences (e.g., at least 2-fold over background). Stringent conditions are sequence-dependent and will be different in different circumstances. Typically, stringent conditions will be those in which the salt concentration is less than about 1.5 M Na ion, typically about 0.01 to 1.0 M Na ion concentration (or other salts) at pH 7.0 to 8.3, and the temperature is at least about 30° C. for short sequences (e.g., 10 to 50 nucleotides) and at least about 60° C. for long sequences (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology-Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

The guide RNA can be a single guide RNA or a dual-guide RNA system. A single guide RNA comprises the crRNA and tracrRNA on a single molecule of RNA, whereas a dual-guide RNA system comprises a crRNA and a tracrRNA present on two distinct RNA molecules, hybridized to one another through at least a portion of the CRISPR repeat sequence of the crRNA and at least a portion of the tracrRNA, which may be fully or partially complementary to the CRISPR repeat sequence of the crRNA. In some of those embodiments wherein the guide RNA is a single guide RNA, the crRNA and tracrRNA are separated by a linker nucleotide sequence. In general, the linker nucleotide sequence is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In particular embodiments, the linker nucleotide sequence of a single guide RNA is at least 4 nucleotides in length. In certain embodiments, the linker nucleotide sequence is the nucleotide sequence set forth as SEQ ID NO: 63, 64, or 65. In other embodiments, the linker nucleotide sequence is at least 6 nucleotides in length. In certain embodiments, the linker nucleotide sequence is the nucleotide sequence set forth as SEQ ID NO: 65.

The single guide RNA or dual-guide RNA can be synthesized chemically or via in vitro transcription. Assays for determining sequence-specific binding between a RGN and a guide RNA are known in the art and include, but are not limited to, in vitro binding assays between an expressed RGN and the guide RNA, which can be tagged with a detectable label (e.g., biotin) and used in a pull-down detection assay in which the guide RNA: RGN complex is captured via the detectable label (e.g., with streptavidin beads). A control guide RNA with an unrelated sequence or structure to the guide RNA can be used as a negative control for non-specific binding of the RGN to RNA. In certain embodiments, the guide RNA is SEQ ID NO: 10, 18, 26, 35, 44, 53, or 62, wherein the spacer sequence can be any sequence and is indicated as a poly-N sequence.

As described in Example 8, certain RGNs of the invention can share certain guide RNAs. APG05083.1, APG07433.1, APG07513.1, and APG08290.1 can each function using guide RNAs comprising a crRNA comprising the nucleotide sequence of SEQ ID NOs: 2, 12, 20, or 28, with the corresponding tracrRNA comprising the nucleotide sequence of SEQ ID NOs: 3, 13, 21, or 29, respectively. Further, APG04583.1 and APG01688.1 can each function using guide RNAs comprising a crRNA comprising the nucleotide sequence of SEQ ID NOs: 46 or 55, with the corresponding tracrRNA comprising the nucleotide sequence of SEQ 47 or 56, respectively.

In certain embodiments, the guide RNA can be introduced into a target cell, organelle, or embryo as an RNA molecule. The guide RNA can be transcribed in vitro or chemically synthesized. In other embodiments, a nucleotide sequence encoding the guide RNA is introduced into the cell, organelle, or embryo. In some of these embodiments, the nucleotide sequence encoding the guide RNA is operably linked to a promoter (e.g., an RNA polymerase III promoter). The promoter can be a native promoter or heterologous to the guide RNA-encoding nucleotide sequence.

In various embodiments, the guide RNA can be introduced into a target cell, organelle, or embryo as a ribonucleoprotein complex, as described herein, wherein the guide RNA is bound to an RNA-guided nuclease polypeptide.

The guide RNA directs an associated RNA-guided nuclease to a particular target nucleotide sequence of interest through hybridization of the guide RNA to the target nucleotide sequence. A target nucleotide sequence can comprise DNA, RNA, or a combination of both and can be single-stranded or double-stranded. A target nucleotide sequence can be genomic DNA (i.e., chromosomal DNA), plasmid DNA, or an RNA molecule (e.g., messenger RNA, ribosomal RNA, transfer RNA, micro RNA, small interfering RNA). The target nucleotide sequence can be bound (and in some embodiments, cleaved) by an RNA-guided nuclease in vitro or in a cell. The chromosomal sequence targeted by the RGN can be a nuclear, plastid or mitochondrial chromosomal sequence. In some embodiments, the target nucleotide sequence is unique in the target genome.

The target nucleotide sequence is adjacent to a protospacer adjacent motif (PAM). A protospacer adjacent motif is generally within about 1 to about 10 nucleotides from the target nucleotide sequence, including about 1, about 2, about 3, about 4, about 5, about 6, about 7, about 8, about 9, or about 10 nucleotides from the target nucleotide sequence. The PAM can be 5′ or 3′ of the target sequence. In some embodiments, the PAM is 3′ of the target sequence for the presently disclosed RGNs. Generally, the PAM is a consensus sequence of about 3-4 nucleotides, but in particular embodiments, can be 2, 3, 4, 5, 6, 7, 8, 9, or more nucleotides in length. In various embodiments, the PAM sequence recognized by the presently disclosed RGNs comprises the consensus sequence set forth as SEQ ID NOs: 6, 32, 41, 50, or 59. Non-limiting exemplary PAM sequences are the nucleotide sequences set forth as SEQ ID NO: 7, 69, 70, 71, and 72.

In particular embodiments, an RNA-guided nuclease having SEQ ID NOs: 1, 11, 19, 27, 36, 45, or 54 or an active variant or fragment thereof binds respectively a target nucleotide sequence adjacent to a PAM sequence set forth as SEQ ID NOs: 6, 32, 41, 50, 59, or 7. In some of these embodiments, the RGN binds to a guide sequence comprising a CRISPR repeat sequence set forth in SEQ ID NOs: 2, 12, 20, 28, 37, 46, or 55, respectively, or an active variant or fragment thereof, and a tracrRNA sequence set forth in SEQ ID NOs: 3, 13, 21, 29, 38, 47, or 56, respectively, or an active variant or fragment thereof. The RGN systems are described further in Example 1 and Table 1 of the present specification.

It is well-known in the art that PAM sequence specificity for a given nuclease enzyme is affected by enzyme concentration (see, e.g., Karvelis et al. (2015) Genome Biol 16:253), which may be modified by altering the promoter used to express the RGN, or the amount of ribonucleoprotein complex delivered to the cell, organelle, or embryo.

Upon recognizing its corresponding PAM sequence, the RGN can cleave the target nucleotide sequence at a specific cleavage site. As used herein, a cleavage site is made up of the two particular nucleotides within a target nucleotide sequence between which the nucleotide sequence is cleaved by an RGN. The cleavage site can comprise the 1^{st} and 2^{nd}, 2^{nd} and 3^{rd}, 3^{rd} and 4th, 4th and 5th, 5th and 6th, 7th and 8th, or 8th and 9th nucleotides from the PAM in either the 5′ or 3′ direction. In some embodiments, the cleavage site may be over 10, 11, 12, 13, 14, 15, 16, 17, 18, 19, or 20 nucleotides from the PAM in either the 5′ or 3′ direction. In some embodiments, the cleavage site is 4 nucleotides away from the PAM. In other embodiments, the cleavage site is at least 15 nucleotides away from the PAM. As RGNs can cleave a target nucleotide sequence resulting in staggered ends, in some embodiments, the cleavage site is defined based on the distance of the two nucleotides from the PAM on the positive (+) strand of the polynucleotide and the distance of the two nucleotides from the PAM on the negative (−) strand of the polynucleotide.

III. Nucleotides Encoding RNA-Guided Nucleases, CRISPR RNA, and/or tracrRNA

The present disclosure provides polynucleotides comprising the presently disclosed CRISPR RNAs, tracrRNAs, and/or sgRNAs and polynucleotides comprising a nucleotide sequence encoding the presently disclosed RNA-guided nucleases, CRISPR RNAs, tracrRNAs, and/or sgRNAs. Presently disclosed polynucleotides include those comprising or encoding a CRISPR repeat sequence comprising the nucleotide sequence of SEQ ID NOs: 2, 12, 20, 28, 37, 46, or 55, or an active variant or fragment thereof that when comprised within a guide RNA is capable of directing the sequence-specific binding of an associated RNA-guided nuclease to a target sequence of interest. Also disclosed are polynucleotides comprising or encoding a tracrRNA comprising the nucleotide sequence of SEQ ID NOs: 3, 13, 21, 29, 38, 47, or 56, or an active variant or fragment thereof that when comprised within a guide RNA is capable of directing the sequence-specific binding of an associated RNA-guided nuclease to a target sequence of interest. Polynucleotides are also provided that encode an RNA-guided nuclease comprising the amino acid sequence set forth as SEQ ID NOs: 1, 11, 19, 27, 36, 45, or 54, and active fragments or variants thereof that retain the ability to bind to a target nucleotide sequence in an RNA-guided sequence-specific manner.

The use of the term “polynucleotide” is not intended to limit the present disclosure to polynucleotides comprising DNA. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. These include peptide nucleic acids (PNAs), PNA-DNA chimers, locked nucleic acids (LNAs), and phosphothiorate linked sequences. The polynucleotides disclosed herein also encompass all forms of sequences including, but not limited to, single-stranded forms, double-stranded forms, DNA-RNA hybrids, triplex structures, stem-and-loop structures, and the like.

The nucleic acid molecules encoding RGNs can be codon optimized for expression in an organism of interest. A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Campbell and Gowri (1990) Plant Physiol. 92:1-11 for a discussion of plant-preferred codon usage). Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Polynucleotides encoding the RGNs, crRNAs, tracrRNAs, and/or sgRNAs provided herein can be provided in expression cassettes for in vitro expression or expression in a cell, organelle, embryo, or organism of interest. The cassette will include 5′ and 3′ regulatory sequences operably linked to a polynucleotide encoding an RGN, crRNA, tracrRNAs, and/or sgRNAs provided herein that allows for expression of the polynucleotide. The cassette may additionally contain at least one additional gene or genetic element to be cotransformed into the organism. Where additional genes or elements are included, the components are operably linked. The term “operably linked” is intended to mean a functional linkage between two or more elements. For example, an operable linkage between a promoter and a coding region of interest (e.g., region coding for an RGN, crRNA, tracrRNAs, and/or sgRNAs) is a functional link that allows for expression of the coding region of interest. Operably linked elements may be contiguous or non-contiguous. When used to refer to the joining of two protein coding regions, by operably linked is intended that the coding regions are in the same reading frame. Alternatively, the additional gene(s) or element(s) can be provided on multiple expression cassettes. For example, the nucleotide sequence encoding a presently disclosed RGN can be present on one expression cassette, whereas the nucleotide sequence encoding a crRNA, tracrRNA, or complete guide RNA can be on a separate expression cassette. Such an expression cassette is provided with a plurality of restriction sites and/or recombination sites for insertion of the polynucleotides to be under the transcriptional regulation of the regulatory regions. The expression cassette may additionally contain a selectable marker gene.

The expression cassette will include in the 5′-3′ direction of transcription, a transcriptional (and, in some embodiments, translational) initiation region (i.e., a promoter), an RGN-, crRNA-, tracrRNA- and/or sgRNA-encoding polynucleotide of the invention, and a transcriptional (and in some embodiments, translational) termination region (i.e., termination region) functional in the organism of interest. The promoters of the invention are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO 0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds. (1980) Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, growth stage-specific, cell type-specific, tissue-preferred, tissue-specific, or other promoters for expression in the organism of interest. See, for example, promoters set forth in WO 99/43838 and in U.S. Pat. Nos. 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); and MAS (Velten et al. (1984) EMBO J. 3:2723-2730).

Examples of inducible promoters are the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the Axig1 promoter which is auxin induced and tapetum specific but also active in callus (PCT US01/22169), the steroid-responsive promoters (see, for example, the ERE promoter which is estrogen induced, and the glucocorticoid-inducible promoter in Schena et al. (1991) Proc. Natl. Acad. Sci. USA 88:10421-10425 and McNellis et al. (1998) Plant J. 14 (2): 247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-specific or tissue-preferred promoters can be utilized to target expression of an expression construct within a particular tissue. In certain embodiments, the tissue-specific or tissue-preferred promoters are active in plant tissue. Examples of promoters under developmental control in plants include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding a RGN, crRNA, and/or tracrRNA comprise a cell type-specific promoter. A “cell type specific” promoter is a promoter that primarily drives expression in certain cell types in one or more organs. Some examples of plant cells in which cell type specific promoters functional in plants may be primarily active include, for example, BETL cells, vascular cells in roots, leaves, stalk cells, and stem cells. The nucleic acid molecules can also include cell type preferred promoters. A “cell type preferred” promoter is a promoter that primarily drives expression mostly, but not necessarily entirely or solely in certain cell types in one or more organs. Some examples of plant cells in which cell type preferred promoters functional in plants may be preferentially active include, for example, BETL cells, vascular cells in roots, leaves, stalk cells, and stem cells.

The nucleic acid sequences encoding the RGNs, crRNAs, tracrRNAs, and/or sgRNAs can be operably linked to a promoter sequence that is recognized by a phage RNA polymerase for example, for in vitro mRNA synthesis. In such embodiments, the in vitro-transcribed RNA can be purified for use in the methods described herein. For example, the promoter sequence can be a T7, T3, or SP6 promoter sequence or a variation of a T7, T3, or SP6 promoter sequence. In such embodiments, the expressed protein and/or RNAs can be purified for use in the methods of genome modification described herein. In certain embodiments, the polynucleotide encoding the RGN, crRNA, tracrRNA, and/or sgRNA also can be linked to a polyadenylation signal (e.g., SV40 polyA signal and other signals functional in plants) and/or at least one transcriptional termination sequence. Additionally, the sequence encoding the RGN also can be linked to sequence(s) encoding at least one nuclear localization signal, at least one cell-penetrating domain, and/or at least one signal peptide capable of trafficking proteins to particular subcellular locations, as described elsewhere herein.

The polynucleotide encoding the RGN, crRNA, tracrRNA, and/or sgRNA can be present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). The vector can comprise additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001. The vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). In some embodiments, the expression cassette or vector comprising the sequence encoding the RGN polypeptide can further comprise a sequence encoding a crRNA and/or a tracrRNA, or the crRNA and tracrRNA combined to create a guide RNA. The sequence(s) encoding the crRNA and/or tracrRNA can be operably linked to at least one transcriptional control sequence for expression of the crRNA and/or tracrRNA in the organism or host cell of interest. For example, the polynucleotide encoding the crRNA and/or tracrRNA can be operably linked to a promoter sequence that is recognized by RNA polymerase III (Pol III). Examples of suitable Pol III promoters include, but are not limited to, mammalian U6, U3, H1, and 7SL RNA promoters and rice U6 and U3 promoters.

As indicated, expression constructs comprising nucleotide sequences encoding the RGNs, crRNA, tracrRNA, and/or sgRNA can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the invention do not require a particular method for introducing a nucleotide construct to a host organism, only that the nucleotide construct gains access to the interior of at least one cell of the host organism. The host cell can be a eukaryotic or prokaryotic cell. In particular embodiments, the eukaryotic host cell is a plant cell, a mammalian cell, or an insect cell. Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic organisms” or “transformed organisms” or “stably transformed” organisms or cells or tissues refers to organisms that have incorporated or integrated a polynucleotide encoding a RGN, crRNA, and/or tracrRNA of the invention. It is recognized that other exogenous or endogenous nucleic acid sequences or DNA fragments may also be incorporated into the host cell. Agrobacterium- and biolistic-mediated transformation remain the two predominantly employed approaches for transformation of plant cells. However, transformation of a host cell may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding an RGN, crRNA, and/or tracrRNA includes retroviral, lentiviral, adenoviral, and adeno-associated viral mediated introduction and expression, as well as the use of Caulimoviruses, Geminiviruses, and RNA plant viruses.

Transformation protocols as well as protocols for introducing polypeptides or polynucleotide sequences into plants may vary depending on the type of host cell (e.g., monocot or dicot plant cell) targeted for transformation. Methods for transformation are known in the art and include those set forth in U.S. Pat. Nos. 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G.W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5;

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation of chloroplasts are known in the art. See, for example, Svab et al. (1990) Proc. Nail. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into a transgenic organism, such as a plant, in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

Alternatively, cells that have been transformed may be introduced into an organism. These cells could have originated from the organism, wherein the cells are transformed in an ex vivo approach.

The sequences provided herein may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassaya, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product or byproduct that retains the sequences disclosed herein, including for example, soymeal.

The polynucleotides encoding the RGNs, crRNAs, and/or tracrRNAs can also be used to transform any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The polynucleotides encoding the RGNs, crRNAs, and/or tracrRNAs can be used to transform any eukaryotic species, including but not limited to animals (e.g., mammals, insects, fish, birds, and reptiles), fungi, amoeba, algae, and yeast.

Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Viral. 66:2731-2739 (1992); Johann et al., J. Viral. 66:1635-1640 (1992); Sommnerfelt et al., Viral. 176:58-59 (1990); Wilson et al., J. Viral. 63:2374-2378 (1989); Miller et al., 1. Viral. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, 1. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., 1. Viral. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLaS3, Huhl, Huh4, Huh7, HUVEC, HASMC, HEKn, HEKa, MiaPaCell, Panel, PC-3, TFI, CTLL-2, CIR, Rat6, CVI, RPTE, AIO, T24, 182, A375, ARH-77, Calul, SW480, SW620, SKOV3, SK-UT, CaCo2, P388DI, SEM-K2, WEHI-231, HB56, TIB55, lurkat, 145.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-LI, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10TI/2, C6/36, Cal-27, CHO, CHO-7, CHO-IR, CHO-KI, CHO-K2, CHO-T, CHO Dhfr-/-, COR-L23, COR-L23/CPR, COR-L235010, CORL23/R23, COS-7, COV-434, CML TI, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, lurkat, IY cells, K562 cells, Ku812, KCL22, KGI, KYOI, LNCap, Ma-Mel 1-48, MC-38, MCF-7, MCF-10A, MDA-MB-231, MDA-MB-468, MDA-MB-435, MDCKII, MDCKII, MOR/0.2R, MONO-MAC 6, MTD-IA, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THPI cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is a mammal, such as a mouse, rat, or rabbit.

### IV. Variants and Fragments of Polypeptides and Polynucleotides

The present disclosure provides active variants and fragments of a naturally-occurring (i.e., wild-type) RNA-guided nuclease, the amino acid sequence of which is set forth as SEQ ID NOs: 1, 11, 19, 27, 36, 45, or 54, as well as active variants and fragments of naturally-occurring CRISPR repeats, such as the sequence set forth as SEQ ID NOs: 2, 12, 20, 28, 37, 46, or 55, and active variant and fragments of naturally-occurring tracrRNAs, such as the sequence set forth as SEQ ID NOs: 3, 13, 21, 29, 38, 47, or 56, and polynucleotides encoding the same.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of naturally-occurring RGN polypeptides, such as those disclosed herein, will retain sequence-specific, RNA-guided DNA-binding activity. In particular embodiments, fragments and variants of naturally-occurring RGN polypeptides, such as those disclosed herein, will retain nuclease activity (single-stranded or double-stranded).

Fragments and variants of naturally-occurring CRISPR repeats, such as those disclosed herein, will retain the ability, when part of a guide RNA (comprising a tracrRNA), to bind to and guide an RNA-guided nuclease (complexed with the guide RNA) to a target nucleotide sequence in a sequence-specific manner.

Fragments and variants of naturally-occurring tracrRNAs, such as those disclosed herein, will retain the ability, when part of a guide RNA (comprising a CRISPR RNA), to guide an RNA-guided nuclease (complexed with the guide RNA) to a target nucleotide sequence in a sequence-specific manner.

The term “fragment” refers to a portion of a polynucleotide or polypeptide sequence of the invention. “Fragments” or “biologically active portions” include polynucleotides comprising a sufficient number of contiguous nucleotides to retain the biological activity (i.e., binding to and directing an RGN in a sequence-specific manner to a target nucleotide sequence when comprised within a guideRNA). “Fragments” or “biologically active portions” include polypeptides comprising a sufficient number of contiguous amino acid residues to retain the biological activity (i.e., binding to a target nucleotide sequence in a sequence-specific manner when complexed with a guide RNA). Fragments of the RGN proteins include those that are shorter than the full-length sequences due to the use of an alternate downstream start site. A biologically active portion of an RGN protein can be a polypeptide that comprises, for example, 10, 25, 50, 100, 150, 200, 250, 300, 350, 400, 450, 500, 550, 600, 650, 700, 750, 800, 850, 900, 950, 1000, 1050 or more contiguous amino acid residues of SEQ ID NOs: 1, 11, 19, 27, 36, 45, or 54. Such biologically active portions can be prepared by recombinant techniques and evaluated for sequence-specific, RNA-guided DNA-binding activity. A biologically active fragment of a CRISPR repeat sequence can comprise at least 8 contiguous amino acids of SEQ ID NOs: 2, 12, 20, 28, 37, 46, or 55. A biologically active portion of a CRISPR repeat sequence can be a polynucleotide that comprises, for example, 8, 9, 10, 11, 12, 13, 14, 15, 16, 17, 18, 19, or 20 contiguous nucleotides of SEQ ID NOs: 2, 12, 20, 28, 37, 46, or 55. A biologically active portion of a tracrRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80 or more contiguous nucleotides of SEQ ID NOs: 3, 13, 21, 29, 38, 47, or 56.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

In particular embodiments, the presently disclosed polynucleotides encode an RNA-guided nuclease polypeptide comprising an amino acid sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to an amino acid sequence of SEQ ID NOs: 1, 11, 19, 27, 36, 45, or 54.

A biologically active variant of an RGN polypeptide of the invention may differ by as few as about 1-15 amino acid residues, as few as about 1-10, such as about 6-10, as few as 5, as few as 4, as few as 3, as few as 2, or as few as 1 amino acid residue. In specific embodiments, the polypeptides can comprise an N-terminal or a C-terminal truncation, which can comprise at least a deletion of 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050 amino acids or more from either the N or C terminus of the polypeptide.

In certain embodiments, the presently disclosed polynucleotides comprise or encode a CRISPR repeat comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as SEQ ID NOs: 2, 12, 20, 28, 37, 46, or 55.

The presently disclosed polynucleotides can comprise or encode a tracrRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to the nucleotide sequence set forth as SEQ ID NOs: 3, 13, 21, 29, 38, 47, or 56.

Biologically active variants of a CRISPR repeat or tracrRNA of the invention may differ by as few as about 1-15 nucleotides, as few as about 1-10, such as about 6-10, as few as 5, as few as 4, as few as 3, as few as 2, or as few as 1 nucleotides. In specific embodiments, the polynucleotides can comprise a 5′ or 3′ truncation, which can comprise at least a deletion of 10, 15, 20, 25, 30, 35, 40, 45, 50, 55, 60, 65, 70, 75, 80 nucleotides or more from either the 5′ or 3′ end of the polynucleotide.

It is recognized that modifications may be made to the RGN polypeptides, CRISPR repeats, and tracrRNAs provided herein creating variant proteins and polynucleotides. Changes designed by man may be introduced through the application of site-directed mutagenesis techniques. Alternatively, native, as yet-unknown or as yet unidentified polynucleotides and/or polypeptides structurally and/or functionally-related to the sequences disclosed herein may also be identified that fall within the scope of the present invention. Conservative amino acid substitutions may be made in nonconserved regions that do not alter the function of the RGN proteins. Alternatively, modifications may be made that improve the activity of the RGN.

Variant polynucleotides and proteins also encompass sequences and proteins derived from a mutagenic and recombinogenic procedure such as DNA shuffling. With such a procedure, one or more different RGN proteins disclosed herein (e.g., SEQ ID NOs: 1, 11, 19, 27, 36, 45, or 54) is manipulated to create a new RGN protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the RGN sequences provided herein and other known RGN genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acids positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (www.ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### V. Antibodies

Antibodies to the RGN polypeptides or ribonucleoproteins comprising the RGN polypeptides of the present invention, including those having the amino acid sequence set forth as SEQ ID NOs: 1, 11, 19, 27, 36, 45, or 54 or active variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of RGN polypeptides or ribonucleoproteins. Thus, this disclosure provides kits comprising antibodies that specifically bind to the polypeptides or ribonucleoproteins described herein, including, for example, polypeptides having the sequence of SEQ ID NOs: 1, 11, 19, 27, 36, 45, or 54.

### VI. Systems and Ribonucleoprotein Complexes for Binding a Target Sequence of Interest and Methods of Making the Same

The present disclosure provides a system for binding a target sequence of interest, wherein the system comprises at least one guide RNA or a nucleotide sequence encoding the same, and at least one RNA-guided nuclease or a nucleotide sequence encoding the same. The guide RNA hybridizes to the target sequence of interest and also forms a complex with the RGN polypeptide, thereby directing the RGN polypeptide to bind to the target sequence. In some of these embodiments, the RGN comprises an amino acid sequence of SEQ ID NOs: 1, 11, 19, 27, 36, 45, or 54, or an active variant or fragment thereof. In various embodiments, the guide RNA comprises a CRISPR repeat sequence comprising the nucleotide sequence of SEQ ID NOs: 2, 12, 20, 28, 37, 46, or 55, or an active variant or fragment thereof. In particular embodiments, the guide RNA comprises a tracrRNA comprising a nucleotide sequence of SEQ ID NOs: 3, 13, 21, 29, 38, 47, or 56, or an active variant or fragment thereof. The guide RNA of the system can be a single guide RNA or a dual-guide RNA. In particular embodiments, the system comprises a RNA-guided nuclease that is heterologous to the guideRNA, wherein the RGN and guideRNA are not naturally complexed in nature.

The system for binding a target sequence of interest provided herein can be a ribonucleoprotein complex, which is at least one molecule of an RNA bound to at least one protein. The ribonucleoprotein complexes provided herein comprise at least one guide RNA as the RNA component and an RNA-guided nuclease as the protein component. Such ribonucleoprotein complexes can be purified from a cell or organism that naturally expresses an RGN polypeptide and has been engineered to express a particular guide RNA that is specific for a target sequence of interest. Alternatively, the ribonucleoprotein complex can be purified from a cell or organism that has been transformed with polynucleotides that encode an RGN polypeptide and a guide RNA and cultured under conditions to allow for the expression of the RGN polypeptide and guide RNA. Thus, methods are provided for making an RGN polypeptide or an RGN ribonucleoprotein complex. Such methods comprise culturing a cell comprising a nucleotide sequence encoding an RGN polypeptide, and in some embodiments a nucleotide sequence encoding a guide RNA, under conditions in which the RGN polypeptide (and in some embodiments, the guide RNA) is expressed. The RGN polypeptide or RGN ribonucleoprotein can then be purified from a lysate of the cultured cells.

Methods for purifying an RGN polypeptide or RGN ribonucleoprotein complex from a lysate of a biological sample are known in the art (e.g., size exclusion and/or affinity chromatography, 2D-PAGE, HPLC, reversed-phase chromatography, immunoprecipitation). In particular methods, the RGN polypeptide is recombinantly produced and comprises a purification tag to aid in its purification, including but not limited to, glutathione-S-transferase (GST), chitin binding protein (CBP), maltose binding protein, thioredoxin (TRX), poly (NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 10×His, biotin carboxyl carrier protein (BCCP), and calmodulin. Generally, the tagged RGN polypeptide or RGN ribonucleoprotein complex is purified using immobilized metal affinity chromatography. It will be appreciated that other similar methods known in the art may be used, including other forms of chromatography or for example immunoprecipitation, either alone or in combination.

An “isolated” or “purified” polypeptide, or biologically active portion thereof, is substantially or essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Particular methods provided herein for binding and/or cleaving a target sequence of interest involve the use of an in vitro assembled RGN ribonucleoprotein complex. In vitro assembly of an RGN ribonucleoprotein complex can be performed using any method known in the art in which an RGN polypeptide is contacted with a guide RNA under conditions to allow for binding of the RGN polypeptide to the guide RNA. As used herein, “contact”, contacting “, “contacted,” refer to placing the components of a desired reaction together under conditions suitable for carrying out the desired reaction. The RGN polypeptide can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGN polypeptide and guide RNA can be brought into contact in solution (e.g., buffered saline solution) to allow for in vitro assembly of the RGN ribonucleoprotein complex.

### VII. Methods of Binding, Cleaving, or Modifying a Target Sequence

The present disclosure provides methods for binding, cleaving, and/or modifying a target nucleotide sequence of interest. The methods include delivering a system comprising at least one guide

RNA or a polynucleotide encoding the same, and at least one RGN polypeptide or a polynucleotide encoding the same to the target sequence or a cell, organelle, or embryo comprising the target sequence. In some of these embodiments, the RGN comprises the amino acid sequence of SEQ ID NOs: 1, 11, 19, 27, 36, 45, or 54, or an active variant or fragment thereof. In various embodiments, the guide RNA comprises a CRISPR repeat sequence comprising the nucleotide sequence of SEQ ID NOs: 2, 12, 20, 28, 37, 46, or 55, or an active variant or fragment thereof. In particular embodiments, the guide RNA comprises a tracrRNA comprising the nucleotide sequence of SEQ ID NOs: 3, 13, 21, 29, 38, 47, or 56, or an active variant or fragment thereof. The guide RNA of the system can be a single guide RNA or a dual-guide RNA. The RGN of the system may be nuclease dead RGN, have nickase activity, or may be a fusion polypeptide. In some embodiments, the fusion polypeptide comprises a base-editing polypeptide, for example a cytidine deaminase or an adenosine deaminase. In particular embodiments, the RGN and/or guide RNA is heterologous to the cell, organelle, or embryo to which the RGN and/or guide RNA (or polynucleotide(s) encoding at least one of the RGN and guide RNA) are introduced.

In those embodiments wherein the method comprises delivering a polynucleotide encoding a guide RNA and/or an RGN polypeptide, the cell or embryo can then be cultured under conditions in which the guide RNA and/or RGN polypeptide are expressed. In various embodiments, the method comprises contacting a target sequence with an RGN ribonucleoprotein complex. The RGN ribonucleoprotein complex may comprise an RGN that is nuclease dead or has nickase activity. In some embodiments, the RGN of the ribonucleoprotein complex is a fusion polypeptide comprising a base-editing polypeptide. In certain embodiments, the method comprises introducing into a cell, organelle, or embryo comprising a target sequence an RGN ribonucleoprotein complex. The RGN ribonucleoprotein complex can be one that has been purified from a biological sample, recombinantly produced and subsequently purified, or in vitro-assembled as described herein. In those embodiments wherein the RGN ribonucleoprotein complex that is contacted with the target sequence or a cell organelle, or embryo has been assembled in vitro, the method can further comprise the in vitro assembly of the complex prior to contact with the target sequence, cell, organelle, or embryo.

A purified or in vitro assembled RGN ribonucleoprotein complex can be introduced into a cell, organelle, or embryo using any method known in the art, including, but not limited to electroporation. Alternatively, an RGN polypeptide and/or polynucleotide encoding or comprising the guide RNA can be introduced into a cell, organelle, or embryo using any method known in the art (e.g., electroporation).

Upon delivery to or contact with the target sequence or cell, organelle, or embryo comprising the target sequence, the guide RNA directs the RGN to bind to the target sequence in a sequence-specific manner. In those embodiments wherein the RGN has nuclease activity, the RGN polypeptide cleaves the target sequence of interest upon binding. The target sequence can subsequently be modified via endogenous repair mechanisms, such as non-homologous end joining, or homology-directed repair with a provided donor polynucleotide.

Methods to measure binding of an RGN polypeptide to a target sequence are known in the art and include chromatin immunoprecipitation assays, gel mobility shift assays, DNA pull-down assays, reporter assays, microplate capture and detection assays. Likewise, methods to measure cleavage or modification of a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010) Methods Mol Biol 649:247-256).

In some embodiments, the methods involve the use of a single type of RGN complexed with more than one guide RNA. The more than one guide RNA can target different regions of a single gene or can target multiple genes.

In those embodiments wherein a donor polynucleotide is not provided, a double-stranded break introduced by an RGN polypeptide can be repaired by a non-homologous end-joining (NHEJ) repair process. Due to the error-prone nature of NHEJ, repair of the double-stranded break can result in a modification to the target sequence. As used herein, a “modification” in reference to a nucleic acid molecule refers to a change in the nucleotide sequence of the nucleic acid molecule, which can be a deletion, insertion, or substitution of one or more nucleotides, or a combination thereof. Modification of the target sequence can result in the expression of an altered protein product or inactivation of a coding sequence.

In those embodiments wherein a donor polynucleotide is present, the donor sequence in the donor polynucleotide can be integrated into or exchanged with the target nucleotide sequence during the course of repair of the introduced double-stranded break, resulting in the introduction of the exogenous donor sequence. A donor polynucleotide thus comprises a donor sequence that is desired to be introduced into a target sequence of interest. In some embodiments, the donor sequence alters the original target nucleotide sequence such that the newly integrated donor sequence will not be recognized and cleaved by the RGN. Integration of the donor sequence can be enhanced by the inclusion within the donor polynucleotide of flanking sequences that have substantial sequence identity with the sequences flanking the target nucleotide sequence, allowing for a homology-directed repair process. In those embodiments wherein the RGN polypeptide introduces double-stranded staggered breaks, the donor polynucleotide can comprise a donor sequence flanked by compatible overhangs, allowing for direct ligation of the donor sequence to the cleaved target nucleotide sequence comprising overhangs by a non-homologous repair process during repair of the double-stranded break.

In those embodiments wherein the method involves the use of an RGN that is a nickase (i.e., is only able to cleave a single strand of a double-stranded polynucleotide), the method can comprise introducing two RGN nickases that target identical or overlapping target sequences and cleave different strands of the polynucleotide. For example, an RGN nickase that only cleaves the positive (+) strand of a double-stranded polynucleotide can be introduced along with a second RGN nickase that only cleaves the negative (−) strand of a double-stranded polynucleotide.

In various embodiments, a method is provided for binding a target nucleotide sequence and detecting the target sequence, wherein the method comprises introducing into a cell, organelle, or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN and further comprises a detectable label, and the method further comprises detecting the detectable label. The detectable label may be fused to the RGN as a fusion protein (e.g., fluorescent protein) or may be a small molecule conjugated to or incorporated within the RGN polypeptide that can be detected visually or by other means.

Also provided herein are methods for modulating the expression of a target sequence or a gene of interest under the regulation of a target sequence. The methods comprise introducing into a cell, organelle, or embryo at least one guide RNA or a polynucleotide encoding the same, and at least one

RGN polypeptide or a polynucleotide encoding the same, expressing the guide RNA and/or RGN polypeptide (if coding sequences are introduced), wherein the RGN polypeptide is a nuclease-dead RGN. In some of these embodiments, the nuclease-dead RGN is a fusion protein comprising an expression modulator domain (i.e., epigenetic modification domain, transcriptional activation domain or a transcriptional repressor domain) as described herein.

The present disclosure also provides methods for binding and/or modifying a target nucleotide sequence of interest. The methods include delivering a system comprising at least one guide RNA or a polynucleotide encoding the same, and at least one fusion polypeptide comprises an RGN of the invention and a base-editing polypeptide, for example a cytidine deaminase or an adenosine deaminase, or a polynucleotide encoding the fusion polypeptide, to the target sequence or a cell, organelle, or embryo comprising the target sequence.

One of ordinary skill in the art will appreciate that any of the presently disclosed methods can be used to target a single target sequence or multiple target sequences. Thus, methods comprise the use of a single RGN polypeptide in combination with multiple, distinct guide RNAs, which can target multiple, distinct sequences within a single gene and/or multiple genes. Also encompassed herein are methods wherein multiple, distinct guide RNAs are introduced in combination with multiple, distinct RGN polypeptides. These guide RNAs and guide RNA/RGN polypeptide systems can target multiple, distinct sequences within a single gene and/or multiple genes.

In one aspect, the invention provides kits containing any one or more of the elements disclosed in the above methods and compositions. In some embodiments, the kit comprises a vector system and instructions for using the kit. In some embodiments, the vector system comprises (a) a first regulatory element operably linked to a traer mate sequence and one or more insertion sites for inserting a guide sequence upstream of the tracr mate sequence, wherein when expressed, the guide sequence directs sequence-specific binding of a CRISPR complex to a target sequence in a eukaryotic cell, wherein the CRISPR complex comprises a CRIS PR enzyme complexed with (1) the guide sequence that is hybridized to the target sequence, and (2) the tracr mate sequence that is hybridized to the tracr sequence; and/or (b) a second regulatory element operably linked to an enzyme coding sequence encoding said CRISPR enzyme comprising a nuclear localization sequence. Elements may be provided individually or in combinations, and may be provided in any suitable container, such as a vial, a bottle, or a tube.

In some embodiments, the kit includes instructions in one or more languages. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating) a target polynucleotide in a multiplicity of cell types. As such the CRISPR complex of the invention has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide.

### VIII. Target Polynucleotides

In one aspect, the invention provides for methods of modifying a target polynucleotide in a eukaryotic cell, which may be in vivo, ex vivo or in vitro. In some embodiments, the method comprises sampling a cell or population of cells from a human or non-human animal or plant (including microalgae) and modifying the cell or cells. Culturing may occur at any stage ex vivo. The cell or cells may even be re-introduced into the non-human animal or plant (including micro-algae).

Using natural variability, plant breeders combine most useful genes for desirable qualities, such as yield, quality, uniformity, hardiness, and resistance against pests. These desirable qualities also include growth, day length preferences, temperature requirements, initiation date of floral or reproductive development, fatty acid content, insect resistance, disease resistance, nematode resistance, fungal resistance, herbicide resistance, tolerance to various environmental factors including drought, heat, wet, cold, wind, and adverse soil conditions including high salinity The sources of these useful genes include native or foreign varieties, heirloom varieties, wild plant relatives, and induced mutations, e.g., treating plant material with mutagenic agents. Using the present invention, plant breeders are provided with a new tool to induce mutations. Accordingly, one skilled in the art can analyze the genome for sources of useful genes, and in varieties having desired characteristics or traits employ the present invention to induce the rise of useful genes, with more precision than previous mutagenic agents and hence accelerate and improve plant breeding programs.

The target polynucleotide of an RGN system can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (protospacer adjacent motif); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence).

The target polynucleotide of a CRISPR complex may include a number of disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease (e.g., a causal mutation). The transcribed or translated products may be known or unknown, and further may be at a normal or abnormal level. Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web.

Although CRISPR systems are particularly useful for their relative ease in targeting to genomic sequences of interest, there still remains an issue of what the RGN can do to address a causal mutation. One approach is to produce a fusion protein between an RGN (preferably an inactive or nickase variant of the RGN) and a base-editing enzyme or the active domain of a base editing enzyme, such as an cytidine deaminase or an adenosine deaminase base editor (U.S. Pat. No. 9,840,699, herein incorporated by reference). In some embodiments, the methods comprise contacting a DNA molecule with (a) a fusion protein comprising an RGN of the invention and a base-editing polypeptide such as a deaminase; and (b) a gRNA targeting the fusion protein of (a) to a target nucleotide sequence of the DNA strand; wherein the DNA molecule is contacted with the fusion protein and the gRNA in an amount effective and under conditions suitable for the deamination of a nucleotide base. In some embodiments, the target DNA sequence comprises a sequence associated with a disease or disorder, and wherein the deamination of the nucleotide base results in a sequence that is not associated with a disease or disorder. In some embodiments, the target DNA sequence resides in an allele of a crop plant, wherein the particular allele of the trait of interest results in a plant of lesser agronomic value. The deamination of the nucleotide base results in an allele that improves the trait and increases the agronomic value of the plant.

In some embodiments, the DNA sequence comprises a T→C or A→G point mutation associated with a disease or disorder, and wherein the deamination of the mutant C or G base results in a sequence that is not associated with a disease or disorder. In some embodiments, the deamination corrects a point mutation in the sequence associated with the disease or disorder.

In some embodiments, the sequence associated with the disease or disorder encodes a protein, and wherein the deamination introduces a stop codon into the sequence associated with the disease or disorder, resulting in a truncation of the encoded protein. In some embodiments, the contacting is performed in vivo in a subject susceptible to having, having, or diagnosed with the disease or disorder. In some embodiments, the disease or disorder is a disease associated with a point mutation, or a single-base mutation, in the genome. In some embodiments, the disease is a genetic disease, a cancer, a metabolic disease, or a lysosomal storage disease.

Further examples of loci which are causal for certain genetic diseases, particularly loci which can be readily targeted by RGNs or RGN-base editor fusion proteins of the invention, can be found in Example 9 and corresponding Table 12.

### Hurler Syndrome

An example of a genetically inherited disease which could be corrected using an approach that relies on an RGN-base editor fusion protein of the invention is Hurler Syndrome. Hurler Syndrome, also known as MPS-1, is the result of a deficiency of α-L-iduronidase (IDUA) resulting in a lysosomal storage disease characterized at the molecular level by the accumulation of dermatan sulfate and heparan sulfate in lysosomes. This disease is generally an inherited genetic disorder caused by mutations in the IDUA gene encoding α-L-iduronidase. Common IDUA mutations are W402× and Q70X, both nonsense mutations resulting in premature termination of translation. Such mutations are well addressed by precise genome editing (PGE) approaches, since reversion of a single nucleotide, for example by a base-editing approach, would restore the wild-type coding sequence and result in protein expression controlled by the endogenous regulatory mechanisms of the genetic locus. Additionally, since heterozygotes are known to be asymptomatic, a PGE therapy that targets one of these mutations would be useful to a large proportion of patients with this disease, as only one of the mutated alleles needs to be corrected (Bunge et al. (1994) Hum. Mol. Genet. 3 (6): 861-866, herein incorporated by reference).

Current treatments for Hurler Syndrome include enzyme replacement therapy and bone marrow transplants (Vellodi et al. (1997) Arch. Dis. Child. 76 (2): 92-99; Peters et al. (1998) Blood 91 (7): 2601-2608, herein incorporated by reference). While enzyme replacement therapy has had a dramatic effect on the survival and quality of life of Hurler Syndrome patients, this approach requires costly and time-consuming weekly infusions. Additional approaches include the delivery of the IDUA gene on an expression vector or the insertion of the gene into a highly expressed locus such as that of serum albumin (U.S. Pat. No. 9,956,247, herein incorporated by reference). However, these approaches do not restore the original IDUA locus to the correct coding sequence. A genome-editing strategy would have a number of advantages, most notably that regulation of gene expression would be controlled by the natural mechanisms present in healthy individuals. Additionally, using base editing does not necessitate causing a double stranded DNA breaks, which could lead to large chromosomal rearrangements, cell death, or oncogenecity by the disruption of tumor suppression mechanisms. An enabling description of a method to correct the causal mutation of this disease is provided in Example 10. The described methods are an example of a general strategy directed toward using RGN-base editor fusion proteins of the invention to target and correct certain disease-causing mutations in the human genome. It will be appreciated that similar approaches to target diseases such as those described in Table 12 may also be pursued. It will be further appreciated that similar approaches to target disease-causing mutations in other species, particularly common household pets or livestock, can also be deployed using the RGNs of the invention. Common household pets and livestock include dogs, cats, horses, pigs, cows, sheep, chickens, donkeys, snakes, ferrets, fish including salmon, and shrimp.

### Friedreich's Ataxia

RGNs of the invention could also be useful in human therapeutic approaches where the causal mutation is more complicated. For example, some diseases such as Friedreich's Ataxia and Huntington's Disease are the result of a significant increase in repeats of a three nucleotide motif at a particular region of a gene, which affects the ability of the expressed protein to function or to be expressed. Friedreich's Ataxia (FRDA) is an autosomal recessive disease resulting in progressive degeneration of nervous tissue in the spinal cord. Reduced levels of the frataxin (FXN) protein in the mitochondria cause oxidative damages and iron deficiencies at the cellular level. The reduced FXN expression has been linked to a GAA triplet expansion within the intron 1 of the somatic and germline FXN gene. In FRDA patients, the GAA repeat frequently consists of more than 70, sometimes even more than 1000 (most commonly 600-900) triplets, whereas unaffected individuals have about 40 repeats or less (Pandolfo et al. (2012) Handbook of Clinical Neurology 103:275-294; Campuzano et al. (1996) Science 271:1423-1427; Pandolfo (2002) Adv. Exp. Med. Biol. 516:99-118; all herein incorporated by reference).

The expansion of the trinucleotide repeat sequence causing Friedreich's Ataxia (FRDA) occurs in a defined genetic locus within the FXN gene, referred to as the FRDA instability region. RNA guided nucleases (RGNs) may be used for excising the instability region in FRDA patient cells. This approach requires 1) an RGN and guide RNA sequence that can be programmed to target the allele in the human genome; and 2) a delivery approach for the RGN and guide sequence. Many nucleases used for genome editing, such as the commonly used Cas9 nuclease from S. pyogenes (SpCas9), are too large to be packaged into adeno-associated viral (AAV) vectors, especially when considering the length of the SpCas9 gene and the guide RNA in addition to other genetic elements required for functional expression cassettes. This makes an approach using SpCas9 more difficult.

The compact RNA guided nucleases of the invention, particularly APG07433.1 and APG08290.1, are uniquely well suited for the excision of the FRDA instability region. Each RGN has a PAM requirement that is in the vicinity of the FRDA instability region. Additionally, each of these RGNs can be packaged into an AAV vector along with a guide RNA. Packing two guide RNAs would likely require a second vector, but this approach still compares favorably to what would be required of a larger nuclease such as SpCas9, which may require splitting the protein sequence between two vectors. An enabling description of a method to correct the causal mutation of this disease is provided in Example 11. The described methods encompass a strategy using RGNs of the invention in which a region of genomic instability is removed. Such a strategy is applicable to other diseases and disorders which have a similar genetic basis, such as Huntington's Disease. Similar strategies using RGNs of the invention may also be applicable to similar diseases and disorders in non-human animals of agronomic or economic importance, including dogs, cats, horses, pigs, cows, sheep, chickens, donkeys, snakes, ferrets, fish including salmon, and shrimp

### Hemoglobinopathies

RGNs of the invention could also be to introduce disruptive mutations that may result in a beneficial effect. Genetic defects in the genes encoding hemoglobin, particularly the beta globin chain (the HBB gene), can be responsible for a number of diseases known as hemoglobinopathies, including sickle cell anemia and thalassemias.

In adult humans, hemoglobin is a heterotetramer comprising two alpha (a)-like globin chains and two beta (β)-like globin chains and 4 heme groups. In adults the α2β2 tetramer is referred to as Hemoglobin A (HbA) or adult hemoglobin. Typically, the alpha and beta globin chains are synthesized in an approximate 1:1 ratio and this ratio seems to be critical in terms of hemoglobin and red blood cell (RBC) stabilization. In a developing fetus, a different form of hemoglobin, fetal hemoglobin (HbF), is produced which has a higher binding affinity for oxygen than Hemoglobin A such that oxygen can be delivered to the baby's system via the mother's blood stream. Fetal hemoglobin also contains two a globin chains, but in place of the adult β-globin chains, it has two fetal gamma (γ)-globin chains (i.e., fetal hemoglobin is α2γ2). The regulation of the switch from production of gamma-to beta-globin is quite complex, and primarily involves a down-regulation of gamma globin transcription with a simultaneous up-regulation of beta globin transcription. At approximately 30 weeks of gestation, the synthesis of gamma globin in the fetus starts to drop while the production of beta globin increases. By approximately 10 months of age, the newborn's hemoglobin is nearly all α2β2 although some HbF persists into adulthood (approximately 1-3% of total hemoglobin). In the majority of patients with hemoglobinopathies, the genes encoding gamma globin remain present, but expression is relatively low due to normal gene repression occurring around parturition as described above.

Sickle cell disease is caused by a V6E mutation in the β globin gene (HBB) (a GAG to GTG at the DNA level), where the resultant hemoglobin is referred to as “hemoglobinS” or “HbS.” Under lower oxygen conditions, HbS molecules aggregate and form fibrous precipitates. These aggregates cause the abnormality or ‘sickling’ of the RBCs, resulting in a loss of flexibility of the cells. The sickling RBCs are no longer able to squeeze into the capillary beds and can result in vaso-occlusive crisis in sickle cell patients. In addition, sickled RBCs are more fragile than normal RBCs, and tend towards hemolysis, eventually leading to anemia in the patient.

Treatment and management of sickle cell patients is a life-long proposition involving antibiotic treatment, pain management and transfusions during acute episodes. One approach is the use of hydroxyurea, which exerts its effects in part by increasing the production of gamma globin. Long term side effects of chronic hydroxyurea therapy are still unknown, however, and treatment gives unwanted side effects and can have variable efficacy from patient to patient. Despite an increase in the efficacy of sickle cell treatments, the life expectancy of patients is still only in the mid to late 50's and the associated morbidities of the disease have a profound impact on a patient's quality of life.

Thalassemias (alpha thalassemias and beta thalassemia) are also diseases relating to hemoglobin and typically involve a reduced expression of globin chains. This can occur through mutations in the regulatory regions of the genes or from a mutation in a globin coding sequence that results in reduced expression or reduced levels or functional globin protein. Treatment of thalassemias usually involves blood transfusions and iron chelation therapy. Bone marrow transplants are also being used for treatment of people with severe thalassemias if an appropriate donor can be identified, but this procedure can have significant risks.

One approach that has been proposed for the treatment of both SCD and beta thalassemias is to increase the expression of gamma globin so that HbF functionally replaces the aberrant adult hemoglobin As mentioned above, treatment of SCD patients with hydroxyurea is thought to be successful in part due to its effect on increasing gamma globin expression (DeSimone (1982) Proc Nat'l Acad Sci USA 79 (14): 4428-31; Ley, et al., (1982) N. Engl. J. Medicine, 307:1469-1475; Ley, et al., (1983) Blood 62:370-380; Constantoulakis et al., (1988) Blood 72 (6): 1961-1967, all herein incorporated by reference). Increasing the expression of HbF involves identification of genes whose products play a role in the regulation of gamma globin expression. One such gene is BCL11A. BCL11A encodes a zinc finger protein that expressed in adult erythroid precursor cells, and down-regulation of its expression leads to an increase in gamma globin expression (Sankaran et at (2008) Science 322:1839, herein incorporated by reference). Use of an inhibitory RNA targeted to the BCL11A gene has been proposed (e.g., U.S. Patent Publication 2011/0182867, herein incorporated by reference) but this technology has several potential drawbacks, including that complete knock down may not be achieved, delivery of such RNAs may be problematic, and the RNAs must be present continuously, requiring multiple treatments for life.

RGNs of the invention may be used to target the BCL11A enhancer region to disrupt expression of BCL11A, thereby increasing gamma globin expression. This targeted disruption can be achieved by non-homologous end joining (NHEJ), whereby an RGN of the invention targets to a particular sequence within the BCL11A enhancer region, makes a double-stranded break, and the cell's machinery repairs the break, typically simultaneously introducing deleterious mutations. Similar to what is described for other disease targets, the RGNs of the invention have advantages over other known RGNs due to their relatively small size, which enables packaging expression cassettes for the RGN and its guide RNA into a single AAV vector for in vivo delivery. An enabling description of this method is provided in Example 12. Similar strategies using RGNs of the invention may also be applicable to similar diseases and disorders in both humans and in non-human animals of agronomic or economic importance.

### IX. Cells Comprising a Polynucleotide Genetic Modification

Provided herein are cells and organisms comprising a target sequence of interest that has been modified using a process mediated by an RGN, crRNA, and/or tracrRNA as described herein. In some of these embodiments, the RGN comprises the amino acid sequence of SEQ ID NOs: 1, 11, 19, 27, 36, 45, or 54, or an active variant or fragment thereof. In various embodiments, the guide RNA comprises a CRISPR repeat sequence comprising the nucleotide sequence of SEQ ID NOs: 2, 12, 20, 28, 37, 46, or 55, or an active variant or fragment thereof. In particular embodiments, the guide RNA comprises a tracrRNA comprising the nucleotide sequence of SEQ ID NOs: 3, 13, 21, 29, 38, 47, or 56, or an active variant or fragment thereof. The guide RNA of the system can be a single guide RNA or a dual-guide RNA.

The modified cells can be eukaryotic (e.g., mammalian, plant, insect cell) or prokaryotic. Also provided are organelles and embryos comprising at least one nucleotide sequence that has been modified by a process utilizing an RGN, crRNA, and/or tracrRNA as described herein. The genetically modified cells, organisms, organelles, and embryos can be heterozygous or homozygous for the modified nucleotide sequence.

The chromosomal modification of the cell, organism, organelle, or embryo can result in altered expression (up-regulation or down-regulation), inactivation, or the expression of an altered protein product or an integrated sequence. In those instances wherein the chromosomal modification results in either the inactivation of a gene or the expression of a non-functional protein product, the genetically modified cell, organism, organelle, or embryo is referred to as a “knock out”. The knock out phenotype can be the result of a deletion mutation (i.e., deletion of at least one nucleotide), an insertion mutation (i.e., insertion of at least one nucleotide), or a nonsense mutation (i.e., substitution of at least one nucleotide such that a stop codon is introduced).

Alternatively, the chromosomal modification of a cell, organism, organelle, or embryo can produce a “knock in”, which results from the chromosomal integration of a nucleotide sequence that encodes a protein. In some of these embodiments, the coding sequence is integrated into the chromosome such that the chromosomal sequence encoding the wild-type protein is inactivated, but the exogenously introduced protein is expressed.

In other embodiments, the chromosomal modification results in the production of a variant protein product. The expressed variant protein product can have at least one amino acid substitution and/or the addition or deletion of at least one amino acid. The variant protein product encoded by the altered chromosomal sequence can exhibit modified characteristics or activities when compared to the wild-type protein, including but not limited to altered enzymatic activity or substrate specificity.

In yet other embodiments, the chromosomal modification can result in an altered expression pattern of a protein. As a non-limiting example, chromosomal alterations in the regulatory regions controlling the expression of a protein product can result in the overexpression or downregulation of the protein product or an altered tissue or temporal expression pattern.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

Non-limiting embodiments include:
1. A nucleic acid molecule comprising a polynucleotide encoding an RNA-guided nuclease (RGN) polypeptide, wherein said polynucleotide comprises a nucleotide sequence encoding an RGN polypeptide comprising an amino acid sequence having at least 95% sequence identity to SEQ ID NOs: 1, 11, 19, 27, 36, 45, or 54;
   wherein said RGN polypeptide binds a target DNA sequence in an RNA-guided sequence specific manner when bound to a guide RNA (gRNA) capable of hybridizing to said target DNA sequence, and wherein said polynucleotide encoding an RGN polypeptide is operably linked to a promoter heterologous to said polynucleotide.
2. The nucleic acid molecule of embodiment 1, wherein said RGN polypeptide is capable of cleaving said target DNA sequence upon binding.
3. The nucleic acid molecule of embodiment 2, wherein cleavage by said RGN polypeptide generates a double-stranded break.
4. The nucleic acid molecule of embodiment 2, wherein cleavage by said RGN polypeptide generates a single-stranded break.
5. The nucleic acid molecule of any one of embodiments 1-4, wherein the RGN polypeptide is operably fused to a base-editing polypeptide.
6. The nucleic acid molecule of any one of embodiments 1-5, wherein the RGN polypeptide comprises one or more nuclear localization signals.
7. The nucleic acid molecule of any one of embodiments 1-6, wherein the RGN polypeptide is codon optimized for expression in a eukaryotic cell.
8. The nucleic acid molecule of any one of embodiments 1-7, wherein said target DNA sequence is located adjacent to a protospacer adjacent motif (PAM).
9. A vector comprising the nucleic acid molecule of any one of embodiments 1-8.
10. The vector of embodiment 9, further comprising at least one nucleotide sequence encoding said gRNA capable of hybridizing to said target DNA sequence.
11. The vector of embodiment 10, where said gRNA is a single guide RNA.
12. The vector of embodiment 10, wherein said gRNA is a dual-guide RNA.
13. The vector of any one of embodiments 10-12, wherein the guide RNA comprises a CRISPR RNA comprising a CRISPR repeat sequence having at least 95% sequence identity to SEQ ID NOs: 2, 12, 20, 28, 37, 46, or 55.
14. The vector of any one of embodiments 10-13, wherein the guide RNA comprises a tracrRNA having at least 95% sequence identity to SEQ ID NOs: 3, 13, 21, 29, 38, 47, or 56.
15. A cell comprising the nucleic acid molecule of any one of embodiments 1-8 or the vector of any one of embodiments 9-14.
16. A method for making an RGN polypeptide comprising culturing the cell of embodiment 15 under conditions in which the RGN polypeptide is expressed.
17. A method for making an RGN polypeptide comprising introducing into a cell a heterologous nucleic acid molecule comprising a nucleotide sequence encoding an RNA-guided nuclease (RGN) polypeptide comprising an amino acid sequence having at least 95% sequence identity to SEQ ID NOs: 1, 11, 19, 27, 36, 45, or 54;
   wherein said RGN polypeptide binds a target DNA sequence in an RNA-guided sequence specific manner when bound to a guide RNA (gRNA) capable of hybridizing to said target DNA sequence;
   and culturing said cell under conditions in which the RGN polypeptide is expressed.
18. The method of embodiment 16 or 17, further comprising purifying said RGN polypeptide.
19. The method of embodiment 16 or 17, wherein said cell further expresses one or more guide RNAs that binds to said RGN polypeptide to form an RGN ribonucleoprotein complex.
20. The method of embodiment 19, further comprising purifying said RGN ribonucleoprotein complex.
21. A nucleic acid molecule comprising a polynucleotide encoding a CRISPR RNA (crRNA), wherein said crRNA comprises a spacer sequence and a CRISPR repeat sequence, wherein said CRISPR repeat sequence comprises a nucleotide sequence having at least 95% sequence identity to SEQ ID NOs: 2, 12, 20, 28, 37, 46, or 55;
   wherein a guide RNA comprising:
      a) said crRNA; and
      b) a trans-activating CRISPR RNA (tracrRNA) hybridized to said CRISPR repeat sequence of said crRNA;
   is capable of hybridizing to a target DNA sequence in a sequence specific manner through the spacer sequence of said crRNA when said guide RNA is bound to an RNA-guided nuclease (RGN) polypeptide, and
      wherein said polynucleotide encoding a crRNA is operably linked to a promoter heterologous to said polynucleotide.
22. A vector comprising the nucleic acid molecule of embodiment 21.
23. The vector of embodiment 22, wherein said vector further comprises a polynucleotide encoding said tracrRNA.
24. The vector of embodiment 23, wherein said tracrRNA comprises a nucleotide sequence having at least 95% sequence identity to SEQ ID NOs: 3, 13, 21, 29, 38, 47, or 56.
25. The vector of embodiment 23 or 24, wherein said polynucleotide encoding said crRNA and said polynucleotide encoding said tracrRNA are operably linked to the same promoter and are encoded as a single guide RNA.
26. The vector of embodiment 23 or 24, wherein said polynucleotide encoding said crRNA and said polynucleotide encoding said tracrRNA are operably linked to separate promoters.
27. The vector of any one of embodiments 22-26, wherein said vector further comprises a polynucleotide encoding said RGN polypeptide, wherein said RGN polypeptide comprises an amino acid sequence having at least 95% sequence identity to SEQ ID NOs: 1, 11, 19, 27, 36, 45, or 54.
28. A nucleic acid molecule comprising a polynucleotide encoding a trans-activating CRISPR RNA (tracrRNA) comprising a nucleotide sequence having at least 95% sequence identity to SEQ ID NOs: 3, 13, 21, 29, 38, 47, or 56;
   wherein a guide RNA comprising:
      a) said tracrRNA; and
      b) a crRNA comprising a spacer sequence and a CRISPR repeat sequence, wherein said tracrRNA hybridizes with said CRISPR repeat sequence of said crRNA;
   is capable of hybridizing to a target DNA sequence in a sequence specific manner through the spacer sequence of said crRNA when said guide RNA is bound to an RNA-guided nuclease (RGN) polypeptide, and
      wherein said polynucleotide encoding a tracrRNA is operably linked to a promoter heterologous to said polynucleotide.
29. A vector comprising the nucleic acid molecule of embodiment 28.
30. The vector of embodiment 29, wherein said vector further comprises a polynucleotide encoding said crRNA.
31. The vector of embodiment 30, wherein the CRISPR repeat sequence of said crRNA comprises a nucleotide sequence having at least 95% sequence identity to SEQ ID NOs: 2, 12, 20, 28, 37, 46, or 55.
32. The vector of embodiment 30 or 31, wherein said polynucleotide encoding said crRNA and said polynucleotide encoding said tracrRNA are operably linked to the same promoter and are encoded as a single guide RNA.
33. The vector of embodiment 30 or 31, wherein said polynucleotide encoding said crRNA and said polynucleotide encoding said tracrRNA are operably linked to separate promoters.
34. The vector of any one of embodiments 29-33, wherein said vector further comprises a polynucleotide encoding said RGN polypeptide, wherein said RGN polypeptide comprises an amino acid sequence having at least 95% sequence identity to SEQ ID NOs: 1, 11, 19, 27, 36, 45, or 54.
35. A system for binding a target DNA sequence, said system comprising:
   a) one or more guide RNAs capable of hybridizing to said target DNA sequence or one or more nucleotide sequences encoding the one or more guide RNAs (gRNAs); and
   b) an RNA-guided nuclease (RGN) polypeptide comprising an amino acid sequence having at least 95% sequence identity to SEQ ID NOs: 1, 11, 19, 27, 36, 45, or 54 or a nucleotide sequence encoding the RGN polypeptide;
wherein said nucleotide sequences encoding the one or more guide RNAs and encoding the RGN polypeptide are each operably linked to a promoter heterologous to said nucleotide sequence;
   wherein the one or more guide RNAs hybridize to the target DNA sequence, and
   wherein the one or more guide RNAs form a complex with the RGN polypeptide, thereby directing said RGN polypeptide to bind to said target DNA sequence.
36. The system of embodiment 35, wherein said gRNA is a single guide RNA (sgRNA).
37. The system of embodiment 35, wherein said gRNA is a dual-guide RNA.
38. The system of any one of embodiments 35-37, wherein said gRNA comprises a CRISPR repeat sequence comprising a nucleotide sequence having at least 95% sequence identity to SEQ ID NOs: 2, 12, 20, 28, 37, 46, or 55.
39. The system of any one of embodiments 35-38, wherein said gRNA comprises a tracrRNA comprising a nucleotide sequence having at least 95% sequence identity to SEQ ID NOs: 3, 13, 21, 29, 38, 47, or 56.
40. The system of any one of embodiments 35-39, wherein said target DNA sequence is located adjacent to a protospacer adjacent motif (PAM).
41. The system of any one of embodiments 35-40, wherein the target DNA sequence is within a cell.
42. The system of embodiment 41, wherein the cell is a eukaryotic cell.
43. The system of embodiment 42, wherein the eukaryotic cell is a plant cell.
44. The system of embodiment 42, wherein the eukaryotic cell is a mammalian cell.
45. The system of embodiment 42, wherein the eukaryotic cell is an insect cell.
46. The system of embodiment 41, wherein the cell is a prokaryotic cell.
47. The system of any one of embodiments 35-46, wherein when transcribed the one or more guide RNAs hybridize to the target DNA sequence and the guide RNA forms a complex with the RGN polypeptide which causes cleavage of the target DNA sequence.
48. The system of embodiment 47, wherein the cleavage generates a double-stranded break.
49. The system of embodiment 47, wherein cleavage by said RGN polypeptide generates a single-stranded break.
50. The system of any one of embodiments 35-49, wherein the RGN polypeptide is operably linked to a base-editing polypeptide.
51. The system of any one of embodiments 35-50, wherein the RGN polypeptide comprises one or more nuclear localization signals.
52. The system of any one of embodiments 35-51, wherein the RGN polypeptide is codon optimized for expression in a eukaryotic cell.
53. The system of any one of embodiments 35-52, wherein nucleotide sequences encoding the one or more guide RNAs and the nucleotide sequence encoding an RGN polypeptide are located on one vector.
54. The system of any one of embodiments 35-53, wherein said system further comprises one or more donor polynucleotides or one or more nucleotide sequences encoding the one or more donor polynucleotides.
55. A method for binding a target DNA sequence comprising delivering a system according to any one of embodiments 35-54, to said target DNA sequence or a cell comprising the target DNA sequence.
56. The method of embodiment 55, wherein said RGN polypeptide or said guide RNA further comprises a detectable label, thereby allowing for detection of said target DNA sequence.
57. The method of embodiment 55, wherein said guide RNA or said RGN polypeptide further comprises an expression modulator, thereby modulating expression of said target DNA sequence or a gene under transcriptional control by said target DNA sequence.
58. A method for cleaving or modifying a target DNA sequence comprising delivering a system according to any one of embodiments 35-54, to said target DNA sequence or a cell comprising the target DNA sequence.
59. The method of embodiment 58, wherein said modified target DNA sequence comprises insertion of heterologous DNA into the target DNA sequence.
60. The method of embodiment 58, wherein said modified target DNA sequence comprises deletion of at least one nucleotide from the target DNA sequence.
61. The method of embodiment 58, wherein said modified target DNA sequence comprises mutation of at least one nucleotide in the target DNA sequence.
62. A method for binding a target DNA sequence comprising:
   a) assembling a RNA-guided nuclease (RGN) ribonucleotide complex in vitro by combining:
      i) one or more guide RNAs capable of hybridizing to the target DNA sequence; and
      ii) an RGN polypeptide comprising an amino acid sequence having at least 95% sequence identity to SEQ ID NOs: 1, 11, 19, 27, 36, 45, or 54;
   under conditions suitable for formation of the RGN ribonucleotide complex; and
      b) contacting said target DNA sequence or a cell comprising said target DNA sequence with the in vitro-assembled RGN ribonucleotide complex;
   wherein the one or more guide RNAs hybridize to the target DNA sequence, thereby directing said RGN polypeptide to bind to said target DNA sequence.
63. The method of embodiment 62, wherein said RGN polypeptide or said guide RNA further comprises a detectable label, thereby allowing for detection of said target DNA sequence.
64. The method of embodiment 62, wherein said guide RNA or said RGN polypeptide further comprises an expression modulator, thereby allowing for the modulation of expression of said target DNA sequence.
65. A method for cleaving and/or modifying a target DNA sequence, comprising contacting the DNA molecule with:
   a) an RNA-guided nuclease (RGN) polypeptide, wherein said RGN comprises an amino acid sequence having at least 95% sequence identity to SEQ ID NOs: 1, 11, 19, 27, 36, 45 or 54; and
   b) one or more guide RNAs capable of targeting the RGN of (a) to the target DNA sequence;
wherein the one or more guide RNAs hybridize to the target DNA sequence, thereby directing said RGN polypeptide to bind to said target DNA sequence and cleavage and/or modification of said target DNA sequence occurs.
66. The method of embodiment 65, wherein said modified target DNA sequence comprises insertion of heterologous DNA into the target DNA sequence.
67. The method of embodiment 65, wherein said modified target DNA sequence comprises deletion of at least one nucleotide from the target DNA sequence.
68. The method of embodiment 65, wherein said modified target DNA sequence comprises mutation of at least one nucleotide in the target DNA sequence.
69. The method of any one of embodiments 62-68, wherein said gRNA is a single guide RNA (sgRNA).
70. The method of any one of embodiments 62-68, wherein said gRNA is a dual-guide RNA.
71. The method of any one of embodiments 62-70, wherein said gRNA comprises a CRISPR repeat sequence comprising a nucleotide sequence having at least 95% sequence identity to SEQ ID NOs: 2, 12, 20, 28, 37, 46, or 55.
72. The method of any one of embodiments 62-71, wherein said gRNA comprises a tracrRNA comprising a nucleotide sequence having at least 95% sequence identity to SEQ ID NOs: 3, 13, 21, 29, 38, 47, or 56.
73. The method of any one of embodiments 62-72, wherein said target DNA sequence is located adjacent to a protospacer adjacent motif (PAM).
74. The method of any one of embodiments 55-73, wherein the target DNA sequence is within a cell.
75. The method of embodiment 74, wherein the cell is a eukaryotic cell.
76. The method of embodiment 75, wherein the eukaryotic cell is a plant cell.
77. The method of embodiment 75, wherein the eukaryotic cell is a mammalian cell.
78. The method of embodiment 75, wherein the eukaryotic cell is an insect cell.
79. The method of embodiment 74, wherein the cell is a prokaryotic cell.
80. The method of any one of embodiments 74-79, further comprising culturing the cell under conditions in which the RGN polypeptide is expressed and cleaves the target DNA sequence to produce a modified DNA sequence; and selecting a cell comprising said modified DNA sequence.
81. A cell comprising a modified target DNA sequence according to the method of embodiment 80.
82. The cell of embodiment 81, wherein the cell is a eukaryotic cell.
83. The cell of embodiment 82, wherein the eukaryotic cell is a plant cell.
84. A plant comprising the cell of embodiment 83.
85. A seed comprising the cell of embodiment 83.
86. The cell of embodiment 82, wherein the eukaryotic cell is a mammalian cell.
87. The cell of embodiment 82, wherein the eukaryotic cell is an insect cell.
88. The cell of embodiment 81, wherein the cell is a prokaryotic cell.
89. A method for producing a genetically modified cell with a correction in a causal mutation for a genetically inherited disease, the method comprising introducing into the cell:
   a) an RNA-guided nuclease (RGN) polypeptide, wherein the RGN polypeptide comprises an amino acid sequence having at least 95% sequence identity to SEQ ID NOs: 1, 11, 19, 27, 36, 45, or 54, or a polynucleotide encoding said RGN polypeptide, wherein said polynucleotide encoding the RGN polypeptide is operably linked to a promoter to enable expression of the RGN polypeptide in the cell; and
   b) a guide RNA (gRNA), wherein the gRNA comprises a CRISPR repeat sequence comprising a nucleotide sequence having at least 95% sequence identity to SEQ ID NOs: 2, 12, 20, 28, 37, 46, or 55, or a polynucleotide encoding said gRNA, wherein said polynucleotide encoding the gRNA is operably linked to a promoter to enable expression of the gRNA in the cell
   whereby the RGN and gRNA target to the genomic location of the causal mutation and modify the genomic sequence to remove the causal mutation.
90. The method of embodiment 89, wherein the RGN is fused to a polypeptide which has base-editing activity.
91. The method of embodiment 90, wherein the polypeptide with base-editing activity is a cytidine deaminase or an adenosine deaminase.
92. The method of embodiment 89, wherein the cell is an animal cell.
93. The method of embodiment 89, wherein the cell is a mammalian cell.
94. The method of embodiment 92, wherein the cell is derived from a dog, cat, mouse, rat, rabbit, horse, cow, pig, or human.
95. The method of embodiment 92, wherein the genetically inherited disease is a disease listed in Table 12.
96. The method of embodiment 92, wherein the genetically inherited disease is Hurler Syndrome.
97. The method of embodiment 96, wherein the gRNA further comprises a spacer sequence that targets SEQ ID NO: 453, 454, or 455.
98. A method for producing a genetically modified cell with a deletion in a disease-causing genomic region of instability, the method comprising introducing into the cell:
   a) an RNA-guided nuclease (RGN) polypeptide, wherein the RGN polypeptide comprises an amino acid sequence having at least 95% sequence identity to SEQ ID NOs: 1, 11, 19, 27, 36, 45, or 54, or a polynucleotide encoding said RGN polypeptide, wherein said polynucleotide encoding the RGN polypeptide is operably linked to a promoter to enable expression of the RGN polypeptide in the cell; and
   b) a guide RNA (gRNA), wherein the gRNA comprises a CRISPR repeat sequence comprising a nucleotide sequence having at least 95% sequence identity to SEQ ID NOs: 2, 12, 20, 28, 37, 46, or 55, or a polynucleotide encoding said gRNA, wherein said polynucleotide encoding the gRNA is operably linked to a promoter to enable expression of the gRNA in the cell, and further wherein the gRNA comprises a spacer sequence that targets the 5′flank of the genomic region of instability; and
   c) a second guide RNA (gRNA), wherein the gRNA comprises a CRISPR repeat sequence comprising a nucleotide sequence having at least 95% sequence identity to SEQ ID NOs: 2, 12, 20, 28, 37, 46, or 55, or a polynucleotide encoding said gRNA, wherein said polynucleotide encoding the gRNA is operably linked to a promoter to enable expression of the gRNA in the cell, and further wherein said second gRNA comprises a spacer sequence that targets the 3′flank of the genomic region of instability; whereby the RGN and the two gRNAs target to the genomic region of instability and at least a portion of the genomic region of instability is removed.
99. The method of embodiment 98, wherein the cell is an animal cell.
100. The method of embodiment 98, wherein the cell is a mammalian cell.
101. The method of embodiment 100, wherein the cell is derived from a dog, cat, mouse, rat, rabbit, horse, cow, pig, or human.
102. The method of embodiment 99, wherein the genetically inherited disease is Friedrich's Ataxia or Huntington's Disease.
103. The method of embodiment 102, wherein the first gRNA further comprises a spacer sequence that targets SEQ ID NO: 468, 469, or 470.
104. The method of embodiment 103, wherein the second gRNA further comprises a spacer sequence that targets SEQ ID NO: 471.
105. A method for producing a genetically modified mammalian hematopoietic progenitor cell having decreased BCL11A mRNA and protein expression, the method comprising introducing into an isolated human hematopoietic progenitor cell:
   a) an RNA-guided nuclease (RGN) polypeptide, wherein the RGN polypeptide comprises an amino acid sequence having at least 95% sequence identity to SEQ ID NOs: 1, 11, 19, 27, 36, 45, or 54, or a polynucleotide encoding said RGN polypeptide, wherein said polynucleotide encoding the RGN polypeptide is operably linked to a promoter to enable expression of the RGN polypeptide in the cell; and
   b) a guide RNA (gRNA), wherein the gRNA comprises a CRISPR repeat sequence comprising a nucleotide sequence having at least 95% sequence identity to SEQ ID NOs: 2, 12, 20, 28, 37, 46, or 55, or a polynucleotide encoding said gRNA, wherein said polynucleotide encoding the gRNA is operably linked to a promoter to enable expression of the gRNA in the cell,
   whereby the RGN and gRNA are expressed in the cell and cleave at the BCL11A enhancer region, resulting in genetic modification of the human hematopoietic progenitor cell and reducing the mRNA and/or protein expression of BCL11A.
106. The method of embodiment 105, wherein the gRNA further comprises a spacer sequence that targets SEQ ID NO: 473, 474, 475, 476, 477, or 478.
107. A system for binding a target DNA sequence, said system comprising:
   a) one or more guide RNAs capable of hybridizing to said target DNA sequence or one or more nucleotide sequences encoding the one or more guide RNAs (gRNAs); and
   b) an RNA-guided nuclease (RGN) polypeptide comprising an amino acid sequence having at least 95% sequence identity to SEQ ID NOs: 1, 11, 19, 27, 36, 45, or 54;
   wherein the one or more guide RNAs hybridize to the target DNA sequence, and
   wherein the one or more guide RNAs forms a complex with the RGN polypeptide, thereby directing said RGN polypeptide to bind to said target DNA sequence.
108. The system of embodiment 107, wherein said RGN polypeptide is nuclease dead or functions as a nickase.
109. The system of embodiment 107 or 108, wherein said RGN polypeptide is operably fused to a base-editing polypeptide.
110. The system of embodiment 109, wherein the base-editing polypeptide is a deaminase.
111. The system of embodiment 110, wherein the deaminase is a cytidine deaminase or an adenosine deaminase.

The following examples are offered by way of illustration and not by way of limitation.

### EXPERIMENTAL

### Example 1. Identification of RNA-Guided Nuclease

Seven distinct CRISPR-associated RNA-guided nucleases (RGN's) were identified and are described in Table 1 below. Table 1 provides the name of each RGN, its amino acid sequence, the source from which it was derived, and processed crRNA and tracrRNA sequences. Table 1 further provides a generic single guide RNA (sgRNA) sequence, where the poly-N indicates the location of the spacer sequence which determines the nucleic acid target sequence of the sgRNA. RGN systems APG systems APG05083.1, APG07433.1, APG08290.1, and APG08290.1 had a conserved sequence in the base of the hairpin stem of the tracrRNA, UNANNG (SEQ ID NO: 68). For the AP05459.1 system, the sequence in the same location is UNANNU (SEQ ID NO: 557). For APG04583.1 and APG01688.1 systems, the sequence is UNANNA (SEQ ID NO: 558).

TABLE 1
Summary of SEQ IDs and CRISPR associated systems

| | | | crRNA | tracrRNA | sgRNA |
|---|---|---|---|---|---|
| | SEQ | | repeat | (SEQ | (SEQ |
| | ID | | seq (SEQ | ID | ID |
| RGN ID | NO. | Source | ID NO.) | NO.) | NO) |
| APG05083.1 | 1 | Bacillus sp. | 2 | 3 | 10 |
| APG07433.1 | 10 | Bacillus sp. | 12 | 13 | 18 |
| APG07513.1 | 18 | Bacillus sp. | 20 | 21 | 26 |
| APG08290.1 | 26 | Bacillus sp. | 28 | 29 | 35 |
| APG05459.1 | 34 | Enterococcus sp. | 37 | 38 | 44 |
| APG04583.1 | 42 | Enterococcus sp. | 46 | 47 | 53 |
| APG01688.1 | 50 | Empedobacter sp. | 55 | 56 | 62 |

### Example 2: Guide RNA Identification and sgRNA Construction

Cultures of bacteria that natively express the RNA-guided nuclease system under investigation were grown to mid-log phase (OD600 of ˜0.600), pelleted, and flash frozen. RNA was isolated from the pellets using a mirVANA miRNA Isolation Kit (Life Technologies, Carlsbad, CA), and sequencing libraries were prepared from the isolated RNA using an NEBNext Small RNA Library Prep kit (NEB, Beverly, MA). The library prep was fractionated on a 6% polyacrylamide gel into 2 size fractions corresponding to 18-65nt and 90-200nt RNA species to detect crRNAs and tracrRNAs, respectively. Deep sequencing (40 bp paired-end for the smaller fraction and 80 bp paired-end for the larger fraction) was performed on a Next Seq 500 (High Output kit) by a service provider (MoGene, St. Louis, MO). Reads were quality trimmed using Cutadapt and mapped to reference genomes using Bowtie2. A custom RNAseq pipeline was written in python to detect the crRNA and tracrRNA transcripts. Processed crRNA boundaries were determined by sequence coverage of the native repeat spacer array. The anti-repeat portion of the tracrRNA was identified using permissive BLASTn parameters. RNA sequencing depth confirmed the boundaries of the processed tracrRNA by identifying the transcript containing the anti-repeat. Manual curation of RNAs was performed using secondary structure prediction by NUPACK, an RNA folding software. sgRNA cassettes were prepared by DNA synthesis and were generally designed as follows (5′→3′): 20-30 bp spacer sequence--processed repeat portion of the crRNA--4 bp noncomplementary linker (AAAG; SEQ ID NO: 63)--processed tracrRNA. Other 4 bp noncomplementary linkers may also be used, for example GAAA (SEQ ID NO: 64) or ACUU (SEQ ID NO: 65). In some instances, a 6 bp nucleotide linker may be used, for example CAAAGG (SEQ ID NO: 66). For in vitro assays, sgRNAs were synthesized by in vitro transcription of the sgRNA cassettes with a GeneArt™ Precision gRNA Synthesis Kit (ThermoFisher). Processed crRNA and tracrRNA sequences for each of the RGN polypeptides are identified and are set forth in Table 1. See below for the sgRNAs constructed for PAM libraries 1 and 2.

### Example 3: Determination of PAM Requirements for Each RGN

PAM requirements for each RGN were determined using a PAM depletion assay essentially adapted from Kleinstiver et al. (2015) Nature 523:481-485 and Zetsche et al. (2015) Cell 163:759-771. Briefly, two plasmid libraries (L1 and L2) were generated in a pUC18 backbone (ampR), with each containing a distinct 30 bp protospacer (target) sequence flanked by 8 random nucleotides (i.e., the PAM region). The target sequence and flanking PAM region of library 1 and library 2 for each RGN are set forth in Table 2.

The libraries were separately electroporated into E. coli BL21 (DE3) cells harboring pRSF-1b expression vectors containing an RGN of the invention (codon optimized for E. coli) along with a cognate sgRNA containing a spacer sequence corresponding to the protospacer in L1 or L2. Sufficient library plasmid was used in the transformation reaction to obtain>10\hat{ }6 cfu. Both the RGN and sgRNA in the pRSF-1b backbone were under the control of T7 promoters. The transformation reaction was allowed to recover for 1 hr after which it was diluted into LB media containing carbenicillin and kanamycin and grown overnight. The following day the mixture was diluted into self-inducing Overnight Express™ Instant TB Medium (Millipore Sigma) to allow expression of the RGN and sgRNA, and grown for an additional 4h or 20h after which the cells were spun down and plasmid DNA was isolated with a Mini-prep kit (Qiagen, Germantown, MD). In the presence of the appropriate sgRNA, plasmids containing a PAM that is recognizable by the RGN will be cleaved resulting in their removal from the population. Plasmids containing PAMs that are not recognizable by the RGN, or that are transformed into bacteria not containing an appropriate sgRNA, will survive and replicate. The PAM and protospacer regions of uncleaved plasmids were PCR-amplified and prepared for sequencing following published protocols (16s-metagenomic library prep guide 15044223B, Illumina, San Diego, CA). Deep sequencing (80 bp single end reads) was performed on a MiSeq (Illumina) by a service provider (MoGene, St. Louis, MO). Typically, 1-4M reads were obtained per amplicon. PAM regions were extracted, counted, and normalized to total reads for each sample. PAMs that lead to plasmid cleavage were identified by being underrepresented when compared to controls (i.e., when the library is transformed into E. coli containing the RGN but lacking an appropriate sgRNA). To represent PAM requirements for a novel RGN, the depletion ratios (frequency in sample/frequency in control) for all sequences in the region in question were converted to enrichment values with a −log base 2 transformation. Sufficient PAMs were defined as those with enrichment values>2.3 (which corresponds to depletion ratios<˜0.2). PAMs above this threshold in both libraries were collected and used to generate web logos, which for example can be generated using a web-based service on the internet known as “weblogo”. PAM sequences were identified and reported when there was a consistent pattern in the top enriched PAMs. A PAM (having an enrichment factor (EF)>2.3) for each RGN is provided in Table 2. For some RGNs, non-limiting exemplary PAMs (having an EF>3.3) were also identified. For APG005083.1, the exemplary PAM is NNRNCC (SEQ ID NO: 69). For APG007433.1, the exemplary PAM is NNNNCCR (SEQ ID NO: 70). For APG007513.1, the exemplary PAM is NNRNCC (SEQ ID NO: 71). For APG001688.1, the exemplary PAM is NNRANC (SEQ ID NO: 72).

TABLE 2
PAM determination

| | | | | Target seq | Target seq |
|---|---|---|---|---|---|
| | sgRNA | sgRNA | | and PAM | and PAM |
| | L1 | L2 | PAM | region of | region of |
| | (SEQ | (SEQ | (SEQ | plasmid | plasmid |
| | ID | ID | ID | library 1 | library 2 |
| RGN ID | NO.) | NO.) | NO.) | (SEQ ID NO.) | (SEQ ID NO.) |
| APG05083.1 | 4 | 5 | 6 | 8 | 9 |
| APG07433.1 | 13 | 14 | 6 | 16 | 17 |
| APG07513.1 | 21 | 22 | 6 | 24 | 25 |
| APG08290.1 | 29 | 30 | 32 | 33 | 34 |
| APG05459.1 | 37 | 38 | 41 | 42 | 43 |
| APG04583.1 | 45 | 46 | 50 | 51 | 52 |
| APG01688.1 | 53 | 54 | 59 | 60 | 61 |

### Example 4: Cleavage Determination

Cleavage sites were determined from in vitro cleavage reactions using RNPs (ribonucleoproteins). Expression plasmids containing an RGN fused to a His6 or a His10 tag were constructed and transformed into BL21 (DE3) strains of E. coli. Expression was performed using self-inducing media or with IPTG induction. After lysis and clarification, the proteins were purified by immobilized metal affinity chromatography.

Ribonucleoprotein complexes (comprising nuclease and an sgRNA or a crRNA and tracrRNA duplex) were formed by incubation of nuclease and the RNA in a buffered solution for 20 min at room temperature. The complex was transferred to a tube containing digestion buffer and a PCR amplified target, referred to as “Sequence 1”. Sequence 1 comprised a nucleotide sequence (SEQ ID NO: 73) directly linked at its 3′ end to the corresponding PAM sequence for each RGN. Each RGN as a ribonucleoprotein complex was incubated with its respective target polynucleotide at 25° C. (APG04583.1) or 37° C. (all others) for 30 min or 60 min (APG05459.1 and APG01688.1 only). The digestion reaction was heat inactivated and run on an agarose gel. The cleavage product bands were excised from the gel and sequenced using Sanger sequencing. Cleavage sites were identified by aligning the sequencing results with the expected sequence of the PCR product. Results are shown in Table 3. As shown in Table 3, RGN APG007433.1 may also produce a blunt cut with a different target sequence.

The cleavage site for Sequence 2 (SEQ ID NO: 559, operably fused at its 3′end to a PAM sequence for RGN APG0733.1) was determined by the following approach for the nuclease APG07433.1. After digestion, the gel purified DNA products were treated with a DNA end repair kit (Thermo Scientific K0771), ligated into linearized blunt vector, and the resulting circular DNA was transformed into E. coli competent cells. A staggered cut with a 5′ overhang would result in detection of overlapping sequences in the clones from both cleavage products. A 3′ overhang would result in missing sequence, and a blunt cut would result in all of the original sequence being detected with no overlap. This experiment also verified the finding from the above described method for sequence 1-most of the clones were detected as having originated from a cut with a 5′ overlap, so it is not expected that the finding of a blunt cut is an artifact of utilizing this method.

TABLE 3
RGN cleavage sites

| | Sequence 1 | | | Sequence 2 | | |
|---|---|---|---|---|---|---|
| | Distance from PAM | | | Distance from PAM | | |
| Nuclease | NTS cut site | TS cut site | Overhang | NTS cut site | TS cut site | Overhang |
| APG07433.1 | 4 | 3 | 1 nt, 5′ | 3 | 3 | None |

| | | | | |
|---|---|---|---|---|
| APG08290.1 | 4 | 3 | 1 nt, 5′ | Not determined |
| APG05459.1 | 3 | 3 | None | Not determined |
| APG04583.1 | 3 | 3 | None | Not determined |
| APG01688.1 | 3 | 3 | None | Not determined |

NTS = non-target strand; TS = target strand

### Example 5: Mismatch Sensitivity Assay

Plasmids were designed and obtained with a target sequence (SEQ ID NO: 73) immediately 5′ to a suitable PAM motif for the nuclease being evaluated. Single mismatch sequences were also generated with an altered sequence at the position indicated (Table 4). RNP complexes of purified nuclease (APG08290.1 or APG05459.1) and guide RNA were formed and incubated with PCR amplified linear DNA from the designed plasmids. After incubation for a designated period of time and nuclease inactivation, the samples were analyzed by agarose gel electrophoresis to determine the fraction of the linear PCR product remaining. The percentage of the intact band cleaved is shown in Table 5 for mismatches in each position.

TABLE 4
Sequences tested for the mismatch sensitivity
assay for APG08290.1 and APG05459.1

| | SEQ | |
|---|---|---|
| | ID | Mismatch |
| Protospacer sequence | NO. | position |
| GAGCGGACAGCAGCTTCCTATATCTCGTAC | 73 | None |
| GAGCGGACAGCAGCTTCCTATATCTCGTAG | 74 | 1 |
| GAGCGGACAGCAGCTTCCTATATCTCGTTC | 75 | 2 |
| GAGCGGACAGCAGCTTCCTATATCTCGAAC | 76 | 3 |
| GAGCGGACAGCAGCTTCCTATATCTCCTAC | 77 | 4 |
| GAGCGGACAGCAGCTTCCTATATCTGGTAC | 78 | 5 |
| GAGCGGACAGCAGCTTCCTATATCACGTAC | 79 | 6 |
| GAGCGGACAGCAGCTTCCTATATGTCGTAC | 80 | 7 |
| GAGCGGACAGCAGCTTCCTATAACTCGTAC | 81 | 8 |
| GAGCGGACAGCAGCTTCCTATTTCTCGTAC | 82 | 9 |
| GAGCGGACAGCAGCTTCCTAAATCTCGTAC | 83 | 10 |
| GAGCGGACAGCAGCTTCCTTTATCTCGTAC | 84 | 11 |
| GAGCGGACAGCAGCTTCCAATATCTCGTAC | 85 | 12 |
| GAGCGGACAGCAGCTTCGTATATCTCGTAC | 86 | 13 |
| GAGCGGACAGCAGCTTGCTATATCTCGTAC | 87 | 14 |
| GAGCGGACAGCAGCTACCTATATCTCGTAC | 88 | 15 |
| GAGCGGACAGCAGCATCCTATATCTCGTAC | 89 | 16 |
| GAGCGGACAGCAGGTTCCTATATCTCGTAC | 90 | 17 |
| GAGCGGACAGCACCTTCCTATATCTCGTAC | 91 | 18 |
| GAGCGGACAGCTGCTTCCTATATCTCGTAC | 92 | 19 |
| GAGCGGACAGGAGCTTCCTATATCTCGTAC | 93 | 20 |
| GAGCGGACACCAGCTTCCTATATCTCGTAC | 94 | 21 |
| GAGCGGACTGCAGCTTCCTATATCTCGTAC | 95 | 22 |
| GAGCGGAGAGCAGCTTCCTATATCTCGTAC | 96 | 23 |
| GAGCGGTCAGCAGCTTCCTATATCTCGTAC | 97 | 24 |
| GAGCGCACAGCAGCTTCCTATATCTCGTAC | 98 | 25 |
| GAGCCGACAGCAGCTTCCTATATCTCGTAC | 99 | 26 |
| GAGGGGACAGCAGCTTCCTATATCTCGTAC | 100 | 27 |
| GACCGGACAGCAGCTTCCTATATCTCGTAC | 101 | 28 |
| GTGCGGACAGCAGCTTCCTATATCTCGTAC | 102 | 29 |
| CAGCGGACAGCAGCTTCCTATATCTCGTAC | 103 | 30 |

TABLE 5
Mismatch sensitivity for RGN
APG08290.1 and RGN APG05459.1

| | % cleaved | |
|---|---|---|
| Mismatch position | APG08290.1 | APG05459.1 |
| Incompatible PAM, | 0 | 0 |
| no mismatch | | |
| No mismatch | 95 | 67 |
| 1 | 0 | 0 |
| 2 | 0 | 74 |
| 3 | 73 | 3 |
| 4 | 0 | 0 |
| 5 | 0 | 0 |
| 6 | 31 | 30 |
| 7 | 0 | 12 |
| 8 | 0 | 51 |
| 9 | 0 | 0 |
| 10 | 75 | 52 |
| 11 | 77 | 5 |
| 12 | 79 | 62 |
| 13 | 28 | 18 |
| 14 | 8 | 5 |
| 15 | 90 | 6 |
| 16 | 85 | 5 |
| 17 | 81 | 4 |
| 18 | 100 | 0 |
| 19 | 100 | 0 |
| 20 | 100 | 2 |
| 21 | 100 | 30 |
| 22 | 100 | 48 |
| 23 | 100 | 40 |
| 24 | 100 | 45 |
| 25 | 100 | 29 |
| 26 | 100 | 33 |
| 27 | 100 | 73 |
| 28 | 100 | 46 |
| 29 | 100 | 59 |
| 30 | 100 | 57 |

A similar mismatch sensitivity experiment was performed for RGN APG07433.1. This experiment was similar to the one described above, except that the alternative base was introduced into the RNA guide rather than the DNA target. DNA sequences for mismatched sgRNA synthesis are shown in Table 6. Results of the mismatch sensitivity assay are shown in Table 7.

TABLE 6
Sequences tested for the mismatch sensitivity
assay for RGN APG07433.1

| | SEQ | |
|---|---|---|
| DNA template for sgRNA | ID | Mismatch |
| synthesis | NO. | position |
| GAGCGGACAGCAGCTTCCTATATCTCGTAC | 73 | None |
| GAGCGGACAGCAGCTTCCTATATCTCGTAT | 104 | 1 |
| GAGCGGACAGCAGCTTCCTATATCTCGTGC | 105 | 2 |
| GAGCGGACAGCAGCTTCCTATATCTCGCAC | 106 | 3 |
| GAGCGGACAGCAGCTTCCTATATCTCATAC | 107 | 4 |
| GAGCGGACAGCAGCTTCCTATATCTTGTAC | 108 | 5 |
| GAGCGGACAGCAGCTTCCTATATCCCGTAC | 109 | 6 |
| GAGCGGACAGCAGCTTCCTATATTTCGTAC | 110 | 7 |
| GAGCGGACAGCAGCTTCCTATACCTCGTAC | 111 | 8 |
| GAGCGGACAGCAGCTTCCTATGTCTCGTAC | 112 | 9 |
| GAGCGGACAGCAGCTTCCTACATCTCGTAC | 113 | 10 |
| GAGCGGACAGCAGCTTCCTGTATCTCGTAC | 114 | 11 |
| GAGCGGACAGCAGCTTCCCATATCTCGTAC | 115 | 12 |
| GAGCGGACAGCAGCTTCTTATATCTCGTAC | 116 | 13 |
| GAGCGGACAGCAGCTTTCTATATCTCGTAC | 117 | 14 |
| GAGCGGACAGCAGCTCCCTATATCTCGTAC | 118 | 15 |
| GAGCGGACAGCAGCCTCCTATATCTCGTAC | 119 | 16 |
| GAGCGGACAGCAGTTTCCTATATCTCGTAC | 120 | 17 |
| GAGCGGACAGCAACTTCCTATATCTCGTAC | 121 | 18 |
| GAGCGGACAGCGGCTTCCTATATCTCGTAC | 122 | 19 |
| GAGCGGACAGTAGCTTCCTATATCTCGTAC | 123 | 20 |
| GAGCGGACAACAGCTTCCTATATCTCGTAC | 124 | 21 |
| GAGCGGACGGCAGCTTCCTATATCTCGTAC | 125 | 22 |
| GAGCGGATAGCAGCTTCCTATATCTCGTAC | 126 | 23 |

TABLE 7
Mismatch sensitivity for RGN APG07433.1

| | % cleaved |
|---|---|
| Mismatch position | APG07433.1 |
| No mismatch | 86 |
| 1 | 6 |
| 2 | 21 |
| 3 | −2 |
| 4 | 1 |
| 5 | −1 |
| 6 | 0 |
| 7 | 7 |
| 8 | 24 |
| 9 | 14 |
| 10 | −1 |
| 11 | 72 |
| 12 | 44 |
| 13 | 54 |
| 14 | 60 |
| 16 | 65 |
| 17 | 76 |
| 18 | 84 |
| 19 | 86 |
| 20 | 83 |
| 21 | 83 |
| 22 | 93 |
| 23 | 80 |

RGNs APG07433.1 and APG08290.1 show significant sensitivity to mismatches in positions 1-10 5′ from the PAM with a few exceptions (Table 5 and Table 7). RGN APG05459.1 is sensitive as well to mismatches in this region, but its ability to cleave dsDNA is also heavily abrogated by mismatches distant from the PAM site (Table 5). The total number of sites with a significant influence on whether or not cleavage occurs is at least 15 positions in the spacer sequence,. This compares favorably to other genome editing tools, such as the well-studied Cas9 nuclease from S. pyogenes, which is generally sensitive to between 10-13 base pairs (Hsu et al., Nat Biotechnol (2013) 31 (9): 827-832). Additionally, many of the critical sites abrogating RGN APG05459.1 mediated cleavage are very far from the PAM sequence, notably in the range of 13-20 bp, where many other nucleases show little if any sensitivity to mismatches. This property could be extraordinarily useful in targeting genetic loci that have close sequence similarity to other sites in the organism of interest.

### Example 6: Demonstration of Gene Editing Activity in Mammalian Cells

RGN expression cassettes were produced and introduced into vectors for mammalian expression. Each RGN was codon-optimized for human expression (SEQ ID NOs 127-133), and operably fused at the 5′end to an SV40 nuclear localization sequence (NLS; SEQ ID NO 134) and to 3×FLAG tags (SEQ ID NO: 135), and operably fused at the 3′end to nucleoplasmin NLS sequences (SEQ ID NO: 136). Each expression cassette was under control of a cytomegalovirus (CMV) promoter (SEQ ID NO: 137). It is known in the art that the CMV transcription enhancer (SEQ ID NO: 138) may also be included in constructs comprising the CMV promoter. Guide RNA expression constructs encoding a single gRNA each under the control of a human RNA polymerase III U6 promoter (SEQ ID NO. 139) were produced and introduced into the pTwist High Copy Amp vector. Sequences for the target sequences for each guide are in Table 9.

The constructs described above were introduced into mammalian cells. One day prior to transfection, 1×10⁵ HEK293T cells/well (Sigma) were plated in 24-well dishes in Dulbecco's modified Eagle medium (DMEM) plus 10% (vol/vol) fetal bovine serum (Gibco) and 1% Penicillin-Streptomycin (Gibco). The next day when the cells were at 50-60% confluency, 500 ng of a RGN expression plasmid plus 500 ng of a single gRNA expression plasmid were co-transfected using 1.5 L of Lipofectamine 3000 (Thermo Scientific) per well, following the manufacturer's instructions. After 48 hours of growth, total genomic DNA was harvested using a genomic DNA isolation kit (Machery-Nagel) according to the manufacturer's instructions.

The total genomic DNA was then analyzed to determine the rate of editing for each RGN for each genomic target. First, oligonucleotides were produced to be used for PCR amplification and subsequent analysis of the amplified genomic target site. Oligonucleotide sequences used are listed in Tables 8.1 to 8.5.

All PCR reactions were performed using 10 μL of 2× Master Mix Phusion High-Fidelity DNA polymerase (Thermo Scientific) in a 20 μL reaction including 0.5 μM of each primer. Large genomic regions encompassing each target gene were first amplified using PCR #1 primers, using a program of: 98° C., 1 min; 30 cycles of [98° C., 10 sec; 62° C., 15 sec; 72° C., 5 min]; 72° C., 5 min; 12° C., forever. One microliter of this PCR reaction was then further amplified using primers specific for each guide (PCR #2 primers), using a program of: 98° C., 1 min; 35 cycles of [98° C., 10 sec; 67° C., 15 sec; 72° C., 30 sec]; 72° C., 5 min; 12° C., forever. Primers for PCR #2 include Nextera Read 1 and Read 2 Transposase Adapter overhang sequences for Illumina sequencing.

TABLE 8.1
Oligonucleotides for detection of gene editing
activity in mammalian cells, PCR #1

| | | SEQ |
|---|---|---|
| | | ID |
| Description | Sequence | NO |
| RelA FWD | 5′-CTT AGT TTC ACC GCA GGT TCT A-3′ | 479 |
| RelA REV | 5′-CTG TGC ACT CAA CAC TGA TCT A-3′ | 480 |
| AurkB FWD | 5′-CCC AGC CCT AGG TTG TTT ATT-3′ | 481 |
| AurkB REV | 5′-CTG GCT ACA TCT TCC TTG ACT | 482 |
| | AC-3′ | |
| HPRT1 FWD | 5′-GTG GCA GAA GCA GTG AGT AA-3′ | 483 |
| HPRT1 REV | 5′-TCC CAT CTA GGC ACT AGG TAA A-3′ | 484 |

TABLE 8.2
Oligonucleotides for detection of gene editing activity
in mammalian cells, PCR#2 for APG05083.1,
APG07433.1, APG07513.1, and APG08290.1

| Description | SEQ ID NO. |
|---|---|
| FWD_Guide 134, Guide 135, Guide 136, Guide 137 | 485 |
| REV_Guide 134, Guide 135, Guide 136, Guide 137 | 486 |
| FWD_Guide 138, Guide 139, Guide 140, Guide 141 | 487 |
| REV_Guide 138, Guide 139, Guide 140, Guide 141 | 488 |
| REV_Guide 142, Guide 143, Guide 144, Guide 145 | 489 |
| FWD_Guide 142, Guide 143, Guide 144, Guide 145 | 490 |
| REV_Guide 164, Guide 165, Guide 166, Guide 167 | 491 |
| FWD_Guide 164, Guide 165, Guide 166, Guide 167 | 492 |
| REV_Guide 168, Guide 169, Guide 170, Guide 171 | 493 |
| FWD_Guide 168, Guide 169, Guide 170, Guide 171 | 494 |
| REV_Guide 172, Guide 173, Guide 174, Guide 175 | 495 |
| FWD_Guide 172, Guide 173, Guide 174, Guide 175 | 496 |
| REV_Guide 185, Guide 186, Guide 187, Guide 188 | 497 |
| FWD_Guide 185, Guide 186, Guide 187, Guide 188 | 498 |
| REV_Guide 189, Guide 190, Guide 191, Guide 192 | 499 |
| FWD_Guide 189, Guide 190, Guide 191, Guide 192 | 500 |
| REV_Guide 193, Guide 194, Guide 195, Guide 196 | 501 |
| FWD_Guide 193, Guide 194, Guide 195, Guide 196 | 502 |

TABLE 8.3
Oligonucleotides
for detection of gene editing
activity in mammalian cells,
PCR#2 for APG005459.1

| Description | SEQ ID NO. |
|---|---|
| FWD_Guide 146 | 503 |
| REV_Guide 146 | 504 |
| FWD_Guide 147 | 505 |
| REV_Guide 147 | 506 |
| REV_Guide 148 | 507 |
| FWD_Guide 148 | 508 |
| REV_Guide 176 | 509 |
| FWD_Guide 176 | 510 |
| REV_Guide 177 | 511 |
| FWD_Guide 177 | 512 |
| REV_Guide 209 | 513 |
| FWD_Guide 209 | 514 |
| REV_Guide 197 | 515 |
| FWD_Guide 197 | 516 |
| REV_Guide 198 | 517 |
| FWD_Guide 198 | 518 |
| REV_Guide 199 | 519 |
| FWD_Guide 199 | 520 |

TABLE 8.4
Oligonucleotides
for detection of gene editing activity
in mammalian cells,
PCR#2 for APG004583.1

| Description | SEQ ID NO. |
|---|---|
| FWD_Guide 149 | 521 |
| REV_Guide 149 | 522 |
| FWD_Guide 150 | 523 |
| REV_Guide 150 | 524 |
| REV_Guide 151 | 525 |
| FWD_Guide 151 | 526 |
| REV_Guide 179 | 527 |
| FWD_Guide 179 | 528 |
| REV_Guide 180 | 529 |
| FWD_Guide 180 | 530 |
| REV_Guide 181 | 531 |
| FWD_Guide 181 | 532 |
| REV_Guide 200 | 533 |
| FWD_Guide 200 | 534 |
| REV_Guide 201 | 535 |
| FWD_Guide 201 | 536 |
| REV_Guide 202 | 537 |
| FWD_Guide 202 | 538 |

TABLE 8.5
Oligonucleotides for detection of gene
editing activity in mammalian cells,
PCR#2 for APG01988.1

| Description | SEQ ID NO. |
|---|---|
| FWD_Guide 152 | 539 |
| REV_Guide 152 | 540 |
| FWD_Guide 153 | 541 |
| REV_Guide 153 | 542 |
| FWD_Guide 154 | 543 |
| REV_Guide 154 | 544 |
| FWD_Guide 182 | 545 |
| REV_Guide 182 | 546 |
| FWD_Guide 183 | 547 |
| REV_Guide 183 | 548 |
| FWD_Guide 184 | 549 |
| REV_Guide 184 | 550 |
| FWD_Guide 203 | 551 |
| REV_Guide 203 | 552 |
| FWD_Guide 204 | 553 |
| REV_Guide 204 | 554 |
| FWD_Guide 205 | 555 |
| REV_Guide 205 | 556 |

Purified genomic DNA was subjected to PCR #1 and PCR #2 as above. Following the second PCR amplification DNA was cleaned using a PCR cleanup kit (Zymo) according to the manufacturer's instructions and eluted in water. 200-500 ng of purified PCR #2 product was combined with 2 μL of 10× NEB Buffer 2 and water in a 20 μL reaction and annealed to form heteroduplex DNA using a program of: 95° C., 5 min; 95-85° C., cooled at a rate of 2° C./see; 85-25° C., cooled at a rate of 0.1° C./sec.; 12° C., forever. Following annealing 5 μL of DNA was removed as a no enzyme control, and 1 μL of T7 Endonuclease I (NEB) was added and the reaction incubated at 37° C. for 1 hr. After incubation 5× FlashGel loading dye (Lonza) was added and 5 μL of each reaction and controls were analyzed by a 2.2% agarose FlashGel (Lonza) using gel electrophoresis. Following visualization of the gel, the percentage of non-homologous end joining (NHEJ) was determined using the following equation: % NHEJ events=100×[1-(1-fraction cleaved)\hat{ }(1/2)], where (fraction cleaved) is defined as: (density of digested products)/(density of digested products+undigested parental band).

For some samples, SURVEYOR® was used to analyze the results following expression in mammalian cells. Cells were incubated at 37° C. for 72 h post-transfection before genomic DNA extraction. Genomic DNA was extracted using the QuickExtract DNA Extraction Solution (Epicentre) following the manufacturer's protocol. The genomic region flanking the RGN target site was PCR amplified, and products were purified using QiaQuick Spin Column (Qiagen) following the manufacturer's protocol. 200-500 ng total of the purified PCR products were mixed with 1 μl 10× Taq DNA Polymerase PCR buffer (Enzymatics) and ultrapure water to a final volume of 10 μl, and subjected to a re-annealing process to enable heteroduplex formation: 95° C. for 10 min, 95° C. to 85° C. ramping at −2° C./s, 85° C. to 25° C. at −0.25° C./s, and 25° C. hold for 1 min.

After reannealing, products were treated with SURVEYOR® nuclease and SURVEYOR® enhancer S (Integrated DNA Technologies) following the manufacturer's recommended protocol and analyzed on 4-20% Novex TBE polyacrylamide gels (Life Technologies). Gels were stained with SYBR Gold DNA stain (Life Technologies) for 10 min and imaged with a Gel Doc gel imaging system (Bio-rad). Quantification was based on relative band intensities. Indel percentage was determined by the formula,

100×(1−(1−(b+c)/(a+b+c))\hat{ }^{1/2}),

where a is the integrated intensity of the undigested PCR product, and b and c are the integrated intensities of each cleavage product.

Additionally, products from PCR #2 containing Illumina overhang sequences underwent library preparation following the Illumina 16S Metagenomic Sequencing Library protocol. Deep sequencing was performed on an Illumina Mi-Seq platform by a service provider (MOGene). Typically 200,000 of 250 bp paired-end reads (2×100,000 reads) are generated per amplicon. The reads were analyzed using CRISPResso (Pinello, et al. 2016 Nature Biotech, 34:695-697) to calculate the rates of editing. Output alignments were hand-curated to confirm insertion and deletion sites as well as identify microhomology sites at the recombination sites. The rates of editing are shown in Table 9. All experiments were performed in human cells. The “target sequence” is the targeted sequence within the gene target. For each target sequence, the guide RNA comprised the complementary RNA target sequence and the appropriate sgRNA depending on the RGN used. A selected breakdown of experiments by guide RNA is shown in Tables 10.1-10.9.

TABLE 9
Overall rates of editing

| | | Target | | | | |
|---|---|---|---|---|---|---|
| | | Sequence | | | | |
| | Guide | (SEQ ID | Gene | Overall Editing | Deletion Rate | Insertion Rate |
| RGN | RNA ID | NO.) | Target | Rate in Sample | in Sample | in Sample |
| APG05083.1 | 189 | 140 | RelA | 6.9% | | 100% |
| APG05083.1 | 185 | 141 | RelA | 8.2% | 79.9% | 20.1% |
| APG05083.1 | 168 | 142 | HPRT1 | 11.3% | 36.3% | 72.4% |
| APG07433.1 | 135 | 143 | AurkB | 1.7% | 88.3% | 11.7% |
| APG07433.1 | 139 | 144 | AurkB | 3.32% | 84.3% | 15.6% |
| APG07433.1 | 143 | 145 | AurkB | 2.2% | 35.1% | 64.9% |
| APG07433.1 | 190 | 146 | RelA | 60.5% | 94.8% | 5.2% |
| APG07433.1 | 194 | 147 | RelA | 6.2% | | 100% |
| APG07433.1 | 165 | 148 | HPRT1 | 3.5% | 68.0% | 32.0% |
| APG07433.1 | 169 | 149 | HPRT1 | 18.1% | 30.3% | 69.7% |
| APG07433.1 | 173 | 150 | HPRT1 | 26.6% | 91.9% | 10.0% |
| APG07513.1 | 144 | 151 | AurkB | 2.4% | 59.1% | 40.9% |
| APG07513.1 | 136 | 152 | AurkB | 0.9% | 80.5% | 19.5% |
| APG08290.1 | 145 | 153 | AurkB | 14.18% | 75.85% | 24.15% |
| APG08290.1 | 188 | 154 | RelA | 21.40% | 99.05% | 50.05% |
| APG08290.1 | 192 | 155 | RelA | 28.98% | 42.05% | 57.95% |
| APG08290.1 | 196 | 156 | RelA | 13.27% | 91.80% | 8.20% |
| APG08290.1 | 167 | 157 | HPRT1 | 14.14% | 65.98% | 34.02% |
| APG08290.1 | 171 | 158 | HPRT1 | 48.23% | 58.26% | 41.74% |
| APG08290.1 | 175 | 159 | HPRT1 | 13.60% | 74.18% | 25.82% |
| APG05459.1 | 197 | 160 | RelA | 12.95% | 92.16% | 7.84% |
| APG05459.1 | 199 | 161 | RelA | 5.19% | 100% | |
| APG05459.1 | 146 | 162 | AurkB | 1.12% | 61.50% | 38.50% |
| APG05459.1 | 148 | 163 | AurkB | 0.78% | 49.47% | 50.53% |
| APG05459.1 | 176 | 164 | HPRT1 | 6.20% | 48.91% | 51.09% |
| APG05459.1 | 177 | 165 | HPRT1 | 9.00% | 9.33% | 90.67% |
| APG05459.1 | 209 | 166 | HPRT1 | 2.50% | | 100% |
| APG04583.1 | 151 | 167 | AurkB | 0.0% | | |
| APG01688.1 | 152 | 168 | AurkB | 0.0% | | |

Specific insertions and deletions for respective guides are shown in Tables 10.1-10.7. In these tables, the target sequence is identified by bold upper case letters. The 8mer PAM regions are double underlined, with the main recognized nucleotides in bold. Insertions are identified by lowercase letters. Deletions are indicated with dashes (- - -). The INDEL location is calculated from the PAM proximal edge of the target sequence, with the edge being location 0. The location is positive (+) if the location is on the target side of the edge; the location is negative (−) if the location is on the PAM side of the edge.

TABLE 10.1
Specific insertions and deletions for Guide 139 using
RGN APG07433.1

| | # | % | % of | | INDEL | |
|---|---|---|---|---|---|---|
| Guide 139 (SEQ ID NO: 144) | Reads | Reads | INDELs | Type | Location | Size |
| CCTGGGTGTGAGGCTGGGCCATTAAAACCTC | 82540 | 95.562 | | | | |
| TCC | | | | | | |
| CCTG------------------- | 170 | 0.199 | 23.16 | Deletion | −6 | 19 |
| AAAACCTCTCC | | | | | | |
| CCTGGGTGTGA- | 132 | 0.155 | 17.98 | Deletion | +1 | 1 |
| GCTGGGCCATTAAAACCTCTCC | | | | | | |
| C------------ | 107 | 0.125 | 14.57 | Deletion | −9 | 12 |
| CTGGGCCATTAAAACCTCTCC | | | | | | |
| CCTGG----------------------- | 101 | 0.118 | 13.76 | Deletion | −5 | 23 |
| CTCTCC | | | | | | |
| C-------------- | 61 | 0.071 | 8.31 | Deletion | −9 | 14 |
| GGGCCATTAAAACCTCTCC | | | | | | |
| CCTGGGTGTGAGGccagacCTGGGCCATTAA | 49 | 0.057 | 6.67 | Insertion | +3 | 6 |
| AACCTCTCC | | | | | | |
| CCTGGGTGTGAGgggaagctgacgtcctttc | 44 | 0.051 | 5.99 | Insertion | +2 | 45 |
| catggctgctcgcctgtgttgccaccGCTGG | | | | | | |
| GCCATTAAAACCTCTCC | | | | | | |
| CCTGGGTGTGA- | 39 | 0.045 | 5.31 | Deletion & | +1 | 1 |
| cCTGGGCCATTAAAACCTCTCC | | | | Mutation | | |
| CCTGGGTGTGAGGaCTGGGCCATTAAAACCT | 31 | 0.036 | 4.22 | Insertion | +3 | 1 |
| CTCC | | | | | | |

TABLE 10.2
Specific insertions and deletions for Guide 143 using RGN APG07433.1

| | # | % | % of | | INDEL | |
|---|---|---|---|---|---|---|
| Guide 143 (SEQ ID NO: 145) | Reads | Reads | INDELs | Type | Location | Size |
| AGTTGGCAGATGCTCTAATGTACTGCCATGGGAA | 84043 | 99.646 | | | | |
| AGTTGGCAGATGC---AATGTACTGCCATGGGAA | 126 | 0.149 | 42.281 | Deletion | +3 | 3 |
| AGTTGGCAGATGC----ATGTACTGCCATGGGAA | 81 | 0.096 | 27.181 | Deletion | +3 | 3 |
| AGTTGGCAGATGCT---ATGTACTGCCATGGGAA | 42 | 0.049 | 14.093 | Deletion | +4 | 3 |
| AGTTGGCAGATGC--TAATGTACTGCCATGGGAA | 34 | 0.040 | 11.409 | Deletion | +3 | 2 |
| AGTTGGCAGATGCT---ATGTAaTGCCATGGGAA | 8 | 0.009 | 2.684 | Deletion & | +4 | 3 |
| | | | | Mutation | | |
| AGTTGGCAGATGCTCT-ATGTACTGCCATGGGAA | 7 | 0.008 | 2.348 | Deletion | +6 | 1 |

TABLE 10.3
Specific insertions and deletions for Guide 190 using RGN APG07433.1

| | # | % | % of | | INDEL | |
|---|---|---|---|---|---|---|
| Guide 190 (SEQ ID NO: 146) | Reads | Reads | INDELs | Type | Location | Size |
| CGACCTGAATGCTGTGC------------------ | 64040 | 55.46 | 91.70 | Deletion | −164 | 170 |
| ----------------------------------- | | | | | | |
| ----------------------------------- | | | | | | |
| ------------GGCGCTCTGGCTTCATTCAATC | | | | | | |
| CGACCTGAATGCTGTGCGGCTCTGCTTCCAGGTGA | 45619 | 39.51 | | WT | | |
| CGACCTGAATGCTGTGCGGCaTCTGCTTCCAGGTG | 3620 | 3.13 | 5.18 | Insertion | +3 | 1 |
| A | | | | | | |
| CGACCTGAATG----------CTGCTTCCAGGTGA | 1110 | 0.96 | 1.58 | Deletion | +2 | 10 |
| CGACCTGAATGCT-------TCTGCTTCCAGGTGA | 858 | 0.74 | 1.22 | Deletion | +3 | 7 |
| CGACCTGAA-------------TGCTTCCAGGTGA | 206 | 0.17 | 0.29 | Deletion | +1 | 13 |

TABLE 10.4
Specific insertions and deletions for Guide 194 using RGN APG07433.1

| | # | % | % of | | INDEL | |
|---|---|---|---|---|---|---|
| Guide 194 (SEQ ID NO: 147) | Reads | Reads | INDELs | Type | Location | Size |
| GCGTGGGGACTACGACCTGAATGCTGTGCGGCTCT | 96635 | 97.318 | | | | |
| GCG-----------ACCTGAATGCTGTGCGGCTCT | 1194 | 1.202 | 44.836 | Deletion | -9 | 11 |
| GCGTGGGGACTACGA-------GCTGTGCGGCTCT | 547 | 0.550 | 20.540 | Deletion | +3 | 7 |
| GCGTGGGGA-------CTGAATGCTGTGCGGCTCT | 473 | 0.476 | 17.761 | Deletion | -3 | 7 |
| GCGTGGGGACT----CCTGAATGCTGTGCGGCTCT | 270 | 0.271 | 10.138 | Deletion | -1 | 4 |
| GCGTGGGGACTACGAaCCTGAaTGCTGTGCGGCTCT | 88 | 0.088 | 3.304 | Insertion | +3 | 1 |
| GCGTGGGGACTACGA-----ATGCTGTGCGGCTCT | 41 | 0.041 | 1.539 | Deletion | +3 | 5 |
| GCGTGGGGACTAC---CTGAATGCTGTGCGGCTCT | 31 | 0.031 | 1.164 | Deletion | +2 | 3 |
| GCG--------------TGAATGCTGTGCGGCTCT | 9 | 0.009 | 0.337 | Deletion | -9 | 14 |
| GCG-----------ACCTGAcTGCTGTGCGGCTCT | 5 | 0.005 | 0.187 | Deletion | -9 | 11 |
| | | | | & | | |
| | | | | Mutation | | |
| GCGTGGGGACTACG-CCTGAATGCTGTGCGGCTCT | 5 | 0.005 | 0.187 | Deletion | +2 | 1 |

TABLE 10.5
Specific insertions and deletions for Guide 145 using RGN APG08290.1

| | # | % | % of | | INDEL | |
|---|---|---|---|---|---|---|
| Guide 145 (SEQ ID NO: 153) | Reads | Reads | INDELs | Type | Location | Size |
| ATGGAGGAGTTGGCAGATGCTCTAATGTACTGC | 62618 | 95.889 | | | | |
| CATGGGAAG | | | | | | |
| ATGGAGGAGTTGGCAGATGC-TAATGTACTGCC | 976 | 1.494 | 36.363 | Deletion | +3 | 2 |
| ATGGGAAG | | | | | | |
| ATGGAGGAGTTGGCAGATG--------TACTGC | 319 | 0.488 | 11.885 | Deletion | +2 | 8 |
| CATGGGAAG | | | | | | |
| ATG------------------------TACTGC | 168 | 0.257 | 6.259 | Deletion | −14 | 24 |
| CATGGGAAG | | | | | | |
| ATGGAGGAGTTGG------------TGTACTGC | 157 | 0.240 | 5.849 | Deletion | −4 | 12 |
| CATGGGAAG | | | | | | |
| ATGGAGGAGTTGGCAGATGCTCTaAATGTACTG | 147 | 0.225 | 5.476 | Insertion | +6 | 1 |
| CCATGGGAAG | | | | | | |
| ATGGAGGAGTTGGCAGATGCtctTCTAATGTAC | 123 | 0.188 | 4.582 | Insertion | +2 | 3 |
| TGCCATGGGAAG | | | | | | |
| ATGGAGGAGTTGGCAGATGccCTCTAATGTACT | 110 | 0.168 | 4.098 | Insertion | +2 | 2 |
| GCCATGGGAAG | | | | | | |
| ATGGAGGAGTTGGCAGAT-----AATGTACTGC | 103 | 0.157 | 3.837 | Deletion | +1 | 5 |
| CATGGGAAGAAG | | | | | | |
| ATGG---------------------cGTACTGC | 96 | 0.147 | 3.57 | Deletion & | −7 | 21 |
| CATGGGAAGAAG | | | | Mutation | | |
| ATGGAGGAGTTGGCAGATGCtTCTAATGTACTG | 85 | 0.130 | 3.166 | Insertion | +3 | 1 |
| CCATGGGAAGAAG | | | | | | |
| ATGGAGGAGTTGGCA-------------TCTGC | 84 | 0.128 | 3.129 | Deletion | −2 | 13 |
| CATGGGAAGAAG | | | | | | |
| ATGGAGGAGTTGGCAGATGC---AATGTACTGC | 79 | 0.120 | 2.943 | Deletion | +3 | 3 |
| CATGGGAAGAAG | | | | | | |
| ATGGAGGAGTTGGCAGATGCcaaactgaaaaac | 58 | 0.0884 | 2.160 | Insertion | +3 | 81 |
| aaatcaaagcactcttattgagtgctggcgatc | | | | | | |
| cccgacgccacgggccgaaacccttatcataga | | | | | | |
| aaCTCTAATGTACTGCCATGGGAAG | | | | | | |
| ATGGAGGAGTTGGCAGATGCtgcttatatagac | 53 | 0.081 | 1.974 | Insertion | +3 | 42 |
| ctcccaccgtacacgcctaccgcccatttTCTA | | | | | | |
| ATGTACTGCCATGGGAAG | | | | | | |
| ATGGAGGAGTTG--------TCTAATGTACTGC | 47 | 0.071 | 1.751 | Deletion | −5 | 8 |
| CATGGGAAG | | | | | | |
| -----------------------------CTGC | 26 | 0.039 | 0.968 | Deletion | | |
| CATGGGAAGAAG | | | | | | |
| ATGGAGGAGTTGGCAGATGCgcggctgttcctg | 21 | 0.032 | 0.782 | Insertion | +3 | 48 |
| tacagaaccgtgggcgagatgtggatcaaggat | | | | | | |
| gcTCTAATGTACTGCCATGGGAAG | | | | | | |
| ATGGAGGAGTTGGCAGATGC-CTAATGTACTGC | 14 | 0.021 | 0.521 | Deletion | +3 | 1 |
| CATGGGAAG | | | | | | |
| ATGGAGGAGTTGGCAGATGCtgtcatgatcttt | 10 | 0.015 | 0.372 | Insertion | +3 | 55 |
| ttccgctcgtcgtgggacttgctcagttctctg | | | | | | |
| gccagctcgTCTAATGTACTGCCATGGGAAG | | | | | | |
| ATGGAGGAGTTGGCAGATGCTCT-ATGTACTGC | 8 | 0.012 | 0.29 | Deletion | +6 | 1 |
| CATGGGAAG | | | | | | |

TABLE 10.6
Specific insertions and deletions for Guide 188 using RGN APG08290.1

| | | | % of | | INDEL | |
|---|---|---|---|---|---|---|
| Guide 188 (SEQ ID NO: 154) | # Reads | % Reads | INDELs | Type | Location | Size |
| CAGGGACAGTGCGCATCTCCCTGGTCACCAAG | 59686 | 97.000 | | | | |
| CAGGGACA---------------GTCACCAAG | 1286 | 2.089 | 69.664 | Deletion | 0 | 15 |
| CAGGGACAGTGCGCATCTC-CTGGTCACCAAG | 473 | 0.768 | 25.622 | Deletion | +3 | 1 |
| CAGGGACAGTGCGCATCT--CTGGTCACCAAG | 57 | 0.092 | 3.087 | Deletion | +3 | 2 |
| CAGGGACAGTGCGCATCTCCtCTGGTCACCAAG | 11 | 0.017 | 0.595 | Insertion | +3 | 1 |
| CAGGGACAGTGCGCATC---CTGGTCACCAAG | 7 | 0.011 | 0.379 | Deletion | +3 | 3 |
| CAGGGAC---------------GGTCACCAAG | 7 | 0.011 | 0.379 | Deletion | +2 | 15 |
| CGGGGACAGgGCGCATCTC-CTGGTCACCAAG | 5 | 0.008 | 0.270 | Deletion & | +3 | 1 |
| | | | | Mutation | | |

TABLE 10.7
Specific insertions and deletions for Guide 192 using RGN APG08290.1

| | | | % of | | INDEL | |
|---|---|---|---|---|---|---|
| Guide 192 (SEQ ID NO: 155) | # Reads | % Reads | INDELs | Type | Location | Size |
| CGACCTGAATGCTGTGCGGCTCTGCTTCCAGG | 62352 | 95.658 | | | | |
| CGACCTGAATGCTGTGCGGCaTCTGCTTCCAGG | 1262 | 1.936 | 44.593 | Insertion | +3 | 1 |
| CGACCTGAATGCTGTGCGGCtTCTGCTTCCAGG | 842 | 1.291 | 29.752 | Insertion | +3 | 1 |
| CGACCTGAATGCTGTG----TCTGCTTCCAGG | 686 | 1.052 | 24.240 | Deletion | +3 | 4 |
| CGACCTGcATGCTGTGCGGCaTCTGCTTCCAGG | 18 | 0.027 | 0.636 | Insertion | +3 | 1 |
| | | | | & Mutation | | |
| CGACCTGcATGCTGTGCGGCtTCTGCTTCCAGG | 11 | 0.016 | 0.388 | Insertion | +3 | 1 |
| | | | | & Mutation | | |
| CGACCTGcATGCTGTG----TCTGCTTCCAGG | 6 | 0.009 | 0.212 | Deletion | +3 | 4 |
| | | | | & Mutation | | |
| CGACCTGAATGCTGTGCGaCaTCTGCTTCCAGG | 5 | 0.007 | 0.176 | Insertion | +3 | 2 |
| | | | | & Mutation | | |

TABLE 10.8
Specific insertions and deletions for Guide 196 using RGN APG08290.1

| | | | % of | | INDEL | |
|---|---|---|---|---|---|---|
| Guide 196 (SEQ ID NO: 156) | # Reads | % Reads | INDELs | Type | Location | Size |
| TGGGGACTACGACCTGAATGCTGTGCGGCTCT | 37206 | 93.073 | | | | |
| TGGGGACTACGA-----ATGCTGTGCGGCTCT | 1288 | 3.222 | 46.514 | Deletion | +3 | 5 |
| TGGGGACTACGAgcaggcagaagtatgcaaagc | 881 | 2.203 | 31.816 | Insertion | +3 | 34 |
| atgcatctcaattCCTGAATGCTGTGCGGCTCT | | | | | | |
| TGGGGACTACGAagaaggcgatagaaggccatg | 302 | 0.755 | 10.906 | Insertion | +3 | 40 |
| cgctgcgaatcgggagcggCCTGAATGCTGTGC | | | | | | |
| GGCTCT | | | | | | |
| TGGGGACTACGAtgactcgctgcgctcggtcgt | 272 | 0.680 | 9.823 | Insertion | +3 | 67 |
| tcggctgcggcgagcggtatcagctcactcaaa | | | | | | |
| ggcggtaatacggCCTGAATGCTGTGCGGCTCT | | | | | | |
| TGGG------------------GTGCGGCTCT | 13 | 0.032 | 0.4694 | Deletion | −5 | 18 |
| TGGGGACTACGAC-----TGCTGTGCGGCTCT | 13 | 0.032 | 0.469 | Deletion | +4 | 5 |

TABLE 10.9
Specific insertions and deletions for Guide 190 using RGN APG07433.1

| | | % | % of | | INDEL | |
|---|---|---|---|---|---|---|
| Guide 190 (SEQ ID NO: 146) | #Reads | Reads | INDELs | Type | Location | Size |
| CGACCTGAATGCTGTGC------------------ | 64040 | 55.46 | 91.70 | Deletion | −164 | 170 |
| ----------------------------------- | | | | | | |
| ----------------------------------- | | | | | | |
| ----------------------------------- | | | | | | |
| ----------------------------------- | | | | | | |
| ------------GGCGCTCTGGCTTCATTCAATC | | | | | | |
| CGACCTGAATGCTGTGCGGCTCTGCTTCCAGGTGA | 45619 | 39.51 | | WT | | |
| CGACCTGAATGCTGTGCGGCaTCTGCTTCCAGGTG | 3620 | 3.13 | 5.18 | Insertion | +3 | 1 |
| A | | | | | | |
| CGACCTGAATG----------CTGCTTCCAGGTGA | 1110 | 0.96 | 1.58 | Deletion | +2 | 10 |
| CGACCTGAATGCT-------TCTGCTTCCAGGTGA | 858 | 0.74 | 1.22 | Deletion | +3 | 7 |
| CGACCTGAA-------------TGCTTCCAGGTGA | 206 | 0.17 | 0.29 | Deletion | +1 | 13 |

### Example 7: Demonstration of Gene Editing Activity in Plant Cells

RNA-guided nuclease activity of the RGNs of the invention is demonstrated in plant cells using protocols adapted from Li, et al. (2013) Nat. Biotech. 31:688-691. Briefly, plant codon optimized versions of each RGN (SEQ ID NOs: 169-182) containing an N-terminal SV40 nuclear localization signal are cloned behind the strong constitutive 35S promoter in a transient transformation vector. sgRNAs targeting one or more sites in the plant PDS gene that flank an appropriate PAM sequence are cloned behind a plant U6 promoter in a second transient expression vector. The expression vectors are introduced into Nicotiana benthamiana mesophyll protoplasts using PEG-mediated transformation. The transformed protoplasts are incubated in the dark for up to 36 hr. Genomic DNA is isolated from the protoplasts using a DNeasy Plant Mini Kit (Qiagen). The genomic region flanking the RGN target site is PCR amplified, and products are purified using QiaQuick Spin Column (Qiagen) following the manufacturer's protocol. 200-500 ng total of the purified PCR products are mixed with 1 μl 10× Taq DNA Polymerase PCR buffer (Enzymatics) and ultrapure water to a final volume of 10 μl, and subjected to a re-annealing process to enable heteroduplex formation: 95° C. for 10 min, 95° C. to 85° C. ramping at −2° C./s, 85° C. to 25° C. at −0.25° C./s, and 25° C. hold for 1 min. After reannealing, products are treated with SURVEYOR nuclease and SURVEYOR enhancer S (Integrated DNA Technologies) following the manufacturer's recommended protocol and analyzed on 4-20% Novex TBE polyacrylamide gels (Life Technologies). Gels are stained with SYBR Gold DNA stain (Life Technologies) for 10 min and imaged with a Gel Doc gel imaging system (Bio-rad). Quantification is based on relative band intensities. Indel percentage is determined by the formula, 100× (1-(1-(b+c)/(a+b+c)) 1/2), where a is the integrated intensity of the undigested PCR product, and b and c are the integrated intensities of each cleavage product.

Alternatively, PCR products derived from the targeted genomic sequence can be subjected to PCR similar to that described in Example 6, so that PCR products contain Illumina overhang sequences and can undergo library preparation and deep sequencing. This method allows determination of the rates of editing as shown in Table 9.

### Example 8: Guide Cross-Compatibility

To determine the cross-compatibility of guide RNAs between RGNs, a two-plasmid interference experiment was performed (Esvelt et al (2013), Nat. Methods 10 (11): 1116-21). The first plasmid contained the RGN with several targets containing defined PAMs on a kanamycin resistant backbone. These plasmids were transformed into E. coli BL21, and the transformed strains were made to be chemically competent. A second plasmid containing a guide RNA on an ampicillin resistance backbone was then introduced. Cells were plated on media containing both antibiotics. If an RGN is able to use the guide on the second plasmid, the kanamycin-resistance plasmid is cleaved and linearized, resulting in little or no colony formation. If an RGN is not able to use the guide on the second plasmid, the kanamycin-resistance plasmid is not be cleaved, resulting in high levels of colony formation. Guide RNAs for Streptococcus pyogenes Cas9 (SpyCas9) and Staphylococcus aureus Cas9 (SauCas9) were also included to determine cross-compatibility with those guide RNAs.

To calculate the depletion percentage, the number of colonies for each guide transformation is compared to the transformation efficiency using a positive control. Based on this comparison, if an RGN can use a guide, the depletion percentage should be 0, as no colonies are able to survive. If an RGN cannot use a guide, the depletion percentage should be 1 as all plasmids remain intact. Results are shown in Table 11 below. “sg” indicates the guide RNA for the recited RGN.

TABLE 11
Cross-compatibility assay

| | APG05083.1 | APG07513.1 | APG08290.1 | APG05459.1 | APG04583.1 | APG01688.1 |
|---|---|---|---|---|---|---|
| sgAPG05083.1 | 0 | 0 | 0 | 0.21 | 1 | 0.74 |
| sgAPG07433.1 | 0 | 0 | 0 | 0.16 | 0.78 | 0.33 |
| sgAPG07513.1 | 0 | 0.01 | 0 | 0.32 | 0.97 | 0.64 |
| sgAPG05459.1 | 0.24 | 0.53 | 0.26 | 0.09 | 1 | 0.49 |
| sgAPG04583.1 | 0.74 | 0.8 | 0.36 | 0.21 | 0 | 0 |
| sgAPG01688.1 | 0.12 | 0.26 | 0.18 | 0.43 | 0 | 0 |
| sgSauCas9 | 1 | 0.23 | 0.27 | 0.53 | 0.51 | 0.92 |
| sg Spy | 0.16 | 0.27 | 0.32 | 0.06 | 1 | 1 |

As Table 11 indicates, there are four groups of orthogonal systems. RGNs can recognize guides from other systems in their groups, but cannot use guides from other groups. The first group contains APG05083.1, APG07433.1, APG07513.1, and APG08290.1. The second group contains SpyCas9 and APG05459.1. The third group contains APG04583.1 and APG01688.1. The fourth group contains SauCas9.

### Example 9: Identification of Disease Targets

A database of clinical variants was obtained from NCBI ClinVar database, which is available through the world wide web at the NCBI Clin Var website. Pathogenic Single Nucleotide Polymorphisms (SNPs) were identified from this list. Using the genomic locus information, CRISPR targets in the region overlapping and surrounding each SNP were identified. A selection of SNPs that can be corrected using base editing in combination with the RGNs of the invention to target the causal mutation is listed in Table 12. In Table 12, only one alias of each disease is listed. The “RS #” corresponds to the RS accession number through the SNP database at the NCBI website. The AlleleID corresponds to a causal allele accession number, and the Chromosome Accession number also provides accession reference information found through the NCBI website. Table 12 also provides genomic target sequence information suitable for the RGN listed for each disease. The target sequence information also provides protospacer sequence for the production of the necessary sgRNA for the corresponding RGN of the invention.

TABLE 12
Disease Targets for RGNs of the invention

| | | | | | | | Target |
|---|---|---|---|---|---|---|---|
| | | | | | | | Seq |
| | RS# | | Causal | | | Gene | (SEQ |
| Disease | (dbSNP) | RGN | Mutation | AlleleID | ChromosomeAccession | Symbol | ID NO) |
| Ataxia-telangiectasia | 1137887 | APG04583.1 | G > A | 18083 | NC_000011.10, NC_000011.9 | ATM | 197 |
| syndrome | | | | | | | |
| Very long chain acyl-CoA | 2309689 | APG05459.1 | G > A | 33868 | NC_000017.10, NC_000017.11 | ACADVL | 198 |
| dehydrogenase deficiency | | | | | | | |
| Abnormality of T cell | 3218716 | APG01688.1 | G > A | 52071 | NC_000014.8, NC_000014.9 | MYH7 | 199 |
| physiology | | | | | | | |
| Cardiovascular phenotype | 5742905 | APG05083, | T > C | 15159 | NC_000021.8, NC_000021.9 | CBS | 200 |
| | | APG07433.1, | | | | | |
| | | APG07513.1, | | | | | |
| | | APG08290.1 | | | | | |
| 3-Oxo-5 alpha-steroid delta 4- | 9332964 | APG04583.1 | G > A | 18390 | NC_000002.11, NC_000002.12 | SRD5A2 | 201 |
| dehydrogenase deficiency | | | | | | | |
| Acute myeloid leukemia | 11540652 | APG05083, | G > A | 27395 | NC_000017.10, NC_000017.11 | TP53 | 202 |
| | | APG07433.1, | | | | | |
| | | APG07513.1, | | | | | |
| | | APG08290.1 | | | | | |
| Acute myeloid leukemia | 11540652 | APG05459.1 | G > A | 27395 | NC_000017.10, NC_000017.11 | TP53 | 203 |
| Cutaneous malignant | 11547328 | APG05459.1 | C > T | 31967 | NC_000012.11, NC_000012.12 | CDK4 | 204 |
| melanoma 3 | | | | | | | |
| Alpha-1-antitrypsin deficiency | 28929474 | APG05459.1 | G > A | 33006 | NC_000014.8, NC_000014.9 | SERPINA1 | 205 |
| Charcot-Marie-Tooth disease, | 28933093 | APG05459.1 | G > A | 29543 | NC_000001.10, NC_000001.11 | LMNA | 206 |
| type 2 | | | | | | | |
| Hereditary cancer- | 28934578 | APG05083, | G > A | 27413 | NC_000017.10, NC_000017.11 | TP53 | 207 |
| predisposing syndrome | | APG07433.1, | | | | | |
| | | APG07513.1, | | | | | |
| | | APG08290.1 | | | | | |
| Hereditary cancer- | 28934578 | APG01688.1 | G > A | 27413 | NC_000017.10, NC_000017.11 | TP53 | 208 |
| predisposing syndrome | | | | | | | |
| Hereditary cancer- | 28934872 | APG05459.1 | G > A | 27436 | NC_000016.9, NC_000016.10 | TSC2 | 209 |
| predisposing syndrome | | | | | | | |
| Brugada syndrome | 28937316 | APG05083, | G > A | 24408 | NC_000003.11, NC_000003.12 | SCN5A | 210 |
| | | APG07433.1, | | | | | |
| | | APG07513.1, | | | | | |
| | | APG08290.1 | | | | | |
| Brugada syndrome | 28937318 | APG05459.1 | G > A | 24429 | NC_000003.11, NC_000003.12 | SCN5A | 211 |
| GRACILE syndrome | 28937590 | APG05459.1 | A > G | 21206 | NC_000002.11, NC_000002.12 | BCS1L | 212 |
| Enhanced s-cone syndrome | 28937873 | APG05459.1 | G > A | 20571 | NC_000015.9, NC_000015.10 | NR2E3 | 213 |
| Charcot-Marie-Tooth disease, | 28940293 | APG05083, | T > C | 17309 | NC_000001.10, NC_000001.11 | MFN2 | 214 |
| type 2 | | APG07433.1, | | | | | |
| | | APG07513.1, | | | | | |
| | | APG08290.1 | | | | | |
| Charcot-Marie-Tooth disease, | 28940293 | APG05459.1 | T > C | 17309 | NC_000001.10, NC_000001.11 | MFN2 | 215 |
| type 2 | | | | | | | |
| Arylsulfatase a, allele a | 28940893 | APG05459.1 | C > T | 18091 | NC_000022.10, NC_000022.11 | ARSA | 216 |
| Familial hypercholesterolemia | 28942078 | APG05459.1 | G > A | 18733 | NC_000019.9, NC_000019.10 | LDLR | 217 |
| Familial hypercholesterolemia | 28942079 | APG05459.1 | G > A | 18734 | NC_000019.9, NC_000019.10 | LDLR | 218 |
| HEMOGLOBIN ARLINGTON | 33930165 | APG05083, | G > A | 30165 | NC_000011.9, NC_000011.10 | HBB | 219 |
| PARK | | APG07433.1, | | | | | |
| | | APG07513.1, | | | | | |
| | | APG08290.1 | | | | | |
| Familial hypertrophic | 36211715 | APG05459.1 | G > A | 29159 | NC_000014.8, NC_000014.9 | MYH7 | 220 |
| cardiomyopathy 1 | | | | | | | |
| Cardiovascular phenotype | 36211723 | APG05459.1 | G > A | 45266 | NC_000011.9, NC_000011.10 | MYBPC3 | 221 |
| Cardiovascular phenotype | 36211723 | APG01688.1 | G > A | 45266 | NC_000011.9, NC_000011.10 | MYBPC3 | 222 |
| Brugada syndrome | 45546039 | APG05083, | G > A | 48043 | NC_000003.11, NC_000003.12 | SCN5A | 223 |
| | | APG07433.1, | | | | | |
| | | APG07513.1, | | | | | |
| | | APG08290.1 | | | | | |
| Brugada syndrome | 45546039 | APG01688.1 | G > A | 48043 | NC_000003.11, NC_000003.12 | SCN5A | 224 |
| Hereditary cancer- | 55863639 | APG05459.1 | G > A | 176641 | NC_000017.10, NC_000017.11 | TP53 | 225 |
| predisposing syndrome | | | | | | | |
| Deficiency of butyryl-CoA | 57443665 | APG05459.1 | T > C | 18867 | NC_000012.11, NC_000012.12 | ACADS | 226 |
| dehydrogenase | | | | | | | |
| Deficiency of butyryl-CoA | 57443665 | APG01688.1 | T > C | 18867 | NC_000012.11, NC_000012.12 | ACADS | 227 |
| dehydrogenase | | | | | | | |
| Benign scapuloperoneal | 59332535 | APG05459.1 | G > A | 77828 | NC_000001.10, NC_000001.11 | LMNA | 228 |
| muscular dystrophy with | | | | | | | |
| cardiomyopathy | | | | | | | |
| Benign scapuloperoneal | 60458016 | APG05459.1 | G > A | 29564 | NC_000001.10, NC_000001.11 | LMNA | 229 |
| muscular dystrophy with | | | | | | | |
| cardiomyopathy | | | | | | | |
| Cone-rod dystrophy 6 | 61750173 | APG05459.1 | G > A | 24396 | NC_000017.10, NC_000017.11 | GUCY2D | 230 |
| Cone-rod dystrophy 6 | 61750173 | APG01688.1 | G > A | 24396 | NC_000017.10, NC_000017.11 | GUCY2D | 231 |
| Stargardt disease 1 | 61750641 | APG05083, | G > A | 105317 | NC_000001.10, NC_000001.11 | ABCA4 | 232 |
| | | APG07433.1, | | | | | |
| | | APG07513.1, | | | | | |
| | | APG08290.1 | | | | | |
| Leber congenital amaurosis 2 | 61751276 | APG05459.1 | G > A | 104715 | NC_000001.10, NC_000001.11 | RPE65 | 233 |
| Cone-rod dystrophy 3 | 61751407 | APG05459.1 | G > A | 105292 | NC_000001.10, NC_000001.11 | ABCA4 | 234 |
| Nonsyndromic | 61754375 | APG05459.1 | G > A | 18835 | NC_000011.9, NC_000011.10 | TYR | 235 |
| Oculocutaneous Albinism | | | | | | | |
| Phenylketonuria | 62508646 | APG05459.1 | T > C | 15654 | NC_000012.11, NC_000012.12 | PAH | 236 |
| Phenylketonuria | 62516101 | APG05083, | G > A | 15658 | NC_000012.11, NC_000012.12 | PAH | 237 |
| | | APG07433.1, | | | | | |
| | | APG07513.1, | | | | | |
| | | APG08290.1 | | | | | |
| Breast-ovarian cancer, familial | 62625303 | APG05459.1 | C > T | 68931 | NC_000017.10, NC_000017.11 | BRCA1 | 238 |
| 1 | | | | | | | |
| Hyperphenylalaninemia, non- | 62644499 | APG05083, | G > A | 15656 | NC_000012.11, NC_000012.12 | PAH | 239 |
| pku | | APG07433.1, | | | | | |
| | | APG07513.1, | | | | | |
| | | APG08290.1 | | | | | |
| Hyperphenylalaninemia, non- | 62644499 | APG05459.1 | G > A | 15656 | NC_000012.11, NC_000012.12 | PAH | 240 |
| pku | | | | | | | |
| Hereditary cancer- | 63750217 | APG05459.1 | G > A | 32138 | NC_000003.11, NC_000003.12 | MLH1 | 241 |
| predisposing syndrome | | | | | | | |
| Hereditary cancer- | 63750741 | APG05083, | T > C | 94663 | NC_000002.11, NC_000002.12 | MSH6 | 242 |
| predisposing syndrome | | APG07433.1, | | | | | |
| | | APG07513.1, | | | | | |
| | | APG08290.1 | | | | | |
| Hereditary cancer- | 63750809 | APG05459.1 | T > C | 95480 | NC_000003.11, NC_000003.12 | MLH1 | 243 |
| predisposing syndrome | | | | | | | |
| Hereditary cancer- | 63751657 | APG05083, | G > A | 95331 | NC_000003.11, NC_000003.12 | MLH1 | 244 |
| predisposing syndrome | | APG07433.1, | | | | | |
| | | APG07513.1, | | | | | |
| | | APG08290.1 | | | | | |
| Hereditary cancer- | 63751711 | APG05083, | G > A | 95792 | NC_000003.11, NC_000003.12 | MLH1 | 245 |
| predisposing syndrome | | APG07433.1, | | | | | |
| | | APG07513.1, | | | | | |
| | | APG08290.1 | | | | | |
| Hereditary cancer- | 63751711 | APG01688.1 | G > A | 95792 | NC_000003.11, NC_000003.12 | MLH1 | 246 |
| predisposing syndrome | | | | | | | |
| Anterior segment dysgenesis 6 | 72549387 | APG05459.1 | G > A | 22776 | NC_000002.11, NC_000002.12 | CYP1B1 | 247 |
| Brugada syndrome | 72549410 | APG05083, | G > A | 78547 | NC_000003.11, NC_000003.12 | SCN5A | 248 |
| | | APG07433.1, | | | | | |
| | | APG07513.1, | | | | | |
| | | APG08290.1 | | | | | |
| Brugada syndrome | 72549410 | APG05459.1 | G > A | 78547 | NC_000003.11, NC_000003.12 | SCN5A | 249 |
| Ornithine | 72554308 | APG01688.1 | G > A | 26053 | NC_000023.10, NC_000023.11 | OTC | 250 |
| carbamoyltransferase | | | | | | | |
| deficiency | | | | | | | |
| Osteogenesis imperfecta type | 72645321 | APG05083, | G > A | 414022 | NC_000017.10, NC_000017.11 | COL1A1 | 251 |
| I | | APG07433.1, | | | | | |
| | | APG07513.1, | | | | | |
| | | APG08290.1 | | | | | |
| Osteogenesis imperfecta type | 72645321 | APG05083, | G > A | 414022 | NC_000017.10, NC_000017.11 | COL1A1 | 252 |
| I | | APG07433.1, | | | | | |
| | | APG07513.1, | | | | | |
| | | APG08290.1 | | | | | |
| Constipation | 74799832 | APG05083, | T > C | 28958 | NC_000010.10, NC_000010.11 | RET | 253 |
| | | APG07433.1, | | | | | |
| | | APG07513.1, | | | | | |
| | | APG08290.1 | | | | | |
| Dopamine beta hydroxylase | 74853476 | APG05083, | T > C | 16789 | NC_000009.11, NC_000009.12 | DBH | 254 |
| deficiency | | APG07433.1, | | | | | |
| | | APG07513.1, | | | | | |
| | | APG08290.1 | | | | | |
| Cystic fibrosis | 75096551 | APG05083, | G > A | 33858 | NC_000007.13, NC_000007.14 | CFTR | 255 |
| | | APG07433.1, | | | | | |
| | | APG07513.1, | | | | | |
| | | APG08290.1 | | | | | |
| Phenylketonuria | 75193786 | APG01688.1 | T > C | 15675 | NC_000012.11, NC_000012.12 | PAH | 256 |
| Deficiency of UDPglucose- | 75391579 | APG05459.1 | A > G | 18653 | NC_000009.11, NC_000009.12 | GALT | 257 |
| hexose-1-phosphate | | | | | | | |
| uridylyltransferase | | | | | | | |
| Amyloid Cardiomyopathy, | 76992529 | APG05459.1 | G > A | 28465 | NC_000018.9, NC_000018.10 | TTR | 258 |
| Transthyretin-related | | | | | | | |
| Carbohydrate-deficient | 80338707 | APG01688.1 | G > A | 22758 | NC_000016.9, NC_000016.10 | PMM2 | 259 |
| glycoprotein syndrome type I | | | | | | | |
| Metachromatic | 80338815 | APG01688.1 | G > A | 18090 | NC_000022.10, NC_000022.11 | ARSA | 260 |
| leukodystrophy | | | | | | | |
| Smith-Lemli-Opitz syndrome | 80338857 | APG05083, | G > A | 34128 | NC_000011.9, NC_000011.10 | DHCR7 | 261 |
| | | APG07433.1, | | | | | |
| | | APG07513.1, | | | | | |
| | | APG08290.1 | | | | | |
| Deafness, autosomal recessive | 80338940 | APG05459.1 | G > A | 32068 | NC_000013.10, NC_000013.11 | GJB2 | 262 |
| 1A | | | | | | | |
| Congenital omphalocele | 80338945 | APG05083, | T > C | 32055 | NC_000013.10, NC_000013.11 | GJB2 | 263 |
| | | APG07433.1, | | | | | |
| | | APG07513.1, | | | | | |
| | | APG08290.1 | | | | | |
| Congenital omphalocele | 80338945 | APG05083, | T > C | 32055 | NC_000013.10, NC_000013.11 | GJB2 | 264 |
| | | APG07433.1, | | | | | |
| | | APG07513.1, | | | | | |
| | | APG08290.1 | | | | | |
| Congenital omphalocele | 80338945 | APG05459.1 | T > C | 32055 | NC_000013.10, NC_000013.11 | GJB2 | 265 |
| Congenital omphalocele | 80338945 | APG05459.1 | T > C | 32055 | NC_000013.10, NC_000013.11 | GJB2 | 266 |
| Congenital myotonia, | 80356701 | APG05083, | T > C | 33902 | NC_000007.13, NC_000007.14 | CLCN1 | 267 |
| autosomal dominant form | | APG07433.1, | | | | | |
| | | APG07513.1, | | | | | |
| | | APG08290.1 | | | | | |
| Breast-ovarian cancer, familial | 80356914 | APG05459.1 | G > A | 70276 | NC_000017.10, NC_000017.11 | BRCA1 | 268 |
| 1 | | | | | | | |
| Breast and/or ovarian cancer | 80356962 | APG05459.1 | G > A | 70247 | NC_000017.10, NC_000017.11 | BRCA1 | 269 |
| Breast-ovarian cancer, familial | 80357212 | APG05459.1 | G > A | 70255 | NC_000017.10, NC_000017.11 | BRCA1 | 270 |
| 1 | | | | | | | |
| Breast-ovarian cancer, familial | 80357281 | APG05459.1 | T > C | 70177 | NC_000017.10, NC_000017.11 | BRCA1 | 271 |
| 1 | | | | | | | |
| Breast-ovarian cancer, familial | 80357307 | APG05459.1 | G > A | 70275 | NC_000017.10, NC_000017.11 | BRCA1 | 272 |
| 1 | | | | | | | |
| Breast-ovarian cancer, familial | 80357352 | APG05459.1 | C > T | 69958 | NC_000017.10, NC_000017.11 | BRCA1 | 273 |
| 1 | | | | | | | |
| Breast-ovarian cancer, familial | 80358145 | APG05459.1 | G > A | 46229 | NC_000017.10, NC_000017.11 | BRCA1 | 274 |
| 1 | | | | | | | |
| Inborn genetic diseases | 80358259 | APG05083, | T > C | 18006 | NC_000018.9, NC_000018.10 | NPC1 | 275 |
| | | APG07433.1, | | | | | |
| | | APG07513.1, | | | | | |
| | | APG08290.1 | | | | | |
| Breast-ovarian cancer, familial | 80358543 | APG05459.1 | G > A | 131539 | NC_000013.10, NC_000013.11 | BRCA2 | 276 |
| 2 | | | | | | | |
| Breast-ovarian cancer, familial | 80358544 | APG05459.1 | G > A | 46368 | NC_000013.10, NC_000013.11 | BRCA2 | 277 |
| 2 | | | | | | | |
| Breast-ovarian cancer, familial | 80358997 | APG05459.1 | G > A | 67062 | NC_000013.10, NC_000013.11 | BRCA2 | 278 |
| 2 | | | | | | | |
| Breast and/or ovarian cancer | 80359003 | APG05083, | G > A | 67069 | NC_000013.10, NC_000013.11 | BRCA2 | 279 |
| | | APG07433.1, | | | | | |
| | | APG07513.1, | | | | | |
| | | APG08290.1 | | | | | |
| Breast-ovarian cancer, familial | 80359004 | APG05083, | G > A | 46672 | NC_000013.10, NC_000013.11 | BRCA2 | 280 |
| 2 | | APG07433.1, | | | | | |
| | | APG07513.1, | | | | | |
| | | APG08290.1 | | | | | |
| Breast-ovarian cancer, familial | 80359071 | APG05459.1 | G > A | 67203 | NC_000013.10, NC_000013.11 | BRCA2 | 281 |
| 2 | | | | | | | |
| Breast-ovarian cancer, familial | 80359112 | APG05459.1 | C > T | 67292 | NC_000013.10, NC_000013.11 | BRCA2 | 282 |
| 2 | | | | | | | |
| Breast-ovarian cancer, familial | 80359115 | APG05459.1 | C > T | 67294 | NC_000013.10, NC_000013.11 | BRCA2 | 283 |
| 2 | | | | | | | |
| Smith-Lemli-Opitz syndrome | 104886033 | APG05459.1 | A > G | 21833 | NC_000011.9, NC_000011.10 | DHCR7 | 284 |
| Alport syndrome 1, X-linked | 104886142 | APG05083, | G > A | 35796 | NC_000023.10, NC_000023.11 | COL4A5 | 285 |
| recessive | | APG07433.1, | | | | | |
| | | APG07513.1, | | | | | |
| | | APG08290.1 | | | | | |
| Acute neuronopathic | 104886460 | APG05459.1 | G > A | 99352 | NC_000001.10, NC_000001.11 | GBA | 286 |
| Gaucher's disease | | | | | | | |
| Gonadotropin deficiency | 104893836 | APG05459.1 | A > G | 31062 | NC_000004.11, NC_000004.12 | GNRHR | 287 |
| Distal arthrogryposis type 1A | 104894129 | APG05459.1 | G > A | 27501 | NC_000009.11, NC_000009.12 | TPM2 | 288 |
| Distal arthrogryposis type 1A | 104894129 | APG05459.1 | G > A | 27501 | NC_000009.11 ,NC_000009.12 | TPM2 | 289 |
| Hereditary cancer- | 104894261 | APG05459.1 | C > T | 31727 | NC_000011.9, NC_000011.10 | MEN1 | 290 |
| predisposing syndrome | | | | | | | |
| Inborn genetic diseases | 104894313 | APG05459.1 | C > T | 18816 | NC_000011.9, NC_000011.10 | TYR | 291 |
| Death in early adulthood | 104894368 | APG05083, | G > A | 29104 | NC_000012.11, NC_000012.12 | MYL2 | 292 |
| | | APG07433.1, | | | | | |
| | | APG07513.1, | | | | | |
| | | APG08290.1 | | | | | |
| Death in early adulthood | 104894368 | APG05459.1 | G > A | 29104 | NC_000012.11, NC_000012.12 | MYL2 | 293 |
| Severe autosomal recessive | 104894423 | APG05459.1 | G > A | 17048 | NC_000013.10, NC_000013.11, | SGCG | 294 |
| muscular dystrophy of | | | | | NC_000013.9 | | |
| childhood-North African type | | | | | | | |
| Cardiovascular phenotype | 104894503 | APG05459.1 | G > A | 27495 | NC_000015.9, NC_000015.10 | TPM1 | 295 |
| Carbohydrate-deficient | 104894525 | APG01688.1 | G > A | 22747 | NC_000016.9, NC_000016.10 | PMM2 | 296 |
| glycoprotein syndrome type I | | | | | | | |
| Charcot-Marie-Tooth disease, | 104894621 | APG05459.1 | C > T | 23472 | NC_000017.10, NC_000017.11 | PMP22 | 297 |
| type I | | | | | | | |
| Inborn genetic diseases | 104894635 | APG05083, | G > A | 20146 | NC_000017.10, NC_000017.11 | SGSH | 298 |
| | | APG07433.1, | | | | | |
| | | APG07513.1, | | | | | |
| | | APG08290.1 | | | | | |
| Inborn genetic diseases | 104894635 | APG05459.1 | G > A | 20146 | NC_000017.10, NC_000017.11 | SGSH | 299 |
| Familial Mediterranean fever | 104895097 | APG05083, | G > A | 17588 | NC_000016.9, NC_000016.10 | MEFV | 300 |
| | | APG07433.1, | | | | | |
| | | APG07513.1, | | | | | |
| | | APG08290.1 | | | | | |
| Deafness, autosomal recessive | 111033178 | APG05083, | G > A | 52388 | NC_000011.9, NC_000011.10 | MYO7A | 301 |
| 2 | | APG07433.1, | | | | | |
| | | APG07513.1, | | | | | |
| | | APG08290.1 | | | | | |
| Deafness, autosomal recessive | 111033178 | APG01688.1 | G > A | 52388 | NC_000011.9, NC_000011.10 | MYO7A | 302 |
| 2 | | | | | | | |
| Deafness, X-linked 2 | 111033299 | APG05083, | G > A | 53902 | NC_000013.10, NC_000013.11 | GJB2 | 303 |
| | | APG07433.1, | | | | | |
| | | APG07513.1, | | | | | |
| | | APG08290.1 | | | | | |
| Enlarged vestibular aqueduct | 111033305 | APG05083, | G > A | 52666 | NC_000007.13, NC_000007.14 | SLC26A4 | 304 |
| | | APG07433.1, | | | | | |
| | | APG07513.1, | | | | | |
| | | APG08290.1 | | | | | |
| Congenital sensorineural | 111033364 | APG05459.1, | G > A | 17396 | NC_000001.10, NC_000001.11 | USH2A | 305 |
| hearing impairment | | APG01688.1 | | | | | |
| Deficiency of UDPglucose- | 111033728 | APG05459.1 | T > C | 36556 | NC_000009.11, NC_000009.12 | GALT | 306 |
| hexose-1-phosphate | | | | | | | |
| uridylyltransferase | | | | | | | |
| Very long chain acyl-CoA | 112406105 | APG05459.1 | G > A | 200333 | NC_000017.10, NC_000017.11 | ACADVL | 307 |
| dehydrogenase deficiency | | | | | | | |
| Cardiovascular phenotype | 112645512 | APG05459.1 | C > T | 178700 | NC_000015.10, NC_000015.9 | FBN1 | 308 |
| Pyruvate kinase deficiency of | 113403872 | APG05459.1 | G > A | 16550 | NC_000001.10, NC_000001.11 | PKLR | 309 |
| red cells | | | | | | | |
| Autosomal dominant | 113994095 | APG05083, | G > A | 28535 | NC_000015.9, NC_000015.10 | POLG | 310 |
| progressive external | | APG07433.1, | | | | | |
| ophthalmoplegia with | | APG07513.1, | | | | | |
| mitochondrial DNA deletions 1 | | APG08290.1 | | | | | |
| Very long chain acyl-CoA | 113994167 | APG05459.1 | T > C | 33877 | NC_000017.10, NC_000017.11 | ACADVL | 311 |
| dehydrogenase deficiency | | | | | | | |
| Cystinosis, ocular | 113994205 | APG05459.1 | G > A | 19482 | NC_000017.10, NC_000017.11 | CTNS | 312 |
| nonnephropathic | | | | | | | |
| Pyruvate kinase deficiency of | 116100695 | APG05459.1 | C > T | 16552 | NC_000001.10, NC_000001.11 | PKLR | 313 |
| red cells | | | | | | | |
| Distal myopathy, Tateyama | 116840778 | APG01688.1 | G > A | 23322 | NC_000003.11, NC_000003.12 | CAV3; | 314 |
| type | | | | | | SSUH2 | |
| Malignant hyperthermia, | 118192122 | APG05083, | G > A | 76888 | NC_000019.9, NC_000019.10 | RYR1 | 315 |
| susceptibility to, 1 | | APG07433.1, | | | | | |
| | | APG07513.1, | | | | | |
| | | APG08290.1 | | | | | |
| Malignant hyperthermia, | 118192122 | APG05083, | G > A | 76888 | NC_000019.9, NC_000019.10 | RYR1 | 316 |
| susceptibility to, 1 | | APG07433.1, | | | | | |
| | | APG07513.1, | | | | | |
| | | APG08290.1 | | | | | |
| Myopathy, Central Core | 118192158 | APG05083, | G > A | 76835 | NC_000019.9, NC_000019.10 | RYR1 | 317 |
| | | APG07433.1, | | | | | |
| | | APG07513.1, | | | | | |
| | | APG08290.1 | | | | | |
| Myopathy, Central Core | 118192158 | APG05459.1 | G > A | 76835 | NC_000019.9, NC_000019.10 | RYR1 | 318 |
| Myopathy, Central Core | 118192158 | APG01688.1 | G > A | 76835 | NC_000019.9, NC_000019.10 | RYR1 | 319 |
| Malignant hyperthermia, | 118192170 | APG05083, | T > C | 28014 | NC_000019.9, NC_000019.10 | RYR1 | 320 |
| susceptibility to, 1 | | APG07433.1, | | | | | |
| | | APG07513.1, | | | | | |
| | | APG08290.1 | | | | | |
| Ceroid lipofuscinosis neuronal | 119455954 | APG05459.1 | G > A | 17681 | NC_000011.9, NC_000011.10 | TPP1 | 321 |
| 2 | | | | | | | |
| Ceroid lipofuscinosis neuronal | 119455954 | APG01688.1 | G > A | 17681 | NC_000011.9, NC_000011.10 | TPP1 | 322 |
| 2 | | | | | | | |
| Niemann-Pick disease type C1 | 120074135 | APG05083, | G > A | 18010 | NC_000018.9, NC_000018.10 | NPC1 | 323 |
| | | APG07433.1, | | | | | |
| | | APG07513.1, | | | | | |
| | | APG08290.1 | | | | | |
| Glutaric aciduria, type 1 | 121434372 | APG05083, | G > A | 17127 | NC_000019.9, NC_000019.10 | GCDH | 324 |
| | | APG07433.1, | | | | | |
| | | APG07513.1, | | | | | |
| | | APG08290.1 | | | | | |
| CAPN3-Related Disorders | 121434548 | APG05083, | G > A | 32661 | NC_000015.9, NC_000015.10 | CAPN3; | 325 |
| | | APG07433.1, | | | | POMT1 | |
| | | APG07513.1, | | | | | |
| | | APG08290.1 | | | | | |
| CAPN3-Related Disorders | 121434548 | APG05459.1 | G > A | 32661 | NC_000015.9, NC_000015.10 | CAPN3; | 326 |
| | | | | | | POMT1 | |
| Glycogen storage disease, type | 121907943 | APG05459.1 | C > T | 19073 | NC_000017.10, NC_000017.11 | GAA | 327 |
| II | | | | | | | |
| Nonsyndromic | 121908011 | APG05083, | G > A | 18814 | NC_000011.9, NC_000011.10 | TYR | 328 |
| Oculocutaneous Albinism | | APG07433.1, | | | | | |
| | | APG07513.1, | | | | | |
| | | APG08290.1 | | | | | |
| Familial hypercholesterolemia | 121908033 | APG05459.1 | G > A | 18765 | NC_000019.9, NC_000019.10 | LDLR | 329 |
| Familial hypercholesterolemia | 121908039 | APG05459.1 | G > A | 18778 | NC_000019.9, NC_000019.10 | LDLR | 330 |
| Deafness, autosomal recessive | 121908073 | APG05459.1 | C > T | 19142 | NC_000009.11, NC_000009.12 | TMC1 | 331 |
| 7 | | | | | | | |
| Chronic infantile neurological, | 121908153 | APG05459.1 | G > A | 19416 | NC_000001.10, NC_000001.11 | NLRP3 | 332 |
| cutaneous and articular | | | | | | | |
| syndrome | | | | | | | |
| Eichsfeld type congenital | 121908185 | APG05459.1 | G > A | 19531 | NC_000001.10, NC_000001.11 | SELENON | 333 |
| muscular dystrophy | | | | | | | |
| Inborn genetic diseases | 121908192 | APG05459.1 | G > A | 23730 | NC_000016.9, NC_000016.10 | GFER | 334 |
| Hyperkalemic Periodic | 121908557 | APG05459.1 | G > A | 20958 | NC_000017.10, NC_000017.11 | SCN4A | 335 |
| Paralysis Type 1 | | | | | | | |
| Inclusion body myopathy 2 | 121908627 | APG05459.1 | G > A | 21067 | NC_000009.11, NC_000009.12 | GNE | 336 |
| Severe | 121908716 | APG05083, | G > A | 16996 | NC_000020.10, NC_000020.11 | ADA | 337 |
| APG05083.1, APG07433.1, | | APG07433.1, | | | | | |
| APG07513.1, APG08290.1 | | APG07513.1, | | | | | |
| immunodeficiency due to ADA | | APG08290.1 | | | | | |
| deficiency | | | | | | | |
| Severe | 121908739 | APG05083, | T > C | 17004 | NC_000020.10, NC_000020.11 | ADA | 338 |
| APG05083.1, APG07433.1, | | APG07433.1, | | | | | |
| APG07513.1, APG08290.1 | | APG07513.1, | | | | | |
| immunodeficiency due to ADA | | APG08290.1 | | | | | |
| deficiency | | | | | | | |
| Cardiovascular phenotype | 121908987 | APG05459.1 | G > A | 21885 | NC_000007.13, NC_000007.14 | PRKAG2 | 339 |
| Cystic fibrosis | 121909019 | APG05459.1 | G > A | 22197 | NC_000007.13, NC_000007.14 | CFTR | 340 |
| Cystic fibrosis | 121909036 | APG05459.1 | T > C | 22247 | NC_000007.13, NC_000007.14 | CFTR | 341 |
| Adrenocortical carcinoma, | 121912664 | APG01688.1 | G > A | 27418 | NC_000017.10, NC_000017.11 | TP53 | 342 |
| pediatric | | | | | | | |
| Fumarase deficiency | 121913123 | APG05459.1 | G > A | 31275 | NC_000001.10, NC_000001.11 | FH | 343 |
| Adenocarcinoma of prostate | 121913272 | APG05459.1 | T > C | 40610 | NC_000003.11, NC_000003.12 | PIK3CA | 344 |
| Familial hypertrophic | 121913638 | APG05459.1 | G > A | 29144 | NC_000014.8, NC_000014.9 | MYH7 | 345 |
| cardiomyopathy 1 | | | | | | | |
| Adult hypophosphatasia | 121918007 | APG05083, | G > A | 28709 | NC_000001.10, NC_000001.11 | ALPL | 346 |
| | | APG07433.1, | | | | | |
| | | APG07513.1, | | | | | |
| | | APG08290.1 | | | | | |
| Adult hypophosphatasia | 121918007 | APG01688.1 | G > A | 28709 | NC_000001.10, NC_000001.11 | ALPL | 347 |
| Adult hypophosphatasia | 121918007 | APG01688.1 | G > A | 28709 | NC_000001.10, NC_000001.11 | ALPL | 348 |
| Inborn genetic diseases | 121918166 | APG05083, | G > A | 15994 | NC_000015.9, NC_000015.10 | OCA2 | 349 |
| | | APG07433.1, | | | | | |
| | | APG07513.1, | | | | | |
| | | APG08290.1 | | | | | |
| Inborn genetic diseases | 121918243 | APG05083, | G > A | 16464 | NC_000001.10, NC_000001.11 | MMACHC | 350 |
| | | APG07433.1, | | | | | |
| | | APG07513.1, | | | | | |
| | | APG08290.1 | | | | | |
| Crouzon syndrome | 121918505 | APG05459.1 | T > C | 28329 | NC_000010.10, NC_000010.11 | FGFR2 | 351 |
| Propionyl-CoA carboxylase | 121964961 | APG05459.1 | A > G | 27057 | NC_000003.11, NC_000003.12 | PCCB | 352 |
| deficiency | | | | | | | |
| Cardiovascular phenotype | 121964962 | APG05083, | G > A | 15156 | NC_000021.8, NC_000021.9 | CBS | 353 |
| | | APG07433.1, | | | | | |
| | | APG07513.1, | | | | | |
| | | APG08290.1 | | | | | |
| Dysostosis multiplex | 121965019 | APG05083, | G > A | 26947 | NC_000004.11, NC_000004.12 | IDUA | 354 |
| | | APG07433.1, | | | | | |
| | | APG07513.1, | | | | | |
| | | APG08290.1 | | | | | |
| Multiple sulfatase deficiency | 137852850 | APG05459.1 | T > C | 17711 | NC_000003.11, NC_000003.12 | SUMF1 | 355 |
| Bifunctional peroxisomal | 137853096 | APG05459.1 | G > A | 22694 | NC_000005.9, NC_000005.10 | HSD17B4 | 356 |
| enzyme deficiency | | | | | | | |
| Bifunctional peroxisomal | 137853096 | APG01688.1 | G > A | 22694 | NC_000005.9, NC_000005.10 | HSD17B4 | 357 |
| enzyme deficiency | | | | | | | |
| Hereditary cancer- | 137853293 | APG05459.1 | C > T | 28112 | NC_000013.10, NC_000013.11 | RB1 | 358 |
| predisposing syndrome | | | | | | | |
| Cardiovascular phenotype | 137854478 | APG05083, | G > A | 31496 | NC_000015.9, NC_000015.10 | FBN1 | 359 |
| | | APG07433.1, | | | | | |
| | | APG07513.1, | | | | | |
| | | APG08290.1 | | | | | |
| Cardiovascular phenotype | 137854478 | APG05083, | G > A | 31496 | NC_000015.9, NC_000015.10 | FBN1 | 360 |
| | | APG07433.1, | | | | | |
| | | APG07513.1, | | | | | |
| | | APG08290.1 | | | | | |
| Limb-girdle muscular | 137854529 | APG05459.1 | C > T | 17205 | NC_000011.9, NC_000011.10 | ANO5 | 361 |
| dystrophy, type 2L | | | | | | | |
| Familial hypercholesterolemia | 137929307 | APG01688.1 | G > A | 171217 | NC_000019.9, NC_000019.10 | LDLR | 362 |
| Spastic Paraplegia, Recessive | 141659620 | APG05459.1 | G > A | 21858 | NC_000016.9, NC_000016.10 | SPG7 | 363 |
| Isovaleryl-CoA dehydrogenase | 142761835 | APG05459.1 | G > A | 177782 | NC_000015.9, NC_000015.10 | IVD | 364 |
| deficiency | | | | | | | |
| Familial hypercholesterolemia | 145787161 | APG05459.1 | G > A | 18783 | NC_000019.10, NC_000019.9 | LDLR | 365 |
| Biotinidase deficiency | 146015592 | APG05459.1 | G > A | 46845 | NC_000003.11, NC_000003.12 | BTD | 366 |
| Biotinidase deficiency | 146015592 | APG05459.1 | G > A | 46845 | NC_000003.11, NC_000003.12 | BTD | 367 |
| Leber congenital amaurosis | 150726175 | APG01688.1 | G > A | 45795 | NC_000001.10, NC_000001.11 | NMNAT1 | 368 |
| Familial hyperinsulinism | 151344623 | APG05083, | G > A | 24127 | NC_000011.9, NC_000011.10 | ABCC8 | 369 |
| | | APG07433.1, | | | | | |
| | | APG07513.1, | | | | | |
| | | APG08290.1 | | | | | |
| Familial cancer of breast | 180177122 | APG05083, | G > A | 132185 | NC_000016.10, NC_000016.9 | PALB2 | 370 |
| | | APG07433.1, | | | | | |
| | | APG07513.1, | | | | | |
| | | APG08290.1 | | | | | |
| Cohen syndrome | 180177366 | APG05459.1 | G > A | 71322 | NC_000008.10, NC_000008.11 | VPS13B | 371 |
| Cardiovascular phenotype | 187830361 | APG05083, | T > C | 45267 | NC_000011.9, NC_000011.10 | MYBPC3 | 372 |
| | | APG07433.1, | | | | | |
| | | APG07513.1, | | | | | |
| | | APG08290.1 | | | | | |
| Wilson disease | 193922103 | APG05459.1 | A > G | 44370 | NC_000013.10, NC_000013.11 | ATP7B | 373 |
| Wilson disease | 193922110 | APG05083, | G > A | 44393 | NC_000013.10, NC_000013.11 | ATP7B | 374 |
| | | APG07433.1, | | | | | |
| | | APG07513.1, | | | | | |
| | | APG08290.1 | | | | | |
| Wilson disease | 193922110 | APG05459.1 | G > A | 44393 | NC_000013.10, NC_000013.11 | ATP7B | 375 |
| Familial hypercholesterolemia | 193922566 | APG05459.1 | G > A | 45113 | NC_000019.9, NC_000019.10 | LDLR | 376 |
| Familial hypercholesterolemia | 193922566 | APG05459.1 | G > A | 45113 | NC_000019.9, NC_000019.10 | LDLR | 377 |
| Floating-Harbor syndrome | 199469464 | APG05459.1 | C > T | 39865 | NC_000016.9, NC_000016.10 | SRCAP | 378 |
| Congenital long QT syndrome | 199472712 | APG05083, | G > A | 67758 | NC_000011.9, NC_000011.10 | KCNQ1 | 379 |
| | | APG07433.1, | | | | | |
| | | APG07513.1, | | | | | |
| | | APG08290.1 | | | | | |
| Congenital long QT syndrome | 199472712 | APG05459.1 | G > A | 67758 | NC_000011.9, NC_000011.10 | KCNQ1 | 380 |
| Andersen Tawil syndrome | 199473384 | APG01688.1 | G > A | 78481 | NC_000017.10, NC_000017.11 | KCNJ2 | 381 |
| Cardiovascular phenotype | 199473460 | APG05083, | T > C | 67776 | NC_000011.9, NC_000011.10 | KCNQ1 | 382 |
| | | APG07433.1, | | | | | |
| | | APG07513.1, | | | | | |
| | | APG08290.1 | | | | | |
| Familial hypercholesterolemia | 200238879 | APG05459.1 | T > C | 18777 | NC_000019.9, NC_000019.10 | LDLR | 383 |
| Cardiovascular phenotype | 200411226 | APG05083, | G > A | 174776 | NC_000011.9, NC_000011.10 | MYBPC3 | 384 |
| | | APG07433.1, | | | | | |
| | | APG07513.1, | | | | | |
| | | APG08290.1 | | | | | |
| Gastrointestinal stroma tumor | 201286421 | APG05459.1 | C > T | 50215 | NC_000001.10, NC_000001.11 | SDHC | 385 |
| Dyskeratosis congenita | 201540674 | APG05459.1 | G > A | 51186 | NC_000020.10, NC_000020.11 | RTEL1 | 386 |
| Dyskeratosis congenita | 201540674 | APG01688.1 | G > A | 51186 | NC_000020.10, NC_000020.11 | RTEL1 | 387 |
| Glycogen storage disease IIIa | 267606640 | APG04583.1 | G > A | 16147 | NC_000001.10, NC_000001.11 | AGL | 388 |
| Dilated cardiomyopathy 1DD | 267607004 | APG05083, | G > A | 15310 | NC_000010.10, NC_000010.11 | RBM20 | 389 |
| | | APG07433.1, | | | | | |
| | | APG07513.1, | | | | | |
| | | APG08290.1 | | | | | |
| Renal carnitine transport | 267607054 | APG05459.1 | C > T | 21466 | NC_000005.9, NC_000005.10 | SLC22A5 | 390 |
| defect | | | | | | | |
| Baraitser-Winter syndrome 1 | 281875334 | APG05083, | G > A | 38553 | NC_000007.13, NC_000007.14 | ACTB | 391 |
| | | APG07433.1, | | | | | |
| | | APG07513.1, | | | | | |
| | | APG08290.1 | | | | | |
| Very long chain acyl-CoA | 369560930 | APG05083, | G > A | 98197 | NC_000017.10, NC_000017.11 | ACADVL | 392 |
| dehydrogenase deficiency | | APG07433.1, | | | | | |
| | | APG07513.1, | | | | | |
| | | APG08290.1 | | | | | |
| Familial hypercholesterolemia | 373822756 | APG05459.1 | A > G | 181233 | NC_000019.9, NC_000019.10 | LDLR | 393 |
| Limb-girdle muscular | 376107921 | APG05459.1 | G > A | 213634 | NC_000015.9, NC_000015.10 | CAPN3 | 394 |
| dystrophy, type 2A | | | | | | | |
| Familial hypercholesterolemia | 376459828 | APG05083, | G > A | 198012 | NC_000019.10, NC_000019.9 | LDLR | 395 |
| | | APG07433.1, | | | | | |
| | | APG07513.1, | | | | | |
| | | APG08290.1 | | | | | |
| Aortic aneurysm, familial | 387906592 | APG05459.1 | G > A | 38552 | NC_000010.10, NC_000010.11 | ACTA2 | 396 |
| thoracic 6 | | | | | | | |
| Acromicric dysplasia | 387906623 | APG05083, | G > A | 38652 | NC_000015.9, NC_000015.10 | FBN1 | 397 |
| | | APG07433.1, | | | | | |
| | | APG07513.1, | | | | | |
| | | APG08290.1 | | | | | |
| Charcot-Marie-Tooth disease | 387906905 | APG01688.1 | G > A | 39430 | NC_000012.11, NC_000012.12 | TRPV4 | 398 |
| type 2C | | | | | | | |
| Breast-ovarian cancer, familial | 397507389 | APG01688.1 | G > A | 46666 | NC_000013.10, NC_000013.11 | BRCA2 | 399 |
| 2 | | | | | | | |
| Breast-ovarian cancer, familial | 397509284 | APG05459.1 | G > A | 70248 | NC_000017.10, NC_000017.11 | BRCA1 | 400 |
| 1 | | | | | | | |
| Charcot-Marie-Tooth disease | 397514494 | APG05083, | G > A | 48018 | NC_000012.11, NC_000012.12 | TRPV4 | 401 |
| type 2C | | APG07433.1, | | | | | |
| | | APG07513.1, | | | | | |
| | | APG08290.1 | | | | | |
| Charcot-Marie-Tooth disease | 397514494 | APG01688.1 | G > A | 48018 | NC_000012.11, NC_000012.12 | TRPV4 | 402 |
| type 2C | | | | | | | |
| Hereditary cancer- | 397514495 | APG05459.1 | G > A | 152034 | NC_000017.10, NC_000017.11 | TP53 | 403 |
| predisposing syndrome | | | | | | | |
| Early infantile epileptic | 397514581 | APG05083, | G > A | 48359 | NC_000020.10, NC_000020.11 | KCNQ2 | 404 |
| encephalopathy | | APG07433.1, | | | | | |
| | | APG07513.1, | | | | | |
| | | APG08290.1 | | | | | |
| Early infantile epileptic | 397514581 | APG05459.1 | G > A | 48359 | NC_000020.10, NC_000020.11 | KCNQ2 | 405 |
| encephalopathy | | | | | | | |
| Early infantile epileptic | 397514581 | APG01688.1 | G > A | 48359 | NC_000020.10, NC_000020.11 | KCNQ2 | 406 |
| encephalopathy | | | | | | | |
| Acromicric dysplasia | 397515757 | APG05459.1 | G > A | 51454 | NC_000015.9, NC_000015.10 | FBN1 | 407 |
| Hypertrophic cardiomyopathy | 397515982 | APG05083, | G > A | 51820 | NC_000011.9, NC_000011.10 | MYBPC3 | 408 |
| | | APG07433.1, | | | | | |
| | | APG07513.1, | | | | | |
| | | APG08290.1 | | | | | |
| Cardiovascular phenotype | 397516031 | APG04583.1 | G > A | 51898 | NC_000011.9, NC_000011.10 | MYBPC3 | 409 |
| Cardiovascular phenotype | 397516074 | APG05083, | G > A | 51962 | NC_000011.9, NC_000011.10 | MYBPC3 | 410 |
| | | APG07433.1, | | | | | |
| | | APG07513.1, | | | | | |
| | | APG08290.1 | | | | | |
| Cardiovascular phenotype | 397516083 | APG01688.1 | G > A | 51977 | NC_000011.9, NC_000011.10 | MYBPC3 | 411 |
| Familial hypertrophic | 397516269 | APG05083, | T > C | 52276 | NC_000014.8, NC_000014.9 | MYH7 | 412 |
| cardiomyopathy 1 | | APG07433.1, | | | | | |
| | | APG07513.1, | | | | | |
| | | APG08290.1 | | | | | |
| Benign scapuloperoneal | 397517889 | APG05459.1 | C > T | 57195 | NC_000001.10, NC_000001.11 | LMNA | 413 |
| muscular dystrophy with | | | | | | | |
| cardiomyopathy | | | | | | | |
| Glycogen storage disease, type | 398123172 | APG05083, | G > A | 415590 | NC_000017.10, NC_000017.11 | GAA | 414 |
| II | | APG07433.1, | | | | | |
| | | APG07513.1, | | | | | |
| | | APG08290.1 | | | | | |
| Diffuse mesangial sclerosis | 587776576 | APG05083, | G > A | 18532 | NC_000011.10, NC_000011.9 | WT1 | 415 |
| | | APG07433.1, | | | | | |
| | | APG07513.1, | | | | | |
| | | APG08290.1 | | | | | |
| Colobomatous | 587776954 | APG05459.1 | A > G | 51108 | NC_000012.11, NC_000012.12 | C12orf57 | 416 |
| microphthalmia | | | | | | | |
| Ataxia-telangiectasia | 587779826 | APG05459.1 | T > C | 132814 | NC_000011.10, NC_000011.9 | ATM | 417 |
| syndrome | | | | | | | |
| Familial cancer of breast | 587780226 | APG05459.1 | C > T | 133611 | NC_000017.10, NC_000017.11 | BRIP1 | 418 |
| Limb-girdle muscular | 587780290 | APG01688.1 | G > A | 134019 | NC_000015.9, NC_000015.10 | CAPN3 | 419 |
| dystrophy, type 2A | | | | | | | |
| Hereditary cancer- | 587781462 | APG05459.1 | C > T | 150772 | NC_000002.11 ,NC_000002.12 | MSH6 | 420 |
| predisposing syndrome | | | | | | | |
| Asymmetric septal | 587782958 | APG01688.1 | G > A | 165560 | NC_000011.10, NC_000011.9 | MYBPC3 | 421 |
| hypertrophy | | | | | | | |
| Hereditary cancer- | 587783050 | APG05459.1 | G > A | 166264 | NC_000016.10, NC_000016.9 | CDH1 | 422 |
| predisposing syndrome | | | | | | | |
| Familial hypertrophic | 727504247 | APG05083, | G > A | 172354 | NC_000001.10, NC_000001.11 | TNNT2 | 423 |
| cardiomyopathy 2 | | APG07433.1, | | | | | |
| | | APG07513.1, | | | | | |
| | | APG08290.1 | | | | | |
| Familial hypertrophic | 727504247 | APG01688.1 | G > A | 172354 | NC_000001.10, NC_000001.11 | TNNT2 | 424 |
| cardiomyopathy 2 | | | | | | | |
| Familial hypertrophic | 727504247 | APG01688.1 | G > A | 172354 | NC_000001.10, NC_000001.11 | TNNT2 | 425 |
| cardiomyopathy 2 | | | | | | | |
| Erythrocytosis, familial, 2 | 730882035 | APG01688.1 | G > A | 180121 | NC_000003.12, NC_000003.11 | VHL | 426 |
| Death in infancy | 730882246 | APG05083, | G > A | 181441 | NC_000014.9, NC_000014.8 | ISCA2 | 427 |
| | | APG07433.1, | | | | | |
| | | APG07513.1, | | | | | |
| | | APG08290.1 | | | | | |
| Muscular Diseases | 751995154 | APG05459.1 | G > A | 200340 | NC_000017.10, NC_000017.11 | ACADVL | 428 |
| Familial hypercholesterolemia | 756039188 | APG04583.1 | G > A | 243266 | NC_000019.9, NC_000019.10 | LDLR | 429 |
| Familial cancer of breast | 761494650 | APG05459.1 | C > T | 185659 | NC_000022.10, NC_000022.11 | CHEK2 | 430 |
| Hereditary cancer- | 762307622 | APG01688.1 | G > A | 232266 | NC_000001.10, NC_000001.11 | MUTYH | 431 |
| predisposing syndrome | | | | | | | |
| Familial hypercholesterolemia | 769370816 | APG05083, | G > A | 228176 | NC_000019.10, NC_000019.9 | LDLR | 432 |
| | | APG07433.1, | | | | | |
| | | APG07513.1, | | | | | |
| | | APG08290.1 | | | | | |
| Familial hypercholesterolemia | 775092314 | APG05083, | T > C | 228197 | NC_000019.9, NC_000019.10 | LDLR | 433 |
| | | APG07433.1, | | | | | |
| | | APG07513.1, | | | | | |
| | | APG08290.1 | | | | | |
| Familial hypercholesterolemia | 775924858 | APG05083, | G > A | 246116 | NC_000019.9, NC_000019.10 | LDLR | 434 |
| | | APG07433.1, | | | | | |
| | | APG07513.1, | | | | | |
| | | APG08290.1 | | | | | |
| Inclusion body myopathy 2 | 779694939 | APG04583.1 | T > C | 214934 | NC_000009.12, NC_000009.11 | GNE | 435 |
| Ataxia-telangiectasia | 780619951 | APG05459.1 | C > T | 212851 | NC_000011.10, NC_000011.9 | ATM | 436 |
| syndrome | | | | | | | |
| Benign familial neonatal- | 794727152 | APG04583.1 | G > A | 191718 | NC_000002.11, NC_000002.12 | SCN2A | 437 |
| infantile seizures | | | | | | | |
| Marfan Syndronne/Loeys-Dietz | 794728228 | APG05459.1 | C > T | 197690 | NC_000015.10, NC_000015.9 | FBN1 | 438 |
| Syndrome/Familial Thoracic | | | | | | | |
| Aortic Aneurysms and | | | | | | | |
| Dissections | | | | | | | |
| Dilated cardiomyopathy 1G | 869320740 | APG01688.1 | T > C | 136355 | NC_000002.11, NC_000002.12 | TTN | 439 |
| Familial hypercholesterolemia | 875989911 | APG05459.1 | G > A | 228151 | NC_000019.9, NC_000019.10 | LDLR | 440 |
| Breast-ovarian cancer, familial | 876657678 | APG05459.1 | C > T | 230443 | NC_000013.10, NC_000013.11 | BRCA2 | 441 |
| 2 | | | | | | | |
| Familial hypercholesterolemia | 879254600 | APG05459.1 | G > A | 245669 | NC_000019.10, NC_000019.9 | LDLR | 442 |
| Familial hypercholesterolemia | 879254803 | APG05083, | T > C | 246008 | NC_000019.10, NC_000019.9 | LDLR | 443 |
| | | APG07433.1, | | | | | |
| | | APG07513.1, | | | | | |
| | | APG08290.1 | | | | | |
| Familial hypercholesterolemia | 879254803 | APG01688.1 | T > C | 246008 | NC_000019.10, NC_000019.9 | LDLR | 444 |
| Familial hypercholesterolemia | 879254849 | APG01688.1 | T > C | 246074 | NC_000019.10, NC_000019.9 | LDLR | 445 |
| Familial cancer of breast | 1057517585 | APG01688.1 | G > A | 358911 | NC_000016.10, NC_000016.9 | PALB2 | 446 |
| Hereditary hemorrhagic | 1057517944 | APG05459.1 | C > T | 360048 | NC_000012.11, NC_000012.12 | ACVRL1 | 447 |
| telangiectasia type 2 | | | | | | | |

### Example 10: Targeting Mutations Responsible for Hurler Syndrome

The following describes a potential treatment for Hurler Syndrome, also referred to as MPS-1, is described, using an RNA directed base editing system that corrects a mutation responsible for Hurler syndrome in a large proportion of patients with the disease. This approach utilizes a base editing fusion protein that is RNA guided and that can be packaged into a single AAV vector for delivery to a wide range of tissue types. Depending on the exact regulatory elements and base editor domain used, it may also be possible to engineer a single vector that encodes for both the base editing fusion protein and a single guide RNA to target the diseased locus.

### Example 10.1: Identifying RGN with Ideal PAM

The genetic disease MPS-1 is a lysosomal storage disease characterized at the molecular level by the accumulation of dermatan sulfate and heparan sulfate in lysosomes. This disease is generally an inherited genetic disorder caused by mutations in the IDUA gene (NCBI Reference sequence NG_008103.1), which encodes α-L-iduronidase. The disease is a result of a deficiency of α-L-iduronidase. The most common IDUA mutations found in studies of individuals of Northern European background are W402× and Q70X, both nonsense mutations resulting in premature termination of translation (Bunge et al. (1994), Hum. Mol. Genet, 3 (6): 861-866, herein incorporated by reference). Reversion of a single nucleotide would restore the wild-type coding sequence and result in protein expression controlled by the endogenous regulatory mechanisms of the genetic locus.

The W402× mutation of the human Idua gene accounts for a high proportion of MPS-1H cases. Base editors can target a narrow sequence window relative to the binding site of the protospacer component of the guide RNA and thus the presence of a PAM sequence a specific distance from the target locus is essential for the success of the strategy. Given the constraints that the target mutation must be on the exposed non-target strand (NTS) during the interaction of the base editing protein and that the footprint of the RGN domain will block access to the region near the PAM, an accessible locus is thought to be 10-30 bp from the PAM. To avoid editing and mutagenesis of other nearby adenosine bases in this window, different linkers are screened. The ideal window is 12-16 bp from the PAM.

A PAM sequence compatible with APG07433.1 and APG08290.1 is readily apparent at the genetic locus and within the ideal base editing window as defined above. These nucleases have a PAM sequence of NNNNCC (SEQ ID NO: 6) and NNRNCC (SEQ ID NO: 32), respectively, and are compact in size-potentially allowing delivery via a single AAV vector. This delivery approach bestows multiple advantages relative to others, such as access to a wide range of tissues (liver, muscle, CNS) and well-established safety profile and manufacturing techniques.

Cas9 from S. pyogenes (SpyCas9) requires a PAM sequence of NGG (SEQ ID NO: 448), which is present near the W402× locus, but the size of SpyCas9 prevents packaging of a gene encoding a fusion protein of a base editing domain and the SpyCas9 nuclease into a single AAV vector, and thus forgoes the aforementioned advantages of this approach. Including a guide RNA encoding sequence on this vector would is even less feasible, even if there are to be significant technological improvements that reduce the size of gene regulatory elements or increase the packaging limits of AAV vectors. While a dual delivery strategy may be employed (for example, Ryu et al, (2018), Nat. Biotechnol., 36 (6): 536-539, herein incorporated by reference), it would add significant manufacturing complexity and cost. Additionally, dual viral vector delivery significantly decreases the efficiency of gene correction, since a successful edit in a given cell requires infection with both vectors and assembly of the fusion protein in the cell. A commonly used Cas9 ortholog from S. aureus (SauCas9) is considerably smaller in size relative to SpyCas9, but has a more complex PAM requirement-NGRRT (SEQ ID NO: 449). This sequence, however, is not within a range expected to be useful for base editing of the causative locus.

### Example 10.2: RGN Fusion Constructs and sgRNA Sequences

A DNA sequence encoding a fusion protein with the following domains is produced using standard molecular biology techniques: 1) an RGN domain with mutations that inactivate the DNA cleavage activity (“dead” or “nickase”); 2) an adenosine deaminase useful for base editing. All constructs described in the table below comprise a fusion protein with the base editing active domain, in this example ADAT (SEQ ID NO: 450) operably fused to the N-terminal end of the RGN APG08290.1. It is known in the art that a fusion protein could also be made with the base-editing enzyme at the C-terminal end of the RGN. Additionally, the RGN and the base editor of the fusion protein are typically separated by a linker amino sequence. It is known in the art that lengths of standard linkers range from 15-30 amino acids. Further, it is known in the art that certain fusion proteins between an RGN and a base-editing enzyme, for example a cytidine deaminase, may also comprise at least one uracil glycosylase inhibitor (UGI) domain, which may increase base editing efficiency (U.S. Pat. No. 10,167,457, herein incorporated by reference). Therefore, a fusion protein may comprise APG08290.1, a base-modifying enzyme, and at least one UGI.

TABLE 13
Constructs for RNA-targeted base editing

| | | | Dead | | |
|---|---|---|---|---|---|
| Seq | | | (D) or | | |
| ID | | | Nickase | Base | |
| No. | Construct | RGN | (N) | editor | Linker |
| 451 | Nuc-ADAT- | APG08290.1 | D | ADAT | XTEN1 |
| | Linker- | | | | |
| | dAPG08290.1- | | | | |
| | Linker-SV40 | | | | |
| 452 | Nuc-ADAT- | APG08290.1 | N | ADAT | XTEN1 |
| | XTEN1- | | | | |
| | nAPG08290.1- | | | | |
| | Linker-SV40 | | | | |

The accessible editing sites of an RGN are determined by the PAM sequence. When combining an RGN with a base editing domain, the target residue for editing must reside on the non-target strand (NTS), since the NTS is single stranded while the RGN is associated with the locus. Evaluating a number of nucleases and corresponding guide RNAs enables the selection of the most appropriate gene editing tool for this particular locus. Several potential PAM sequences that can be targeted by the constructs described above in the human Idua gene are in the proximity of the mutant nucleotide responsible for the W402× mutation. A sequence encoding a guide RNA transcript containing 1) a “spacer” that is complementary to the non-coding DNA strand at the disease locus; and 2) RNA sequence required for association of the guide RNA with the RGN is also produced. Useful guide RNA sequences (sgRNA) are shown in Table 14 below. These guide RNA sequences can be evaluated for their efficiency in directing the base editors above to the locus of interest.

TABLE 14
Sequence of guide RNAs

| | | Coding |
|---|---|---|
| | SEQ | sequence |
| Sequence of target | ID | of sgRNA |
| genomic sequence | NO. | (SEQ ID NO.) |
| 5′-GGAGCAGCTCTAGGCCGAAGTGTCG-3′ | 453 | 456 |
| 5′-TAGGCCGAAGTGTCGCAGGCCGGGA-3′ | 454 | 457 |
| 5′-GCTCTAGGCCGAAGTGTCGCAGGCC-3′ | 455 | 458 |

### Example 10.3: Assay for Activity in Cells from Hurler Disease Patients

To verify the genotype strategy and evaluate the constructs described above, fibroblasts from Hurler disease patients are used. A vector is designed containing appropriate promoters upstream of the fusion protein coding sequence and the sgRNA encoding sequence for expression of these in human cells, similar to those vectors described in Example 5. It is recognized that promoters and other DNA elements (for example enhancers, or terminators) which either are known for high levels of expression in human cells or may specifically express well in fibroblast cells may also be used. The vector is transfected into the fibroblasts using standard techniques, for example transfection similar to what is described in Example 6. Alternatively, electroporation may be used. The cells are cultured for 1-3 days. Genomic DNA (gDNA) is isolated using standard techniques. The editing efficiency is determined by performing a qPCR genotyping assay and/or next generation sequencing on the purified gDNA, as described further below.

Taqman™ qPCR analysis utilizes probes specific for the wild-type and mutant allele. These probes bear fluorophores which are resolved by their spectral excitation and/or emission properties using a qPCR instrument. A genotyping kit containing PCR primers and probes can be obtained commercially (i.e. Thermo Fisher Taqman™ SNP genotyping assayID C_27862753_10 for SNP ID rs121965019) or designed. An example of a designed primer and probe set is shown in Table 15.

TABLE 15
RT-PCR primers and probes

| | | SEQ ID |
|---|---|---|
| Description | Sequence | NO. |
| Forward Amplification | 5′-GACTCCTTCACCAAG-3′ | 459 |
| Primer | | |
| Reverse Amplification | 5′-GTAGATCAGCACCG-3′ | 460 |
| Primer | | |
| Wild Type Probe | 5′-CTCTGGGCCGAAGT-3′ | 461 |
| W402X Probe | 5′-CTCTAGGCCGAAGT-3′ | 462 |

Following the editing experiment, the gDNA is subjected to qPCR analysis using standard methods and the primers and probes described above. Expected results are shown in Table 16. This in vitro system can be used to expediently evaluate constructs and choose one with high editing efficiency for further studies. The systems will be evaluated in comparison with cells with and without the W402× mutation, and preferably with some that are heterozygous for this mutation. The Ct values will be compared to either a reference gene or the total amplification of the locus using a dye such as Sybr green.

TABLE 16
Expected qPCR results

| | Transfected with | |
|---|---|---|
| Genotype | base editor | Expected PCR result |
| Idua^{WT/WT} | No | Homozygous WT |
| Idua^{WT/W402X} | No | Heterozygous: 50% WT, |
| | | 50% W402X |
| Idua^{W402X/W402X} | No | Homozygous W402X |
| Idua^{W402X/W402X} | Yes | Variable |

The tissues can also be analyzed by next generation sequencing. Primer binding sites such as the ones shown below (Table 17), or other suitable primer binding sites that can be identified by a person of skill in the art, can be used. Following PCR amplification, products containing Illumina Nextera XT overhang sequences undergo library preparation following the Illumina 16S Metagenomic Sequencing Library protocol. Deep sequencing is performed on an Illumina Mi-Seq platform. Typically, 200,000 of 250 bp paired-end reads (2×100,000 reads) are generated per amplicon. The reads are analyzed using CRISPResso (Pinello et al., 2016) to calculate the rates of editing. Output alignments are hand-curated to confirm insertion and deletion sites as well as identify microhomology sites at the recombination sites.

TABLE 17
NGS primer binding sites

| | | SEQ ID |
|---|---|---|
| Direction | Sequence | NO. |
| Forward | 5′-ACTTCCTCCAGCC-3′ | 463 |
| Reverse | 5′-GAACCCCGGCTTA-3′ | 464 |

Western blotting of cell lysate of transfected cells and control cells using an anti-IDUA antibody is performed to verify expression of the full-length protein and an enzyme activity assay on the cell lysate using substrate 4-methylumbelliferyl α-L-iduronide verifies that the enzyme is catalytically active (Hopwood et al., Clin.Chim. ACta (1979), 92 (2): 257-265, incorporated by reference herein). These experiments are performed in comparison with the original Idua^{W402X/W402X} cell line (without transfection), the Idua^{W402X/W402X} cell line transfected with the base editing construct and a random guide sequence, and a cell line expressing wild-type IDUA.

### Example 10.4: Disease Treatment Validation in a Murine Model

To verify the efficacy of this therapeutic approach, a mouse model with a nonsense mutation in the analogous amino acid is used. The mouse strain bears a W392× mutation in its Idua gene (Gene ID: 15932) which corresponds to the homologous mutation in Hurler syndrome patients (Bunge et al., (1994), Hum. Mol. Genet. 3 (6): 861-866, incorporated by reference herein). This locus comprises a distinct nucleotide sequence relative to that in humans, which lacks the PAM sequence necessary for correction with the base editors described in the previous examples, and thus necessitates design of a distinct fusion protein to perform the nucleotide correction. Amelioration of the disease in this animal can validate the therapeutic approach of correcting the mutation in tissues accessible by a gene delivery vector.

Mice homozygous for this mutation display a number of phenotypic characteristics similar to Hurler syndrome patients. A base editing-RGN fusion protein as described above (Table 13) along with an RNA guide sequence are incorporated into an expression vector that allows protein expression and RNA transcription in mice. A study design is shown below in Table 18. The study includes groups that are treated with a high dose of the expression vector comprising the base-editing fusion protein and RNA guide sequence, a low dose of same expression vector, control which is the model mouse treated with an expression vector that does not comprise the base editing fusion protein or the guide RNA, and a second control which is a wild type mouse treated with the same empty vector.

TABLE 18
Genome editing experiment in murine model

| Group | Mouse strain | N | Treatment |
|---|---|---|---|
| 1 | Idua-W392X¹ | ≥5 | Low dose of vector |
| 2 | Idua-W392X | ≥5 | High dose of vector |
| 3 | Idua-W392X | ≥5 | Vehicle |
| 4 | 129/Sv (WT) | 5 | Vehicle |

Endpoints to evaluate include body weight, urine GAG excretion, serum IDUA enzymatic activity, IDUA activity in tissues of interest, tissue pathology, genotyping of tissues of interest to verify correction of the SNP, and behavioral and neurological evaluation. Since some endpoints are terminal, additional groups may be added for evaluation of, for example, tissue pathology and tissue IDUA activities before the end of the study. Additional examples of endpoints can be found in published papers establishing Hurler syndrome animal models (Shull et al. (1994), Proc. Natl. Acad. Sci. U.S.A., 91 (26): 12937-12941; Wang et al. (2010), Mol. Genet. Metab., 99 (1): 62-71; Hartung et al. (2004), Mol. Ther., 9 (6): 866-875; Liu et al. (2005), Mol. Ther., 11 (1): 35-47; Clarke et al. (1997), Hum. Mol. Genet. 6 (4): 503-511; all herein incorporated by reference).

One possible delivery vector utilizes the adeno associated virus (AAV). A vector is produced to include a base editor-dRGN fusion protein coding sequence (for example, SEQ ID NO: 452) preceded by a CMV enhancer (SEQ ID NO: 138) and promoter (SEQ ID NO: 137), or other suitable enhancer and promoter combination), optionally a Kozak sequence, and operably fused at the 3′ end to a terminator sequence and a poly adenlylation sequence such as the minimal sequence described in Levitt, N.; Briggs, D.; Gil, A.; Proudfoot, N. J. Definition of an Efficient Synthetic Poly (A) Site. Genes Dev. 1989, 3 (7), 1019-1025. The vector may further comprise an expression cassette encoding for a single guide RNA operably linked at its 5′ end to a human U6 promoter (SEQ ID NO: 139), or another promoter suitable for production of small non-coding RNAs, and further comprising inverted terminal repeat (ITR) sequences necessary and well-known in the art for packaging into the AAV capsid. Production and viral packaging is performed by standard methods, such as those described in U.S. Pat. No. 9,587,250, herein incorporated by reference.

Other possible viral vectors include adenovirus and lentivirus vectors, which are commonly used and would contain similar elements, with different packaging capabilities and requirements. Non-viral delivery methods also be used, such as mRNA and sgRNA encapsulated by lipid nanoparticles (Cullis, P. R. and Allen, T. M. (2013), Adv. Drug Deliv. Rev. 65 (1): 36-48; Finn et al. (2018), Cell Rep. 22 (9): 2227-2235, both incorporated by reference) hydrodynamic injection of plasmid DNA (Suda T and Liu D,) 2007) Mol. Ther. 15 (12): 2063-2069, herein incorporated by reference), or ribonucleoprotein complexes of sgRNA and associated with gold nanoparticles (Lee, K.; Conboy, M.; Park, H. M.; Jiang, F.; Kim, H. J.; Dewitt, M. A.; Mackley, V. A.; Chang, K.; Rao, A.; Skinner, C.; et al. Nanoparticle Delivery of Cas9 Ribonucleoprotein and Donor DNA in Vivo Induces Homology-Directed DNA Repair. Nat. Biomed. Eng. 2017, 1 (11), 889-90).

### Example 10.5: Disease Correction in a Murine Model with a Humanized Locus

To evaluate the efficacy of an identical base editor construct as would be used for human therapy, a mouse model in which the nucleotides near W392 are altered to match the sequence in humans around W402 is needed. This can be accomplished by a variety of techniques, including use of an RGN and an HDR template to cut and replace the locus in mouse embryos.

Due to the high degree of amino acid conservation, most nucleotides in the mouse locus can be altered to those of the human sequence with silent mutations as shown in Table 19. The only base changes resulting in altered coding sequence in the resulting engineered mouse genome occur after the introduced stop codon.

TABLE 19
Nucleotide mutations to generate a humanized mouse locus

| | Human (W402X) | | Mouse (W392X) | | Humanized Mouse | |
|---|---|---|---|---|---|---|
| | Nucleotide | | Nucleotide | | Nucleotide | |
| | (SEQ ID | Encoded | (SEQ ID | Encoded | (SEQ ID | Encoded |
| Feature | NO: 465) | AA | NO: 466) | AA | NO: 467) | AA |
| Protospacer | G | E | A | G | G | G |
| | G | E | G | E | G | E |
| | A | | A | | A | |
| | G | | A | | G | |
| | C | Q | C | Q | C | Q |
| | A | | A | | A | |
| | G | | A | | G | |
| | C | L | C | L | C | L |
| | T | | T | | T | |
| | C | | C | | C | |
| | T | STOP | T | STOP | T | STOP |
| | A | | A | | A | |
| | G | | G | | G | |
| | G | A | G | A | G | A |
| | C | | C | | C | |
| | C | | A | | C | |
| | G | E | G | E | G | E |
| | A | | A | | A | |
| | A | | G | | A | |
| | G | V | G | V | G | V |
| | T | | T | | T | |
| | G | | C | | G | |
| | T | S | T | S | T | S |
| | C | | C | | C | |
| | G | | A | | G | |
| PAM, non- | C | Q | A | K | C | Q |
| critical | A | | A | | A | |
| | G | | G | | G | |
| | G | A | G | A | G | A |
| PAM, | C | | C | | C | |
| critical | C | | T | | C | |

Upon engineering of this mouse strain, similar experiments will be performed as described in Example 10.4.

### Example 11: Targeting Mutations Responsible for Friedreich Ataxia

The expansion of the trinucleotide repeat sequence causing Friedreich's Ataxia (FRDA) occurs in a defined genetic locus within the FXN gene, referred to as the FRDA instability region. RNA guided nucleases (RGNs) may be used for excising the instability region in FRDA patient cells. This approach requires 1) an RGN and guide RNA sequence that can be programmed to target the allele in the human genome; and 2) a delivery approach for the RGN and guide sequence. Many nucleases used for genome editing, such as the commonly used Cas9 nuclease from S. pyogenes (SpCas9), are too large to be packaged into adeno-associated viral (AAV) vectors, especially when considering the length of the SpCas9 gene and the guide RNA in addition to other genetic elements required for functional expression cassettes. This makes a viable approach using SpCas9 unlikely.

The compact RNA guided nucleases of the invention, particularly APG07433.1 and APG08290.1, are uniquely well suited for the excision of the FRDA instability region. Each RGN has a PAM requirement that is in the vicinity of the FRDA instability region. Additionally, each of these RGNs can be packaged into an AAV vector along with a guide RNA. Packing two guide RNAs would likely require a second vector, but this approach still compares favorably to what would be required of a larger nuclease such as SpCas9, which would require splitting the protein sequence between two vectors.

Table 20 shows the location of genomic target sequences suitable for targeting APG07433.1 or APG08290.1 to the 5′ and 3′ flanks of the FRDA instability region. Once at the locus, the RGN would excise the FA instability region. Excision of the region can be verified with Illumina sequencing of the locus.

TABLE 20
Genomic target sequences for RGN systems

| | Location | | |
|---|---|---|---|
| | relative | | |
| | to FRDA | | SEQ |
| Guide | instability | | ID |
| No. | region | Genome target sequence | NO. |
| 1 | 5′ | ATCACCTGAGGTCCGGAGTTCAAGA | 468 |
| 2 | 5′ | GTCTTGAACTCCGGACCTCAGGTGA | 469 |
| 3 | 5′ | TGAACTCCGGACCTCAGGTGATCCA | 470 |
| 4 | 3′ | GAAAAGTTAGCCGGGCGTGGTGTCG | 471 |

### Example 12: Targeting Mutations Responsible for Sickle Cell Diseases

Targeting sequences within the BCL11A enhancer region (SEQ ID NO: 472) may provide a mechanism for increasing fetal hemoglobulin (HbF) to either cure or alleviate the symptoms of sickle cell diseases. For example, genome wide association studies have identified a set of genetic variations at BCL11A that are associated with increased HbF levels. These variations are a collection of SNPs found in non-coding regions of BCL11A that function as a stage-specific, lineage-restricted enhancer region. Further investigation revealed that this BCL11A enhancer is required in erythroid cells for BCL11A expression (Bauer et al, (2013) Science 343:253-257, incorporated by reference herein). The enhancer region was found within intron 2 of the BCL11A gene, and three areas of DNAsel hypersensitivity (often indicative of a chromatin state that is associated with regulatory potential) in intron 2 were identified. These three areas were identified as “+62”, “+58” and “+55” in accordance with the distance in kilobases from the transcription start site of BCL11A. These enhancer regions are roughly 350 (+55); 550 (+58); and 350 (+62) nucleotides in length (Bauer et al., 2013).

### Example 12.1: Identifying Preferred RGN Systems

Here we describe a potential treatment for beta-hemoglobinopathies using an RGN system that disrupts BCL11A binding to its binding site within the HBB locus, which is the gene responsible for making beta-globin in adult hemoglobin. This approach uses NHEJ which is more efficient in mammalian cells. In addition, this approach uses a nuclease of sufficiently small size that can be packaged into a single AAV vector for in vivo delivery.

The GATA1 enhancer motif in the human BCL11A enhancer region (SEQ ID NO: 472) is an ideal target for disruption using RNA guided nucleases (RGNs) to reduce BCL11A expression with concurrent re-expression of HbF in adult human erythrocytes (Wu et al. (2019) Nat Med 387:2554). Several PAM sequences compatible with APG07433.1 and APG08290.1 are readily apparent at the genetic locus surrounding this GATA1 site. These nucleases have a PAM sequence of 5′-NNNNCC-3′(SEQ ID NO: 6) and are compact in size, potentially allowing their delivery along with an appropriate guide RNA in a single AAV or adenoviral vector. This delivery approach bestows multiple advantages relative to others, such as access to hematopoietic stem cells and a well-established safety profile and manufacturing techniques.

The commonly used Cas9 nuclease from S. pyogenes (SpyCas9) requires a PAM sequence of 5′-NGG-3′, (SEQ ID NO: 448) several of which are present near the GATA1 motif. However, the size of SpyCas9 prevents packaging into a single AAV or adenoviral vector and thus forgoes the aforementioned advantages of this approach. While a dual delivery strategy may be employed, it would add significant manufacturing complexity and cost. Additionally, dual viral vector delivery significantly decreases the efficiency of gene correction, since a successful edit in a given cell requires infection with both vectors.

An expression cassette encoding a human codon optimized APG07433.1 (SEQ ID NO: 128) or APG08290.1 (SEQ ID NO: 130) is produced, similar to those described in Example 6. Expression cassettes which express guide RNAs for RGNs APG07433.1 and APG08290.1 are also produced. These guide RNAs comprise 1) a protospacer sequence that is complementary to either the non-coding or coding DNA strand within the BCL11A enhancer locus (the target sequence) and 2) an RNA sequence required for association of the guide RNA with the RGN (SEQ ID NO. 18 for APG07433.1 and SEQ ID NO: 35 for APG08290.1). Because several potential PAM sequences for targeting by APG07433.1 or APG08290.1 surround the BCL11A GATA1 enhancer motif, several potential guide RNA constructs are produced to determine the best protospacer sequence that produces robust cleavage and NHEJ mediated disruption of the BCL11A GATA1 enhancer sequence. The target genomic sequences in the table below (Table 21) are evaluated to direct the RGN to this locus.

TABLE 21
Target Sequences for BCL11A GATA1 enhancer locus

| | | | Target |
|---|---|---|---|
| | | | SEQ ID |
| Guide | Nuclease | Target genomic sequence | NO. |
| 1 | APG07433.1 | GCACTAGACTAGCTTCAAAGTTGTAG | 473 |
| 2 | APG07433.1 | CCTAATCAGAGGCCAAACCCTTCCTG | 474 |
| 3 | APG07433.1 | CAAGCTAACAGTTGCTTTTATCACAG | 475 |
| 4 | APG08290.1 | GCACTAGACTAGCTTCAAAGTTGTAG | 476 |
| 5 | APG08290.1 | CCTAATCAGAGGCCAAACCCTTCCTG | 477 |
| 6 | APG08290.1 | CAAGCTAACAGTTGCTTTTATCACAG | 478 |

To evaluate the efficiency with which APG07433.1 or APG08290.1 generates insertions or deletions that disrupt the BCL11A enhancer region, human cell lines such as human embryonic kidney cells (HEK cells) are used. A DNA vector comprising an RGN expression cassette (for example, as described in Example 6) is produced. A separate vector comprising an expression cassette comprising a coding sequence for a guide RNA sequence of Table 21 is also produced. Such an expression cassette may further comprise a human RNA polymerase III U6 promoter (SEQ ID NO: 139), as described in Example 6. Alternatively, a single vector comprising expression cassettes of both the RGN and guide RNA may be used. The vector is introduced into HEK cells using standard techniques such as those described in Example 6, and the cells are cultured for 1-3 days. Following this culture period, genomic DNA is isolated and the frequency of insertions or deletions is determined by using T7 Endonuclease I digestion and/or direct DNA sequencing, as described in Example 6.

A region of DNA encompassing the target BCL11A region is amplified by PCR with primers containing Illumina Nextera XT overhang sequences. These PCR amplicons are either examined for NHEJ formation using T7 Endonuclease I digestion, or undergo library preparation following the Illumina 16S Metagenomic Sequencing Library protocol or a similar Next Generation Sequencing (NGS) library preparation. Following deep sequencing, the reads generated are analyzed by CRISPResso to calculate rates of editing. Output alignments are hand-curated to confirm insertion and deletion sites. This analysis identifies the preferred RGN and the corresponding preferred guide RNA (sgRNA). The analysis may result in both APG07433.1 and APG08290.1 being equally preferred. Additionally, the analysis may determine there is more than one preferred guide RNA, or that all target genomic sequences in Table 21 are equally preferred.

### Example 12.2: Assay for Expression of Fetal Hemoglobin

In this example, APG07433.1 or APG08290.1 generated insertions or deletions disrupting the BCL11A enhancer region are assayed for expression of fetal hemoglobin. Healthy human donor CD34+ hematopoietic stem cells (HSCs) are used. These HSCs are cultured and vector(s) comprising expression cassettes comprising the coding regions of the preferred RGN and the preferred sgRNA are introduced using methods similar to those described in Example 11.1. Following electroporation, these cells are differentiated in vitro into erythrocytes using established protocols (for example, Giarratana et al. (2004) Nat Biotechnology 23:69-74, herein incorporated by reference). The expression of HbF is then measured using western blotting with an anti-human HbF antibody, or quantified via High Performance Liquid Chromatography (HPLC). It is expected that successful disruption of the BCL11A enhancer locus will lead to an increase in HbF production when compared to HSCs electroporated with only the RGN but no guide.

### Example 12.3: Assay for Decreased Sickle Cell Formation

In this example, APG07433.1 or APG08290.1 generated insertions or deletions disrupting the BCL11A enhancer region are assayed for decreased sickle-cell formation. Donor CD34+ hematopoietic stem cells (HSCs) from patients afflicted with sickle cell disease are used. These HSCs are cultured and vector(s) comprising expression cassettes comprising the coding regions of preferred RGN and the preferred sgRNA are introduced using methods similar to those described in Example 11.1. Following electroporation, these cells are differentiated in vitro into erythrocytes using established protocols (Giarratana et al. (2004) Nat Biotechnology 23:69-74). The expression of HbF is then measured using western blotting with an anti-human HbF antibody, or quantified via High Performance Liquid Chromatography (HPLC). It is expected that successful disruption of the BCL11A enhancer locus will lead to an increase in HbF production when compared to HSCs electroporated with only the RGN but no guide.

Sickle cell formation is induced in these differentiated erythrocytes by the addition of metabisulfite. The numbers of sickled vs normal erythrocytes are counted using a microscope. It is expected that the numbers of sickled cells are less in cells treated with APG07433.1 or APG08290.1 plus sgRNAs than with cells untreated, or treated with RGNs alone.

### Example 12.4: Disease treatment validation in a murine model

To evaluate the efficacy of using APG07433.1 or APG08290.1 disruption of the BCL11A locus, suitable humanized mouse models of sickle cell anemia are used. Expression cassettes encoding for the preferred RGN and for the preferred sgRNA are packaged into AAV vectors or adenovirus vectors. In particular, adenovirus type Ad5/35 is effective at targeting HSCs. A suitable mouse model containing a humanized HBB locus with sickle cell alleles is chosen such as B6;FVB-Tg (LCR-HBA2,LCR-HBB*E26K) 53Hhb/J or B6.Cg-Hbatm1Paz HbbtmlTow Tg (HBA-HBBs) 41Paz/HhbJ. These mice are treated with granulocyte colony-stimulating factor alone or in combination with plerixafor to mobilize HSCs into circulation. AAVs or adenoviruses carrying the RGN and guide plasmid are then injected intravenously, and the mice are allowed to recover for a week. Blood obtained from these mice is tested in an in vitro sickling assay using metabisulfite, and the mice are followed longitudinally to monitor mortality rates and hematopoietic function. It is expected that treatment with AAVs or adenoviruses carrying an RGN and guide RNA will reduce sickling, mortality, and improve hematopoietic function when compared to mice treated with viruses lacking both expression cassettes, or with viruses carrying the RGN expression cassette alone.

That which is claimed:

1. A nucleic acid molecule comprising a polynucleotide encoding an RNA-guided nuclease (RGN) polypeptide, wherein said RGN polypeptide comprises an amino acid sequence having at least 90% sequence identity to SEQ ID NO: 11, 45, or 54;
wherein said polynucleotide encoding an RGN polypeptide is operably linked to a promoter heterologous to said polynucleotide.

2. The nucleic acid molecule of claim 1, wherein said RGN polypeptide comprises the amino acid sequence set forth as SEQ ID NO: 11, 45, or 54.

3. The nucleic acid molecule of claim 1, wherein said RGN polypeptide is nuclease dead or functions as a nickase.

4. The nucleic acid molecule of claim 1, wherein the RGN polypeptide is operably fused to a base-editing polypeptide.

5. The nucleic acid molecule of claim 4, wherein the base-editing polypeptide is a deaminase.

6. The nucleic acid molecule of claim 1, wherein the RGN polypeptide is operably fused to an effector domain.

7. The nucleic acid molecule of claim 6, wherein the effector domain is a cleavage domain, a deaminase domain, or an expression modulator domain.

8. The nucleic acid molecule of claim 7, wherein the expression modulator domain is an epigenetic modification domain, a transcriptional repressor domain, or a transcriptional activation domain.

9. The nucleic acid molecule of claim 6, wherein the effector domain is operably fused at the N-terminus or at the C-terminus of said RGN polypeptide.

10. The nucleic acid molecule of claim 6, wherein the effector domain is operably fused at an internal location of said RGN polypeptide.

11. The nucleic acid molecule of claim 1, wherein the RGN polypeptide is capable of binding a guide RNA capable of hybridizing to a target DNA sequence.

12. The nucleic acid molecule of claim 11, wherein said target DNA sequence is located adjacent to a protospacer adjacent motif (PAM) site of:
(a) nnnncc (SEQ ID NO: 6), wherein the RGN polypeptide comprises an amino acid sequence having at least 90% sequence identity to SEQ ID NO: 11;
(b) nnraaty (SEQ ID NO: 50), wherein the RGN polypeptide comprises an amino acid sequence having at least 90% sequence identity to SEQ ID NO: 45; or
(c) nnrnnc (SEQ ID NO: 59), wherein the RGN polypeptide comprises an amino acid sequence having at least 90% sequence identity to SEQ ID NO: 54.

13. A vector comprising the nucleic acid molecule of claim 1.

14. The vector of claim 13, wherein said vector further comprises at least one nucleotide sequence encoding a guide RNA, and wherein the guide RNA comprises:
(a) a CRISPR RNA comprising a CRISPR repeat sequence having at least 95% sequence identity to SEQ ID NO: 12, wherein the RGN polypeptide comprises an amino acid sequence having at least 90% sequence identity to SEQ ID NO: 11;
(b) a CRISPR RNA comprising a CRISPR repeat sequence having at least 95% sequence identity to SEQ ID NO: 46, wherein the RGN polypeptide comprises an amino acid sequence having at least 90% sequence identity to SEQ ID NO: 45; or
(c) a CRISPR RNA comprising a CRISPR repeat sequence having at least 95% sequence identity to SEQ ID NO: 55, wherein the RGN polypeptide comprises an amino acid sequence having at least 90% sequence identity to SEQ ID NO: 54.

15. The vector of claim 14, wherein the guide RNA comprises:
(a) a tracrRNA comprising a nucleotide sequence having at least 95% sequence identity to SEQ ID NO: 13, wherein the RGN polypeptide comprises an amino acid sequence having at least 90% sequence identity to SEQ ID NO: 11;
(b) a tracrRNA comprising a nucleotide sequence having at least 95% sequence identity to SEQ ID NO: 47, wherein the RGN polypeptide comprises an amino acid sequence having at least 90% sequence identity to SEQ ID NO: 45; or
(c) a tracrRNA comprising a nucleotide sequence having at least 95% sequence identity to SEQ ID NO: 56, wherein the RGN polypeptide comprises an amino acid sequence having at least 90% sequence identity to SEQ ID NO: 54.

16. A cell comprising the nucleic acid molecule of claim 1.

17. A system, said system comprising:
a) one or more guide RNAs (gRNAs), or one or more nucleotide sequences encoding the one or more gRNAs; and
b) an RNA-guided nuclease (RGN) polypeptide comprising an amino acid sequence having at least 90% sequence identity to SEQ ID NO: 11, 45, or 54, or a nucleotide sequence encoding the RGN polypeptide;
wherein at least one of said nucleotide sequence encoding the one or more gRNAs or encoding the RGN polypeptide is operably linked to a promoter heterologous to said nucleotide sequence.

18. The system of claim 17, wherein said RGN polypeptide comprises the amino acid sequence set forth as SEQ ID NO: 11, 45, or 54.

19. The system of claim 17, wherein the one or more gRNAs hybridize to a target DNA sequence within a eukaryotic cell.

20. The system of claim 17, wherein said RGN polypeptide is nuclease dead or functions as a nickase.

21. The system of claim 17, wherein the RGN polypeptide is operably linked to a base-editing polypeptide.

22. The system of claim 21, wherein the base-editing polypeptide is a deaminase.

23. The system of claim 17, wherein the RGN polypeptide is operably fused to an effector domain.

24. The system of claim 23, wherein the effector domain is a cleavage domain, a deaminase domain, or an expression modulator domain.

25. The system of claim 24, wherein the expression modulator domain is an epigenetic modification domain, a transcriptional repressor domain, or a transcriptional activation domain.

26. The system of claim 23, wherein the effector domain is operably fused at the N-terminus or at the C-terminus of said RGN polypeptide.

27. The system of claim 23, wherein the effector domain is operably fused at an internal location of said RGN polypeptide.

28. The system of claim 17, wherein said system further comprises one or more donor polynucleotides.

29. A method for binding a target DNA sequence comprising delivering a system of claim 17, to said target DNA sequence or a cell comprising the target DNA sequence.

30. The method of claim 29, wherein said system binds to and cleaves and/or modifies said target DNA sequence.

31. A method for cleaving and/or modifying a target DNA sequence, comprising contacting the target DNA sequence with:
a) an RNA-guided nuclease (RGN) polypeptide, wherein said RGN polypeptide comprises an amino acid sequence having at least 90% sequence identity to SEQ ID NO: 11, 45, or 54; and
b) one or more guide RNAs capable of hybridizing to the target DNA sequence, thereby cleaving and/or modifying said target DNA sequence to obtain a modified target DNA sequence.

32. The method of claim 31, wherein said modified target DNA sequence comprises insertion of heterologous DNA into the target DNA sequence, deletion of at least one nucleotide from the target DNA sequence, or mutation of at least one nucleotide in the target DNA sequence.

33. The method of claim 31, wherein the target DNA sequence is within a cell.

34. The method of claim 33, wherein the cell is a eukaryotic cell.

35. The method of claim 33, further comprising culturing the cell; and selecting cells comprising said modified DNA sequence.

36. An RNA polynucleotide comprising at least one synthetic ribonucleotide analogue, wherein said RNA polynucleotide comprises a nucleotide sequence encoding an RNA-guided nuclease (RGN) polypeptide comprising an amino acid sequence having at least 90% sequence identity to SEQ ID NO: 11, 45, or 54.

37. The RNA polynucleotide of claim 36, wherein said RGN polypeptide comprises the amino acid sequence set forth as SEQ ID NO: 11, 45, or 54.

38. The RNA polynucleotide of claim 36, wherein the RNA polynucleotide further comprises a nucleotide sequence encoding a base-editing polypeptide operably linked to the nucleotide sequence encoding the RGN polypeptide.

39. The RNA polynucleotide of claim 36, wherein the RNA polynucleotide is an mRNA.

40. A eukaryotic cell comprising a nucleic acid molecule comprising a polynucleotide encoding an RNA-guided nuclease (RGN) polypeptide, wherein said RGN polypeptide comprises an amino acid sequence having at least 90% to SEQ ID NO: 11, 45, or 54.

41. The eukaryotic cell of claim 40, wherein said RGN polypeptide comprises the amino acid sequence set forth as SEQ ID NO: 11, 45, or 54.

42. The eukaryotic cell of claim 40, wherein said polynucleotide is an mRNA.

43. The eukaryotic cell of claim 40, wherein the RGN polypeptide is operably fused to a base-editing polypeptide.

44. The eukaryotic cell of claim 43, wherein the base-editing polypeptide is a deaminase.

45. The eukaryotic cell of claim 40, wherein the RGN polypeptide is operably fused to an effector domain.

46. The eukaryotic cell of claim 45, wherein the effector domain is a cleavage domain, a deaminase domain, or an expression modulator domain.

47. The eukaryotic cell of claim 46, wherein the expression modulator domain is an epigenetic modification domain, a transcriptional repressor domain, or a transcriptional activation domain.

48. The eukaryotic cell of claim 45, wherein the effector domain is operably fused at the N-terminus or at the C-terminus of said RGN polypeptide.

49. The eukaryotic cell of claim 45, wherein the effector domain is operably fused at an internal location of said RGN polypeptide.

50. A eukaryotic cell comprising an RNA-guided nuclease (RGN) polypeptide, wherein said RGN polypeptide comprises an amino acid sequence having at least 90% to SEQ ID NO: 11, 45, or 54.

51. The eukaryotic cell of claim 50, wherein said RGN polypeptide comprises an amino acid sequence having at least 95% to SEQ ID NO: 11, 45, or 54.

52. The eukaryotic cell of claim 50, wherein said RGN polypeptide comprises the amino acid sequence set forth as SEQ ID NO: 11, 45, or 54.

53. The eukaryotic cell of claim 50, wherein said RGN polypeptide is capable of cleaving a target DNA sequence of a DNA molecule upon binding said target DNA molecule.

54. The eukaryotic cell of claim 53, wherein said target DNA sequence is located adjacent to a protospacer adjacent motif (PAM).

55. The eukaryotic cell of claim 53, wherein cleavage by said RGN polypeptide generates a double-stranded break.

56. The eukaryotic cell of claim 53, wherein cleavage by said RGN polypeptide generates a single-stranded break.

57. The eukaryotic cell of claim 50, wherein said RGN polypeptide is nuclease inactive or a nickase.

58. The eukaryotic cell of claim 50, wherein the RGN polypeptide is operably fused to a base-editing polypeptide.

59. The eukaryotic cell of claim 58, wherein the base-editing polypeptide is a deaminase.

60. The eukaryotic cell of claim 50, wherein the RGN polypeptide is operably fused to an effector domain.

61. The eukaryotic cell of claim 60, wherein the effector domain is a cleavage domain, a deaminase domain, or an expression modulator domain.

62. The eukaryotic cell of claim 61, wherein the expression modulator domain is an epigenetic modification domain, a transcriptional repressor domain, or a transcriptional activation domain.

63. The eukaryotic cell of claim 60, wherein the effector domain is operably fused at the N-terminus or at the C-terminus of said RGN polypeptide.

64. The eukaryotic cell of claim 60, wherein the effector domain is operably fused at an internal location of said RGN polypeptide.

65. The eukaryotic cell of claim 50, wherein the RGN polypeptide comprises one or more nuclear localization signals.

66. A delivery vector comprising: (A) a polynucleotide encoding an RNA-guided nuclease (RGN) polypeptide; or (β) an RGN polypeptide,
wherein said RGN polypeptide comprises an amino acid sequence:
(i) having at least 95% sequence identity to SEQ ID NO: 11, 45, or 54;
(ii) having at least 98% sequence identity to SEQ ID NO: 1 or 19; or
(iii) set forth as SEQ ID NO: 36;

67. The delivery vector of claim 66, wherein the delivery vector comprises said polynucleotide of (A), and wherein the delivery vector further comprises a polynucleotide encoding a guide RNA capable of binding to said RGN polypeptide.

68. The delivery vector of claim 67, wherein at least one of said polynucleotide encoding an RGN polypeptide and said polynucleotide encoding a guide RNA is operably linked to a promoter heterologous to said polynucleotide.

69. The delivery vector of claim 67, wherein the delivery vector further comprises one or more donor polynucleotides.

70. The delivery vector of claim 66, wherein the delivery vector comprises said polynucleotide of (A), and wherein the delivery vector is an adeno-associated virus (AAV) particle.

71. The delivery vector of claim 66, wherein the delivery vector comprises a lipid nanoparticle.

72. The delivery vector of claim 71, wherein the delivery vector comprises said polynucleotide of (A), wherein said polynucleotide of (A) is an mRNA, wherein the delivery vector further comprises a polynucleotide encoding a guide RNA capable of binding to said RGN polypeptide, and wherein said polynucleotide encoding a guide RNA is an mRNA.

73. The delivery vector of claim 71, wherein the delivery vector comprises said RGN polypeptide of (β), wherein the delivery vector further comprises a guide RNA, and wherein said guide RNA is capable of binding to said RGN polypeptide to form a ribonucleoprotein complex.

74. The delivery vector of claim 66, wherein said RGN polypeptide comprises the amino acid sequence set forth as SEQ ID NO: 11, 45, 54, 1, or 19.

75. A nucleic acid molecule comprising a polynucleotide encoding a CRISPR RNA (crRNA), wherein the crRNA comprises a spacer and a CRISPR repeat, wherein the CRISPR repeat comprises a nucleotide sequence having at least 95% sequence identity to SEQ ID NO: 2, 28, 37, 46, or 55; and wherein said polynucleotide encoding a crRNA is operably linked to a promoter heterologous to said polynucleotide.

76. The nucleic acid molecule of claim 75, wherein the crRNA comprises:
(a) a CRISPR repeat comprising a nucleotide sequence having at least 95% sequence identity to SEQ ID NO: 2, wherein the RGN polypeptide comprises an amino acid sequence having at least 98% sequence identity to SEQ ID NO: 1;
(b) a CRISPR repeat comprising a nucleotide sequence having at least 95% sequence identity to SEQ ID NO: 28, wherein the RGN polypeptide comprises an amino acid sequence having at least 95% sequence identity to SEQ ID NO: 27;
(c) a CRISPR repeat comprising a nucleotide sequence having at least 95% sequence identity to SEQ ID NO: 37, wherein the RGN polypeptide comprises an amino acid sequence set forth as SEQ ID NO: 36;
(d) a CRISPR repeat comprising a nucleotide sequence having at least 95% sequence identity to SEQ ID NO: 46, wherein the RGN polypeptide comprises an amino acid sequence having at least 95% sequence identity to SEQ ID NO: 45; or
(e) a CRISPR repeat comprising a nucleotide sequence having at least 95% sequence identity to SEQ ID NO: 55, wherein the RGN polypeptide comprises an amino acid sequence having at least 95% sequence identity to SEQ ID NO: 54.

77. A vector comprising the nucleic acid molecule of claim 75.

78. The vector of claim 77, wherein said vector further comprises a polynucleotide encoding a trans-activating CRISPR RNA (tracrRNA).

79. The vector of claim 78, wherein said tracrRNA comprises:
(a) a nucleotide sequence having at least 95% sequence identity to SEQ ID NO: 3, wherein the crRNA comprises a CRISPR repeat comprising a nucleotide sequence having at least 95% sequence identity to SEQ ID NO: 2;
(b) a nucleotide sequence having at least 95% sequence identity to SEQ ID NO: 29, wherein the crRNA comprises a CRISPR repeat comprising a nucleotide sequence having at least 95% sequence identity to SEQ ID NO: 28;
(c) a nucleotide sequence having at least 95% sequence identity to SEQ ID NO: 38, wherein the crRNA comprises a CRISPR repeat comprising a nucleotide sequence having at least 95% sequence identity to SEQ ID NO: 37;
(d) a nucleotide sequence having at least 95% sequence identity to SEQ ID NO: 47, wherein the crRNA comprises a CRISPR repeat comprising a nucleotide sequence having at least 95% sequence identity to SEQ ID NO: 46; or
(e) a nucleotide sequence having at least 95% sequence identity to SEQ ID NO: 56, wherein the crRNA comprises a CRISPR repeat comprising a nucleotide sequence having at least 95% sequence identity to SEQ ID NO: 55.

80. A nucleic acid molecule comprising a polynucleotide encoding a trans-activating CRISPR RNA (tracrRNA) comprising a nucleotide sequence having at least 95% sequence identity to SEQ ID NOs: 13, 21, 29, 38, 47, or 56; and wherein said polynucleotide encoding a tracrRNA is operably linked to a promoter heterologous to said polynucleotide.

81. The nucleic acid molecule of claim 80, wherein the tracrRNA comprises:
(a) a nucleotide sequence having at least 95% sequence identity to SEQ ID NO: 13, wherein the RGN polypeptide comprises an amino acid sequence having at least 95% sequence identity to SEQ ID NO: 11;
(b) a nucleotide sequence having at least 95% sequence identity to SEQ ID NO: 21, wherein the RGN polypeptide comprises an amino acid sequence having at least 98% sequence identity to SEQ ID NO: 19;
(c) a nucleotide sequence having at least 95% sequence identity to SEQ ID NO: 29, wherein the RGN polypeptide comprises an amino acid sequence having at least 95% sequence identity to SEQ ID NO: 27;
(d) a nucleotide sequence having at least 95% sequence identity to SEQ ID NO: 38, wherein the RGN polypeptide comprises an amino acid sequence set forth as SEQ ID NO: 36;
(e) a nucleotide sequence having at least 95% sequence identity to SEQ ID NO: 47, wherein the RGN polypeptide comprises an amino acid sequence having at least 95% sequence identity to SEQ ID NO: 45; or
(f) a nucleotide sequence having at least 95% sequence identity to SEQ ID NO: 56, wherein the RGN polypeptide comprises an amino acid sequence having at least 95% sequence identity to SEQ ID NO: 54.

82. A vector comprising the nucleic acid molecule of claim 80.

83. The vector of claim 82, wherein the vector further comprises a polynucleotide encoding a CRISPR RNA (crRNA) comprising a spacer and a CRISPR repeat.

84. The vector of claim 83, wherein the CRISPR repeat of the crRNA comprises:
(a) a nucleotide sequence having at least 95% sequence identity to SEQ ID NO: 12, wherein the tracrRNA comprises a nucleotide sequence having at least 95% sequence identity to SEQ ID NO: 13;
(b) a nucleotide sequence having at least 95% sequence identity to SEQ ID NO: 20, wherein the tracrRNA comprises a nucleotide sequence having at least 95% sequence identity to SEQ ID NO: 21;
(c) a nucleotide sequence having at least 95% sequence identity to SEQ ID NO: 28, wherein the tracrRNA comprises a nucleotide sequence having at least 95% sequence identity to SEQ ID NO: 29;
(d) a nucleotide sequence having at least 95% sequence identity to SEQ ID NO: 37, wherein the tracrRNA a nucleotide sequence having at least 95% sequence identity to SEQ ID NO: 38;
(e) a nucleotide sequence having at least 95% sequence identity to SEQ ID NO: 46, wherein the tracrRNA comprises a nucleotide sequence having at least 95% sequence identity to SEQ ID NO: 47; or
(f) a nucleotide sequence having at least 95% sequence identity to SEQ ID NO: 55, wherein the tracrRNA comprises a nucleotide sequence having at least 95% sequence identity to SEQ ID NO: 56.

85. A method for producing a genetically modified mammalian hematopoietic progenitor cell having decreased BCL11A mRNA and protein expression, the method comprising introducing into an isolated human hematopoietic progenitor cell:
a) an RNA-guided nuclease (RGN) polypeptide, wherein the RGN polypeptide comprises an amino acid sequence having at least 95% sequence identity to SEQ ID NO: 1, 11, 19, 27, 36, 45, or 54, or a polynucleotide encoding said RGN polypeptide, wherein said polynucleotide encoding the RGN polypeptide is operably linked to a promoter to enable expression of the RGN polypeptide in the cell; and
b) a guide RNA (gRNA), wherein the gRNA comprises a CRISPR repeat comprising a nucleotide sequence having at least 95% sequence identity to SEQ ID NO: 2, 12, 20, 28, 37, 46, or 55, or a polynucleotide encoding said gRNA, wherein said polynucleotide encoding the gRNA is operably linked to a promoter to enable expression of the gRNA in the cell,
wherein the RGN polypeptide and gRNA are expressed in the cell and cleave at the BCL11A enhancer region, resulting in genetic modification of the human hematopoietic progenitor cell and reducing the mRNA and/or protein expression of BCL11A.

86. The method of claim 85, wherein the gRNA further comprises a spacer sequence that targets SEQ ID NO: 473, 474, 475, 476, 477, or 478.
